SEQUENCE LISTING (139 sequences)

SEQ ID NO: 1 - protein, 29 aa, Homo sapiens
DTQALLRNDQVYQPLRDRDDAQYSHLGGN

SEQ ID NO: 2 - protein, 28 aa, Homo sapiens
ERPPPVPNPDYEPIRKGQRDLYSGLNQR

SEQ ID NO: 3 - protein, 29 aa, Homo sapiens
DKQTLLPNDQLYQPLKDREDDQYSHLQGN

SEQ ID NO: 4 - protein, 29 aa, Homo sapiens
APAYQQGQNQLYNELNLGRREEYDVLDKR

SEQ ID NO: 5 - protein, 29 aa, Homo sapiens
PRRKNPQEGLYNELQKDKMAEAYSEIGMK

SEQ ID NO: 6 - protein, 29 aa, Homo sapiens
ERRRGKGHDGLYQGLSTATKDTYDALHMQ

SEQ ID NO: 7 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 29 aa, Homo sapiens
RKQRITETESPYQELQGQRSDVYSDLNTQ

SEQ ID NO: 9 - protein, 29 aa, Homo sapiens
DAGDEYEDENLYEGLNLDDCSMYEDISRG

SEQ ID NO: 10 - protein, 29 aa, Homo sapiens
DSKAGMEEDHTYEGLDIDQTATYEDIVTL

SEQ ID NO: 11 - protein, 29 aa, Homo sapiens
AITSYEKSDGVYTGLSTRNQETYETLKHE

SEQ ID NO: 12 - protein, 17 aa, Homo sapiens
DRVYEELNIYSATYSEL

SEQ ID NO: 13 - protein, 29 aa, Homo sapiens
SSPKQHPSESVYTALQRRETEVYACIENE

SEQ ID NO: 14 - protein, 18 aa, Homo sapiens
LYTKLMNEDPMYSMYAKL

SEQ ID NO: 15 - protein, 18 aa, Homo sapiens
LYNKLVNEAPVYSVYSKL

SEQ ID NO: 16 - protein, 21 aa, Homo sapiens
VLEYLKIAQDLEMYGVNYFSI

SEQ ID NO: 17 - protein, 10 aa, Artificial Sequence
  Synthetic Construct
GENLYFQSGG

SEQ ID NO: 18 - protein, 4 aa, Artificial Sequence
  Synthetic Construct
GGSG

SEQ ID NO: 19 - protein, 2 aa, Artificial Sequence
  Synthetic Construct
GS

SEQ ID NO: 20 - protein, 6 aa, Artificial Sequence
  Synthetic Construct
GSGSGS

SEQ ID NO: 21 - protein, 12 aa, Artificial Sequence
  Synthetic Construct
PPPYQPLGGGGS

SEQ ID NO: 22 - protein, 10 aa, Artificial Sequence
  Synthetic Construct
GGGGSGGGGS

SEQ ID NO: 23 - protein, 1 aa, Artificial Sequence
  Synthetic Construct
G

SEQ ID NO: 24 - protein, 10 aa, Artificial Sequence
  Synthetic Construct
EPKSCDKTHT

SEQ ID NO: 25 - protein, 5 aa, Artificial Sequence
  Synthetic Construct
CPPCP

SEQ ID NO: 26 - protein, 5 aa, Artificial Sequence
  Synthetic Construct
CPRCP

SEQ ID NO: 27 - protein, 5 aa, Artificial Sequence
  Synthetic Construct
CPSCP

SEQ ID NO: 28 - protein, 5 aa, Artificial Sequence
  Synthetic Construct
TVAAP

SEQ ID NO: 29 - protein, 5 aa, Artificial Sequence
  Synthetic Construct
GGGGS

SEQ ID NO: 30 - protein, 10 aa, Artificial Sequence
  Synthetic Construct
GSGSGSGSGS

SEQ ID NO: 31 - protein, 8 aa, Artificial Sequence
  Synthetic Construct
SACYCELS

SEQ ID NO: 32 - protein, 18 aa, Artificial Sequence
  Synthetic Construct
GSTSGSGKPGSGEGSTKG

SEQ ID NO: 33 - protein, 12 aa, Artificial Sequence
  Synthetic Construct
GGGSGGGSGGGS

SEQ ID NO: 34 - protein, 16 aa, Artificial Sequence
  Synthetic Construct
GGGSGGGSGGGSGGGS

SEQ ID NO: 35 - protein, 24 aa, Artificial Sequence
  Synthetic Construct
GGGGSGGGGSGGGGGGSGSGGGGS

SEQ ID NO: 36 - protein, 34 aa, Artificial Sequence
  Synthetic Construct
GGGGSGGGGSGGGGGGSGSGGGGSGGGGSGGGGS

SEQ ID NO: 37 - protein, 15 aa, Artificial Sequence
  Synthetic Construct
GGGGSGGGGSGGGGS

SEQ ID NO: 38 - protein, 20 aa, Artificial Sequence
  Synthetic Construct
GGGGSGGGGSGGGGSGGGGS

SEQ ID NO: 39 - protein, 16 aa, Artificial Sequence
  Synthetic Construct
GGGGGSGGRASGGGGS

SEQ ID NO: 40 - protein, 54 aa, Artificial Sequence
  Synthetic Construct
RVKFSRSADRGRDPEMGGKGALPPRGGSGRVKFSRSADRGRDPEMGGKGALPPR

SEQ ID NO: 41 - protein, 112 aa, Artificial Sequence
  Synthetic Construct
GGGGSGGGGSAPAYQQGQNQLYNELNLGRREEYDVLDKRGENLYFQSGGPRRKNPQEGLYNELQKDKMAEAYSEIGMKGERRRGKGHDGLYQGLSTATKDTYDALHMQGGSG

SEQ ID NO: 42 - protein, 112 aa, Artificial Sequence
  Synthetic Construct
GGGGSGGGGSAPAYQQGQNQLYNELNLGRREEYDVLDKRGENLYFQSGGAPAYQQGQNQLYNELNLGRREEYDVLDKRGAPAYQQGQNQLYNELNLGRREEYDVLDKRGGSG

SEQ ID NO: 43 - protein, 112 aa, Artificial Sequence
  Synthetic Construct
GGGGSGGGGSPRRKNPQEGLYNELQKDKMAEAYSEIGMKGENLYFQSGGPRRKNPQEGLYNELQKDKMAEAYSEIGMKGPRRKNPQEGLYNELQKDKMAEAYSEIGMKGGSG

SEQ ID NO: 44 - protein, 112 aa, Artificial Sequence
  Synthetic Construct
GGGGSGGGGSERRRGKGHDGLYQGLSTATKDTYDALHMQGENLYFQSGGERRRGKGHDGLYQGLSTATKDTYDALHMQGERRRGKGHDGLYQGLSTATKDTYDALHMQGGSG

SEQ ID NO: 45 - protein, 112 aa, Artificial Sequence
  Synthetic Construct
GGGGSGGGGSDTQALLRNDQVYQPLRDRDDAQYSHLGGNGENLYFQSGGDTQALLRNDQVYQPLRDRDDAQYSHLGGNGDTQALLRNDQVYQPLRDRDDAQYSHLGGNGGSG

SEQ ID NO: 46 - protein, 109 aa, Artificial Sequence
  Synthetic Construct
GGGGSGGGGSERPPPVPNPDYEPIRKGQRDLYSGLNQRGENLYFQSGGERPPPVPNPDYEPIRKGQRDLYSGLNQRGERPPPVPNPDYEPIRKGQRDLYSGLNQRGGSG

SEQ ID NO: 47 - protein, 112 aa, Artificial Sequence
  Synthetic Construct
GGGGSGGGGSDKQTLLPNDQLYQPLKDREDDQYSHLQGNGENLYFQSGGDKQTLLPNDQLYQPLKDREDDQYSHLQGNGDKQTLLPNDQLYQPLKDREDDQYSHLQGNGGSG

SEQ ID NO: 48 - protein, 112 aa, Artificial Sequence
  Synthetic Construct
GGGGSGGGGSRKQRITETESPYQELQGQRSDVYSDLNTQGENLYFQSGGRKQRITETESPYQELQGQRSDVYSDLNTQGRKQRITETESPYQELQGQRSDVYSDLNTQGGSG

SEQ ID NO: 49 - protein, 112 aa, Artificial Sequence
  Synthetic Construct
GGGGSGGGGSDAGDEYEDENLYEGLNLDDCSMYEDISRGGENLYFQSGGDAGDEYEDENLYEGLNLDDCSMYEDISRGGDAGDEYEDENLYEGLNLDDCSMYEDISRGGGSG

SEQ ID NO: 50 - protein, 112 aa, Artificial Sequence
  Synthetic Construct
GGGGSGGGGSDSKAGMEEDHTYEGLDIDQTATYEDIVTLGENLYFQSGGDSKAGMEEDHTYEGLDIDQTATYEDIVTLGDSKAGMEEDHTYEGLDIDQTATYEDIVTLGGSG

SEQ ID NO: 51 - protein, 76 aa, Artificial Sequence
  Synthetic Construct
GGGGSGGGGSDRVYEELNIYSATYSELGENLYFQSGGDRVYEELNIYSATYSELGDRVYEELNIYSATYSELGGSG

SEQ ID NO: 52 - protein, 112 aa, Artificial Sequence
  Synthetic Construct
GGGGSGGGGSAITSYEKSDGVYTGLSTRNQETYETLKHEGENLYFQSGGAITSYEKSDGVYTGLSTRNQETYETLKHEGAITSYEKSDGVYTGLSTRNQETYETLKHEGGSG

SEQ ID NO: 53 - protein, 112 aa, Artificial Sequence
  Synthetic Construct
GGGGSGGGGSSSPKQHPSESVYTALQRRETEVYACIENEGENLYFQSGGSSPKQHPSESVYTALQRRETEVYACIENEGSSPKQHPSESVYTALQRRETEVYACIENEGGSG

SEQ ID NO: 54 - protein, 117 aa, Artificial Sequence
  Synthetic Construct
PPPYQPLGGGGSAPAYQQGQNQLYNELNLGRREEYDVLDKRGENLYFQSGGPRRKNPQEGLYNELQKDKMAEAYSEIGMKGSERRRGKGHDGLYQGLSTATKDTYDALHMQGSGSGS

SEQ ID NO: 55 - protein, 109 aa, Artificial Sequence
  Synthetic Construct
GENLYFQSGGAPAYQQGQNQLYNELNLGRREEYDVLDKRGGSGAPAYQQGQNQLYNELNLGRREEYDVLDKRGGSGAPAYQQGQNQLYNELNLGRREEYDVLDKRGGSG

SEQ ID NO: 56 - protein, 106 aa, Artificial Sequence
  Synthetic Construct
GENLYFQSGGERPPPVPNPDYEPIRKGQRDLYSGLNQRGGSGERPPPVPNPDYEPIRKGQRDLYSGLNQRGGSGERPPPVPNPDYEPIRKGQRDLYSGLNQRGGSG

SEQ ID NO: 57 - protein, 141 aa, Artificial Sequence
  Synthetic Construct
GENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 58 - protein, 115 aa, Artificial Sequence
  Synthetic Construct
DTQALLRNDQVYQPLRDRDDAQYSHLGGNERPPPVPNPDYEPIRKGQRDLYSGLNQRDKQTLLPNDQLYQPLKDREDDQYSHLQGNRKQRITETESPYQELQGQRSDVYSDLNTQ

SEQ ID NO: 59 - protein, 145 aa, Artificial Sequence
  Synthetic Construct
GSGSGSGSGSDTQALLRNDQVYQPLRDRDDAQYSHLGGNGGGGSERPPPVPNPDYEPIRKGQRDLYSGLNQRGGGGSDKQTLLPNDQLYQPLKDREDDQYSHLQGNGGGGSRKQRITETESPYQELQGQRSDVYSDLNTQGGGGS

SEQ ID NO: 60 - protein, 143 aa, Artificial Sequence
  Synthetic Construct
SACYCELSDTQALLRNDQVYQPLRDRDDAQYSHLGGNCPSCPERPPPVPNPDYEPIRKGQRDLYSGLNQRCPSCPDKQTLLPNDQLYQPLKDREDDQYSHLQGNCPSCPRKQRITETESPYQELQGQRSDVYSDLNTQCPSCP

SEQ ID NO: 61 - protein, 145 aa, Artificial Sequence
  Synthetic Construct
EPKSCDKTHTDTQALLRNDQVYQPLRDRDDAQYSHLGGNCPPCPERPPPVPNPDYEPIRKGQRDLYSGLNQRCPPCPDKQTLLPNDQLYQPLKDREDDQYSHLQGNCPPCPRKQRITETESPYQELQGQRSDVYSDLNTQCPPCP

SEQ ID NO: 62 - protein, 145 aa, Artificial Sequence
  Synthetic Construct
GGGGSGGGGSDTQALLRNDQVYQPLRDRDDAQYSHLGGNCPRCPERPPPVPNPDYEPIRKGQRDLYSGLNQRTVAAPDKQTLLPNDQLYQPLKDREDDQYSHLQGNCPSCPRKQRITETESPYQELQGQRSDVYSDLNTQCPPCP

SEQ ID NO: 63 - protein, 145 aa, Artificial Sequence
  Synthetic Construct
EPKSCDKTHTDTQALLRNDQVYQPLRDRDDAQYSHLGGNCPPCPERPPPVPNPDYEPIRKGQRDLYSGLNQRCPSCPDKQTLLPNDQLYQPLKDREDDQYSHLQGNCPRCPRKQRITETESPYQELQGQRSDVYSDLNTQTVAAP

SEQ ID NO: 64 - protein, 141 aa, Artificial Sequence
  Synthetic Construct
GENLYFQSGGERPPPVPNPDYEPIRKGQRDLYSGLNQRGGSGDTQALLRNDQVYQPLRDRDDAQYSHLGGNGGSGRKQRITETESPYQELQGQRSDVYSDLNTQGGSGDKQTLLPNDQLYQPLKDREDDQYSHLQGNGGSG

SEQ ID NO: 65 - protein, 141 aa, Artificial Sequence
  Synthetic Construct
GENLYFQSGGDKQTLLPNDQLYQPLKDREDDQYSHLQGNGGSGRKQRITETESPYQELQGQRSDVYSDLNTQGGSGDTQALLRNDQVYQPLRDRDDAQYSHLGGNGGSGERPPPVPNPDYEPIRKGQRDLYSGLNQRGGSG

SEQ ID NO: 66 - protein, 141 aa, Artificial Sequence
  Synthetic Construct
GENLYFQSGGRKQRITETESPYQELQGQRSDVYSDLNTQGGSGDKQTLLPNDQLYQPLKDREDDQYSHLQGNGGSGERPPPVPNPDYEPIRKGQRDLYSGLNQRGGSGDTQALLRNDQVYQPLRDRDDAQYSHLGGNGGSG

SEQ ID NO: 67 - protein, 42 aa, Artificial Sequence
  Synthetic Construct
GENLYFQSGGERPPPVPNPDYEPIRKGQRDLYSGLNQRGGSG

SEQ ID NO: 68 - protein, 43 aa, Artificial Sequence
  Synthetic Construct
GENLYFQSGGDTQALLRNDQVYQPLRDRDDAQYSHLGGNGGSG

SEQ ID NO: 69 - protein, 75 aa, Artificial Sequence
  Synthetic Construct
GENLYFQSGGDTQALLRNDQVYQPLRDRDDAQYSHLGGNGGSGERPPPVPNPDYEPIRKGQRDLYSGLNQRGGSG

SEQ ID NO: 70 - protein, 76 aa, Artificial Sequence
  Synthetic Construct
GENLYFQSGGDKQTLLPNDQLYQPLKDREDDQYSHLQGNGGSGRKQRITETESPYQELQGQRSDVYSDLNTQGGSG

SEQ ID NO: 71 - protein, 107 aa, Artificial Sequence
  Synthetic Construct
GENLYFQSGGDTQALLRNDQVYQPLRDRDDAQYSHLGGNGGSGERPPPVPNPDYEPIRKGQRDLYSGLNQRGGSGERPPPVPNPDYEPIRKGQRDLYSGLNQRGGSG

SEQ ID NO: 72 - protein, 108 aa, Artificial Sequence
  Synthetic Construct
GENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSG

SEQ ID NO: 73 - protein, 111 aa, Artificial Sequence
  Synthetic Construct
GGGGSGGGGSRKQRITETESPYQELQGQRSDVYSDLNTQGENLYFQSGGERPPPVPNPDYEPIRKGQRDLYSGLNQRGDTQALLRNDQVYQPLRDRDDAQYSHLGGNGGSG

SEQ ID NO: 74 - protein, 111 aa, Artificial Sequence
  Synthetic Construct
GGGGSGGGGSRKQRITETESPYQELQGQRSDVYSDLNTQGENLYFQSGGDTQALLRNDQVYQPLRDRDDAQYSHLGGNGERPPPVPNPDYEPIRKGQRDLYSGLNQRGGSG

SEQ ID NO: 75 - protein, 499 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 504 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELPPPYQPLGGGGSAPAYQQGQNQLYNELNLGRREEYDVLDKRGENLYFQSGGPRRKNPQEGLYNELQKDKMAEAYSEIGMKGSERRRGKGHDGLYQGLSTATKDTYDALHMQGSGSGS

SEQ ID NO: 77 - protein, 496 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGAPAYQQGQNQLYNELNLGRREEYDVLDKRGGSGAPAYQQGQNQLYNELNLGRREEYDVLDKRGGSGAPAYQQGQNQLYNELNLGRREEYDVLDKRGGSG

SEQ ID NO: 78 - protein, 493 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGERPPPVPNPDYEPIRKGQRDLYSGLNQRGGSGERPPPVPNPDYEPIRKGQRDLYSGLNQRGGSGERPPPVPNPDYEPIRKGQRDLYSGLNQRGGSG

SEQ ID NO: 79 - protein, 528 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 80 - protein, 502 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDTQALLRNDQVYQPLRDRDDAQYSHLGGNERPPPVPNPDYEPIRKGQRDLYSGLNQRDKQTLLPNDQLYQPLKDREDDQYSHLQGNRKQRITETESPYQELQGQRSDVYSDLNTQ

SEQ ID NO: 81 - protein, 532 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGSGSGSGSGSDTQALLRNDQVYQPLRDRDDAQYSHLGGNGGGGSERPPPVPNPDYEPIRKGQRDLYSGLNQRGGGGSDKQTLLPNDQLYQPLKDREDDQYSHLQGNGGGGSRKQRITETESPYQELQGQRSDVYSDLNTQGGGGS

SEQ ID NO: 82 - protein, 530 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSACYCELSDTQALLRNDQVYQPLRDRDDAQYSHLGGNCPSCPERPPPVPNPDYEPIRKGQRDLYSGLNQRCPSCPDKQTLLPNDQLYQPLKDREDDQYSHLQGNCPSCPRKQRITETESPYQELQGQRSDVYSDLNTQCPSCP

SEQ ID NO: 83 - protein, 532 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELEPKSCDKTHTDTQALLRNDQVYQPLRDRDDAQYSHLGGNCPPCPERPPPVPNPDYEPIRKGQRDLYSGLNQRCPPCPDKQTLLPNDQLYQPLKDREDDQYSHLQGNCPPCPRKQRITETESPYQELQGQRSDVYSDLNTQCPPCP

SEQ ID NO: 84 - protein, 532 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSGGGGSDTQALLRNDQVYQPLRDRDDAQYSHLGGNCPRCPERPPPVPNPDYEPIRKGQRDLYSGLNQRTVAAPDKQTLLPNDQLYQPLKDREDDQYSHLQGNCPSCPRKQRITETESPYQELQGQRSDVYSDLNTQCPPCP

SEQ ID NO: 85 - protein, 532 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELEPKSCDKTHTDTQALLRNDQVYQPLRDRDDAQYSHLGGNCPPCPERPPPVPNPDYEPIRKGQRDLYSGLNQRCPSCPDKQTLLPNDQLYQPLKDREDDQYSHLQGNCPRCPRKQRITETESPYQELQGQRSDVYSDLNTQTVAAP

SEQ ID NO: 86 - protein, 528 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGERPPPVPNPDYEPIRKGQRDLYSGLNQRGGSGDTQALLRNDQVYQPLRDRDDAQYSHLGGNGGSGRKQRITETESPYQELQGQRSDVYSDLNTQGGSGDKQTLLPNDQLYQPLKDREDDQYSHLQGNGGSG

SEQ ID NO: 87 - protein, 528 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDKQTLLPNDQLYQPLKDREDDQYSHLQGNGGSGRKQRITETESPYQELQGQRSDVYSDLNTQGGSGDTQALLRNDQVYQPLRDRDDAQYSHLGGNGGSGERPPPVPNPDYEPIRKGQRDLYSGLNQRGGSG

SEQ ID NO: 88 - protein, 528 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGRKQRITETESPYQELQGQRSDVYSDLNTQGGSGDKQTLLPNDQLYQPLKDREDDQYSHLQGNGGSGERPPPVPNPDYEPIRKGQRDLYSGLNQRGGSGDTQALLRNDQVYQPLRDRDDAQYSHLGGNGGSG

SEQ ID NO: 89 - protein, 429 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGERPPPVPNPDYEPIRKGQRDLYSGLNQRGGSG

SEQ ID NO: 90 - protein, 430 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSG

SEQ ID NO: 91 - protein, 462 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSG

SEQ ID NO: 92 - protein, 463 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDKQTLLPNDQLYQPLKDREDDQYSHLQGNGGSGRKQRITETESPYQELQGQRSDVYSDLNTQGGSG

SEQ ID NO: 93 - protein, 494 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGERPPPVPNPDYEPIRKGQRDLYSGLNQRGGSG

SEQ ID NO: 94 - protein, 495 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSG

SEQ ID NO: 95 - protein, 498 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSGGGGSRKQRITETESPYQELQGQRSDVYSDLNTQGENLYFQSGGERPPPVPNPDYEPIRKGQRDLYSGLNQRGDTQALLRNDQVYQPLRDRDDAQYSHLGGNGGSG

SEQ ID NO: 96 - protein, 498 aa, Artificial Sequence
  Synthetic Construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSGGGGSRKQRITETESPYQELQGQRSDVYSDLNTQGENLYFQSGGDTQALLRNDQVYQPLRDRDDAQYSHLGGNGERPPPVPNPDYEPIRKGQRDLYSGLNQRGGSG

SEQ ID NO: 97 - protein, 489 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 518 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 99 - protein, 452 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSG

SEQ ID NO: 100 - protein, 453 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDKQTLLPNDQLYQPLKDREDDQYSHLQGNGGSGRKQRITETESPYQELQGQRSDVYSDLNTQGGSG

SEQ ID NO: 101 - protein, 488 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSGGGGSRKQRITETESPYQELQGQRSDVYSDLNTQGENLYFQSGGERPPPVPNPDYEPIRKGQRDLYSGLNQRGDTQALLRNDQVYQPLRDRDDAQYSHLGGNGGSG

SEQ ID NO: 102 - protein, 488 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSGGGGSRKQRITETESPYQELQGQRSDVYSDLNTQGENLYFQSGGDTQALLRNDQVYQPLRDRDDAQYSHLGGNGERPPPVPNPDYEPIRKGQRDLYSGLNQRGGSG

SEQ ID NO: 103 - protein, 518 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGERPPPVPNPDYEPIRKGQRDLYSGLNQRGGSGDTQALLRNDQVYQPLRDRDDAQYSHLGGNGGSGRKQRITETESPYQELQGQRSDVYSDLNTQGGSGDKQTLLPNDQLYQPLKDREDDQYSHLQGNGGSG

SEQ ID NO: 104 - protein, 518 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGRKQRITETESPYQELQGQRSDVYSDLNTQGGSGDKQTLLPNDQLYQPLKDREDDQYSHLQGNGGSGERPPPVPNPDYEPIRKGQRDLYSGLNQRGGSGDTQALLRNDQVYQPLRDRDDAQYSHLGGNGGSG

SEQ ID NO: 105 - protein, 487 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 516 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 107 - protein, 450 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSG

SEQ ID NO: 108 - protein, 451 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDKQTLLPNDQLYQPLKDREDDQYSHLQGNGGSGRKQRITETESPYQELQGQRSDVYSDLNTQGGSG

SEQ ID NO: 109 - protein, 486 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSGGGGSRKQRITETESPYQELQGQRSDVYSDLNTQGENLYFQSGGERPPPVPNPDYEPIRKGQRDLYSGLNQRGDTQALLRNDQVYQPLRDRDDAQYSHLGGNGGSG

SEQ ID NO: 110 - protein, 486 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSGGGGSRKQRITETESPYQELQGQRSDVYSDLNTQGENLYFQSGGDTQALLRNDQVYQPLRDRDDAQYSHLGGNGERPPPVPNPDYEPIRKGQRDLYSGLNQRGGSG

SEQ ID NO: 111 - protein, 516 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGERPPPVPNPDYEPIRKGQRDLYSGLNQRGGSGDTQALLRNDQVYQPLRDRDDAQYSHLGGNGGSGRKQRITETESPYQELQGQRSDVYSDLNTQGGSGDKQTLLPNDQLYQPLKDREDDQYSHLQGNGGSG

SEQ ID NO: 112 - protein, 516 aa, Artificial Sequence
  Synthetic Construct
MALPVTALLLPLALLLHAARPAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSGGGGSQVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGRKQRITETESPYQELQGQRSDVYSDLNTQGGSGDKQTLLPNDQLYQPLKDREDDQYSHLQGNGGSGERPPPVPNPDYEPIRKGQRDLYSGLNQRGGSGDTQALLRNDQVYQPLRDRDDAQYSHLGGNGGSG

SEQ ID NO: 113 - protein, 517 aa, Artificial Sequence
  Synthetic Construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 114 - protein, 16 aa, Artificial Sequence
  Synthetic Construct
  VARIANT 2, 3, 14, 15: Xaa = Any Amino Acid
  VARIANT 4, 16: Xaa = Leu or Ile
  VARIANT 5, 6, 7, 8, 9, 10, 11, 12: Xaa = Any Amino Acid, and up to two can be present or absent
YXXXXXXXXXXXYXXX

SEQ ID NO: 115 - protein, 19 aa, Artificial Sequence
  Synthetic Construct
  VARIANT 1: Xaa = Asp or Glu
  VARIANT 2, 3: Xaa = Any Amino Acid, and up to two can be present or absent
  VARIANT 5, 6, 17, 18: Xaa = Any Amino Acid
  VARIANT 7, 19: Xaa = Leu or Ile
  VARIANT 8, 9, 10, 11, 12, 13, 14, 15: Xaa = Any Amino Acid, and up to two can be present or absent
XXXYXXXXXXXXXXXYXXX

SEQ ID NO: 116 - protein, 1 aa, Artificial Sequence
  Synthetic Construct
  VARIANT 1: Can be present in repeats of any integer
G

SEQ ID NO: 117 - protein, 2 aa, Artificial Sequence
  Synthetic Construct
  VARIANT (1)..(2): Can be present in repeats of any integer
GS

SEQ ID NO: 118 - protein, 4 aa, Artificial Sequence
  Synthetic Construct
  VARIANT (1)..(4): Can be present in repeats of any integer
GGGS

SEQ ID NO: 119 - protein, 5 aa, Artificial Sequence
  Synthetic Construct
  VARIANT (1)..(5): Can be present in repeats of any integer
GGGGS

SEQ ID NO: 120 - protein, 13 aa, Artificial Sequence
  Synthetic Construct
  VARIANT (1)..(12): Up to nine can be present or absent
  VARIANT (1)..(13): Can be present in repeats of any integer of at least 3 and up to 12 (with up to nine Gly in positions 1 through 12 present or absent)
GGGGGGGGGGGGS

SEQ ID NO: 121 - protein, 223 aa, Artificial Sequence
  Synthetic Construct
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEARNCERHCAAHPAQMGEDPDGIDHGEVLVWKFDPKLAVEYRPDMFKDMHEHAKR

SEQ ID NO: 122 - protein, 181 aa, Artificial Sequence
  Synthetic Construct
MGSSNSKRQQQGLLKLWRGLRGKPGADWVLLSDPLIGQSSTVQEECGKALRQMTYKLAVDFSHFLKSKGGLDGIYYSERREKILNLYALNEWGIIDDWQAYSPGPGIRYPRVFGFCFKLVPVDLHEEARNCERHCLMHPAQMGEDPDGIDHGEVLVWKFDPKLAVEYRPDMFKDMHEHAKR

SEQ ID NO: 123 - DNA, 585 bp, Artificial Sequence
  Synthetic Construct
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 124 - protein, 42 aa, Homo sapiens
KRGRKKLLYIFKQPFMRPVQTTQEEDGCSCRFPEEEEGGCEL

SEQ ID NO: 125 - protein, 45 aa, Homo sapiens
TTTPAPRPPTPAPTIASQPLSLRPEACRPAAGGAVHTRGLDFACD

SEQ ID NO: 126 - protein, 24 aa, Homo sapiens
IYIWAPLAGTCGVLLLSLVITLYC

SEQ ID NO: 127 - protein, 21 aa, Homo sapiens
MALPVTALLLPLALLLHAARP

SEQ ID NO: 128 - protein, 119 aa, Artificial Sequence
  Synthetic Construct
AVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSS

SEQ ID NO: 129 - protein, 117 aa, Artificial Sequence
  Synthetic Construct
QVQLEESGGGSVQAGGSLRLSCAYTYSTYSNYYMGWFREAPGKARTSVAIISSDTTITYKDAVKGRFTISKDNAKNTLYLQMNSLKPEDSAMYRCAAWTSDWSVAYWGQGTQVTVSS

SEQ ID NO: 130 - protein, 6 aa, Artificial Sequence
  Synthetic Construct
STYFMA

SEQ ID NO: 131 - protein, 17 aa, Artificial Sequence
  Synthetic Construct
GIAWSGGSTAYADSVKG

SEQ ID NO: 132 - protein, 11 aa, Artificial Sequence
  Synthetic Construct
SRGIEVEEFGA

SEQ ID NO: 133 - protein, 6 aa, Artificial Sequence
  Synthetic Construct
SNYYMG

SEQ ID NO: 134 - protein, 16 aa, Artificial Sequence
  Synthetic Construct
IISSDTTITYKDAVKG

SEQ ID NO: 135 - protein, 10 aa, Artificial Sequence
  Synthetic Construct
AWTSDWSVAY

SEQ ID NO: 136 - protein, 240 aa, Simian immunodeficiency virus
MGSVKSKLLGETASQRLQGSHGGGRMVYWRLRDGQVKRSQLSPGARGKDLNIALLDEEWQATGADYMGKALTQGEVWYEKEQEENDMELVGFPVRPKVPLRTCTYKLGIDFSFFLKEKGGLKGIFYNKRRHAILNLYAHNEWGILPDWQNYTEGPGTRYPLCFGILWKLCPVEIHEDDTEDGHLLPHPAYDGQAEDPWGESLVWVFDEKLAYTPGAKMAEWDRLEREKRMLLAPPQTASS

SEQ ID NO: 137 - protein, 134 aa, Artificial Sequence
  Synthetic Construct
KRGRKKLLYIFKQPFMRPVQTTQEEDGCSCRFPEEEEGGCELGGSGKRGRKKLLYIFKQPFMRPVQTTQEEDGCSCRFPEEEEGGCELGGSGKRGRKKLLYIFKQPFMRPVQTTQEEDGCSCRFPEEEEGGCEL

SEQ ID NO: 138 - DNA, 20 bp, Artificial Sequence
  Synthetic Construct
actcacgctggatagcctcc

SEQ ID NO: 139 - protein, 223 aa, Simian immunodeficiency virus
MGSSNSKRQQQGLLKLWRGLRGKPGADWVLLSDPLIGQSSTVQEECGKALKKSWGKGKMTPDGRRLQEGDTFDEWDDDEEEVGFPVQPRVPLRQMTYKLAVDFSHFLKSKGGLDGIYYSERREKILNLYALNEWGIIDDWQAYSPGPGIRYPRVFGFCFKLVPVDLHEEARNCERHCLMHPAQMGEDPDGIDHGEVLVWKFDPKLAVEYRPDMFKDMHEHAKR

(19) United States
(12) Patent Application Publication
(10) Pub. No.: US 2022/0289814 A1
(43) Pub. Date: Sep. 15, 2022

(54) ENGINEERED T CELLS AND METHODS OF PRODUCING THEREOF

(71) Applicant: Nanjing Legend Bioteh Co., Ltd., Nanjing, Jiangsu (CN)

(72) Inventors: Xiaohu FAN, Edmonton (CA); Yuncheng ZHAO, Nanjing, Jiangsu (CN); Bing WANG, Nanjing, Jiangsu (CN); Dawei YU, Hefei, Anhui (CN); Xin HUANG, Nanjing, Jiangsu (CN); Pingyan WANG, Fengyang, Anhui (CN); Qiuchuan ZHUANG, Nanjing, Jiangsu (CN)

(73) Assignee: Nanjing Legend Bioteh Co., Ltd., Nanjing, Jiangsu (CN)

(21) Appl. No.: 17/639,249

(22) PCT Filed: Aug. 28, 2020
(86) PCT No.: PCT/CN2020/112182

§ 371 Date: Feb. 28, 2022

(30) Foreign Application Priority Data
Aug. 28, 2019 (CN) PCT/CN2019/103041
Dec. 16, 2019 (CN) PCT/CN2019/125681

(51) Int. Cl. C07K 14/725; C07K 14/705; A61K 35/17; C07K 16/28; A61P 35/00

(52) U.S. Cl. CPC C07K 14/7051; C07K 14/70517; C07K 14/70521; C07K 14/70578; A61K 35/17; C07K 16/2878; C07K 16/2887; A61P 35/00; C07K 2319/02; C07K 2319/03; C07K 2317/622; C07K 2317/569

(57) ABSTRACT

A modified T cell comprising a functional exogenous receptor is provided. The functional exogenous receptor comprises: (a) an extracellular ligand binding domain, (b) a transmembrane domain, and (c) an intracellular signaling domain (ISD) comprising a chimeric signaling domain (CMSD), wherein the CMSD comprises a plurality of Immune-receptor Tyrosine-based Activation Motifs (ITAMs) optionally connected by one or more linkers. Further provided are vectors, methods of producing, pharmaceutical compositions, kits, and methods of treatment thereof.

### CROSS REFERENCE TO RELATED APPLICATIONS

This application claims priority benefit from International Patent Application Nos. PCT/CN2019/103041 filed on Aug. 28, 2019, and PCT/CN2019/125681 filed on Dec. 16, 2019, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422002042ITAMSEQLIST.TXT, date recorded: Aug. 28, 2020, size: 221 KB).

### FIELD OF THE PRESENT APPLICATION

The present application relates to a functional exogenous receptor comprising a chimeric signaling domain (CMSD) and T cells containing such functional exogenous receptor.

### BACKGROUND OF THE PRESENT APPLICATION

CAR-T cell therapy utilizes genetically modified T cells carrying an engineered receptor specifically recognizing a target antigen (e.g., tumor antigen) to direct T cells to tumor site. It has shown promising results in treating hematological cancer and multiple myeloma (MM). CAR usually comprises an extracellular ligand binding domain, a transmembrane (TM) domain, and an intracellular signaling domain (ISD). The extracellular ligand binding domain may comprise an antigen-binding fragment (e.g., single-chain variable fragment, scFv) targeting a desired target antigen (e.g., tumor antigen). Upon binding to the target antigen, CAR can activate T cells to launch specific anti-target (e.g., tumor) response mediated by the ISD (e.g., activation signal via CD3ζISD, mimicking TCR signal transmission) in an antigen-dependent manner without being limited by the availability of major histocompatibility complexes (MHC) specific to the target antigen.

Immune-receptor Tyrosine-based Activation Motifs (ITAMs) reside in the cytoplasmic domain of many cell surface receptors or subunits they associate with, and play an important regulatory role in signal transmission. For example, upon TCR ligation, phosphorylation of ITAMs of the TCR complex creates docking sites to recruit molecules essential for initiating signaling cascade, leading to T-cell activation and differentiation. ITAM functions are not restricted to T cells, as components of the B-cell receptor (BCR, CD79a/Igα and CD79b/Igβ), selected natural killer (NK) cell receptor (DAP-12), and particular FcεR, all require ITAMs to propagate intracellular signals. To date, most clinical studies have used CD3ζ as primary ISD of CAR, but its limitations as signaling domain have been reported. Expression analysis identified significant upregulation of gene sets associated with inflammation, cytokine, and chemokine activity for the second generation anti-CD19 CAR comprising an intact CD3ζISD, and enhanced effector differentiation was also observed (Feucht, J et. al., 2019). CD3ζISD was also found to promote mature T cell apoptosis (Combadiere, B et al., 1996). Further, CAR-T immunotherapy associated cytokine release syndrome (CRS) may limit its clinical implementation in some cases.

Due to individual differences, autologous CAR-T or TCR-T therapy (using patient's own T cells) presents significant challenges in manufacturing and standardization, with extremely expensive cost for manufacturing and treatment. Furthermore, cancer patients usually have lower immune function, with lymphocytes having reduced number, lower immune activity, and hard to expand in vitro. Universal allogeneic CAR-T or TCR-T therapy is considered as an ideal model, with T cells derived from healthy donors. However, the key challenge is how to effectively eliminate graft-versus-host disease (GvHD) during treatment due to histoincompatibility. TCR is a cell surface receptor involved in T cell activation in response to antigen presentation. 95% of T cells in human have TCR consisting of an alpha (α) chain and a beta (β) chain. TCRα and TCRβ chains combine to form a heterodimer and associate with CD3 subunits to form a TCR complex present on the cell surface. GvHD happens when donor's T cells recognize non-self MHC molecules via TCR and perceive host (transplant recipient) tissues as antigenically foreign and attack them. In order to eliminate endogenous TCR from donor T cells thereby preventing GvHD, people have been using gene editing technologies such as Zinc Finger Nuclease (ZFN), transcription activator-like effector nucleases (TALEN), and Clustered Regularly Interspaced Short Palindromic Repeats (CRISPR)-CRISPR associated (Cas) (CRISPR/Cas) for endogenous TCRα or TCRβ gene knockout (KO), then enriching TCR-negative T cells for allogeneic CAR-T or TCR-T production. However, TCR deletion may lead to impaired CD3 downstream signal transduction pathway, and affect T cell expansion.

The disclosures of all publications, patents, patent applications and published patent applications referred to herein are hereby incorporated herein by reference in their entirety.

### BRIEF SUMMARY OF THE PRESENT APPLICATION

The present invention in one aspect provides modified T cells (e.g., allogeneic T cells), comprising: a functional exogenous receptor comprising: (a) an extracellular ligand binding domain, (b) a transmembrane domain (e.g., derived from CD8α), and (c) an intracellular signaling domain (ISD) comprising a chimeric signaling domain (CMSD), wherein the CMSD comprises one or a plurality of ITAMs (“CMSD ITAMs”), wherein the plurality of CMSD ITAMs are optionally connected by one or more linkers (“CMSD linkers”). In some embodiments, the CMSD comprises one or more of the characteristics selected from the group consisting of: (a) the plurality (e.g., 2, 3, 4, or more) of CMSD ITAMs are directly linked to each other; (b) the CMSD comprises two or more (e.g., 2, 3, 4, or more) CMSD ITAMs connected by one or more linkers not derived from an ITAM-containing parent molecule (e.g., G/S linker); (c) the CMSD comprises one or more CMSD linkers derived from an ITAM-containing parent molecule that is different from the ITAM-containing parent molecule from which one or more of the CMSD ITAMs are derived from; (d) the CMSD comprises two or more (e.g., 2, 3, 4, or more) identical CMSD ITAMs; (e) at least one of the CMSD ITAMs is not derived from CD3ζ; (f) at least one of the CMSD ITAMs is not ITAM1 or ITAM2 of CD3ζ; (g) the plurality of CMSD ITAMs are each derived from a different ITAM-containing parent molecule; and/or (h) at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, the CMSD consists essentially of (e.g., consists of) one CMSD ITAM. In some embodiments, the CMSD consists essentially of (e.g., consists of) one CMSD ITAM and a CMSD N-terminal sequence and/or a CMSD C-terminal sequence that is heterologous to the ITAM-containing parent molecule (e.g., a G/S linker). In some embodiments, the plurality (e.g., 2, 3, 4, or more) of CMSD ITAMs are directly linked to each other. In some embodiments, the CMSD comprises two or more (e.g., 2, 3, 4, or more) CMSD ITAMs connected by one or more linkers not derived from an ITAM-containing parent molecule (e.g., G/S linker). In some embodiments, the CMSD comprises one or more CMSD linkers derived from an ITAM-containing parent molecule that is different from the ITAM-containing parent molecule from which one or more of the CMSD ITAMs are derived from. In some embodiments, the CMSD comprises two or more (e.g., 2, 3, 4, or more) identical CMSD ITAMs. In some embodiments, at least one of the CMSD ITAMs is not derived from CD3ζ. In some embodiments, at least one of the CMSD ITAMs is not ITAM1 or ITAM2 of CD3ζ. In some embodiments, the plurality of CMSD ITAMs are each derived from a different ITAM-containing parent molecule. In some embodiments, at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin.

In some embodiments according to any one of the modified T cells described above, at least one of the plurality of CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin. In some embodiments, the CMSD does not comprise ITAM1 and/or ITAM2 of CD3ζ. In some embodiments, the CMSD comprises ITAM3 of CD3ζ. In some embodiments, at least two of the CMSD ITAMs are derived from the same ITAM-containing parent molecule. In some embodiments, at least two of the CMSD ITAMs are different from each other. In some embodiments, at least one of the CMSD linkers is derived from CD3ζ. In some embodiments, at least one of the CMSD linkers is heterologous to the ITAM-containing parent molecule. In some embodiments, the heterologous CMSD linker is selected from the group consisting of SEQ ID NOs: 17-39 and 116-120, such as any of SEQ ID NOs: 17-31. In some embodiments, the heterologous CMSD linker is a G/S linker. In some embodiments, the CMSD comprises two or more heterologous CMSD linkers. In some embodiments, the two or more heterologous CMSD linker sequences are identical to each other. In some embodiments, the two or more heterologous CMSD linker sequences are different from each other. In some embodiments, the CMSD linker sequence is about 1 to about 15 amino acids long.

In some embodiments according to any of the modified T cells described above, the CMSD further comprises a CMSD C-terminal sequence at the C-terminus of the most C-terminal ITAM. In some embodiments, the CMSD C-terminal sequence is derived from CD3ζ. In some embodiments, the CMSD C-terminal sequence is heterologous to the ITAM-containing parent molecule. In some embodiments, the CMSD C-terminal sequence is selected from the group consisting of SEQ ID NOs: 17-39 and 116-120, such as any of SEQ ID NOs: 17-31. In some embodiments, the CMSD C-terminal sequence is about 1 to about 15 amino acids long.

In some embodiments according to any of the modified T cells described above, the CMSD further comprises a CMSD N-terminal sequence at the N-terminus of the most N-terminal ITAM. In some embodiments, the CMSD N-terminal sequence is derived from CD3ζ. In some embodiments, the CMSD N-terminal sequence is heterologous to the ITAM-containing parent molecule. In some embodiments, the CMSD N-terminal sequence is selected from the group consisting of SEQ ID NOs: 17-39 and 116-120, such as any of SEQ ID NOs: 17-31. In some embodiments, the CMSD N-terminal sequence is about 1 to about 15 amino acids long.

In some embodiments according to any of the modified T cells described above, the CMSD comprises from N-terminus to C-terminus: optional CMSD N-terminal sequence—CD3ζ ITAM1—optional first CMSD linker—CD3ζ ITAM2—optional second CMSD linker—CD3ζ ITAM3—optional CMSD C-terminal sequence. In some embodiments, the CMSD comprises the sequence of SEQ ID NO: 41 or 54.

In some embodiments according to any of the modified T cells described above, the CMSD comprises from N-terminus to C-terminus: optional CMSD N-terminal sequence—CD3ζ ITAM1—optional first CMSD linker—CD3ζ ITAM1—optional second CMSD linker—CD3ζ ITAM1—optional CMSD C-terminal sequence. In some embodiments, the CMSD comprises the sequence of SEQ ID NO: 42 or 55.

In some embodiments according to any of the modified T cells described above, the CMSD comprises from N-terminus to C-terminus: optional CMSD N-terminal sequence—CD3ζ ITAM2—optional first CMSD linker—CD3ζ ITAM2—optional second CMSD linker—CD3ζ ITAM2—optional CMSD C-terminal sequence. In some embodiments, the CMSD comprises the sequence of SEQ ID NO: 43.

In some embodiments according to any of the modified T cells described above, the CMSD comprises from N-terminus to C-terminus: optional CMSD N-terminal sequence—CD3ζ ITAM3—optional first CMSD linker—CD3ζ ITAM3—optional second CMSD linker—CD3ζ ITAM3—optional CMSD C-terminal sequence. In some embodiments, the CMSD comprises the sequence of SEQ ID NO: 44.

In some embodiments according to any of the modified T cells described above, the CMSD comprises from N-terminus to C-terminus: optional CMSD N-terminal sequence—CD3ε ITAM—optional first CMSD linker—CD3ε ITAM—optional second CMSD linker—CD3ε ITAM—optional CMSD C-terminal sequence. In some embodiments, the CMSD comprises the sequence of SEQ ID NO: 46 or 56.

In some embodiments according to any of the modified T cells described above, the CMSD comprises from N-terminus to C-terminus: optional CMSD N-terminal sequence—DAP12 ITAM—optional first CMSD linker—DAP12 ITAM—optional second CMSD linker—DAP12 ITAM—optional CMSD C-terminal sequence. In some embodiments, the CMSD comprises the sequence of SEQ ID NO: 48.

In some embodiments according to any of the modified T cells described above, the CMSD comprises from N-terminus to C-terminus: optional CMSD N-terminal sequence—Igα ITAM—optional first CMSD linker—Igα ITAM—optional second CMSD linker—Igα ITAM—optional CMSD C-terminal sequence. In some embodiments, the CMSD comprises the sequence of SEQ ID NO: 49.

In some embodiments according to any of the modified T cells described above, the CMSD comprises from N-terminus to C-terminus: optional CMSD N-terminal sequence—Igβ ITAM—optional first CMSD linker—Igβ ITAM—optional second CMSD linker—Igβ ITAM—optional CMSD C-terminal sequence. In some embodiments, the CMSD comprises the sequence of SEQ ID NO: 50.

In some embodiments according to any of the modified T cells described above, the CMSD comprises from N-terminus to C-terminus: optional CMSD N-terminal sequence—FcεRIγITAM—optional first CMSD linker—FcεRIγITAM—optional second CMSD linker—FcεRIγITAM—optional CMSD C-terminal sequence. In some embodiments, the CMSD comprises the sequence of SEQ ID NO: 52.

In some embodiments according to any of the modified T cells described above, the CMSD comprises from N-terminus to C-terminus: optional CMSD N-terminal sequence—CD36 ITAM—optional first CMSD linker—CD3ε ITAM—optional second CMSD linker—CD3γ ITAM—optional third CMSD linker—DAP12 ITAM—optional CMSD C-terminal sequence. In some embodiments, the CMSD comprises the sequence of SEQ ID NO: 57.

In some embodiments according to any of the modified T cells described above, the CMSD comprises from N-terminus to C-terminus: optional CMSD N-terminal sequence—CD36 ITAM—optional first CMSD linker—CD36 ITAM—optional second CMSD linker—CD36 ITAM—optional CMSD C-terminal sequence. In some embodiments, the CMSD comprises the sequence of SEQ ID NO: 45.

In some embodiments according to any of the modified T cells described above, the CMSD comprises from N-terminus to C-terminus: optional CMSD N-terminal sequence—CD3γ ITAM—optional first CMSD linker—CD3γ ITAM—optional second CMSD linker—CD3γ ITAM—optional CMSD C-terminal sequence. In some embodiments, the CMSD comprises the sequence of SEQ ID NO: 47.

In some embodiments according to any of the modified T cells described above, the CMSD comprises from N-terminus to C-terminus: optional CMSD N-terminal sequence—FcεRIβITAM—optional first CMSD linker—FcεRIβITAM—optional second CMSD linker—FcεRIβITAM—optional CMSD C-terminal sequence. In some embodiments, the CMSD comprises the sequence of SEQ ID NO: 51.

In some embodiments according to any of the modified T cells described above, the CMSD comprises from N-terminus to C-terminus: optional CMSD N-terminal sequence—CNAIP/NFAM1 ITAM—optional first CMSD linker—CNAIP/NFAM1 ITAM—optional second CMSD linker—CNAIP/NFAM1 ITAM—optional CMSD C-terminal sequence. In some embodiments, the CMSD comprises the sequence of SEQ ID NO: 53.

In some embodiments according to any of the modified T cells described above, the CMSD comprises from N-terminus to C-terminus: optional CMSD N-terminal sequence—CD3ε ITAM—optional first CMSD linker—CD36 ITAM—optional second CMSD linker—DAP12 ITAM—optional third CMSD linker—CD3γ ITAM—optional CMSD C-terminal sequence. In some embodiments, the CMSD comprises the sequence of SEQ ID NO: 64.

In some embodiments according to any of the modified T cells described above, the CMSD comprises from N-terminus to C-terminus: optional CMSD N-terminal sequence—CD3γ ITAM—optional first CMSD linker—DAP12 ITAM—optional second CMSD linker—CD36 ITAM—optional third CMSD linker—CD3ε ITAM—optional CMSD C-terminal sequence. In some embodiments, the CMSD comprises the sequence of SEQ ID NO: 65.

In some embodiments according to any of the modified T cells described above, the CMSD comprises from N-terminus to C-terminus: optional CMSD N-terminal sequence—DAP12 ITAM—optional first CMSD linker—CD3γ ITAM—optional second CMSD linker—CD3ε ITAM—optional third CMSD linker—CD36 ITAM—optional CMSD C-terminal sequence. In some embodiments, the CMSD comprises the sequence of SEQ ID NO: 66.

In some embodiments according to any of the modified T cells described above, the CMSD comprises from N-terminus to C-terminus: optional CMSD N-terminal sequence—CD36 ITAM—optional first CMSD linker—CD3ε ITAM—optional CMSD C-terminal sequence. In some embodiments, the CMSD comprises the sequence of SEQ ID NO: 69.

In some embodiments according to any of the modified T cells described above, the CMSD comprises from N-terminus to C-terminus: optional CMSD N-terminal sequence—CD3γ ITAM—optional first CMSD linker—DAP12 ITAM—optional CMSD C-terminal sequence. In some embodiments, the CMSD comprises the sequence of SEQ ID NO: 70.

In some embodiments according to any of the modified T cells described above, the CMSD comprises from N-terminus to C-terminus: optional CMSD N-terminal sequence—CD36 ITAM—optional first CMSD linker—CD3ε ITAM—optional second CMSD linker—CD3ε ITAM—optional CMSD C-terminal sequence. In some embodiments, the CMSD comprises the sequence of SEQ ID NO: 71.

In some embodiments according to any of the modified T cells described above, the CMSD comprises from N-terminus to C-terminus: optional CMSD N-terminal sequence—CD36 ITAM—optional first CMSD linker—CD3ε ITAM—optional second CMSD linker—CD3γ ITAM—optional CMSD C-terminal sequence. In some embodiments, the CMSD comprises the sequence of SEQ ID NO: 72.

In some embodiments according to any of the modified T cells described above, the CMSD comprises from N-terminus to C-terminus: optional CMSD N-terminal sequence—DAP12 ITAM—optional first CMSD linker—CD3ε ITAM—optional second CMSD linker—CD36 ITAM—optional CMSD C-terminal sequence. In some embodiments, the CMSD comprises the sequence of SEQ ID NO: 73.

In some embodiments according to any of the modified T cells described above, the CMSD comprises from N-terminus to C-terminus: optional CMSD N-terminal sequence—DAP12 ITAM—optional first CMSD linker—CD36 ITAM—optional second CMSD linker—CD3ε ITAM—optional CMSD C-terminal sequence. In some embodiments, the CMSD comprises the sequence of SEQ ID NO: 74.

In some embodiments according to any of the modified T cells described above, the CMSD comprises from N-terminus to C-terminus: optional CMSD N-terminal sequence—CD3ε ITAM—optional CMSD C-terminal sequence. In some embodiments, the CMSD comprises the sequence of SEQ ID NO: 67.

In some embodiments according to any of the modified T cells described above, the CMSD comprises from N-terminus to C-terminus: optional CMSD N-terminal sequence—CD36 ITAM—optional CMSD C-terminal sequence. In some embodiments, the CMSD comprises the sequence of SEQ ID NO: 68.

In some embodiments according to any of the modified T cells described above, the CMSD comprises from N-terminus to C-terminus: optional CMSD N-terminal sequence—CD36 ITAM—optional first CMSD linker—CD3ε ITAM—optional second CMSD linker—CD3γ ITAM—optional third CMSD linker—DAP12 ITAM—optional CMSD C-terminal sequence. In some embodiments, the CMSD comprises the sequence of any of SEQ ID NOs: 58-63.

In some embodiments according to any of the modified T cells described above, the functional exogenous receptor is an ITAM-modified T cell receptor (TCR), an ITAM-modified chimeric antigen receptor (CAR), an ITAM-modified chimeric TCR (cTCR), or an ITAM-modified T cell antigen coupler (TAC)-like chimeric receptor.

In some embodiments according to any of the modified T cells described above, the functional exogenous receptor is an ITAM-modified CAR. In some embodiments, the transmembrane domain is derived from CD8α. In some embodiments, the ISD further comprises a co-stimulatory signaling domain. In some embodiments, the co-stimulatory signaling domain is derived from 4-1BB or CD28. In some embodiments, the co-stimulatory signaling domain comprises the amino acid sequence of SEQ ID NO: 124. In some embodiments, the co-stimulatory domain is N-terminal to the CMSD. In some embodiments, the co-stimulatory domain is C-terminal to the CMSD.

In some embodiments according to any of the modified T cells described above, the functional exogenous receptor is an ITAM-modified cTCR. In some embodiments the ITAM-modified cTCR comprises: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional receptor domain linker, (c) an optional extracellular domain of a first TCR subunit (e.g., CD3ε) or a portion thereof, (d) a transmembrane domain comprising a transmembrane domain of a second TCR subunit (e.g., CD3ε), and (e) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the first and second TCR subunits are selected from the group consisting of TCRα, TCRβ, TCRγ, TCRδ, CD3ε, CD3γ, and CD3δ. In some embodiments, the first and second TCR subunits are both CD3ε. In some embodiments, wherein the one or plurality of CMSD ITAMs are derived from one or more of CD3ε, CD3δ, and CD3γ.

In some embodiments according to any of the modified T cells described above, the functional exogenous receptor is an ITAM-modified TAC-like chimeric receptor. In some embodiments, the ITAM-modified TAC-like chimeric receptor comprises: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional first receptor domain linker, (c) an extracellular TCR binding domain that specifically recognizes the extracellular domain of a first TCR subunit (e.g., CD3ε), (d) an optional second receptor domain linker, (e) an optional extracellular domain of a second TCR subunit (e.g., CD3ε) or a portion thereof, (f) a transmembrane domain comprising a transmembrane domain of a third TCR subunit (e.g., CD3ε), and (g) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the first, second, and third TCR subunits are all selected from the group consisting of TCRα, TCRβ, TCRγ, TCRδ, CD3ε, CD3γ, and CD3δ. In some embodiments, the second and third TCR subunits are both CD3ε. In some embodiments, the one or plurality of CMSD ITAMs are derived from one or more of CD3ε, CD3δ, and CD3γ.

In some embodiments according to any of the modified T cells described above, the extracellular ligand binding domain comprises one or more antigen-binding fragments that specifically recognizing one or more epitopes of one or more target (e.g., tumor) antigens. In some embodiments, the extracellular ligand binding domain is an sdAb or an scFv. In some embodiments, the target (e.g., tumor) antigen is BCMA, CD19, or CD20.

In some embodiments according to any of the modified T cells described above, the functional exogenous receptor further comprises a hinge domain located between the C-terminus of the extracellular ligand binding domain and the N-terminus of the transmembrane domain. In some embodiments, the hinge domain is derived from CD8α. In some embodiments, the functional exogenous receptor further comprises a signal peptide located at the N-terminus of the functional exogenous receptor, such as a signal peptide derived from CD8α.

In some embodiments according to any of the modified T cells described above, the effector function of the functional exogenous receptor comprising the ISD that comprises the CMSD is at most about 80% (such as at most about any of 70%, 60%, 50%, 40%, 30%, 20%, 10%, or 5%) less than a functional exogenous receptor comprising an ISD that comprises an intracellular signaling domain of CD3ζ.

In some embodiments according to any of the modified T cells described above, the effector function of the functional exogenous receptor comprising the ISD that comprises the CMSD is at least about 20% (such as at least about any of 30%, 40%, 50%, 60%, 70%, 80%, 90%, or 100%) active relative to a functional exogenous receptor comprising an ISD that comprises an intracellular signaling domain of CD3ζ.

In some embodiments according to any of the modified T cells described above, the modified T cell further expresses an exogenous Nef protein (e.g., wildtype, subtype, mutant, or non-naturally occurring Nef). In some embodiments, the exogenous Nef protein down-modulates (e.g., down-regulates cell surface expression and/or effector function of) endogenous TCR, CD3, and/or MHC I of the modified T cell, such as down-modulates (e.g., down-regulates cell surface expression and/or effector function of) the endogenous TCR, CD3, and/or MHC I by at least about 40% (such as at least about any of 50%, 60%, 70%, 80%, 90%, or 95%). In some embodiments, the exogenous Nef protein down-modulates (e.g., down-regulate cell surface expression and/or effector function such as signal transduction related to cytolytic activity of) the CMSD-containing functional exogenous receptor by at most about 80% (such as at most about any of 70%, 60%, 50%, 40%, 30%, 20%, 10%, or 5%).

The present invention in another aspect provides a method of producing a modified T cell (e.g., allogeneic or autologous T cell), comprising introducing into a precursor T cell a nucleic acid encoding a functional exogenous receptor (e.g., ITAM-modified CAR, ITAM-modified TCR, ITAM-modified cTCR, or ITAM-modified TAC-like chimeric receptor), wherein the functional exogenous receptor comprises: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the nucleic acid is on a vector, such as a viral vector (e.g., lentiviral vector). In some embodiments, the method further comprises isolating and/or enriching functional exogenous receptor-positive T cells from the modified T cells. In some embodiments, the method further comprises formulating the modified T cell with at least one pharmaceutically acceptable carrier. In some embodiments, the plurality (e.g., 2, 3, 4, or more) of CMSD ITAMs are directly linked to each other. In some embodiments, the CMSD comprises two or more (e.g., 2, 3, 4, or more) CMSD ITAMs connected by one or more linkers not derived from an ITAM-containing parent molecule (e.g., G/S linker). In some embodiments, the CMSD comprises one or more CMSD linkers derived from an ITAM-containing parent molecule that is different from the ITAM-containing parent molecule from which one or more of the CMSD ITAMs are derived from. In some embodiments, the CMSD comprises two or more (e.g., 2, 3, 4, or more) identical CMSD ITAMs. In some embodiments, at least one of the CMSD ITAMs is not derived from CD3ζ. In some embodiments, at least one of the CMSD ITAMs is not ITAM1 or ITAM2 of CD3ζ. In some embodiments, the plurality of CMSD ITAMs are each derived from a different ITAM-containing parent molecule. In some embodiments, at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, the CMSD consists essentially of (e.g., consists of) one CMSD ITAM. In some embodiments, the CMSD consists essentially of (e.g., consists of) one CMSD ITAM and a CMSD N-terminal sequence and/or a CMSD C-terminal sequence that is heterologous to the ITAM-containing parent molecule (e.g., a G/S linker). In some embodiments, at least one of the plurality of CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin.

In another aspect, there is also provided a modified T cell (e.g., allogeneic or autologous T cell) obtained by any of the methods described above.

In a further aspect, there is provided a viral vector (e.g., lentiviral vector) comprising a nucleic acid encoding a functional exogenous receptor (e.g., ITAM-modified CAR, ITAM-modified TCR, ITAM-modified cTCR, or ITAM-modified TAC-like chimeric receptor), wherein the functional exogenous receptor comprises: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, at least one of the CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin.

Pharmaceutical compositions comprising any of the modified T cells (e.g., allogeneic T cells) described herein, methods of treating a disease (e.g., cancer, infectious disease, autoimmune disorders, or radiation sickness) using any of the modified T cells described herein or pharmaceutical compositions thereof are also provided. In some embodiments, the individual (e.g., human) for treatment is histoincompatible with the donor of the precursor T cell from which the modified T cell is derived.

The present invention further provides kits and articles of manufacture that are useful for the methods described herein.

### BRIEF DESCRIPTION OF THE DRAWINGS

FIG. 1 demonstrates CMSD ITAMs in CAR-T cells possesses CAR-mediated specific activation activity. FIGS. 1A-1C show activation molecule expression of CD69 (FIG. 1A), CD25 (FIG. 1B), and HLA-DR (FIG. 1C) in Jurkat-ISD-modified BCMA CAR cells incubated with target cell lines RPMI8226 and non-target cell lines K562, respectively. “Jurakt” indicates untransduced Jurkat cells served as control. FIGS. 1D-1I demonstrate the interaction between SIV Nef and SIV Nef M116 with BCMA CARs comprising various modified intracellular signaling domains (ISDs). FIG. 1D shows high CAR positive rates in Jurkat-ISD-modified CAR-empty vector cells, as controls. FIG. 1E shows BCMA CAR expression reduced in Jurkat-M663-SIV Nef cells, Jurkat-M665-SIV Nef cells, and Jurkat-M666-SIV Nef cells. FIG. 1F shows BCMA CAR expression reduced in Jurkat-M663-SIV Nef M116 cells, Jurkat-M665-SIV Nef M116 cells, and Jurkat-M666-SIV M116 Nef cells. FIG. 1G shows high BCMA CAR positive rate in Jurkat-ITAM-modified BCMA CAR-empty vector cells, as controls. FIGS. 1H-1I show no significant reduction of BCMA CAR expression in Jurkat-M678 cells, Jurkat-M680 cells, Jurkat-M684 cells, and Jurkat-M799 cells transduced with SIV Nef and SIV Nef M116, respectively. FIGS. 1H-1I show significant reduction of BCMA CAR expression in Jurkat-M663-SIV Nef cells and Jurkat M663-SIV Nef M116 cells.

FIGS. 2A-2B show specific cytotoxicity of various ITAM-modified CAR-T cells on target cells. FIG. 2A shows relative killing efficiency of modified T cells expressing BCMA-BBz, BCMA-BB007, BCMA-BB008, BCMA-BB009, and BCMA-BB010, respectively, on multiple myeloma cell line RPMI8226.Luc at E:T ratio of 40:1. T cells expressing BCMA-BB (only has 4-1BB co-stimulatory signaling domain, no CD3ζ intracellular signaling domain) served as negative control. FIG. 2B shows relative killing efficiency of modified T cells separately expressing LCAR-L186S and CD20-BB010, on lymphoma Raji.Luc cell lines at E:T ratio of 20:1. “UnT” indicates untransduced T cells served as control.

FIG. 3 demonstrates impact of CMSD linker on CAR-T cells activity. FIG. 3 shows relative killing efficiency of modified T cells expressing different ITAM-modified BCMA CARs on multiple myeloma cell line RPMI8226.Luc at E:T ratio of 2.5:1, such as ISD consists of traditional CD3ζ (BCMA-BBz), CMSD ITAMs directly linked to each other (BCMA-BB024), CMSD ITAMs connected by one or more CMSD linkers (BCMA-BB010, BCMA-BB025, BCMA-BB026, BCMA-BB027, BCMA-BB028, and BCMA-BB029), respectively. “UnT” indicates untransduced T cell served as control.

FIG. 4 demonstrates impact of order of CMSD ITAMs on CAR-T cells activity. FIG. 4 shows relative killing efficiency of modified T cells expressing BCMA-BBz, BCMA-BB010, BCMA-BB030, BCMA-BB031, and BCMA-BB032, respectively, on multiple myeloma cell line RPMI8226.Luc at E:T ratio of 2.5:1. “UnT” indicates untransduced T cell served as control.

FIG. 5 demonstrates impact of quantity and source of CMSD ITAM on CAR-T cells activity. FIG. 5 shows relative killing efficiency of modified T cells separately expressing traditional CD3ζ CAR (BCMA-BBz) and different ITAM-modified BCMA CARs on multiple myeloma cell line RPMI8226.Luc at E:T ratio of 2.5:1, such as ISD comprising 1 CMSD ITAM (BCMA-BB033 and BCAM-BB034), 2 CMSD ITAMs (BCMA-BB035 and BCMA-BB036), 3 CMSD ITAMs (BCMA-BB037 and BCMA-BB038), and 4 CMSD ITAMs (BCMA-BB010, BCMA-BB030, BCMA-BB031, and BCMA-BB032), respectively. “UnT” indicates untransduced T cell served as control.

FIG. 6 shows T cell proliferation of ITAM-modified BCMA CAR-T cells post target tumor cells re-challenge. “UnT” indicates untransduced T cell.

FIGS. 7A-7D show ITAM-modified CAR-T cells' phenotype post target tumor cells re-challenge. FIG. 7A shows PD-1 and LAG-3 expression of T cell exhausted markers in CAR-T cells. FIGS. 7B-7C show cell ratio of TEMRA cells (CD45RA+/CCR7-), TEM cells (CD45RA−/CCR7-), TCM cells (CD45RA−/CCR7+), and Naive cells (CD45RA+/CCR7+) among CAR+ T cells, CAR+/CD8+ T cells, and CAR+/CD4+ T cells.

FIG. 8 depicts ITAM-containing parent molecule (e.g., CD3ζ, CD3ε) intracellular signaling domain structure and exemplary CMSD structures.

FIG. 9A shows CD20 CAR positive rates by FACS analysis after transducing primary T cells with lentiviruses carrying LCAR-UL186S (SIV Nef M116-IRES-CD8a SP-CD20 scFv (Leu16)-CD8a hinge-CD8a TM-4-1BB-ITAM010) and LCAR-L186S (CD8a SP-CD20 scFv (Leu16)-CD8a hinge-CD8a TM-4-1BB-CD3ζ) sequences, respectively. “CAR pos” means CAR positive rate. “UnT” indicates untransduced T cells. FIG. 9B shows cytotoxicity of LCAR-UL186S T cells and LCAR-L186S T cells on lymphoma Raji.Luc cell line (CD20+) at different E:T ratios of 20:1, 10:1 and 5:1, respectively, on day 3 of the killing assay. Untransduced T cells (UnT) served as control.

FIGS. 10A-10C demonstrate the levels of pro-inflammatory factors (FIG. 10A), chemokines (FIG. 10B), and cytokines (FIG. 10C) released by LCAR-L186S T cells (CD20 CAR with traditional CD3ζ intracellular signaling domain) and LCAR-UL186S T cells (ITAM-modified CD20 CAR/SIV Nef M116 co-expression) when killing lymphoma Raji.Luc cell line at different E:T ratios of 20:1, 10:1 and 5:1, on day 3 of the killing assay. Untransduced T cells (UnT) served as control.

FIGS. 11A-11D show in vivo efficacy of LCAR-L186S T cells and TCRαβ MACS sorted LCAR-UL186S CAR+/TCRαβ− T cells. Immuno-deficient NCG mice were engrafted with human Raji.Luc tumor cells (CD20+) on day −4, and subsequently treated with HBSS, untransduced T cells (UnT), LCAR-L186S T cells, and TCRαβ MACS sorted LCAR-UL186S CAR+/TCRαβ− T cells on day 0. Mice were assessed on a weekly basis to monitor tumor growth by bioluminescence imaging (FIGS. 11A-11B), body weight (FIG. 11C), and survival (FIG. 11D).

FIGS. 12A-12D show in vivo efficacy of LCAR-L186S T cells and TCRαβ MACS sorted LCAR-UL186S CAR+/TCRαβ− T cells following tumor re-challenge, mimicking tumor recurrence model. 41 days post CAR-T administration, non-relapsed mice were further injected with 3×10⁴ Raji.Luc tumor cells (denoted as day 0). Mice were assessed on a regular basis to monitor tumor growth by bioluminescence imaging (FIGS. 12A-12B), body weight (FIG. 12C), and survival (FIG. 12D).

FIG. 13 shows BCMA CAR positive rates for LIC948A22 CAR-T cells (86.5% CAR+) and TCRαβ MACS sorted LUC948A22 UCAR-T cells (85.9% CAR+). “UnT” represents untransduced T lymphocytes and served as control. “LIC948A22 CAR-T” represents T lymphocytes expressing an autologous BCMA CAR and enriched by BCMA+ MACS. “LUC948A22 UCAR-T” represents T lymphocytes expressing a universal BCMA CAR and enriched by TCRαβ− MACS.

FIG. 14 shows specific tumor cytotoxicity of LIC948A22 CAR-T cells and TCRαβ MACS sorted LUC948A22 UCAR-T cells (CAR+/TCRαβ−) on RPMI8226.Luc cell lines at different E:T cell ratios of 2.5:1 and 1.25:1. “UnT” represents untransduced T lymphocytes and served as control. “LIC948A22 CAR-T” represents T lymphocytes expressing autologous BCMA CAR and enriched by BCMA+ MACS. “LUC948A22 UCAR-T” represents T lymphocytes expressing universal BCMA CAR and enriched by TCRαβ− MACS.

FIGS. 15A-15C demonstrate the levels of pro-inflammatory factors (FIG. 15A), chemokines (FIG. 15B), and cytokines (FIG. 15C) released in vitro by LIC948A22 CAR-T cells and TCRαβ MACS sorted LUC948A22 UCAR-T cells (CAR+/TCRαβ−) when killing RPMI8226.Luc cell lines at different E:T ratios of 2.5:1 and 1.25:1. “UnT” represents untransduced T lymphocytes and served as control. “LIC948A22 CAR-T” represents T lymphocytes expressing autologous BCMA CAR and enriched by BCMA+ MACS. “LUC948A22 UCAR-T” represents T lymphocytes expressing universal BCMA CAR and enriched by TCRαβ− MACS.

FIG. 16A shows TCRα3 expression of Jurkat cells transduced with SIV Nef M116+ITAM-modified CD20 CAR and SIV Nef M116+CD3ζ CD20 CAR (M1185) all-in-one construct, respectively. FIG. 16B shows relative killing efficiency of T cells transduced with SIV Nef M116+ITAM-modified CD20 CAR all-in-one construct and SIV Nef M116+CD3ζ CD20 CAR (M1185), respectively, on lymphoma cell line Raji.Luc at E:T ratio of 20:1. “TCRαβ pos” indicates TCRαβ positive rate. “Jurkat” indicates untransduced Jurkat cells served as control. “UnT” indicates untransduced T cells served as control.

FIG. 17A shows TCRα3 expression of Jurkat cells transduced with SIV Nef M116+ITAM-modified BCMA CAR and SIV Nef M116+CD3ζ BCMA CAR (M1215) all-in-one construct, respectively. FIG. 17B shows relative killing efficiency of T cells transduced with SIV Nef M116+ITAM-modified BCMA CAR and SIV Nef M116+CD3ζ BCMA CAR (M1215) all-in-one construct, respectively, on multiple myeloma cell line RPMI8226.Luc at E:T ratio of 4:1. “TCRαβ pos” indicates TCRαβ positive rate. “Jurkat” indicates untransduced Jurkat cells served as control. “UnT” indicates untransduced T cells served as control.

FIG. 18A shows TCRα3 expression of M598-T cells and MACS sorted TCRα3 negative M598-T cells. FIG. 18B shows BCMA CAR expression of M598-T cells and MACS sorted TCRαβ negative M598-T cells. FIG. 18C shows relative killing efficiency of MACS sorted TCRαβ negative M598-T cells on multiple myeloma cell line RPMI8226.Luc at different E:T ratios of 2.5:1, 1.25:1, and 1:1.25, respectively. “TCRαβ pos” indicates TCRαβ positive rate. “CAR pos” indicates CAR positive rate. “UnT” indicates untransduced T cells. “TCRαβ− M598-T” indicates MACS sorted TCRαβ negative M598-T cells.

FIGS. 19A-19D show SIV Nef subtype with dual regulation on TCRαβ and MHC expression in CAR-T cell immunotherapy. FIGS. 19A-19B show expression rate of CD20 CAR, TCRαβ, and HLA-B7 in modified T cells expressing LCAR-UL186S and M1392, respectively. FIG. 19C shows MHC class I cross-reactivity based on Mixed Lymphocyte Reaction of LCAR-L186S T cells, B2M KO LCAR-L186S T cells, and TCRαβ− M1392-T cells, 48 hours post incubation with effector cells at E:T ratio of 1:1. FIG. 19D shows relative killing efficiency of TCRαβ− M1392-T cells on lymphoma cell line Raji.Luc at different E:T ratios of 20:1, 10:1, and 5:1. UnT indicates untransduced T cells served as control.

### DETAILED DESCRIPTION OF THE PRESENT APPLICATION

The present application provides modified T cells comprising a functional exogenous receptor comprising a chimeric signaling domain (“CMSD”). The CMSD described herein comprises one or a plurality of Immune-receptor Tyrosine-based Activation Motifs (“ITAMs”), and optional linkers arranged in a configuration that is different than any of the naturally occurring ITAM-containing parent molecules, such as CD3ζ. It was surprisingly found that, like traditional functional exogenous receptors containing naturally-occurring ITAM-based signaling domains, receptors containing the CMSD are capable of activating T cells upon binding of the receptor to a cognate ligand. Compared to a traditional functional exogenous receptor (such as a chimeric antigen receptor (CAR) comprising CD3ζ intracellular signaling domain (ISD)), receptors comprising CMSDs described herein (e.g., a CAR comprising a CMSD) demonstrate superior tumor cytotoxicity in both tumor xenograft mice model and tumor recurrence mice model, while having significantly reduced induction in the release of cytokines, chemokines, and pro-inflammatory factors.

It was further surprisingly found that receptors containing certain types of CMSD (for example CMSDs not containing ITAM1 and ITAM2 of CD3), when co-expressed with a Nef protein capable of down-regulating endogenous T cell receptors (TCRs) in a T cell (also referred herein as “TCR-deficient T cells” or “GvHD-minimized T cells”), showed no or reduced down-regulation by the Nef protein. This property makes the CMSD-containing functional exogenous receptors particularly suitable for use in conjunction with a Nef protein, for example for allogeneic T cell therapy.

Thus, the present invention in one aspect provides a modified T cell comprising a functional exogenous receptor comprising: (a) an extracellular ligand binding domain; (b) a transmembrane domain; and (c) an intracellular signaling domain (“ISD”) comprising a CMSD comprising one or a plurality of ITAMs (referred to as “CMSD ITAMs”), wherein the plurality of CMSD ITAMs are optionally connected by one or more linkers (referred to as “CMSD linkers”). The functional exogenous receptor (herein after referred to as “ITAM-modified functional exogenous receptor” or “CMSD-containing functional exogenous receptor”) can have a structure that is similar to a chimeric antigen receptor (“CAR”), an engineered T cell receptor (“engineered TCR”), a chimeric T cell receptor (“cTCR”), and T cell antigen coupler (“TAC”)-like chimeric receptor, with the exception that the ISD comprises a CMSD. These functional exogenous receptor are herein referred to as “ITAM-modified CAR,” “ITAM-modified TCR,” “ITAM-modified cTCR,” and “ITAM-modified TAC-like chimeric receptor,” respectively. Modified T cells comprising the functional exogenous receptor comprising a CMSD described herein are referred to as “ITAM-modified TCR-T cells”, “ITAM-modified cTCR-T cells”, “ITAM-modified TAC-like-T cells”, or “ITAM-modified CAR-T cells.”

Also provided are functional exogenous receptors to be included in the modified T cells, nucleic acids encoding such functional exogenous receptors, and method of making the modified T cells. Further provided are methods of using the modified T cells for treating various diseases, such as cancer.

### I. Definitions

The term “functional exogenous receptor” as used herein, refers to an exogenous receptor (e.g., ITAM-modified TCR, ITAM-modified cTCR, ITAM-modified TAC-like chimeric receptor, or ITAM-modified CAR) that retains its biological activity after being introduced into a T cell. The biological activity include but are not limited to the ability of the exogenous receptor in specifically binding to a molecule, properly transducing downstream signals, such as inducing cellular proliferation, cytokine production and/or performance of regulatory or cytolytic effector functions.

As use herein, the term “specifically binds,” “specifically recognizes,” or is “specific for” refers to measurable and reproducible interactions such as binding between a target and an antigen binding protein (such as an antigen-binding domain, a ligand-receptor, any of the functional exogenous receptor comprising a CMSD described herein), which is determinative of the presence of the target in the presence of a heterogeneous population of molecules including biological molecules. For example, an antigen binding protein that specifically binds a target (which can be an epitope) is an antigen binding protein that binds this target with greater affinity, avidity, more readily, and/or with greater duration than it binds other targets. In some embodiments, the extent of binding of an antigen binding protein to an unrelated target is less than about 10% of the binding of the antigen binding protein to the target as measured, e.g., by a radioimmunoassay (RIA). In some embodiments, an antigen binding protein that specifically binds a target has a dissociation constant (Kd) of ≤1 μM, ≤100 nM, ≤10 nM, ≤1 nM, or ≤0.1 nM. In some embodiments, an antigen binding protein specifically binds an epitope on a protein that is conserved among the protein from different species. In some embodiments, specific binding can include, but does not require exclusive binding.

The term “specificity” refers to selective recognition of an antigen binding protein (e.g., any of the functional exogenous receptor comprising a CMSD described herein, sdAb, scFv, or ligand-receptor) for a particular epitope of an antigen. Natural antibodies, for example, are monospecific. The term “multispecific” as used herein denotes that an antigen binding protein (e.g., any of the functional exogenous receptor comprising a CMSD described herein, sdAb, scFv, or ligand-receptor) has two or more antigen-binding sites of which at least two bind different antigens or epitopes. “Bispecific” as used herein denotes that an antigen binding protein (e.g., any of the functional exogenous receptor comprising a CMSD described herein, sdAb, scFv, or ligand-receptor) has two different antigen-binding specificities. The term “monospecific” as used herein denotes an antigen binding protein (e.g., any of the functional exogenous receptor comprising a CMSD described herein, sdAb, scFv, or ligand-receptor) that has one or more binding sites each of which bind the same epitope of an antigen.

“Binding affinity” generally refers to the strength of the sum total of non-covalent interactions between a single binding site of a molecule (e.g., an antibody, a ligand-receptor, any of the functional exogenous receptor comprising a CMSD described herein) and its binding partner (e.g., an antigen, a ligand). Unless indicated otherwise, as used herein, “binding affinity” refers to intrinsic binding affinity that reflects a 1:1 interaction between members of a binding pair (e.g., antibody and antigen, or any of the functional exogenous receptor comprising a CMSD described herein and an antigen, such as an ITAM-modified CAR and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present application. Specific illustrative and exemplary embodiments for measuring binding affinity are described in the following.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An “isolated” nucleic acid molecule (e.g., encoding any of the functional exogenous receptor comprising a CMSD described herein) described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell (e.g., T cell). A “transfected” or “transformed” or “transduced” cell is one which has been transfected, transformed or transduced with exogenous nucleic acid. The cell includes the primary subject cell and its progeny.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present application contemplate any one or more of these aspects of treatment.

As used herein, an “individual” or a “subject” refers to a mammal, including, but not limited to, human, bovine, horse, feline, canine, rodent, or primate. In some embodiments, the individual is a human.

The term “effective amount” used herein refers to an amount of an agent, such as a modified T cell described herein (e.g., ITAM-modified T cell), or a pharmaceutical composition thereof, sufficient to treat a specified disorder, condition or disease such as ameliorate, palliate, lessen, and/or delay one or more of its symptoms (e.g., cancer, infectious disease, autoimmune disorders, or radiation sickness). In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the agent (e.g., modified T cell) or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. In the case of infectious disease, such as viral infection, the therapeutically effective amount of a modified T cell described herein or composition thereof can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient.

As used herein, the term “autologous” is meant to refer to any material derived from the same individual to whom it is later to be re-introduced into the individual.

“Allogeneic” refers to a graft derived from a different individual of the same species. “Allogeneic T cell” refers to a T cell from a donor having a tissue human leukocyte antigen (HLA) type that matches the recipient. Typically, matching is performed on the basis of variability at three or more loci of the HLA gene, and a perfect match at these loci is preferred. In some instances allogeneic transplant donors may be related (usually a closely HLA matched sibling), syngeneic (a monozygotic “identical” twin of the patient) or unrelated (donor who is not related and found to have very close degree of HLA matching). The HLA genes fall in two categories (Type I and Type II). In general, mismatches of the Type-I genes (i.e., HLA-A, HLA-B, or HLA-C) increase the risk of graft rejection. A mismatch of an HLA Type II gene (i.e., HLA-DR, or HLA-DQB1) increases the risk of GvHD.

A “patient” as used herein includes any human who is afflicted with a disease (e.g., cancer, viral infection, GvHD). The terms “subject,” “individual,” and “patient” are used interchangeably herein. The term “donor subject” or “donor” refers to herein a subject whose cells are being obtained for further in vitro engineering. The donor subject can be a patient that is to be treated with a population of cells generated by the methods described herein (i.e., an autologous donor), or can be an individual who donates a blood sample (e.g., lymphocyte sample) that, upon generation of the population of cells generated by the methods described herein, will be used to treat a different individual or patient (i.e., an allogeneic donor). Those subjects who receive the cells that were prepared by the present methods can be referred to as “recipient” or “recipient subject.”

The term “stimulation”, as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex, or binding any of the functional exogenous receptor comprising a CMSD described herein. Further, the stimulation event may activate a cell and upregulate or down-regulate expression or secretion of a molecule, such as down-regulation of TGF-β. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

The term “activation”, as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process. The term “activated T cells” indicates T cells that are currently undergoing cell division, cytokine production, performance of regulatory or cytolytic effector functions, and/or has recently undergone the process of “activation.”

The term “down-modulation” of a molecule (e.g., endogenous TCR (e.g., TCRα and/or TCRβ), CD4, CD28, MHC I, CD3ε, CD3δ, CD3γ, CD3ζ, functional extracellular receptor comprising a CMSD described herein) in T cells refers to down-regulate cell surface expression of the molecule, and/or interfering with its signal transduction (e.g., CMSD-containing functional extracellular receptor, TCR, CD3, CD4, CD28-mediated signal transduction), T cell activation, T cell stimulation, and/or T cell proliferation. Down modulation of the target receptors via e.g., internalization, stripping, capping or other forms of changing receptors rearrangements on the cell surface may also be encompassed.

It is understood that embodiments of the present application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. T Cells Comprising a CMSD-Containing Functional Exogenous Receptor

The present application provides a modified T cell (e.g., allogeneic or autologous T cell) comprising: a functional exogenous receptor comprising a CMSD described herein (e.g., ITAM-modified CAR, ITAM-modified TCR, ITAM-modified cTCR, or ITAM-modified TAC-like chimeric receptor). In some embodiments, the ITAM-modified T cell described herein further expresses an exogenous Nef protein (e.g., wildtype Nef or mutant Nef). Modified T cells co-expressing exogenous Nef protein and CMSD-containing functional exogenous receptors are referred to as “Nef-containing ITAM-modified T cells” or “GvHD-minimized ITAM-modified T cells”, such as “Nef-containing ITAM-modified TCR-T cells”, “Nef-containing ITAM-modified cTCR-T cells”, “Nef-containing ITAM-modified TAC-like-T cells”, or “Nef-containing ITAM-modified CAR-T cells.”

In some embodiments, there is provided a modified T cell (e.g., allogeneic or autologous T cell) comprising: a functional exogenous receptor (e.g., ITAM-modified CAR, ITAM-modified TCR, ITAM-modified cTCR, or ITAM-modified TAC-like chimeric receptor) comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, there is provided a modified T cell (e.g., allogeneic or autologous T cell) comprising a nucleic acid encoding a functional exogenous receptor (e.g., ITAM-modified CAR, ITAM-modified TCR, ITAM-modified cTCR, or ITAM-modified TAC-like chimeric receptor) comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the modified T cell comprises a modified endogenous TCR or B2M locus.

In some embodiments, the functional exogenous receptor is an ITAM-modified CAR. Thus in some embodiments, there is provided a modified T cell (e.g., allogeneic or autologous T cell) comprising: an ITAM-modified CAR comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the ITAM-modified CAR comprises from N′ to C′: (a) an extracellular ligand binding domain comprising an antigen-binding fragment (e.g., scFv, sdAb) specifically recognizing one or more epitopes of one or more target antigens (e.g., tumor antigen such as BCMA, CD19, CD20), (b) an optional hinge domain (e.g., derived from CD8α), (c) a transmembrane domain (e.g., derived from CD8α), and (d) an ISD comprising an optional co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the co-stimulatory signaling domain is N-terminal to the CMSD. In some embodiments, the co-stimulatory signaling domain is C-terminal to the CMSD. In some embodiments, the ITAM-modified CAR further comprises a signal peptide (e.g., derived from CD8α) located at the N-terminus of the ITAM-modified CAR. In some embodiments, the ITAM-modified CAR is an ITAM-modified BCMA CAR, comprising: (a) an extracellular ligand binding domain comprising i) an anti-BCMA scFv; or ii) a first sdAb moiety (e.g., V_{H}H) that specifically binds to BCMA, an optional linker, and a second sdAb moiety (e.g., V_{H}H) that specifically binds to BCMA, (b) an optional hinge domain (e.g., derived from CD8α), (c) a transmembrane domain (e.g., derived from CD8α), and (d) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, wherein the co-stimulatory signaling domain is N-terminal to the CMSD. In some embodiments, the ITAM-modified BCMA CAR comprises from N′ to C′: (a) a CD8a signal peptide, (b) an extracellular ligand binding domain comprising i) an anti-BCMA scFv or ii) a first sdAb moiety (e.g., V_{H}H) that specifically binds to BCMA, an optional linker, and a second sdAb moiety (e.g., V_{H}H) that specifically binds to BCMA, (c) a CD8a hinge domain, (d) a CD8a transmembrane domain, (e) a 4-1BB co-stimulatory signaling domain, and (f) a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the ITAM-modified CAR is an ITAM-modified CD20 CAR, comprising: (a) an extracellular ligand binding domain comprising an anti-CD20 scFv, (b) an optional hinge domain (e.g., derived from CD8α), (c) a transmembrane domain (e.g., derived from CD8α), and (d) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, wherein the co-stimulatory signaling domain is N-terminal to the CMSD. In some embodiments, the ITAM-modified CD20 CAR comprises from N′ to C′: (a) a CD8a signal peptide, (b) an extracellular ligand binding domain comprising an anti-CD20 scFv, (c) a CD8a hinge domain, (d) a CD8a transmembrane domain, (e) a 4-1BB co-stimulatory signaling domain, and (f) a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the signal peptide comprises the amino acid sequence of SEQ ID NO: 127. In some embodiments, the hinge domain comprises the amino acid sequence of SEQ ID NO: 125. In some embodiments, the transmembrane domain comprises the amino acid sequence of SEQ ID NO: 126. In some embodiments, the co-stimulatory signaling domain comprises the amino acid sequence of SEQ ID NO: 124. In some embodiments, the one or more CMSD linkers and the linker between anti-BCMA sdAbs are independently selected from the group consisting of SEQ ID NOs: 17-39 and 116-120. In some embodiments, the ITAM-modified BCMA CAR comprises the amino acid sequence of any of SEQ ID NOs: 76-96 and 106-113. In some embodiments, the anti-CD20 scFv is derived from Leu16. In some embodiments, the ITAM-modified CD20 CAR comprises the amino acid sequence of any of SEQ ID NOs: 98-104. Thus in some embodiments, there is provided a modified T cell (e.g., allogeneic or autologous T cell) comprising: an ITAM-modified BCMA CAR comprising the amino acid sequence of any of SEQ ID NOs: 76-96 and 106-113. In some embodiments, there is provided a modified T cell (e.g., allogeneic or autologous T cell) comprising: an ITAM-modified CD20 CAR comprising the amino acid sequence of any of SEQ ID NOs: 98-104.

In some embodiments, there is provided a modified T cell (e.g., allogeneic or autologous T cell) comprising: an ITAM-modified TCR comprising: (a) an extracellular ligand binding domain comprising a Vα and a Vβ derived from a wildtype TCR together specifically recognizing one or more epitopes of one or more target antigens (e.g., tumor antigen such as BCMA, CD19, CD20) or target antigen peptide/MHC complex (e.g., BCMA/MHC complex), wherein the Vα, the Vβ, or both, comprise one or more mutations in one or more CDRs relative to the wildtype TCR, (b) a transmembrane domain comprising a transmembrane domain of TCRα and a transmembrane domain of TCRβ, and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the ITAM-modified TCR further comprises a signal peptide (e.g., derived from CD8α) located at the N-terminus of the ITAM-modified TCR. In some embodiments, the signal peptide comprises the amino acid sequence of SEQ ID NO: 127. In some embodiments, the one or more CMSD linkers are independently selected from the group consisting of SEQ ID NOs: 17-39 and 116-120.

In some embodiments, there is provided a modified T cell (e.g., allogeneic or autologous T cell) comprising: an ITAM-modified cTCR comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional receptor domain linker, (c) an optional extracellular domain of a first TCR subunit (e.g., CD3ε) or a portion thereof, (d) a transmembrane domain comprising a transmembrane domain of a second TCR subunit (e.g., CD3ε), and (e) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, wherein the first and second TCR subunits are independently selected from the group consisting of TCRα, TCRβ, TCRγ, TCRδ, CD3ε, CD3γ, and CD3δ. In some embodiments, the extracellular ligand binding domain comprises an anti-BCMA scFv or an anti-CD20 scFv. In some embodiments, the extracellular ligand binding domain comprises a first sdAb moiety (e.g., V_{H}H) that specifically binds to BCMA, an optional linker, and a second sdAb moiety (e.g., V_{H}H) that specifically binds to BCMA. In some embodiments, the first and second TCR subunits are the same. In some embodiments, the first and second TCR subunits are different. In some embodiments, the receptor domain linker and/or the linker between two anti-BCMA sdAbs are selected from the group consisting of SEQ ID NOs: 17-39 and 116-120. In some embodiments, the CMSD consists essentially of (e.g., consists of) one CD3ε/6/γ ITAM. In some embodiments, the first and second TCR subunits are both CD3. In some embodiments, the one or more of CMSD ITAMs are derived from one or more of CD3ε, CD3δ, and CD3γ. In some embodiments, the CMSD linkers are derived from CD3ε, CD3δ, or CD3γ, or selected from the group consisting of SEQ ID NOs: 17-39 and 116-120. In some embodiments, the CMSD comprises at least two CD3ε ITAMs, at least two CD36 ITAMs, or at least two CD3γ ITAMs. In some embodiments, the ITAM-modified cTCR further comprises a hinge domain (e.g., derived from CD8α) located between the C-terminus of the extracellular ligand binding domain and the N-terminus of the transmembrane domain (if the optional extracellular domain of a first TCR subunit or a portion thereof is absent). In some embodiments, the ITAM-modified cTCR further comprises a signal peptide (e.g., derived from CD8α) located at the N-terminus of the ITAM-modified cTCR. In some embodiments, the signal peptide comprises the amino acid sequence of SEQ ID NO: 127. In some embodiments, the hinge domain comprises the amino acid sequence of SEQ ID NO: 125.

In some embodiments, there is provided a modified T cell (e.g., allogeneic or autologous T cell) comprising: an ITAM-modified TAC-like chimeric receptor comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional first receptor domain linker, (c) an extracellular TCR binding domain that specifically recognizes the extracellular domain of a first TCR subunit (e.g., CD3ε), (d) an optional second receptor domain linker, (e) an optional extracellular domain of a second TCR subunit (e.g., CD3ε) or a portion thereof, (f) a transmembrane domain comprising a transmembrane domain of a third TCR subunit (e.g., CD3ε), and (g) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, wherein the first, second, and third TCR subunits are independently selected from the group consisting of TCRα, TCRβ, TCRγ, TCRδ, CD3ε, CD3γ, and CD3δ. In some embodiments, the extracellular ligand binding domain comprises an anti-BCMA scFv or an anti-CD20 scFv. In some embodiments, the extracellular ligand binding domain comprises a first sdAb moiety (e.g., V_{H}H) that specifically binds to BCMA, an optional linker, and a second sdAb moiety (e.g., V_{H}H) that specifically binds to BCMA. In some embodiments, the first, second, and third TCR subunits are the same. In some embodiments, the first, second, and third TCR subunits are all different. In some embodiments, the second and third TCR subunits are the same, but different from the first TCR subunit. In some embodiments, the ITAM-modified TAC-like chimeric receptor further comprises a hinge domain (e.g., derived from CD8α) located between the C-terminus of the extracellular ligand binding domain and the N-terminus of the transmembrane domain (if the extracellular TCR binding domain is N-terminal to the extracellular ligand binding domain, and the optional extracellular domain of a second TCR subunit or a portion thereof is absent). In some embodiments, the ITAM-modified TAC-like chimeric receptor further comprises a hinge domain (e.g., derived from CD8α) located between the C-terminus of the extracellular TCR binding domain and the N-terminus of the transmembrane domain (if the extracellular TCR binding domain is C-terminal to the extracellular ligand binding domain, and the optional extracellular domain of a second TCR subunit or a portion thereof is absent). In some embodiments, the ITAM-modified TAC-like chimeric receptor further comprises a signal peptide (e.g., derived from CD8α) located at the N-terminus of the ITAM-modified TAC-like chimeric receptor. In some embodiments, the first and/or second receptor domain linkers, the linker between two anti-BCMA sdAbs, and the one or more CMSD linkers are independently selected from the group consisting of SEQ ID NOs: 17-39 and 116-120. In some embodiments, the second and third TCR subunits are both CD3. In some embodiments, the one or more CMSD ITAMs are derived from one or more of CD3ε, CD3δ, and CD3γ. In some embodiments, the CMSD linkers are derived from CD3ε, CD3δ, or CD3γ, or selected from the group consisting of SEQ ID NOs: 17-39 and 116-120. In some embodiments, the CMSD comprises at least two CD3ε ITAMs, at least two CD36 ITAMs, or at least two CD3γ ITAMs. In some embodiments, the signal peptide comprises the amino acid sequence of SEQ ID NO: 127. In some embodiments, the hinge domain comprises the amino acid sequence of SEQ ID NO: 125.

In some embodiments, the nucleic acid encoding the CMSD-containing functional exogenous receptor described herein is operably linked to a promoter. In some embodiments, the promoter is selected from the group consisting of a Rous Sarcoma Virus (RSV) promoter, a Simian Virus 40 (SV40) promoter, a cytomegalovirus immediate early gene promoter (CMV IE), an elongation factor 1 alpha promoter (EF1-α), a phosphoglycerate kinase-1 (PGK) promoter, a ubiquitin-C (UBQ-C) promoter, a cytomegalovirus enhancer/chicken beta-actin (CAG) promoter, a polyoma enhancer/herpes simplex thymidine kinase (MC1) promoter, a beta actin ((3-ACT) promoter, a “myeloproliferative sarcoma virus enhancer, negative control region deleted, d1587rev primer-binding site substituted (MND)” promoter, an NFAT promoter, a TETON® promoter, and an NFκB promoter. In some embodiments, the promoter is EF1-α or PGK promoter. In some embodiments, the vector is a viral vector. In some embodiments, the viral vector is selected from the group consisting of an adenoviral vector, an adeno-associated virus vector, a retroviral vector, a lentiviral vector, an episomal vector expression vector, a herpes simplex viral vector, and derivatives thereof. In some embodiments, the vector is a lentiviral vector. In some embodiments, the vector is a non-viral vector. In some embodiments, the vector is a Piggybac vector or a Sleeping Beauty vector.

Thus in some embodiments, there is provided a modified T cell (e.g., allogeneic or autologous T cell) comprising: a second vector (e.g., a viral vector, such as a lentiviral vector) comprising a nucleic acid encoding a functional exogenous receptor (e.g., ITAM-modified CAR, ITAM-modified TCR, ITAM-modified cTCR, or ITAM-modified TAC-like chimeric receptor) comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, there is provided a modified T cell (e.g., allogeneic or autologous T cell) comprising: a vector (e.g., a viral vector, such as a lentiviral vector) comprising a nucleic acid encoding an ITAM-modified CAR comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional hinge domain (e.g., derived from CD8α), (c) a transmembrane domain (e.g., derived from CD8α), and (d) an ISD comprising an optional co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the ITAM-modified CAR is an ITAM-modified BCMA CAR, comprising: (a) an extracellular ligand binding domain comprising i) an anti-BCMA scFv or ii) a first sdAb moiety (e.g., V_{H}H) that specifically binds to BCMA, an optional linker, and a second sdAb moiety (e.g., V_{H}H) that specifically binds to BCMA, (b) a hinge domain (e.g., derived from CD8α), (c) a transmembrane domain (e.g., derived from CD8α), and (d) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, wherein the co-stimulatory signaling domain is N-terminal to the CMSD. In some embodiments, the ITAM-modified CAR is an ITAM-modified CD20 CAR, comprising: (a) an extracellular ligand binding domain comprising an anti-CD20 scFv, (b) a hinge domain (e.g., derived from CD8α), (c) a transmembrane domain (e.g., derived from CD8α), and (d) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, wherein the co-stimulatory signaling domain is N-terminal to the CMSD. In some embodiments, the hinge domain comprises the amino acid sequence of SEQ ID NO: 125. In some embodiments, the transmembrane domain comprises the amino acid sequence of SEQ ID NO: 126. In some embodiments, the co-stimulatory signaling domain comprises the amino acid sequence of SEQ ID NO: 124. In some embodiments, the ITAM-modified BCMA CAR comprises the sequence of any of SEQ ID NOs: 76-96 and 106-113. In some embodiments, the ITAM-modified CD20 CAR comprises the sequence any of SEQ ID NOs: 98-104. In some embodiments, there is provided a modified T cell (e.g., allogeneic or autologous T cell) comprising: a vector (e.g., a viral vector, such as a lentiviral vector) comprising a nucleic acid encoding an ITAM-modified BCMA CAR, wherein the ITAM-modified BCMA CAR comprises the sequence of any of SEQ ID NOs: 76-96 and 106-113. In some embodiments, there is provided a modified T cell (e.g., allogeneic or autologous T cell) comprising: a vector (e.g., a viral vector, such as a lentiviral vector) comprising a nucleic acid encoding an ITAM-modified CD20 CAR, wherein the ITAM-modified CD20 CAR comprises the amino acid sequence of any of SEQ ID NOs: 98-104. In some embodiments, the vector is a viral vector (e.g., lentiviral vector). In some embodiments, the vector promoter is EF1-α or PGK promoter.

In some embodiments, the ITAM-modified functional exogenous receptor-T cell (e.g., ITAM-modified CAR-T cell, ITAM-modified TCR-T cell, ITAM-modified cTCR-T cell, or ITAM-modified TAC-like chimeric receptor-T cell) comprises unmodified endogenous TCR (e.g., TCRα and/or TCRβ) loci and/or B2M locus. In some embodiments, the ITAM-modified functional exogenous receptor-T cell comprises a modified endogenous TCR (e.g., TCRα and/or TCRβ) locus and/or a modified endogenous B2M locus. In some embodiments, the endogenous TCR locus is modified by a gene editing system selected from CRISPR-Cas, TALEN, shRNA, and ZFN. In some embodiments, the endogenous TCR locus is modified by a CRISPR-Cas system. In some embodiments, the nucleic acid(s) encoding the gene editing system and the nucleic acid encoding a functional exogenous receptor comprising a CMSD (e.g., ITAM-modified CAR, ITAM-modified TCR, ITAM-modified cTCR, or ITAM-modified TAC-like chimeric receptor) are on the same vector (e.g., under the same promoter control or separate promoter control). In some embodiments, the nucleic acid(s) encoding the gene editing system and the nucleic acid encoding a functional exogenous receptor comprising a CMSD are on different vectors.

Further provided are modified T cells (e.g., allogeneic or autologous T cell) obtained by introducing any of the vectors (e.g., viral vector such as lentiviral vector) described herein. Further provided are modified T cells (e.g., allogeneic or autologous T cell) obtained by any of the methods described herein.

### Effector Function

“Effector function” as used herein refers to biological activity of a molecule (e.g., TCR (e.g., TCRα and/or TCRβ), MHC I, CD3ε, CD3δ, CD3γ, CD3ζ, CD4, CD28, or functional extracellular receptor comprising a CMSD described herein). For example, the effector function of TCR (e.g., TCRα and/or TCRβ), CD3ε, CD3δ, CD3γ, CD3ζ, CD4, CD28, functional extracellular receptor comprising a CMSD described herein, ITAM-containing molecule, CD3ζ ISD-containing molecule (e.g., traditional CAR), or CMSD-containing molecule (or modified T cell comprising thereof) can be signal transduction, such as signal transduction related to T cell stimulation, T cell activation, T cell proliferation, cytokine production, regulatory or cytolytic activity of a T cell, etc. The effector function of an ITAM-containing molecule, CMSD-containing molecule, or CMSD can be signal transduction aforementioned, and/or can be serving as a docking site for other signaling molecules. The effector function of MHC I can be epitope presentation, etc.

Down-modulation of a molecule (e.g., TCR (e.g., TCRα and/or TCRβ), MHC I, CD3ε, CD3δ, CD3γ, CD3ζ, CD4, CD28, or functional extracellular receptor comprising a CMSD described herein) encompass down-regulation of cell surface expression of a molecule, and/or down-regulation of effector function of a molecule or a cell (e.g., modified T cell) comprising such molecule. To test if the expression of an exogenous Nef protein (e.g., wt or mutant Nef) down-modulates (e.g., down-regulate cell surface expression and/or effector function of) TCR (e.g., TCRα and/or TCRβ), MHC I, CD3ε, CD3δ, CD3γ, CD3ζ, CD4, CD28, functional extracellular receptor comprising a CMSD described herein, etc., or to test if the exogenous Nef protein interacts with (e.g., binds to) the aforementioned molecules, one can either test if there is down-regulation of cell surface expression of the protein, or if signaling molecule-mediated signal transduction (e.g., TCR/CD3 complex-mediated signal transduction) is affected (e.g., abolished or attenuated). The effector function of TCR, CMSD-containing functional extracellular receptors, or modified T cells comprising thereof, etc. can be measured by various methods known in the art for studying effector functions of traditional CAR, traditional CAR-T, or cell receptors (e.g., by measuring cytokine release or receptor-mediated cytotoxicity). Also see Examples for exemplary testing methods.

For example, receptor-mediated cytotoxicity on target cells (e.g., tumor cells) can be measured for T cells expressing CMSD-containing functional extracellular receptors, for example, by using target cells with a luciferase label (e.g., Raji.Luc) for in vitro testing, or for in vivo testing on tumor size. In some embodiments, the extracellular receptor-mediated release of pro-inflammatory factor, chemokine and/or cytokine can be measured. If receptor-mediated cytotoxicity and/or release of pro-inflammatory factor, chemokine and/or cytokine is less than that of a same functional extracellular receptor comprising a CD3ζISD (e.g., a same traditional CAR with everything else the same but with a CD3ζISD), it indicates that the CMSD-containing functional extracellular receptor has less effector function compared to that of a same functional extracellular receptor comprising a CD3ζISD; vice versa. Or, if receptor-mediated cytotoxicity and/or release of pro-inflammatory factor, chemokine and/or cytokine is reduced with the presence of an exogenous Nef protein co-expressed in the modified T cell, it reflects interaction between the exogenous Nef protein and the functional extracellular receptor, or that the exogenous Nef protein down-modulates (e.g., down-regulate cell surface expression and/or effector function of) the functional exogenous receptor.

To test if the expression of an exogenous Nef protein down-modulates signaling molecule-mediated signal transduction, e.g., TCR/CD3 complex-mediated signal transduction, cells (e.g., T cells) transduced/transfected with a vector encoding the exogenous Nef protein can be induced with phytohemagglutinin (PHA) for T cell activation. PHA binds to sugars on glycosylated surface proteins, including TCRs, and thereby crosslinks them. This triggers calcium-dependent signaling pathways leading to nuclear factor of activated T cells (NFATs) activation. These cells can then be tested for CD69+ rate using FACS using e.g., PE anti-human CD69 Antibody, to detect PHA-mediated T cell activation under the influence of the exogenous Nef protein.

In some embodiments, the binding of a Nef protein with a signaling molecule, such as CMSD of the functional exogenous receptor described herein or TCR, can also be determined using regular biochemical methods, such as immunoprecipitation and immunofluorescence. Also see Examples for exemplary testing methods.

To test if the expression of an exogenous Nef protein down-regulates cell surface expression of TCR (e.g., TCRα and/or TCRβ), cells (e.g., T cells) transduced/transfected with a vector encoding the exogenous Nef protein can be subjected to FACS or MACS sorting using anti-TCRα and/or anti-TCRβ antibody (also see Examples). For example, transduced/transfected cells can be incubated with PE/Cy5 anti-human TCRαβ antibody (e.g., Biolegend, #306710) for FACS to detect TCRαβ positive rate, or incubated with biotinylated human TCRαp antibody (Miltenyi, 200-070-407) for biotin labeling then subject to magnetic separation and enrichment according to the MACS kit protocols.

To test if the expression of an exogenous Nef protein down-regulates cell surface expression of a functional extracellular receptor comprising a CMSD described herein, one can use labeled antigen recognized by the functional extracellular receptor, for example, FITC-Labeled Human BCMA protein (e.g., ACROBIOSYSTEM, BCA-HF254-200UG) for FACS to detect ITAM-modified BCMA CAR expression. Also see Examples for exemplary testing methods.

### III. CMSD-Containing Functional Exogenous Receptors

The T cells described herein comprise a CMSD-containing functional exogenous receptor. The present application in one aspect also provides such CMSD-containing functional exogenous receptors and cells (e.g., effector cells such as T cells) expressing such.

In some embodiments, the functional exogenous receptor comprises: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD, wherein the CMSD comprises one or a plurality of ITAMs (“CMSD ITAMs”), wherein the plurality of CMSD ITAMs are optionally connected by one or more linkers (“CMSD linkers”). In some embodiments, the plurality (e.g., 2, 3, 4, or more) of CMSD ITAMs are directly linked to each other. In some embodiments, the CMSD comprises two or more (e.g., 2, 3, 4, or more) CMSD ITAMs connected by one or more CMSD linkers not derived from an ITAM-containing parent molecule (e.g., G/S linker). In some embodiments, the CMSD comprises one or more CMSD linkers derived from an ITAM-containing parent molecule that is different from the ITAM-containing parent molecule from which one or more of the CMSD ITAMs are derived from. In some embodiments, the CMSD comprises two or more (e.g., 2, 3, 4, or more) identical CMSD ITAMs. In some embodiments, at least one of the CMSD ITAMs is not derived from CD3ζ. In some embodiments, at least one of the CMSD ITAMs is not ITAM1 or ITAM2 of CD3ζ. In some embodiments, at least two of the CMSD ITAMs are different from each other. In some embodiments, the plurality of CMSD ITAMs are each derived from a different ITAM-containing parent molecule. In some embodiments, at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, the CMSD consists essentially of (e.g., consists of) one CMSD ITAM. In some embodiments, the CMSD comprises (e.g., consists essentially of or consists of) one CMSD ITAM (e.g., derived from CD3ε, CD3δ, or CD3γ) and a CMSD N-terminal sequence and/or a CMSD C-terminal sequence that is heterologous to the ITAM-containing parent molecule (e.g., a G/S linker). In some embodiments, at least one or plurality of CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin. In some embodiments, the plurality of CMSD ITAMs are derived from one or more of CD3ε, CD3δ, CD3γ, CD3ζ, DAP12, Igα (CD79a), Igβ (CD79b), and FcεRIγ. In some embodiments, the CMSD does not comprise CD3ζ ITAM1 and/or CD3ζ ITAM2. In some embodiments, at least one of the CMSD ITAMs is CD3ζ ITAM3. In some embodiments, the CMSD does not comprise any ITAMs from CD3ζ. In some embodiments, at least two of the CMSD ITAMs are derived from the same ITAM-containing parent molecule. In some embodiments, the CMSD comprises the amino acid sequence of any of SEQ ID NOs: 41-74. In some embodiments, the ISD further comprises a co-stimulatory signaling domain (e.g., derived from CD28 or 4-1BB). In some embodiments, the co-stimulatory domain is N-terminal to the CMSD. In some embodiments, the co-stimulatory domain is C-terminal to the CMSD. In some embodiments, the co-stimulatory signaling domain comprises the amino acid sequence of SEQ ID NO: 124. In some embodiments, the transmembrane domain comprises a sequence of SEQ ID NO: 126. In some embodiments, the hinge domain comprises the sequence of SEQ ID NO: 125. In some embodiments, the CMSD-containing functional exogenous receptor further comprises a signal peptide (e.g., derived from CD8α) located at the N-terminus of the functional exogenous receptor. In some embodiments, the signal peptide comprises the sequence of SEQ ID NO: 127.

In some embodiments, the functional exogenous receptor comprising a CMSD described herein is not down-modulated (e.g., down-regulated for cell surface expression and/or effector function such as signal transduction involved in cytolytic activity) by a Nef protein (e.g., wildtype Nef such as wildtype SIV Nef, or mutant Nef such as mutant SIV Nef). In some embodiments, the functional exogenous receptor comprising a CMSD described herein is at most about 80% (such as at most about any of 70%, 60%, 50%, 40%, 30%, 20%, 10%, or 5%) down-modulated (e.g., down-regulated for cell surface expression and/or effector function such as signal transduction involved in cytolytic activity) by a Nef protein (e.g., wildtype Nef such as wildtype SIV Nef, or mutant Nef such as mutant SIV Nef) compared to when the Nef is absent. In some embodiments, the functional exogenous receptor comprising a CMSD is down-modulated (e.g., down-regulated for cell surface expression and/or effector function such as signal transduction involved in cytolytic activity) by a Nef protein (e.g., wt, subtype, or mutant Nef) the same or similarly as a same exogenous receptor comprising a CD3ζISD (e.g., traditional CAR comprising everything the same but with a CD3ζISD). In some embodiments, the functional exogenous receptor comprising a CMSD is at least about 3% less (e.g., at least about any of 4%, 5%, 6%, 7%, 8%, 9%, 10%, 15%, 20%, 30%, 40%, 50%, 60%, 70%, 80%, 90%, or 95% less) down-modulated (e.g., down-regulated for cell surface expression and/or effector function such as signal transduction involved in cytolytic activity) by a Nef protein (e.g., wildtype Nef such as wildtype SIV Nef, or mutant Nef such as mutant SIV Nef) than a same exogenous receptor comprising a CD3ζISD (e.g., traditional CAR with CD3ζISD).

The various components of the CMSD-containing functional exogenous receptors, as well as specific functional exogenous receptors (such as ITAM-modified CAR, ITAM-modified TCR, ITAM-modified cTCR, and ITAM-modified TAC-like chimer receptor), are further described below in more details.

### CMSD

Chimeric signaling domain (“CMSD”) described herein comprises one or more ITAMs (also referred to herein as “CMSD ITAMs”) and optional linkers (also referred to herein as “CMSD linkers”) arranged in a configuration that is different than any of the naturally occurring ITAM-containing parent molecules. For example, in some embodiments, the CMSD comprises two or more ITAMs directly linked to each other. In some embodiments, the CMSD comprises ITAMs connected by one or more “heterologous linkers”, namely, linker sequences which are either not derived from an ITAM-containing parent molecule (e.g., G/S linkers), or derive from an ITAM-containing parent molecule that is different from the ITAM-containing parent molecule from which one or more of the CMSD ITAMs are derived from. In some embodiments, the CMSD comprises two or more (such as 2, 3, 4, or more) identical ITAMs. In some embodiments, at least two of the CMSD ITAMs are different from each other. In some embodiments, at least one of the CMSD ITAMs is not derived from CD3ζ. In some embodiments, at least one of the CMSD ITAMs is not ITAM1 or ITAM2 of CD3ζ. In some embodiments, the CMSD does not comprise CD3ζ ITAM1 and/or CD3ζ ITAM2. In some embodiments, at least one of the CMSD ITAMs is CD3ζ ITAM3. In some embodiments, the CMSD does not comprise any ITAMs from CD3ζ. In some embodiments, at least two of the CMSD ITAMs are derived from the same ITAM-containing parent molecule. In some embodiments, the CMSD comprises two or more (such as 2, 3, 4, or more) ITAMs, wherein at least two of the CMSD ITAMs are each derived from a different ITAM-containing parent molecule. In some embodiments, at least one of the CMSD ITAMs is derived from an ITAM-containing parent molecule selected from the group consisting of: CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, the CMSD consists essentially of (e.g., consists of) one CMSD ITAM. In some embodiments, the CMSD consists essentially of (e.g., consists of) one CMSD ITAM (e.g., derived from CD3ε, CD3δ, or CD3γ) and a CMSD N-terminal sequence and/or a CMSD C-terminal sequence that is “heterologous” to the ITAM-containing parent molecule (e.g., a G/S linker), i.e., the CMSD N-terminal sequence and/or the CMSD C-terminal sequence is either not derived from an ITAM-containing parent molecule (e.g., G/S containing sequence), or derive from an ITAM-containing parent molecule that is different from the ITAM-containing parent molecule from which the CMSD ITAM (e.g., one or more CMSD ITAMs) is derived from. In some embodiments, the CMSD comprises ITAM1, ITAM2, and ITAM3 of CD3ε, but a) two or three of the ITAMs are not connected by linker; b) the three ITAMs are not arranged in the right order compared to that in CD3ζ; c) at least one of the ITAMs is at a different location compared to the corresponding ITAM in CD3ζ; d) at least two of the ITAMs are connected by a heterologous linker; and/or e) the CMSD further comprises an additional CMSD ITAM.

Thus, for example, in some embodiments, the CMSD comprises one or a plurality of CMSD ITAMs, wherein the plurality of CMSD ITAMs are optionally connected by one or more linkers (“CMSD linkers”), wherein:
(a) the plurality (e.g., 2, 3, 4, or more) of CMSD ITAMs are directly linked to each other;
(b) the CMSD comprises two or more (e.g., 2, 3, 4, or more) CMSD ITAMs connected by one or more linkers not derived from an ITAM-containing parent molecule (e.g., G/S linker);
(c) the CMSD comprises one or more CMSD linkers derived from an ITAM-containing parent molecule that is different from the ITAM-containing parent molecule from which one or more of the CMSD ITAMs are derived from;
(d) the CMSD comprises two or more (e.g., 2, 3, 4, or more) identical CMSD ITAMs;
(e) at least one of the CMSD ITAMs is not derived from CD3ζ;
(f) at least one of the CMSD ITAMs is not ITAM1 or ITAM2 of CD3ζ;
(g) the plurality of CMSD ITAMs are each derived from a different ITAM-containing parent molecule;
(h) at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin;
(i) the CMSD consists of one CMSD ITAM; and/or
(j) the CMSD consists essentially of (e.g., consists of) one CMSD ITAM and a CMSD N-terminal sequence and/or a CMSD C-terminal sequence that is heterologous to the ITAM-containing parent molecule (e.g., a G/S linker).

In some embodiments, the CMSD possesses two or more of the characteristics described above. For example, in some embodiments, (a) the plurality (e.g., 2, 3, 4, or more) of CMSD ITAMs are directly linked to each other, and (d) the CMSD comprises two or more (e.g., 2, 3, 4, or more) identical CMSD ITAMs. In some embodiments, (b) the CMSD comprises two or more (e.g., 2, 3, 4, or more) CMSD ITAMs connected by one or more linkers not derived from an ITAM-containing parent molecule (e.g., G/S linker), and (d) the CMSD comprises two or more (e.g., 2, 3, 4, or more) identical CMSD ITAMs. In some embodiments, (c) the CMSD comprises one or more CMSD linkers derived from an ITAM-containing parent molecule that is different from the ITAM-containing parent molecule from which one or more of the CMSD ITAMs are derived from, and (d) the CMSD comprises two or more (e.g., 2, 3, 4, or more) identical CMSD ITAMs. In some embodiments, (f) at least one of the CMSD ITAMs is not ITAM1 or ITAM2 of CD3ζ, and (h) at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, (b) the CMSD comprises two or more (e.g., 2, 3, 4, or more) CMSD ITAMs connected by one or more linkers not derived from an ITAM-containing parent molecule (e.g., G/S linker), and (f) at least one of the CMSD ITAMs is not ITAM1 or ITAM2 of CD3ζ. In some embodiments, (b) the CMSD comprises two or more (e.g., 2, 3, 4, or more) CMSD ITAMs connected by one or more linkers not derived from an ITAM-containing parent molecule (e.g., G/S linker), and (h) at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, (b) the CMSD comprises two or more (e.g., 2, 3, 4, or more) CMSD ITAMs connected by one or more linkers not derived from an ITAM-containing parent molecule (e.g., G/S linker), (d) the CMSD comprises two or more (e.g., 2, 3, 4, or more) identical CMSD ITAMs, and (h) at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, (c) the CMSD comprises one or more CMSD linkers derived from an ITAM-containing parent molecule that is different from the ITAM-containing parent molecule from which one or more of the CMSD ITAMs are derived from, and (e) at least one of the CMSD ITAMs is not derived from CD3ζ.

In some embodiments, the ISD of the functional exogenous receptors described herein (e.g., an ITAM-modified TCR, an ITAM-modified CAR, an ITAM-modified cTCR, or an ITAM-modified TAC-like chimeric receptor) consists essentially of (such as consists of) the CMSD. In some embodiments, the ISD of the functional exogenous receptors described herein (e.g., ITAM-modified CAR) further comprises a co-stimulatory signaling domain (e.g., 4-1BB or CD28 co-stimulatory signaling domain), which can be positioned either N-terminal or C-terminal to the CMSD, and is connected to the CMSD via an optional connecting peptide within the CMSD (e.g. connected via the optional CMSD N-terminal sequence or optional CMSD C-terminal sequence).

The CMSD described herein functions as a primary signaling domain in the ISD which acts in a stimulatory manner to induce immune effector functions. For example, effector function of a T cell may be cytolytic activity or helper activity including the secretion of cytokines. An “ITAM” as used herein, refers to a conserved protein motif that can be found in the tail portion of signaling molecules expressed in many immune cells (e.g., T cell). ITAMs reside in the cytoplasmic domain of many cell surface receptors (e.g., TCR complex) or subunits they associate with, and play an important regulatory role in signal transmission. Traditional CAR usually comprises a primary ISD of CD3 that contains 3 ITAMs, CD3ζ ITAM1, CD3ζ ITAM2, and CD3ζ ITAM3. However, limitations of using CD3ζ as ISD of CAR have been reported. The ITAMs described herein in some embodiments are naturally occurring, i.e., can be found in a naturally occurring ITAM-containing parent molecule. In some embodiments, the ITAM is further modified, e.g., by making one, two, or more amino acid substitutions, deletions, additions, or relocations relative to a naturally occurring ITAM. In some embodiments, the modified ITAM (hereinafter also referred to as “non-naturally occurring ITAM”) has the same or similar ITAM function (e.g., signal transduction, or as docking site) as compared to the parental ITAM.

ITAM usually comprises two repeats of the amino acid sequence YXXL/I separated by 6-8 amino acid residues, wherein each x is independently any amino acid residue, resulting the conserved motif Y_{XX}L/I-_{X6-8}-YXXL/I (SEQ ID NO: 114). In some embodiments, the ITAM contains a negatively charged amino acid (D/E) in the +2 position relative to the first ITAM tyrosine (Y), resulting a consensus sequence of D/E-_{X0-2}-Y_{XX}L/I-_{X6-8}-Y_{XX}L/I (SEQ ID NO: 115). Exemplary ITAM-containing signaling molecules include CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin, also referred to as “ITAM-containing parent molecule” herein. ITAMs present in an ITAM-containing parent molecule are known to be involved in signal transduction within the cell upon ligand engagement, which is mediated at least in part by phosphorylation of tyrosine residues in the ITAM following activation of the signaling molecule. ITAMs may also function as docking sites for other proteins involved in signaling pathways.

In some embodiments, the ITAM-containing parent molecule is CD3ζ. In some embodiments, the CD3ζISD has the sequence of SEQ ID NO: 7, which comprises CD3ζ ITAM1 (SEQ ID NO: 4), CD3ζ ITAM2 (SEQ ID NO: 5), CD3ζ ITAM3 (SEQ ID NO: 6), and non-ITAM sequences at N-terminal of CD3ζ ITAM1, at C-terminal of CD3ζ ITAM3, and connecting the three ITAMs. In some embodiments, the ITAM-containing parent molecule comprises an ITAM with a sequence selected from the group consisting of SEQ ID NOs: 1-6 and 8-16.

In some embodiments, the CMSD comprises a plurality (e.g., 2, 3, 4, or more) of ITAMs, wherein at least two of which are directly connected with each other. In some embodiments, the CMSD comprises a plurality of ITAMs, wherein at least two of the ITAMs are connected by a heterologous linker. In some embodiments, the CMSD further comprises an N-terminal sequence at the N-terminus of the most N-terminal CMSD ITAM (herein also referred to as “CMSD N-terminal sequence”). In some embodiments, the CMSD further comprises a C-terminal sequence at the C-terminus of the most C-terminal CMSD ITAM (herein also referred to as “CMSD C-terminal sequence”). In some embodiments, the CMSD linker(s), CMSD N-terminal sequence, and/or C-terminal CMSD sequence are selected from the group consisting of SEQ ID NOs: 17-39 and 116-120, such as any of SEQ ID NOs: 17-31. In some embodiments, the CMSD linker(s), CMSD N-terminal sequence, and/or CMSD C-terminal sequence are about 1 to about 15 (such as about any of 1, 2, 3, 4, 5, 6, 7, 8, 9, 10, 11, 12, 13, 14, 15, or any ranges in-between) amino acids long. In some embodiments, the heterologous linker is a G/S linker. In some embodiments, the heterologous linker(s) is selected from the group consisting of SEQ ID NOs: 17-19, 23, 25-29. In some embodiments, the CMSD C-terminal sequence is selected from the group consisting of SEQ ID NOs: 18, 20, 25, and 27-29. In some embodiments, the CMSD N-terminal sequence is selected from the group consisting of SEQ ID NOs: 17, 21, 22, 24, 30, and 31. In some embodiments, the heterologous linker is derived from an ITAM-containing parent molecule that is different from the ITAM-containing parent molecule from which one or more of the CMSD ITAMs are derived from. In some embodiments, the CMSD does not comprise any CMSD linker, CMSD N-terminal sequence, and/or C-terminal CMSD sequence.

In some embodiments, a one-ITAM containing CMSD comprises from N′ to C′: optional CMSD N-terminal sequence—CMSD ITAM—optional CMSD C-terminal sequence. In some embodiments, the CMSD described herein comprises from N′ to C′: optional CMSD N-terminal sequence—CD3ε ITAM—optional CMSD C-terminal sequence. In some embodiments, the CMSD described herein comprises from N′ to C′: optional CMSD N-terminal sequence—CD36 ITAM—optional CMSD C-terminal sequence. In some embodiments, the CMSD comprises a sequence of SEQ ID NO: 67 (hereinafter also referred to as “ITAM033” or “ITAM033 construct”). In some embodiments, the CMSD comprises a sequence of SEQ ID NO: 68 (hereinafter also referred to as “ITAM034” or “ITAM034 construct”).

In some embodiments, a two-ITAM containing CMSD comprises from N′ to C′: optional CMSD N-terminal sequence—first CMSD ITAM—optional CMSD linker—second CMSD ITAM—optional CMSD C-terminal sequence. In some embodiments, the CMSD described herein comprises from N′ to C′: optional CMSD N-terminal sequence—CD36 ITAM—optional CMSD linker—CD3ε ITAM—optional CMSD C-terminal sequence. In some embodiments, the CMSD described herein comprises from N′ to C′: optional CMSD N-terminal sequence—CD3γ ITAM—optional CMSD linker—DAP12 ITAM—optional CMSD C-terminal sequence. In some embodiments, the CMSD linker is identical to CD3ζ first linker or CD3ζ second linker. In some embodiments, the CMSD comprises a sequence of SEQ ID NO: 69 (hereinafter also referred to as “ITAM035” or “ITAM035 construct”). In some embodiments, the CMSD comprises a sequence of SEQ ID NO: 70 (hereinafter also referred to as “ITAM036” or “ITAM036 construct”).

In some embodiments, a three-ITAM containing CMSD comprises from N′ to C′: optional CMSD N-terminal sequence—first CMSD ITAM—optional first CMSD linker—second CMSD ITAM—optional second CMSD linker—third CMSD ITAM—optional CMSD C-terminal sequence. See, FIG. 8 for exemplary structures. In some embodiments, the CMSD described herein comprises from N′ to C′: optional CMSD N-terminal sequence—CD3ζ ITAM1—optional first CMSD linker—CD3ζ ITAM2—optional second CMSD linker—CD3ζ ITAM3—optional CMSD C-terminal sequence, wherein at least one of the first CMSD linker and the second CMSD linker is absent or heterologous to CD3ζ. In some embodiments, the first CMSD linker can be identical to CD3ζ second linker, and the second CMSD linker can be identical to CD3ζ first linker. In some embodiments, the first CMSD linker and the second CMSD linker can be both identical to CD3ζ first linker. In some embodiments, the first CMSD linker and the second CMSD linker can be both identical to CD3ζ second linker. In some embodiments, the CMSD described herein comprises a sequence of SEQ ID NO: 41 (hereinafter also referred to as “M663 CMSD”). In some embodiments, the CMSD described herein comprises a sequence of SEQ ID NO: 54 (hereinafter also referred to as “ITAM007” or “ITAM007 construct”).

In some embodiments, the CMSD described herein comprises from N′ to C′: optional CMSD N-terminal sequence—CD3ζ ITAM1—optional first CMSD linker—CD3ζ ITAM1—optional second CMSD linker—CD3ζ ITAM1—optional CMSD C-terminal sequence, wherein the optional first CMSD linker and/or second CMSD linker can be either absent or of any linker sequence suitable for the effector function signal transduction of the CMSD (e.g., the first CMSD linker can be identical to CD3ζ first linker, the second CMSD linker can be identical to CD3ζ second linker, see FIG. 8). In some embodiments, the CMSD described herein comprises a sequence of SEQ ID NO: 42 (hereinafter also referred to as “M665 CMSD”). In some embodiments, the CMSD described herein comprises a sequence of SEQ ID NO: 55 (hereinafter also referred to as “ITAM008” or “ITAM008 construct”).

In some embodiments, the CMSD described herein comprises from N′ to C′: optional CMSD N-terminal sequence—CD3ζ ITAM2—optional first CMSD linker—CD3ζ ITAM2—optional second CMSD linker—CD3ζ ITAM2—optional CMSD C-terminal sequence, wherein the optional first CMSD linker and/or second CMSD linker can be either absent or of any linker sequence suitable for the effector function signal transduction of the CMSD (e.g., the first CMSD linker can be identical to CD3ζ first linker, the second CMSD linker can be identical to CD3ζ second linker). In some embodiments, the CMSD described herein comprises a sequence of SEQ ID NO: 43 (hereinafter also referred to as “M666 CMSD”).

In some embodiments, the CMSD described herein comprises from N′ to C′: optional CMSD N-terminal sequence—CD3ζ ITAM3—optional first CMSD linker—CD3ζ ITAM3—optional second CMSD linker—CD3ζ ITAM3—optional CMSD C-terminal sequence, wherein the optional first CMSD linker and/or second CMSD linker can be either absent or of any linker sequence suitable for the effector function signal transduction of the CMSD (e.g., the first CMSD linker can be identical to CD3ζ first linker, the second CMSD linker can be identical to CD3ζ second linker). In some embodiments, the CMSD described herein comprises a sequence of SEQ ID NO: 44 (hereinafter also referred to as “M667 CMSD”).

In some embodiments, the CMSD described herein comprises from N′ to C′: optional CMSD N-terminal sequence—CD3ζ ITAM1—optional first CMSD linker—CD3ζ ITAM2—optional second CMSD linker—CD3ζ ITAM2—optional CMSD C-terminal sequence. In some embodiments, the CMSD described herein comprises from N′ to C′: optional CMSD N-terminal sequence—CD3ζ ITAM1—optional first CMSD linker—CD3ζ ITAM3—optional second CMSD linker—CD3ζ ITAM3—optional CMSD C-terminal sequence. In some embodiments, the CMSD described herein comprises from N′ to C′: optional CMSD N-terminal sequence—CD3ζ ITAM1—optional first CMSD linker—CD3ζ ITAM3—optional second CMSD linker—CD3ζ ITAM2—optional CMSD C-terminal sequence. In some embodiments, the CMSD described herein comprises from N′ to C′: optional CMSD N-terminal sequence—CD3ζ ITAM2—optional first CMSD linker—CD3ζ ITAM1—optional second CMSD linker—CD3ζ ITAM1—optional CMSD C-terminal sequence. In some embodiments, the CMSD described herein comprises from N′ to C′: optional CMSD N-terminal sequence—CD3ζ ITAM2—optional first CMSD linker—CD3ζ ITAM1—optional second CMSD linker—CD3ζ ITAM2—optional CMSD C-terminal sequence. In some embodiments, the CMSD described herein comprises from N′ to C′: optional CMSD N-terminal sequence—CD3ζ ITAM2—optional first CMSD linker—CD3ζ ITAM1—optional second CMSD linker—CD3ζ ITAM3—optional CMSD C-terminal sequence. In some embodiments, the CMSD described herein comprises from N′ to C′: optional CMSD N-terminal sequence—CD3ζ ITAM2—optional first CMSD linker—CD3ζ ITAM3—optional second CMSD linker—CD3ζ ITAM3—optional CMSD C-terminal sequence. In some embodiments, the CMSD described herein comprises from N′ to C′: optional CMSD N-terminal sequence—CD3ζ ITAM3—optional first CMSD linker—CD3ζ ITAM1—optional second CMSD linker—CD3ζ ITAM1—optional CMSD C-terminal sequence. In some embodiments, the CMSD described herein comprises from N′ to C′: optional CMSD N-terminal sequence—CD3ζ ITAM3—optional first CMSD linker—CD3ζ ITAM1—optional second CMSD linker—CD3ζ ITAM2—optional CMSD C-terminal sequence. In some embodiments, the CMSD described herein comprises from N′ to C′: optional CMSD N-terminal sequence—CD3ζ ITAM3—optional first CMSD linker—CD3ζ ITAM1—optional second CMSD linker—CD3ζ ITAM3—optional CMSD C-terminal sequence. In some embodiments, the CMSD described herein comprises from N′ to C′: optional CMSD N-terminal sequence—CD3ζ ITAM3—optional first CMSD linker—CD3ζ ITAM2—optional second CMSD linker—CD3ζ ITAM2—optional CMSD C-terminal sequence. In some embodiments, the CMSD described herein comprises from N′ to C′: optional CMSD N-terminal sequence—CD3ζ ITAM3—optional first CMSD linker—CD3ζ ITAM2—optional second CMSD linker—CD3ζ ITAM3—optional CMSD C-terminal sequence. In some embodiments, the CMSD does not comprise any ITAM (e.g., ITAM1, ITAM2, or ITAM3) of CD3ζ. In some embodiments, the 3-ITAM containing CMSD comprises one or more (e.g., 1, 2, or 3) ITAMs derived from a non-CD3ζ ITAM-containing parent molecule (e.g., CD3ε, CD3δ, CD3γ, Igα (CD79a), Igβ (CD79b), FcεRIβ, FcεRIγ, DAP12, CNAIP/NFAM1, STAM-1, STAM-2, or Moesin), and the optional linker(s) connecting them can be absent or of any linker sequence suitable for the effector function signal transduction of the CMSD (e.g., the first CMSD linker can be identical to CD3ζ first linker, the second CMSD linker can be identical to CD3ζ second linker, or G/S linker).

Thus in some embodiments, the CMSD described herein comprises from N′ to C′: optional CMSD N-terminal sequence—CD3ε ITAM—optional first CMSD linker—CD3ε ITAM—optional second CMSD linker—CD3ε ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 46 (hereinafter also referred to as “M679 CMSD”). In some embodiments, the CMSD described herein comprises a sequence of SEQ ID NO: 56 (hereinafter also referred to as “ITAM009” or “ITAM009 construct”).

In some embodiments, the CMSD described herein comprises from N′ to C′: optional CMSD N-terminal sequence—DAP12 ITAM—optional first CMSD linker—DAP12 ITAM—optional second CMSD linker—DAP12 ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 48 (hereinafter also referred to as “M681 CMSD”).

In some embodiments, the CMSD described herein comprises from N′ to C′: optional CMSD N-terminal sequence—Igα ITAM—optional first CMSD linker—Igα ITAM—optional second CMSD linker—Igα ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 49 (hereinafter also referred to as “M682 CMSD”).

In some embodiments, the CMSD described herein comprises from N′ to C′: optional CMSD N-terminal sequence—Igβ ITAM—optional first CMSD linker—Igβ ITAM—optional second CMSD linker—Igβ ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 50 (hereinafter also referred to as “M683 CMSD”).

In some embodiments, the CMSD described herein comprises from N′ to C′: optional CMSD N-terminal sequence—FcεRIγITAM—optional first CMSD linker—FcεRIγITAM—optional second CMSD linker—FcεRIγITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 52 (hereinafter also referred to as “M685 CMSD”).

In some embodiments, the CMSD described herein comprises from N′ to C′: optional CMSD N-terminal sequence—CD36 ITAM—optional first CMSD linker—CD36 ITAM—optional second CMSD linker—CD36 ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 45 (hereinafter also referred to as “M678 CMSD”).

In some embodiments, the CMSD described herein comprises from N′ to C′: optional CMSD N-terminal sequence—CD3γ ITAM—optional first CMSD linker—CD3γ ITAM—optional second CMSD linker—CD3γ ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 47 (hereinafter also referred to as “M680 CMSD”).

In some embodiments, the CMSD described herein comprises from N′ to C′: optional CMSD N-terminal sequence—FcεRIβITAM—optional first CMSD linker—FcεRIβITAM—optional second CMSD linker—FcεRIβITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 51 (hereinafter also referred to as “M684 CMSD”).

In some embodiments, the CMSD described herein comprises from N′ to C′: optional CMSD N-terminal sequence—CNAIP/NFAM1 ITAM—optional first CMSD linker—CNAIP/NFAM1 ITAM—optional second CMSD linker—CNAIP/NFAM1 ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 53 (hereinafter also referred to as “M799 CMSD”).

In some embodiments, the CMSD described herein comprises from N′ to C′: optional CMSD N-terminal sequence—CD36 ITAM—optional first CMSD linker—CD3ε ITAM—optional second CMSD linker—CD3ε ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 71 (hereinafter also referred to as “ITAM037” or “ITAM037 construct”).

In some embodiments, the CMSD described herein comprises from N′ to C′: optional CMSD N-terminal sequence—CD36 ITAM—optional first CMSD linker—CD3ε ITAM—optional second CMSD linker—CD3γ ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 72 (hereinafter also referred to as “ITAM038” or “ITAM038 construct”).

In some embodiments, the CMSD described herein comprises from N′ to C′: optional CMSD N-terminal sequence—DAP12 ITAM—optional first CMSD linker—CD3ε ITAM—optional second CMSD linker—CD36 ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 73 (hereinafter also referred to as “ITAM045” or “ITAM045 construct”).

In some embodiments, the CMSD described herein comprises from N′ to C′: optional CMSD N-terminal sequence—DAP12 ITAM—optional first CMSD linker—CD36 ITAM—optional second CMSD linker—CD3ε ITAM—optional CMSD C-terminal sequence. In some embodiments, the one or more CMSD linkers is identical to CD3ζ first linker or CD3ζ second linker. In some embodiments, the CMSD described herein comprises a sequence of SEQ ID NO: 74 (hereinafter also referred to as “ITAM046” or “ITAM046 construct”).

In some embodiments, the CMSD described herein comprises from N′ to C′: cytoplasmic CD3ζ N-terminal sequence—first CMSD ITAM—CD3ζ first linker—second CMSD ITAM—CD3ζ second linker—third CMSD ITAM—CD3ζ C-terminal sequence, wherein all non-ITAM sequences (cytoplasmic CD3ζ N-terminal sequence, CD3ζ first linker, CD3ζ second linker, and CD3ζ C-terminal sequence) within the CMSD are identical to and at the same position as they naturally reside in the parent CD3ζISD, such CMSD is also referred to as “CMSD comprising a non-ITAM CD3ζISD framework” (see FIG. 8). For a CMSD comprising a non-ITAM CD3ζISD framework, the first/second/third CMSD ITAMs can be independently selected from the group consisting of CD36 ITAM, CD3γ ITAM, CD3ζ ITAM11, CD3ζ ITAM2, CD3ζ ITAM3, DAP12 ITAM, CNAIP/NFAM1 ITAM, Igα ITAM, Igβ ITAM, and FcεRIγITAM (SEQ ID NOs: 1, 3-6, 8-11, and 13; all 29 amino acids long), except the combination where the first CMSD ITAM is CD3ζ ITAM1, the second CMSD ITAM is CD3ζ ITAM2, and the third CMSD ITAM is CD3ζ ITAM3. For example, in some embodiments, the CMSD described herein comprises (e.g., consisting of) from N′ to C′: cytoplasmic CD3 N-terminal sequence—DAP12 ITAM—CD3ζ first linker—DAP12 ITAM—CD3ζ second linker—DAP12 ITAM—CD3ζ C-terminal sequence. In some embodiments, the CMSD described herein comprises (e.g., consisting of) from N′ to C′: cytoplasmic CD3 N-terminal sequence—CD3γ ITAM—CD3ζ first linker—CD3γ ITAM—CD3ζ second linker—CD3γ ITAM—CD3ζ C-terminal sequence.

In some embodiments, a four-ITAM containing CMSD comprises from N′ to C′: optional CMSD N-terminal sequence—first CMSD ITAM—optional first CMSD linker—second CMSD ITAM—optional second CMSD linker—third CMSD ITAM—optional third CMSD linker —fourth CMSD ITAM—optional CMSD C-terminal sequence. And so on for 5-ITAM containing, 6-ITAM containing, etc., CMSDs. For CMSDs comprising four or more (e.g., 4, 5, or more) ITAMs, since ITAM-containing parent molecules usually comprise 1 ITAM (e.g., non-CD3ζ ITAM-containing molecules, such as CD3ε, CD3δ, CD3γ, Igα (CD79a), Igβ (CD79b), FcεRIβ, FcεRIγ, DAP12, CNAIP/NFAM1, STAM-1, STAM-2, or Moesin) or 3 ITAMs (e.g., CD3ζ), at least one ITAM within the CMSD will be different from one ITAM-containing parent molecule, either derived from a molecule different from the ITAM-containing parent molecule, or reside at a different position from where the ITAM naturally resides in the ITAM-containing parent molecule, thus CMSDs comprising four or more (e.g., 4, 5, or more) ITAMs can comprise ITAMs derived from any ITAM-containing parent molecule described herein (e.g., CD3), the optional linkers can be absent, derived from cytoplasmic non-ITAM sequence of ITAM-containing parent molecules, or of heterologous sequence from ITAM-containing parent molecule (e.g., can be G/S linkers). In some embodiments, the CMSD described herein comprises from N′ to C′: optional CMSD N-terminal sequence—CD36 ITAM (SEQ ID NO: 1)—optional first CMSD linker—CD3ε ITAM (SEQ ID NO: 2)—optional second CMSD linker—CD3γ ITAM (SEQ ID NO: 3)—optional third CMSD linker—DAP12 ITAM (SEQ ID NO: 8)—optional CMSD C-terminal sequence. In some embodiments, the optional CMSD linker(s), CMSD N-terminal sequence, and CMSD C-terminal sequence are derived from cytoplasmic non-ITAM sequence of ITAM-containing parent molecules. In some embodiments, the optional first, second, and third CMSD linkers, optional CMSD N-terminal sequence, and optional CMSD C-terminal sequence are heterologous, and are independently selected from the group consisting of SEQ ID NOs: 17-39 and 116-120, such as any of SEQ ID NOs: 17-31. In some embodiments, the CMSD comprises a sequence of SEQ ID NO: 57 (hereinafter also referred to as “ITAM010” or “ITAM010 construct”). In some embodiments, the CMSD comprises a sequence of SEQ ID NO: 59 (hereinafter also referred to as “ITAM025” or “ITAM025 construct”). In some embodiments, the CMSD comprises a sequence of SEQ ID NO: 60 (hereinafter also referred to as “ITAM026” or “ITAM026 construct”). In some embodiments, the CMSD comprises a sequence of SEQ ID NO: 61 (hereinafter also referred to as “ITAM027” or “ITAM027 construct”). In some embodiments, the CMSD comprises a sequence of SEQ ID NO: 62 (hereinafter also referred to as “ITAM028” or “ITAM028 construct”). In some embodiments, the CMSD comprises a sequence of SEQ ID NO: 63 (hereinafter also referred to as “ITAM029” or “ITAM029 construct”). In some embodiments, the CMSD described herein consists of from N′ to C′: CD36 ITAM—CD3ε ITAM—CD3γ ITAM—DAP12 ITAM. In some embodiments, the CMSD comprises a sequence of SEQ ID NO: 58 (hereinafter also referred to as “ITAM024” or “ITAM024 construct”).

In some embodiments, the CMSD described herein comprises from N′ to C′: optional CMSD N-terminal sequence—CD3ε ITAM—optional first CMSD linker—CD36 ITAM—optional second CMSD linker—DAP12 ITAM—optional third CMSD linker—CD3γ ITAM—optional CMSD C-terminal sequence. In some embodiments, the optional CMSD linker(s), CMSD N-terminal sequence, and CMSD C-terminal sequence are derived from cytoplasmic non-ITAM sequence of ITAM-containing parent molecules. In some embodiments, the CMSD comprises a sequence of SEQ ID NO: 64 (hereinafter also referred to as “ITAM030” or “ITAM030 construct”).

In some embodiments, the CMSD described herein comprises from N′ to C′: optional CMSD N-terminal sequence—CD3γ ITAM—optional first CMSD linker—DAP12 ITAM—optional second CMSD linker—CD36 ITAM—optional third CMSD linker—CD3ε ITAM—optional CMSD C-terminal sequence. In some embodiments, the optional CMSD linker(s), CMSD N-terminal sequence, and CMSD C-terminal sequence are derived from cytoplasmic non-ITAM sequence of ITAM-containing parent molecules. In some embodiments, the CMSD comprises a sequence of SEQ ID NO: 65 (hereinafter also referred to as “ITAM031” or “ITAM031 construct”).

In some embodiments, the CMSD described herein comprises from N′ to C′: optional CMSD N-terminal sequence—DAP12 ITAM—optional first CMSD linker—CD3γ ITAM—optional second CMSD linker—CD3ε ITAM—optional third CMSD linker—CD36 ITAM—optional CMSD C-terminal sequence. In some embodiments, the optional CMSD linker(s), CMSD N-terminal sequence, and CMSD C-terminal sequence are derived from cytoplasmic non-ITAM sequence of ITAM-containing parent molecules. In some embodiments, the CMSD comprises a sequence of SEQ ID NO: 66 (hereinafter also referred to as “ITAM032” or “ITAM032 construct”).

The CMSD described herein in some embodiments has no or reduced binding (such as at least about any of 3%, 4%, 5%, 6%, 7%, 8%, 9%, 10%, 20%, 30%, 40%, 50%, 60%, 70%, 80%, or 90% reduced binding) to a Nef protein described herein (e.g., wt, subtype, mutant, or non-naturally occurring Nef), as compared to CD3ζISD. In some embodiments, the CMSD described herein has the same or similar binding to a Nef protein described herein as compared to CD3ζISD. In some embodiments, the function (e.g., signal transduction and/or as a docking site) of CMSD is down-modulated by a Nef protein described herein the same or similarly as compared to CD3ζISD. In some embodiments, the function (e.g., signal transduction and/or as a docking site) of CMSD is at least about 3% less (e.g., at least about any of 4%, 5%, 6%, 7%, 8%, 9%, 1, 1%, 20%, 30%, 40%, 50%, 60%, 70%, 80%, 90%, or 95% less) down-modulated by a Nef protein described herein as compared to CD3ζISD. In some embodiments, the function (e.g., signal transduction and/or as a docking site) of CMSD is at most about 80% (e.g., at most about any of 70%, 60%, 50%, 40%, 30%, 20%, 10%, or 5%) more down-modulated by a Nef protein described herein as compared to CD3ζISD. In some embodiments, the CMSD does not bind Nef (e.g., wildtype Nef such as wildtype SIV Nef, or mutant Nef such as mutant SIV Nef). In some embodiments, the CMSD does not comprise CD3ζ ITAM1 and CD3ζ ITAM2. In some embodiments, the plurality (e.g., 2, 3, 4, 5, or more) of CMSD ITAMs are selected from CD3ζ ITAM3, DAP12, CD3ε, Igα (CD79a), Igβ (CD79b), or FcεRIγ. In some embodiments, the ITAMs within the CMSD are all CD3ζ ITAM3. In some embodiments, the ITAMs within the CMSD are all CD3ε ITAMs. In some embodiments, the CMSD comprises 3 ITAMs which are DAP12 ITAM, CD3ε ITAM, and CD3ζ ITAM3. In some embodiments, the binding between a Nef (e.g., wildtype Nef such as wildtype SIV Nef, or mutant Nef such as mutant SIV Nef) and a CMSD is at least about 3% less (e.g., at least about any of 4%, 5%, 6%, 7%, 8%, 9%, 10%, 15%, 20%, 30%, 40%, 50%, 60%, 70%, 80%, 90%, or 95% less) than that between the Nef and the ITAM-containing parent molecule (e.g., CD3ζ, CD3ε). In some embodiments, the CMSD has the same or similar activity (e.g., signal transduction and/or as a docking site) compared to that of CD3ζISD. In some embodiments, the CMSD has at most about 80% (e.g., at most about any of 70%, 60%, 50%, 40%, 30%, 20%, 10%, or 5%) less activity (e.g., signal transduction and/or as a docking site) compared to that of CD3ζISD. In some embodiments, the CMSD has at least about 3% (e.g., at least about any of 4%, 5%, 6%, 7%, 8%, 9%, 10%, 15%, 20%, 30%, 40%, 50%, 60%, 70%, 80%, 90%, or 95%) stronger activity (e.g., signal transduction and/or as a docking site) compared to that of CD3ζISD. In some embodiments, the effector function of the functional exogenous receptor comprising the ISD that comprises the CMSD is at least about 20% (such as at least about any of 30%, 40%, 50%, 60%, 70%, 80%, 90%, or 100%) active relative to a functional exogenous receptor comprising an ISD that comprises an intracellular signaling domain of CD3ζ.

Isolated nucleic acids encoding any of the CMSDs described herein are also provided.

CMSD Linker, CMSD C-Terminal Sequence, CMSD N-Terminal Sequence

As discussed above, the CMSD described herein can comprise optional CMSD linker(s), optional CMSD C-terminal sequence, and/or optional CMSD N-terminal sequence. In some embodiments, at least one of the CMSD linker(s), CMSD C-terminal sequence, and/or CMSD N-terminal sequence are derived from an ITAM-containing parent molecule, for example are linker sequences in the ITAM-containing parent molecule. In some embodiments, the CMSD linker(s), the CMSD C-terminal sequence, and/or CMSD N-terminal sequence are heterologous, i.e., they are either not derived from an ITAM-containing parent molecule (e.g., G/S linkers) or derived from an ITAM-containing parent molecule that is different from the ITAM-containing parent molecule from which one or more of the CMSD ITAMs are derived from. In some embodiments, at least one of the CMSD linker(s), CMSD C-terminal sequence, and/or CMSD N-terminal sequence is heterologous to an ITAM-containing parent molecule, for example may comprise a sequence different from any portion of an ITAM-containing parent molecule (e.g., G/S linkers). In some embodiments, the CMSD comprises two or more heterologous CMSD linkers. In some embodiments, the two or more heterologous CMSD linkers are identical to each other. In some embodiments, at least two of the two or more (e.g., 2, 3, 4, or more) heterologous CMSD linkers are identical to each other. In some embodiments, the two or more heterologous CMSD linkers are all different from each other. In some embodiments, at least one of the CMSD linkers, the CMSD C-terminal sequence, and/or the CMSD N-terminal sequence is derived from CD3ζ. In some embodiments, the CMSD linker(s), CMSD C-terminal sequence, and/or CMSD N-terminal sequence are identical to each other. In some embodiments, at least one of CMSD linker(s), CMSD C-terminal sequence, and CMSD N-terminal sequence is different from the others.

The linker(s), C-terminal sequence, and N-terminal sequence within the CMSD may have the same or different length and/or sequence depending on the structural and/or functional features of the CMSD. The CMSD linker, CMSD C-terminal sequence, and CMSD N-terminal sequence may be selected and optimized independently. In some embodiments, longer CMSD linkers (e.g., a linker that is at least about any of 5, 10, 15, 20, 25 or more amino acids long) may be selected to ensure that two adjacent ITAMs do not sterically interfere with one another. In some embodiments, a longer CMSD N-terminal sequence (e.g., a CMSD N-terminal sequence that is at least about any of 5, 10, 15, 20, 25, or more amino acids long) is selected to provide enough space for signal transduction molecules to bind to the most N-terminal ITAM. In some embodiments, the CMSD linker(s), C-terminal CMSD sequence, and/or N-terminal CMSD sequence are no more than about any of 30, 25, 20, 15, 10, 5, or 1 amino acids long. CMSD linker length can also be designed to be the same as that of endogenous linker connecting the ITAMs within the ISD of an ITAM-containing parent molecule. CMSD N-terminal sequence length can also be designed to be the same as that of cytoplasmic N-terminal sequence of an ITAM-containing parent molecule, between the most N-terminal ITAM and the membrane. CMSD C-terminal sequence length can also be designed to be the same as that of cytoplasmic C-terminal sequence of an ITAM-containing parent molecule that is at C-terminus of the last ITAM.

In some embodiments, the CMSD linker is a flexible linker (e.g., comprising flexible amino acid residues such as Gly and Ser, e.g., Gly-Ser doublet). Exemplary flexible linkers include glycine polymers (G)ₙ(SEQ ID NO: 116), glycine-serine polymers (including, for example, (GS)ₙ (SEQ ID NO: 117), (GGGS)ₙ (SEQ ID NO: 118), and (GGGGS)ₙ (SEQ ID NO: 119), where n is an integer of at least one; (GxS)ₙ(SEQ ID NO: 120, wherein n and x are integer independently selected from 3-12)), glycine-alanine polymers, alanine-serine polymers, and other flexible linkers known in the art. In some embodiments, the CMSD linker is a G/S linker. In some embodiments, the flexible linker comprises the amino acid sequence GENLYFQSGG (SEQ ID NO: 17), GGSG (SEQ ID NO: 18), GS (SEQ ID NO: 19), GSGSGS (SEQ ID NO: 20), PPPYQPLGGGGS (SEQ ID NO: 21), GGGGSGGGGS (SEQ ID NO: 22), G (SEQ ID NO: 23), GGGGS (SEQ ID NO: 29), GSTSGSGKPGSGEGSTKG (SEQ ID NO: 32), (GGGS)₃ (SEQ ID NO: 33), (GGGS)₄ (SEQ ID NO: 34), GGGGSGGGGSGGGGGGSGSGGGGS (SEQ ID NO: 35), GGGGSGGGGSGGGGGGSGSGGGGSGGGGSGGGGS (SEQ ID NO: 36), (GGGGS)₃ (SEQ ID NO: 37), (GGGGS)₄ (SEQ ID NO: 38), GGGGGSGGRASGGGGS (SEQ ID NO: 39), or GSGSGSGSGS (SEQ ID NO: 30). In some embodiments, the CMSD linker is selected from the group consisting of SEQ ID NOs: 17-19, 23, 25-29. In some embodiments, the one or more CMSD linkers, the CMSD N-terminal sequence and/or the CMSD C-terminal sequence are flexible (e.g., comprising flexible amino acid residues such as Gly and Ser, e.g., Gly-Ser doublet). In some embodiments, the CMSD N-terminal sequence and/or CMSD C-terminal sequence are independently selected from the group consisting of SEQ ID NOs: 17-39 and 116-120, such as any of SEQ ID NOs: 17-31. In some embodiments, the CMSD C-terminal sequence is selected from the group consisting of SEQ ID NOs: 18, 20, 25, and 27-29. In some embodiments, the CMSD N-terminal sequence is selected from the group consisting of SEQ ID NOs: 17, 21, 22, 24, 30, and 31.

The optional CMSD linker(s), CMSD N-terminal sequence, and/or CMSD C-terminal sequence can be of any suitable length. In some embodiments, the CMSD linker, CMSD N-terminal sequence, and/or CMSD C-terminal sequence is independently no more than about any of 30, 25, 20, 19, 18, 17, 16, 15, 14, 13, 12, 11, 10, 9, 8, 7, 6, 5, 4, 3, 2, or 1 amino acids long. In some embodiments, the length of the CMSD linker(s), CMSD N-terminal sequence, and/or CMSD C-terminal sequence is independently any of about 1 amino acid to about 10 amino acids, about 4 amino acids to about 6 amino acids, about 1 amino acids to about 20 amino acids, about 1 amino acid to about 30 amino acids, about 5 amino acids to about 15 amino acids, about 10 amino acids to about 15 amino acids, about 10 amino acids to about 25 amino acids, about 5 amino acids to about 30 amino acids, about 10 amino acids to about 30 amino acids long, or about 1 amino acid to about 15 amino acids. In some embodiments, the length of the CMSD linker(s), CMSD N-terminal sequence, and/or CMSD C-terminal sequence is about 1 amino acid to about 15 amino acids.

### Extracellular Ligand Binding Domain

The extracellular ligand binding domain of the functional exogenous receptors described herein comprises one or more (such as any one of 1, 2, 3, 4, 5, 6 or more) binding moieties, e.g., antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF). In some embodiments, the one or more binding moieties are antibodies or antigen-binding fragments (e.g., scFv, sdAb) thereof. In some embodiments, the one or more binding moieties are derived from four-chain antibodies. In some embodiments, the one or more binding moieties are derived from camelid antibodies. In some embodiments, the one or more binding moieties are derived from human antibodies. In some embodiments, the one or more binding moieties are selected from the group consisting of a Camel Ig, Ig NAR, Fab fragments, Fab′ fragments, F(ab)′2 fragments, F(ab)′3 fragments, Fv, single chain Fv antibody (scFv), bis-scFv, (scFv)₂, minibody, diabody, triabody, tetrabody, disulfide stabilized Fv protein (dsFv), and single-domain antibody (e.g., sdAb, nanobody, V_{H}H). In some embodiments, the one or more binding moieties are sdAbs (e.g., anti-BCMA sdAbs). In some embodiments, the one or more binding moieties are scFvs (e.g., anti-CD19 scFv, anti-CD20 scFv, anti-BCMA scFv). In some embodiments, the one or more binding moieties are non-antibody binding proteins, such as polypeptide ligands/receptors or engineered proteins that bind to an antigen. In some embodiments, the one or more non-antibody binding moieties comprise at least one domain derived from a ligand or the extracellular domain of a cell surface receptor. In some embodiments, the ligand or receptor is selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is APRIL or BAFF, which can bind to BCMA receptor. In some embodiments, the receptor is an Fc receptor (FcR) and the ligand is an Fc-containing molecule (e.g., full length monoclonal antibody). In some embodiments, the one or more binding moieties are derived from extracellular domain (or portion thereof) of an FcR. In some embodiments, the FcR is an Fcγ receptor (FcγR). In some embodiments, the FcγR is selected from the group consisting of FcγRIA (CD64A), FcγRIB (CD64B), FcγRIC (CD64C), FcγRIIA (CD32A), FcγRIIB (CD32B), FcγRIIIA (CD16a), and FcγRIIIB (CD16b). The two or more binding moieties (e.g., sdAbs) can be fused to each other directly via peptide bonds, or via peptide linkers (see receptor domain linkers subsection below). In some embodiments, the peptide linker comprises the amino acid sequence of SEQ ID NO: 29.

Single-Domain Antibodies (sdAbs)

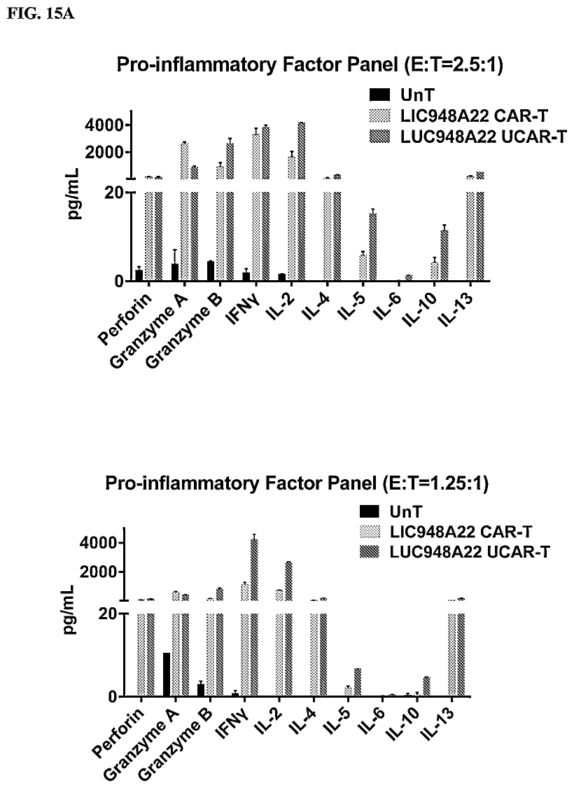

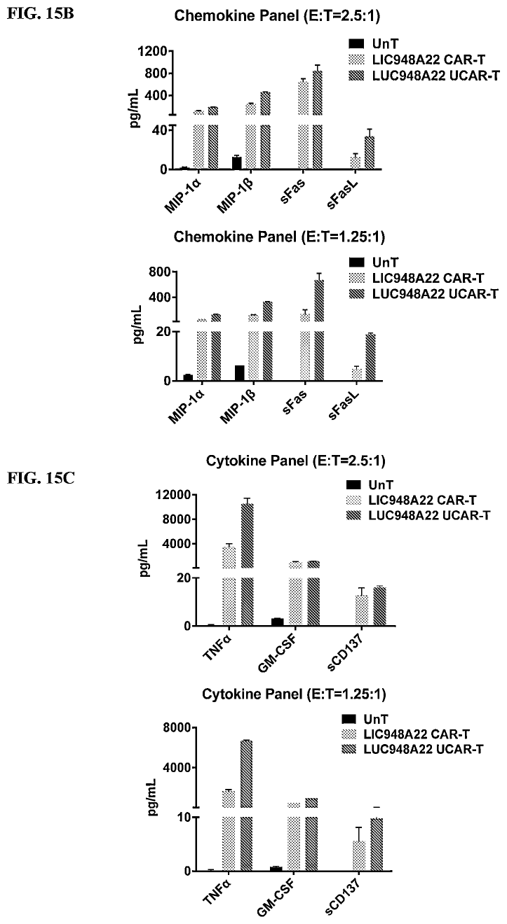

In some embodiments, the extracellular ligand binding domain comprising one or more sdAbs (e.g., anti-BCMA sdAbs). The sdAbs may be of the same or different origins, and of the same or different sizes. Exemplary sdAbs include, but are not limited to, heavy chain variable domains from heavy-chain only antibodies (e.g., V_{H}H or V_{NAR}), binding molecules naturally devoid of light chains, single domains (such as V_{H} or V_{L}) derived from conventional 4-chain antibodies, humanized heavy-chain only antibodies, human sdAbs produced by transgenic mice or rats expressing human heavy chain segments, and engineered domains and single domain scaffolds other than those derived from antibodies. Any sdAbs known in the art or developed by the Applicant, including the sdAbs described in PCT/CN2017/096938 and PCT/CN2016/094408 (the contents of each of which are incorporated herein by reference in their entireties) may be used to construct the functional exogenous receptor comprising a CMSD described herein. Exemplary structures of CARs (e.g., ITAM-modified CARs) are shown in FIGS. 15A-15D of PCT/CN2017/096938. The sdAbs may be derived from any species including, but not limited to mouse, rat, human, camel, llama, lamprey, fish, shark, goat, rabbit, and bovine. SdAbs contemplated herein also include naturally occurring sdAb molecules from species other than Camelidae and sharks.

In some embodiments, the sdAb is derived from a naturally occurring single-domain antigen binding molecule known as heavy chain antibody devoid of light chains (also referred herein as “heavy chain only antibodies”). Such single domain molecules are disclosed in WO 94/04678 and Hamers-Casterman, C. et al. (1993) Nature 363:446-448, for example. For clarity reasons, the variable domain derived from a heavy chain molecule naturally devoid of light chain is known herein as a V_{H}H to distinguish it from the conventional V_{H} of four chain immunoglobulins. Such a V_{H}H molecule can be derived from antibodies raised in Camelidae species, for example, camel, llama, vicuna, dromedary, alpaca and guanaco. Other species besides Camelidae may produce heavy chain molecules naturally devoid of light chain, and such VHHs are within the scope of the present application.

V_{H}H molecules from Camelids are about 10 times smaller than IgG molecules. They are single polypeptides and can be very stable, resisting extreme pH and temperature conditions. Moreover, they can be resistant to the action of proteases which is not the case for conventional 4-chain antibodies. Furthermore, in vitro expression of V_{H}H s produces high yield, properly folded functional V_{H}Hs. In addition, antibodies generated in Camelids can recognize epitopes other than those recognized by antibodies generated in vitro through the use of antibody libraries or via immunization of mammals other than Camelids (see, for example, WO9749805). As such, multispecific and/or multivalent functional exogenous receptor comprising a CMSD described herein (e.g., ITAM-modified TCR, ITAM-modified CAR, an ITAM-modified cTCR, or ITAM-modified TAC-like chimeric receptor) comprising one or more V_{H}H domains may interact more efficiently with targets than multispecific and/or multivalent functional exogenous receptors comprising antigen-binding fragments derived from conventional 4-chain antibodies. Since V_{H}Hs are known to bind into “unusual” epitopes such as cavities or grooves, the affinity of functional exogenous receptors comprising such V_{H}Hs may be more suitable for therapeutic treatment than conventional multispecific non-V_{H}H containing chimeric receptors (e.g., non-V_{H}H containing CAR).

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) V_{H} domain from a conventional 4-chain antibody by one or more of the amino acid residues that occur at the corresponding position(s) in a V_{H}H domain of a heavy chain antibody. This can be performed in a manner known per se, which will be clear to the skilled person. Such “camelizing” substitutions are preferably inserted at amino acid positions that form and/or are present at the V_{H}-V_{L} interface, and/or at the so-called Camelidae hallmark residues (see for example WO 94/04678, Davies and Riechmann FEBS Letters 339: 285-290, 1994; Davies and Riechmann Protein Engineering 9 (6): 531-537, 1996; Riechmann J. Mol. Biol. 259: 957-969, 1996; and Riechmann and Muyldermans J. Immunol. Meth. 231: 25-38, 1999).

In some embodiments, the sdAb is a human sdAb produced by transgenic mice or rats expressing human heavy chain segments. See, e.g., US20090307787A1, U.S. Pat. No. 8,754,287, US20150289489A1, US20100122358A1, and WO2004049794. In some embodiments, the sdAb is affinity matured.

In some embodiments, naturally occurring V_{H}H domains against a particular antigen or target, can be obtained from (naïve or immune) libraries of Camelid V_{H}H sequences. Such methods may or may not involve screening such a library using said antigen or target, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from (naïve or immune) V_{H}H libraries may be used, such as V_{H}H libraries obtained from (naïve or immune) V_{H}H libraries by techniques such as random mutagenesis and/or CDR shuffling, as for example described in WO 00/43507.

In some embodiments, the sdAbs are generated from conventional four-chain antibodies. See, for example, EP 0 368 684, Ward et al. (Nature 1989 Oct. 12; 341 (6242): 544-6), Holt et al. (Trends Biotechnol., 2003, 21(11):484-490), WO 06/030220, and WO 06/003388.

In some embodiments, the sdAb specifically binds to BCMA. In some embodiments, the anti-BCMA sdAb (e.g., V_{H}H) comprises a CDR1 comprising the amino acid sequence of SEQ ID NO: 130, a CDR2 comprising the amino acid sequence of SEQ ID NO: 131, and a CDR3 comprising the amino acid sequence of SEQ ID NO: 132. In some embodiments, the sdAb (e.g., V_{H}H) comprises CDR1, CDR2, and CDR3 of an anti-BCMA sdAb comprising the amino acid sequence of SEQ ID NO: 128. In some embodiments, the anti-BCMA sdAb binds to the same epitope as an anti-BCMA sdAb (e.g., V_{H}H) comprising a CDR1 comprising the amino acid sequence of SEQ ID NO: 130, a CDR2 comprising the amino acid sequence of SEQ ID NO: 131, and a CDR3 comprising the amino acid sequence of SEQ ID NO: 132.

In some embodiments, the anti-BCMA sdAb (e.g., V_{H}H) comprises a CDR1 comprising the amino acid sequence of SEQ ID NO: 133, a CDR2 comprising the amino acid sequence of SEQ ID NO: 134, and a CDR3 comprising the amino acid sequence of SEQ ID NO: 135. In some embodiments, the anti-BCMA sdAb comprises CDR1, CDR2, and CDR3 of an anti-BCMA sdAb comprising the amino acid sequence of SEQ ID NO: 129. In some embodiments, the anti-BCMA sdAb binds to the same epitope as an anti-BCMA sdAb moiety (e.g., V_{H}H) comprising a CDR1 comprising the amino acid sequence of SEQ ID NO: 133, a CDR2 comprising the amino acid sequence of SEQ ID NO: 134, and a CDR3 comprising the amino acid sequence of SEQ ID NO: 135.

In some embodiments, the CMSD-containing functional exogenous receptor in some embodiments comprises an extracellular ligand binding domain comprising a first sdAb moiety that specifically binds to BCMA and a second sdAb moiety that specifically binds to BCMA (hereinafter referred to as “anti-BCMA sdAb” such as “anti-BCMA V_{H}H”). The first sdAb moiety and the second sdAb moiety may bind to different epitopes of BCMA. The two sdAb may be arranged in tandem, optionally linked by a linker sequence. Any of the linker sequences as described in “CMSD linker” and “receptor domain linker” sections can be used herein. In some embodiments, the CMSD-containing functional exogenous receptor comprises an extracellular ligand binding domain comprising 3 or more sdAbs (e.g., specifically recognizing BCMA).

Target Antigens and Target Molecules

The extracellular ligand binding domain of the functional exogenous receptor comprising a CMSD described herein (e.g., ITAM-modified TCR, ITAM-modified CAR, ITAM-modified cTCR, or ITAM-modified TAC-like chimeric receptor) can specifically recognize any antigen (or any epitope of any antigen) on a target cell (e.g., tumor cell), or a target molecule (e.g., Fc-containing molecule such as monoclonal antibody). In some embodiments, the target antigen is a cell surface molecule (e.g., extracellular domain of a receptor/ligand). In some embodiments, the target antigen acts as a cell surface marker on target cells associated with a special disease state. In some embodiments, the target antigen is a tumor antigen. In some embodiments, the extracellular ligand binding domain specifically recognizes a single target (e.g., tumor) antigen. In some embodiments, the extracellular ligand binding domain specifically recognizes one or more epitopes of a single target (e.g., tumor) antigen. In some embodiments, the extracellular ligand binding domain specifically recognizes two or more target (e.g., tumor) antigens. In some embodiments, the tumor antigen is associated with a B cell malignancy, such as B-cell lymphoma or multiple myeloma (MM). Tumors express a number of proteins that can serve as a target antigen for an immune response, particularly T cell mediated immune responses. The target antigens (e.g., tumor antigen, extracellular domain of a receptor/ligand) specifically recognized by the extracellular ligand binding domain may be antigens on a single diseased cell or antigens that are expressed on different cells that each contribute to the disease. The antigens specifically recognized by the extracellular ligand binding domain may be directly or indirectly involved in the diseases.

Tumor antigens are proteins that are produced by tumor cells that can elicit an immune response, particularly T cell mediated immune responses. The selection of the targeted antigen of the invention will depend on the particular type of cancer to be treated. Exemplary tumor antigens include, for example, a glioma-associated antigen, BCMA (B-cell maturation antigen), carcinoembryonic antigen (CEA), β-human chorionic gonadotropin, alpha-fetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, and mesothelin. In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells. Non-limiting examples of TSA or TAA antigens include the following: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p15, p16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68P1, CO-029, FGF-5, G250, Ga733\EpCAM, HTgp-175, M344, MA-50, MG7-Ag, MOV18, NB/70K, NY-CO-1, RCAS 1, SDCCAGI6, TA-90\Mac-2 binding protein\cyclophilin C-associated protein, TAAL6, TAG72, TLP, and TPS.

In some embodiments, the tumor antigen is selected from the group consisting of Mesothelin, TSHR, CD19, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha, ERBB2 (Her2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, CLDN18.2, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p⁵³ mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In some embodiments, the tumor antigen is selected from the group consisting of CD19, CD20, CD22, CD30, CD33, CD38, BCMA, CS1, CD138, CD123/IL3Ru, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Glycolipid F77, PD-L1, PD-L2, and any combination thereof. In some embodiments, the tumor antigen is expressed on a B cell. In some embodiments, the tumor antigen is BCMA, CD19, or CD20.

In some embodiments, the target antigen is a pathogen antigen, such as a fungal, viral, or bacterial antigen. In some embodiments, the fungal antigen is from Aspergillus or Candida. In some embodiments, the viral antigen is from Herpes simplex virus (HSV), respiratory syncytial virus (RSV), metapneumovirus (hMPV), rhinovirus, parainfluenza (PIV), Epstein-Barr virus (EBV), Cytomegalovirus (CMV), JC virus (John Cunningham virus), BK virus, HIV, Zika virus, human coronavirus, norovirus, encephalitis virus, or Ebola.

In some embodiments, the target antigen is a cell surface molecule. In some embodiments, the cell surface antigen is a ligand or receptor. In some embodiments, the extracellular ligand binding domain comprises one or more binding moieties comprising at least one domain derived from a ligand or the extracellular domain of a receptor. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL and/or BAFF, which can bind to BCMA. In some embodiments, the receptor is an FcR and the ligand is an Fc-containing molecule. In some embodiments, the FcR is an Fcγ receptor (FcγR). In some embodiments, the FcγR is selected from the group consisting of FcγRIA (CD64A), FcγRIB (CD64B), FcγRIC (CD64C), FcγRIIA (CD32A), FcγRIIB (CD32B), FcγRIIIA (CD16a), and FcγRIIIB (CD16b).

### Hinge

The functional exogenous receptor comprising a CMSD described herein (e.g., ITAM-modified TCR, ITAM-modified CAR, an ITAM-modified cTCR, or ITAM-modified TAC-like chimeric receptor) in some embodiments comprises a hinge domain located between the C-terminus of the extracellular ligand binding domain and the N-terminus of the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand binding domain relative to the transmembrane domain can be used.

The hinge domain can contain about 10-100 amino acids, e.g., about any one of 15-75 amino acids, 20-50 amino acids, or 30-60 amino acids. In some embodiments, the hinge domain is at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, or 95 amino acids in length.

In some embodiments, the hinge domain is a hinge domain of a naturally occurring protein. Hinge domains of any protein known in the art to comprise a hinge domain are compatible for use in the functional exogenous receptor comprising a CMSD described herein. In some embodiments, the hinge domain is at least a portion of a hinge domain of a naturally occurring protein and confers flexibility to the functional exogenous receptor comprising a CMSD. In some embodiments, the hinge domain is derived from CD8α. In some embodiments, the hinge domain is a portion of the hinge domain of CD8α, e.g., a fragment comprising at least about 15 (e.g., at least about any of 20, 25, 30, 35, 40, or 45) consecutive amino acids of the hinge domain of CD8α. In some embodiments, the hinge domain comprises a sequence of SEQ ID NO: 125.

Hinge domains of antibodies, such as IgG, IgA, IgM, IgE, or IgD antibodies, are also compatible for use in the functional exogenous receptor comprising a CMSD described herein (e.g., ITAM-modified TCR, ITAM-modified CAR, an ITAM-modified cTCR, or ITAM-modified TAC-like chimeric receptor). In some embodiments, the hinge domain of the functional exogenous receptor is the hinge domain that connects the constant domains CH1 and CH2 of an antibody. In some embodiments, the hinge domain is derived from an antibody, and comprises the hinge domain of the antibody and one or more constant regions of the antibody. In some embodiments, the hinge domain of the functional exogenous receptor comprises the hinge domain of an antibody and the CH3 constant region of the antibody. In some embodiments, the hinge domain of the functional exogenous receptor comprises the hinge domain of an antibody and the CH2 and CH3 constant regions of the antibody. In some embodiments, the antibody is an IgG, an IgA, an IgM, an IgE, or an IgD antibody. In some embodiments, the antibody is an IgG antibody. In some embodiments, the antibody is an IgG1, IgG2, IgG3, or IgG4 antibody. In some embodiments, the hinge region of the functional exogenous receptor comprises the hinge region and the CH2 and CH3 constant regions of an IgG1 antibody. In some embodiments, the hinge region of the functional exogenous receptor comprises the hinge region and the CH3 constant region of an IgG1 antibody.

Non-naturally occurring peptides may also be used as hinge domains of the functional exogenous receptors comprising a CMSD described herein. In some embodiments, the hinge domain located between the C-terminus of the extracellular ligand binding domain and the N-terminus of the transmembrane domain is a flexible linker (e.g., G/S linker), such as a (GₓS)ₙ linker, wherein x and n, independently can be an integer between 3 and 12 (e.g., 3, 4, 5, 6, 7, 8, 9, 10, 11, 12) (SEQ ID NO: 120). In some embodiments, the hinge domain can be a flexible linker described in the “CMSD linker” and “receptor domain linker” subsections above, such as selected from the group consisting of SEQ ID NOs: 17-39 and 116-120. In some embodiments, the hinge is at least about 10 amino acids long, e.g., GENLYFQSGG (SEQ ID NO: 17), PPPYQPLGGGGS (SEQ ID NO: 21), GGGGSGGGGS (SEQ ID NO: 22), GSTSGSGKPGSGEGSTKG (SEQ ID NO: 32), (GGGS)₃ (SEQ ID NO: 33), (GGGS)₄ (SEQ ID NO: 34), GGGGSGGGGSGGGGGGSGSGGGGS (SEQ ID NO: 35), GGGGSGGGGSGGGGGGSGSGGGGSGGGGSGGGGS (SEQ ID NO: 36), (GGGGS)₃ (SEQ ID NO: 37), (GGGGS)₄ (SEQ ID NO: 38), GGGGGSGGRASGGGGS (SEQ ID NO: 39), or GSGSGSGSGS (SEQ ID NO: 30).

### Transmembrane Domain

The functional exogenous receptor comprising a CMSD described herein (e.g., ITAM-modified TCR, ITAM-modified CAR, an ITAM-modified cTCR, or ITAM-modified TAC-like chimeric receptor) comprises a transmembrane domain that can be directly or indirectly fused to the extracellular ligand binding domain. The transmembrane domain may be derived from either a natural source or a synthetic source. For example, the transmembrane domain can be a synthetic, non-naturally occurring protein segment, e.g., a hydrophobic protein segment that is thermodynamically stable in a cell membrane. As used herein, a “transmembrane domain” refers to any protein structure that is thermodynamically stable in a cell membrane, preferably a eukaryotic cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the functional exogenous receptor described herein is derived from a Type I single-pass membrane protein. In some embodiments, transmembrane domains from multi-pass membrane proteins may also be compatible for use in the functional exogenous receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the functional exogenous receptor comprising a CMSD described herein comprises a transmembrane domain selected from any transmembrane domain (or portion thereof) of TCRα, TCRβ, TCRγ, TCRδ, CD3ζ, CD3γ, CD3δ, CD3ε, CD2, CD45, CD4, CD5, CD8 (e.g., CD8α), CD9, CD16, LFA-1 (CDIIa, CD18), CD19, CD22, CD27, CD28, CD29, CD33, CD37, CD40, CD45, CD64, CD80, CD84, CD86, CD96 (Tactile), CD100 (SEMA4D), CD103, CD134, CD137 (4-1BB), SLAM (SLAMF1, CD150, IPO-3), CD152, CD154, CD160 (BY55), SELPLG (CD162), DNAM1 (CD226), Ly9 (CD229), SLAMF4 (CD244, 2B4), ICOS (CD278), KIRDS2, OX40, PD-1, GITR, BAFFR, HVEM (LIGHTR), SLAMF7, NKp80 (KLRF1), IL-2Rβ, IL-2Rγ, IL-7Ra, ITGA1, VLA1, CD49a, ITGA4, IA4, CD49D, ITGA6, VLA-6, CD49f, ITGAD, ITGAE, ITGAL, CDIIa, ITGAM, CD11 b, CD11c, CD11d, ITGAX, ITGB1, ITGB2, ITGB7, TNFR2, CEACAMI, CRT AM, PSGL1, SLAMF6 (NTB-A, Ly108), BLAME (SLAMF8), LTBR, PAG/Cbp, NKp44, NKp30, NKp46, NKG2D, and/or NKG2C. In some embodiments, the transmembrane domain is derived from a molecule selected from the group consisting of TCRα, TCRβ, TCRγ, TCRδ, CD3ζ, CD3ε, CD3γ, CD3δ, CD4, CD5, CD8α, CD9, CD16, CD22, CD27, CD28, CD33, CD37, CD45, CD64, CD80, CD86, CD134, CD137 (4-1BB), CD152, CD154, and PD-1. In some embodiments, the transmembrane domain is derived from CD28. In some embodiments, the transmembrane domain is derived from CD8α. In some embodiments, the transmembrane domain comprises a sequence of SEQ ID NO: 126. In some embodiments, the hinge and transmembrane domain are derived from the same molecule, e.g., CD8α.

Transmembrane domains for use in the functional exogenous receptor comprising a CMSD described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least about approximately 18 amino acids, e.g., at least about any of 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated herein by reference in their entireties.

The transmembrane domain of the functional exogenous receptor comprising a CMSD described herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the functional exogenous receptor comprising a CMSD described herein comprises hydrophobic amino acid residues. In some embodiments, the transmembrane domain of the functional exogenous receptor comprising a CMSD described herein comprises an artificial hydrophobic sequence. For example, a triplet of phenylalanine, tryptophan, and valine may be present at the C-terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte-Doolittle hydropathy analysis.

### Functional Exogenous Receptor Domain Linkers (“Receptor Domain Linkers”)

In some embodiments, various domains of the functional exogenous receptor comprising a CMSD described herein (e.g., ITAM-modified TCR, ITAM-modified CAR, an ITAM-modified cTCR, or ITAM-modified TAC-like chimeric receptor), such as two or more binding moieties (e.g., antigen-binding fragments such as scFvs or sdAbs, ligand/receptor domains) within the extracellular ligand binding domain, the extracellular ligand binding domain and the optional hinge domain, the extracellular ligand binding domain and the transmembrane domain, the transmembrane domain and the ISD, may be fused to each other via peptide linkers, hereinafter also referred to as “receptor domain linkers”, to distinguish from optional CMSD linkers described above within the CMSD. In some embodiments, various domains of the functional exogenous receptor comprising a CMSD described herein, e.g., the two or more binding moieties (e.g., antigen-binding fragments such as scFvs or sdAbs, ligand/receptor domains) within the extracellular ligand binding domain, are directly fused to each other without any peptide linkers. The receptor domain peptide linkers connecting various domains of the functional exogenous receptor comprising a CMSD described herein, e.g., between the two or more binding moieties (e.g., antigen-binding fragments such as scFvs or sdAbs, ligand/receptor domains) within the extracellular ligand binding domain, between the extracellular ligand binding domain and the optional hinge domain, between the extracellular ligand binding domain and the transmembrane domain, between the transmembrane domain and the ISD, may be the same or different.

Each receptor domain peptide linker in a functional exogenous receptor comprising a CMSD described herein (e.g., ITAM-modified TCR, ITAM-modified CAR, an ITAM-modified cTCR, or ITAM-modified TAC-like chimeric receptor) may have the same or different length and/or sequence depending on the structural and/or functional features of the various domains of the functional exogenous receptor. Each receptor domain peptide linker may be selected and optimized independently. The length, the degree of flexibility and/or other properties of the receptor domain peptide linker(s) used in the functional exogenous receptor comprising a CMSD described herein, e.g., peptide linkers connecting the two or more binding moieties (e.g., antigen-binding fragments such as scFvs or sdAbs, ligand/receptor domains) within the extracellular ligand binding domain, may have some influence on properties, including but not limited to the affinity, specificity or avidity for one or more particular antigens or epitopes. For example, longer peptide linkers may be selected to ensure that two adjacent domains (or binding moieties) do not sterically interfere with one another. For example, in a multivalent and/or multispecific functional exogenous receptor comprising a CMSD described herein (e.g., ITAM-modified TCR, ITAM-modified CAR, an ITAM-modified cTCR, or ITAM-modified TAC-like chimeric receptor) that comprises sdAbs directed against a multimeric antigen, the length and flexibility of the receptor domain peptide linkers are preferably such that it allows each sdAb within the extracellular ligand binding domain to bind to the antigenic determinant on each subunit of the multimer. In some embodiments, a short peptide linker may be disposed between the transmembrane domain and the ISD. In some embodiment, a peptide linker comprises flexible residues (such as glycine and serine) so that the adjacent domains (or binding moieties) are free to move relative to each other. For example, a glycine-serine doublet can be a suitable peptide linker.

The receptor domain peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 60, 70, 80, 90, 100 or more amino acids long. In some embodiments, the receptor domain peptide linker is no more than about any of 100, 90, 80, 70, 60, 50, 40, 35, 30, 25, 20, 19, 18, 17, 16, 15, 14, 13, 12, 11, 10, 9, 8, 7, 6, 5 or fewer amino acids long. In some embodiments, the length of the receptor domain peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The receptor domain peptide linker may have a naturally occurring sequence, or a non-naturally occurring sequence. For example, a sequence derived from the hinge region of heavy chain only antibodies may be used as the receptor domain peptide linker. See, for example, WO1996/34103. In some embodiments, the receptor domain peptide linker is a flexible linker. Exemplary flexible linkers include glycine polymers (G)ₙ(SEQ ID NO: 116), glycine-serine polymers (including, for example, (GS)ₙ (SEQ ID NO: 117), (GGGS)ₙ (SEQ ID NO: 118), and (GGGGS)ₙ (SEQ ID NO: 119), where n is an integer of at least one), glycine-alanine polymers, alanine-serine polymers, and other flexible linkers known in the art. In some embodiments, the receptor domain peptide linker is a (GₓS)ₙ linker, wherein x and n independently can be an integer between 3 and 12 (e.g., 3, 4, 5, 6, 7, 8, 9, 10, 11, 12) (SEQ ID NO: 120). In some embodiments, the receptor domain peptide linker comprises the amino acid sequence of any of SEQ ID NOs: 17-39 and 116-120. In some embodiments, the receptor domain peptide linker comprises the amino acid sequence of SEQ ID NO: 29.

### Signal Peptide

The functional exogenous receptor comprising a CMSD described herein (e.g., ITAM-modified TCR, ITAM-modified CAR, an ITAM-modified cTCR, or ITAM-modified TAC-like chimeric receptor) may comprise a signal peptide (also known as a signal sequence) at the N-terminus of the functional exogenous receptor polypeptide. In general, signal peptides are peptide sequences that target a polypeptide to the desired site in a cell. In some embodiments, the signal peptide targets functional exogenous receptor to the secretory pathway of the cell and will allow for integration and anchoring of the functional exogenous receptor into the lipid bilayer. Signal peptides including signal sequences of naturally occurring proteins or synthetic, non-naturally occurring signal sequences, which are compatible for use in the functional exogenous receptor comprising a CMSD described herein, will be evident to one of skill in the art. In some embodiments, the signal peptide is derived from a molecule selected from the group consisting of CD8α, GM-CSF receptor α, and IgG1 heavy chain. In some embodiments, the signal peptide is derived from CD8α. In some embodiments, the signal peptide comprises the sequence of SEQ ID NO: 127.

### ITAM-Modified Chimeric Antigen Receptors (CARs)

In some embodiments, the functional exogenous receptor comprising a CMSD described herein is an ITAM-modified CAR, i.e., a CAR comprising an ISD that comprises a CMSD described herein. In some embodiments, the ITAM-modified CAR comprises an ISD comprising any of the CMSDs described herein. In some embodiments, there is provided an ITAM-modified CAR comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the plurality (e.g., 2, 3, 4, or more) of CMSD ITAMs are directly linked to each other. In some embodiments, the CMSD comprises two or more (e.g., 2, 3, 4, or more) CMSD ITAMs connected by one or more linkers not derived from an ITAM-containing parent molecule (e.g., G/S linker). In some embodiments, the CMSD comprises one or more CMSD linkers derived from an ITAM-containing parent molecule that is different from the ITAM-containing parent molecule from which one or more of the CMSD ITAMs are derived from. In some embodiments, the CMSD comprises two or more (e.g., 2, 3, 4, or more) identical CMSD ITAMs. In some embodiments, at least one of the CMSD ITAMs is not derived from CD3ζ. In some embodiments, at least one of the CMSD ITAMs is not ITAM1 or ITAM2 of CD3ζ. In some embodiments, the plurality of CMSD ITAMs are each derived from a different ITAM-containing parent molecule. In some embodiments, at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, at least one of the plurality of CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin. In some embodiments, the plurality of CMSD ITAMs are derived from one or more of CD3ε, CD3δ, CD3γ, CD3ζ, DAP12, Igα (CD79a), Igβ (CD79b), and FcεRIγ. In some embodiments, the CMSD does not comprise CD3ζ ITAM1 and/or CD3ζ ITAM2. In some embodiments, the CMSD comprises CD3ζ ITAM3. In some embodiments, the CMSD does not comprise any CD3ζ ITAMs. In some embodiments, the transmembrane domain is derived from a molecule selected from the group consisting of TCRα, TCRβ, TCRγ, TCRδ, CD3ζ, CD3ε, CD3γ, CD3δ, CD4, CD5, CD8α, CD9, CD16, CD22, CD27, CD28, CD33, CD37, CD45, CD64, CD80, CD86, CD134, CD137 (4-1BB), CD152, CD154, and PD-1. In some embodiments, the transmembrane domain is derived from CD8α. In some embodiments, the transmembrane domain comprises a sequence of SEQ ID NO: 126. In some embodiments, the ISD further comprises a co-stimulatory signaling domain. In some embodiments, the co-stimulatory signaling domain is derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, BLAME (SLAMF8), LTBR, LAT, GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof. In some embodiments, the co-stimulatory signaling domain is derived from CD137 (4-1BB) or CD28. In some embodiments, the co-stimulatory signaling domain comprises the sequence of SEQ ID NO: 124. In some embodiments, the co-stimulatory domain is N-terminal to the CMSD. In some embodiments, the co-stimulatory domain is C-terminal to the CMSD. In some embodiments, the extracellular ligand binding domain comprises an antigen-binding fragment (e.g., one or more scFv, sdAb) that specifically recognizing one or more epitopes of one or more target antigens (e.g., tumor antigen such as CD19, CD20, or BCMA). ITAM-modified CAR comprising one or more antigen-binding fragments within the extracellular ligand binding domain is hereinafter referred to as “ITAM-modified antibody-based CAR.” In some embodiments, the antigen-binding fragment is selected from the group consisting of a Camel Ig, an Ig NAR, a Fab fragment, a single chain Fv antibody, and a single-domain antibody (sdAb, nanobody). In some embodiments, the antigen-binding fragment is an sdAb or an scFv. In some embodiments, the tumor antigen is selected from the group consisting of Mesothelin, TSHR, CD19, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha, ERBB2 (Her2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, CLDN18.2, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p⁵³ mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In some embodiments, the tumor antigen is CD19, CD20, or BCMA. In some embodiments, the extracellular ligand binding domain comprises (e.g., consists essentially of) one or more non-antibody binding moieties, such as polypeptide ligands or engineered proteins that bind to an antigen. In some embodiments, the one or more non-antibody binding moieties comprise at least one domain derived from a cell surface ligand or the extracellular domain of a cell surface receptor. In some embodiments, the extracellular ligand binding domain comprises an extracellular domain of a receptor or a portion thereof (e.g., one or more extracellular domains of one or more receptors, or a portion thereof) that specifically recognizing one or more ligands. In some embodiments, the ligand and/or receptor is selected from the group consisting of NKG2A, NKG2C, NKG2F, NKG2D, BCMA, APRIL, BAFF, IL-3, IL-13, LLT1, AICL, DNAM-1, and NKp80. In some embodiments, the receptor is BCMA. ITAM-modified CAR comprising one or more extracellular domains (or portion thereof) of one or more receptors within the extracellular ligand binding domain is hereinafter referred to as “ITAM-modified ligand/receptor-based CAR.” In some embodiments, the receptor is an Fc receptor (FcR) and the ligand is an Fc-containing molecule. ITAM-modified CAR comprising one or more FcRs within the extracellular ligand binding domain is hereinafter referred to as “ITAM-modified Antibody-Coupled T Cell Receptor (ACTR).” Modified T cells expressing an ITAM-modified ACTR can bind to an Fc-containing molecule, such as a monoclonal antibody specifically recognizing a target (e.g., tumor) antigen (e.g., anti-BCMA, anti-CD19, or anti-CD20 full length antibody), which acts as a bridge directing the modified T cells to tumor cells. In some embodiments, the receptor is an Fcγ receptor (FcγR). In some embodiments, the FcγR is selected from the group consisting of FcγRIA (CD64A), FcγRIB (CD64B), FcγRIC (CD64C), FcγRIIA (CD32A), FcγRIIB (CD32B), FcγRIIIA (CD16a), and FcγRIIIB (CD16b). In some embodiments, the Fc-containing molecule is a full length antibody. In some embodiments, the extracellular ligand binding domain is monovalent (or monospecific), i.e., the ITAM-modified CAR is monovalent (or monospecific). In some embodiments, the extracellular ligand binding domain is multivalent (e.g., bivalent) and monospecific, i.e., the ITAM-modified CAR is multivalent (e.g., bivalent) and monospecific. In some embodiments, the extracellular ligand binding domain is multivalent (e.g., bivalent) and multispecific (e.g., bispecific), i.e., the ITAM-modified CAR is multivalent (e.g., bivalent) and multispecific (e.g., bispecific). In some embodiments, the ITAM-modified CAR further comprises a hinge domain located between the C-terminus of the extracellular ligand binding domain (e.g., scFv, sdAb) and the N-terminus of the transmembrane domain. In some embodiments, the hinge domain is derived from CD8α. In some embodiments, the hinge domain comprises the sequence of SEQ ID NO: 125. In some embodiments, the ITAM-modified CAR further comprises a signal peptide (SP) located at the N-terminus of the ITAM-modified CAR (i.e., N-terminus of the extracellular ligand binding domain). In some embodiments, the signal peptide is derived from CD8α. In some embodiments, the signal peptide comprises the sequence of SEQ ID NO: 127. In some embodiments, the signal peptide is removed after the exportation to the cell surface of the ITAM-modified CAR. In some embodiments, the ITAM-modified CAR comprises the amino acid sequence of any of SEQ ID NOs: 76-96, 98-104, and 106-113. In some embodiments, the ITAM-modified CAR is not down-modulated (e.g., down-regulated for cell surface expression and/or effector function such as signal transduction related to cytolytic activity) by a Nef protein (e.g., wildtype Nef such as wildtype SIV Nef, Nef subtype, or mutant Nef such as mutant SIV Nef). In some embodiments, the ITAM-modified CAR is at most about 80% (such as at most about any of 70%, 60%, 50%, 40%, 30%, 20%, 10%, or 5%) down-modulated (e.g., down-regulated for cell surface expression and/or effector function such as signal transduction involved in cytolytic activity) by a Nef protein compared to when the Nef is absent. In some embodiments, the ITAM-modified CAR is down-modulated (e.g., down-regulated for cell surface expression and/or effector function such as signal transduction involved in cytolytic activity) by a Nef protein the same or similarly as a same CAR comprising a CD3ζISD (e.g., traditional CAR comprising everything the same but with a CD3ζISD). In some embodiments, the ITAM-modified CAR is at least about 3% less (e.g., at least about any of 4%, 5%, 6%, 7%, 8%, 9%, 10%, 15%, 20%, 30%, 40%, 50%, 60%, 70%, 80%, 90%, or 95% less) down-modulated (e.g., down-regulated for cell surface expression and/or effector function such as signal transduction involved in cytolytic activity) by a Nef protein than a traditional CAR comprising a CD3ζISD. In some embodiments, the ITAM-modified CAR is at most about 80% (e.g., at most about any of 70%, 60%, 50%, 40%, 30%, 20%, 10%, or 5%) more down-modulated (e.g., down-regulated for cell surface expression and/or effector function such as signal transduction involved in cytolytic activity) by a Nef protein than a same CAR comprising a CD3ζISD (e.g., traditional CAR with CD3ζISD). In some embodiments, the ITAM-modified CAR has the same or similar effector function (e.g., signal transduction involved in cytolytic activity) compared to that of a same CAR comprising a CD3ζISD (e.g., traditional CAR with a CD3ζISD). In some embodiments, the ITAM-modified CAR has at least about 3% (e.g., at least about any of 4%, 5%, 6%, 7%, 8%, 9%, 10%, 15%, 20%, 30%, 40%, 50%, 60%, 70%, 80%, 90%, or 95%) stronger effector function (e.g., signal transduction involved in cytolytic activity) compared to that of a same CAR comprising a CD3ζ ISD (e.g., traditional CAR with CD3ζISD). In some embodiments, the ITAM-modified CAR has at most about 80% (e.g., at most about any of 70%, 60%, 50%, 40%, 30%, 20%, 10%, or 5%) less effector function (e.g., signal transduction involved in cytolytic activity) compared to that of a same CAR comprising a CD3ζISD (e.g., traditional CAR with CD3ζISD). In some embodiments, the ITAM-modified CAR has at least about 20% (such as at least about any of 30%, 40%, 50%, 60%, 70%, 80%, 90%, or 100%) activity compared to that of a same CAR comprising a CD3ζISD (e.g., traditional CAR with CD3ζISD).

In some embodiments, there is provided an ITAM-modified CAR comprising from N′ to C′: (a) an extracellular ligand binding domain comprising an antigen-binding fragment (e.g., scFv, sdAb) that specifically recognizing one or more epitopes of one or more target antigens (e.g., tumor antigen such as CD19, CD20, or BCMA), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, there is provided an ITAM-modified CAR comprising from N′ to C′: (a) an extracellular ligand binding domain comprising an antigen-binding fragment (e.g., scFv, sdAb) that specifically recognizing one or more epitopes of one or more target antigens (e.g., tumor antigen such as CD19, CD20, or BCMA), (b) a hinge domain (e.g., derived from CD8α), (c) a transmembrane domain (e.g., derived from CD8α), and (d) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, there is provided an ITAM-modified CAR comprising from N′ to C′: (a) an extracellular ligand binding domain comprising an antigen-binding fragment (e.g., scFv, sdAb) that specifically recognizing one or more epitopes of one or more target antigens (e.g., tumor antigen such as CD19, CD20, or BCMA), (b) an optional hinge domain (e.g., derived from CD8α), (c) a transmembrane domain (e.g., derived from CD8α), and (d) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the co-stimulatory signaling domain is N-terminal to the CMSD. In some embodiments, there is provided an ITAM-modified CAR comprising from N′ to C′: (a) an extracellular ligand binding domain comprising an antigen-binding fragment (e.g., scFv, sdAb) that specifically recognizing one or more epitopes of one or more target antigens (e.g., tumor antigen such as CD19, CD20, or BCMA), (b) an optional hinge domain (e.g., derived from CD8α), (c) a transmembrane domain (e.g., derived from CD8α), and (d) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the co-stimulatory signaling domain is C-terminal to the CMSD. In some embodiments, there is provided an ITAM-modified CAR comprising from N′ to C′: (a) an extracellular ligand binding domain comprising one or more scFvs or sdAbs specifically recognizing one or more epitopes of one or more target antigens (e.g., tumor antigen such as CD19, CD20, or BCMA), (b) an optional hinge domain (e.g., derived from CD8α), (c) a transmembrane domain (e.g., derived from CD8α), and (c) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the co-stimulatory signaling domain is N-terminal to the CMSD. In some embodiments, there is provided an ITAM-modified CAR comprising from N′ to C′: (a) an extracellular ligand binding domain comprising one or more scFvs or sdAbs specifically recognizing one or more epitopes of one or more target antigens (e.g., tumor antigen such as CD19, CD20, or BCMA), (b) an optional hinge domain (e.g., derived from CD8α), (c) a transmembrane domain (e.g., derived from CD8α), and (d) an ISD comprising a co-stimulatory signaling domain (e.g., derived from 4-1BB or CD28) and a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the co-stimulatory signaling domain is C-terminal to the CMSD. In some embodiments, the extracellular ligand binding domain comprises one or more sdAbs that specifically bind BCMA (i.e., anti-BCMA sdAb), such as any of the anti-BCMA sdAbs disclosed in PCT/CN2016/094408 and PCT/CN2017/096938, the contents of each of which are incorporated herein by reference in their entirety. In some embodiments, the one or more anti-BCMA sdAb moieties (e.g., V_{H}H) comprise a CDR1 comprising the amino acid sequence of SEQ ID NO: 130, a CDR2 comprising the amino acid sequence of SEQ ID NO: 131, and a CDR3 comprising the amino acid sequence of SEQ ID NO: 132. In some embodiments, the one or more anti-BCMA sdAb moieties (e.g., V_{H}H) comprise the amino acid sequence of SEQ ID NO: 128. In some embodiments, the one or more anti-BCMA sdAb moieties (e.g., V_{H}H) comprise a CDR1 comprising the amino acid sequence of SEQ ID NO: 133, a CDR2 comprising the amino acid sequence of SEQ ID NO: 134, and a CDR3 comprising the amino acid sequence of SEQ ID NO: 135. In some embodiments, the one or more anti-BCMA sdAb moieties (e.g., V_{H}H) comprise the amino acid sequence of SEQ ID NO: 129. In some embodiments, the co-stimulatory signaling domain comprises the sequence of SEQ ID NO: 124. In some embodiments, the transmembrane domain comprises a sequence of SEQ ID NO: 126. In some embodiments, the hinge domain comprises the sequence of SEQ ID NO: 125. In some embodiments, the ITAM-modified CAR further comprises a signal peptide located at the N-terminus of the ITAM-modified CAR (i.e., N-terminus of the extracellular ligand binding domain). In some embodiments, the signal peptide is derived from CD8α. In some embodiments, the signal peptide comprises the sequence of SEQ ID NO: 127. In some embodiments, the signal peptide is removed after the exportation to the cell surface of the ITAM-modified CAR. In some embodiments, the extracellular ligand binding domain (or the ITAM-modified CAR) is monovalent, i.e., comprising one antigen-binding fragment (e.g., scFv, sdAb) specifically recognizing one epitope of a target e.g., tumor) antigen. In some embodiments, the extracellular ligand binding domain (or the ITAM-modified CAR) is multivalent (e.g., bivalent) and multispecific (e.g., bispecific), i.e., comprising two or more (e.g., 2, 3, 4, 5, or more) antigen-binding fragments (e.g., scFv, sdAb) that specifically recognizing two or more (e.g., 2, 3, 4, 5, or more) epitopes of a target (e.g., tumor) antigen. In some embodiments, the two or more epitopes are from the same target (e.g., tumor) antigen. In some embodiments, the two or more epitopes are from different target (e.g., tumor) antigens. In some embodiments, the extracellular ligand binding domain (or the ITAM-modified CAR) is multivalent (e.g., bivalent) and monospecific, comprising two or more (e.g., 2, 3, 4, 5, or more) antigen-binding fragments (e.g., scFv, sdAb) that specifically recognizing the same epitope of a target (e.g., tumor) antigen. In some embodiments, the extracellular ligand binding domain comprises two or more antigen-binding fragments (e.g., scFv, sdAb) specifically recognizing one or more epitopes of one or more target antigens (e.g., tumor antigen such as CD19, CD20, or BCMA). In some embodiments, the two or more antigen-binding fragments (e.g., scFv, sdAb) are the same, e.g., two or more identical anti-BCMA sdAbs or anti-BCMA scFvs. In some embodiments, the two or more antigen-binding fragments (e.g., scFv, sdAb) are different from each other, e.g., two or more anti-BCMA sdAbs or anti-BCMA scFvs specifically recognizing the same BCMA epitope, or two or more anti-BCMA sdAbs or anti-BCMA scFvs specifically recognizing different BCMA epitopes. In some embodiments, the one or more antigen-binding fragments are derived from four-chain antibodies. In some embodiments, the one or more antigen-binding fragments are derived from camelid antibodies. In some embodiments, the one or more antigen-binding fragments are derived from human antibodies. In some embodiments, the one or more antigen-binding fragments are selected from the group consisting of a Camel Ig, an Ig NAR, a Fab, an scFv, and a sdAb. In some embodiments, the antigen-binding fragment is an sdAb (e.g., anti-BCMA sdAb) or an scFv (e.g., anti-BCMA scFv, anti-CD20 scFv, anti-CD19 scFv). In some embodiments, the extracellular ligand binding domain comprises two or more sdAbs (e.g., anti-BCMA sdAbs) linked together, either linked directly or via a peptide linker. In some embodiments, there is provided an ITAM-modified CAR comprising the amino acid sequence of any of SEQ ID NOs: 76-96, 98-104, and 106-113. In some embodiments, the ITAM-modified CAR is an ITAM-modified BCMA CAR. Thus in some embodiments, there is provided an ITAM-modified BCMA CAR comprising from N′ to C′: (a) a CD8a signal peptide, (b) an extracellular ligand binding domain comprising an anti-BCMA scFv, (c) a CD8a hinge domain, (d) a CD8α transmembrane domain, (e) a 4-1BB co-stimulatory signaling domain, and (f) a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, there is provided an ITAM-modified BCMA CAR comprising the amino acid sequence of any of SEQ ID NOs: 76-96. In some embodiments, the ITAM-modified CAR is an ITAM-modified CD20 CAR. Thus in some embodiments, there is provided an ITAM-modified CD20 CAR comprising from N′ to C′: (a) a CD8α signal peptide, (b) an extracellular ligand binding domain comprising an anti-CD20 scFv, (c) a CD8α hinge domain, (d) a CD8α transmembrane domain, (e) a 4-1BB co-stimulatory signaling domain, and (f) a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the anti-CD20 scFv is derived from an anti-CD20 antibody such as rituximab (e.g., Rituxan®, MabThera®) or Leu16. In some embodiments, there is provided an ITAM-modified CD20 CAR comprising the amino acid sequence of any of SEQ ID NOs: 98-104. In some embodiments, the ITAM-modified CAR is not down-modulated (e.g., down-regulated for cell surface expression and/or effector function such as signal transduction related to cytolytic activity) by a Nef protein (e.g., wildtype Nef such as wildtype SIV Nef, Nef subtype, or mutant Nef such as mutant SIV Nef). In some embodiments, the ITAM-modified CAR is at most about 80% (such as at most about any of 70%, 60%, 50%, 40%, 30%, 20%, 10%, or 5%) down-modulated (e.g., down-regulated for cell surface expression and/or effector function such as signal transduction involved in cytolytic activity) by a Nef protein compared to when the Nef is absent. In some embodiments, the ITAM-modified CAR is down-modulated (e.g., down-regulated for cell surface expression and/or effector function such as signal transduction involved in cytolytic activity) by a Nef protein the same or similarly as a same CAR comprising a CD3ζISD (e.g., traditional CAR comprising everything the same but with a CD3ζISD). In some embodiments, the ITAM-modified CAR is at least about 3% less (e.g., at least about any of 4%, 5%, 6%, 7%, 8%, 9%, 10%, 15%, 20%, 30%, 40%, 50%, 60%, 70%, 80%, 90%, or 95% less) down-modulated (e.g., down-regulated for cell surface expression and/or effector function such as signal transduction involved in cytolytic activity) by a Nef protein than a traditional CAR comprising a CD3ζISD. In some embodiments, the ITAM-modified CAR is at most about 80% (e.g., at most about any of 70%, 60%, 50%, 40%, 30%, 20%, 10%, or 5%) more down-modulated (e.g., down-regulated for cell surface expression and/or effector function such as signal transduction involved in cytolytic activity) by a Nef protein than a same CAR comprising a CD3ζISD (e.g., traditional CAR with CD3ζISD). In some embodiments, the ITAM-modified CAR has the same or similar effector function (e.g., signal transduction involved in cytolytic activity) compared to that of a same CAR comprising a CD3ζISD (e.g., traditional CAR with a CD3ζISD). In some embodiments, the ITAM-modified CAR has at least about 3% (e.g., at least about any of 4%, 5%, 6%, 7%, 8%, 9%, 10%, 15%, 20%, 30%, 40%, 50%, 60%, 70%, 80%, 90%, or 95%) stronger effector function (e.g., signal transduction involved in cytolytic activity) compared to that of a same CAR comprising a CD3ζISD (e.g., traditional CAR with CD3ζISD). In some embodiments, the ITAM-modified CAR has at most about 80% (e.g., at most about any of 70%, 60%, 50%, 40%, 30%, 20%, 10%, or 5%) less effector function (e.g., signal transduction involved in cytolytic activity) compared to that of a same CAR comprising a CD3ζISD (e.g., traditional CAR with CD3ζISD). In some embodiments, the ITAM-modified CAR has at least about 20% (such as at least about any of 30%, 40%, 50%, 60%, 70%, 80%, 90%, or 100%) activity compared to that of a same CAR comprising a CD3ζISD (e.g., traditional CAR with CD3ζISD).

In some embodiments, the ITAM-modified CAR is an “ITAM-modified BCMA (ligand/receptor-based) CAR.” Thus in some embodiments, there is provided an ITAM-modified BCMA (ligand/receptor-based) CAR comprising from N′ to C′: (a) a CD8α signal peptide, (b) an extracellular ligand binding domain comprising one or more domains derived from APRIL and/or BAFF, (c) a CD8α hinge domain, (d) a CD8α transmembrane domain, (e) a 4-1BB co-stimulatory signaling domain, and (f) a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the extracellular ligand binding domain comprises an extracellular APRIL domain (or functional portion thereof). In some embodiments, the extracellular ligand binding domain comprises an extracellular BAFF domain (or functional portion thereof). In some embodiments, the extracellular ligand binding domain comprises an extracellular APRIL domain and an extracellular BAFF domain (or functional portions thereof). In some embodiments, the extracellular ligand binding domain comprises two or more extracellular domains derived from APRIL and/or BAFF, which are identical to each other. In some embodiments, the extracellular ligand binding domain comprises two or more domains derived from APRIL and/or BAFF, which are different from each other.

In some embodiments, the ITAM-modified CAR is an ITAM-modified ACTR. Thus in some embodiments, there is provided an ITAM-modified ACTR from N′ to C′: (a) a CD8α signal peptide, (b) an extracellular ligand binding domain comprising an FcR (e.g., FcγR), (c) a CD8α hinge domain, (d) a CD8α transmembrane domain, (e) a 4-1BB co-stimulatory signaling domain, and (f) a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the FcγR is selected from the group consisting of FcγRIA (CD64A), FcγRIB (CD64B), FcγRIC (CD64C), FcγRIIA (CD32A), FcγRIIB (CD32B), FcγRIIIA (CD16a), and FcγRIIIB (CD16b). In some embodiments, the FcR specifically recognizing an Fc-containing molecule (e.g., full length antibody). In some embodiments, the modified T cell comprising an ITAM-modified ACTR further expresses an Fc-containing molecule (e.g., anti-BCMA, anti-CD19, or anti-CD20 full length antibody). In some embodiments, the modified T cell comprising an ITAM-modified ACTR when used for treatment is administered in combination with an Fc-containing molecule (e.g., anti-BCMA, anti-CD19, or anti-CD20 full length antibody).

Any CAR known in the art or developed by the Applicant, including the CARs described in PCT/CN2017/096938 and PCT/CN2016/094408 (the contents of each of which are incorporated herein by reference in their entireties), may be used to construct the ITAM-modified CARs described herein, i.e., can contain any structural components except for the CMSD of ITAM-modified CAR. Exemplary structures of ITAM-modified CARs are shown in FIGS. 15A-15D of PCT/CN2017/096938 (ISD will be switched to ISD comprising CMSD described herein).

Isolated nucleic acids encoding any of the ITAM-modified CARs described herein are also provided.

ITAM-Modified BCMA VHH-VHH CAR

In some embodiments, the ITAM-modified BCMA CAR comprises: a) an extracellular ligand binding domain comprising a first sdAb moiety that specifically binds to BCMA and a second sdAb moiety that specifically binds to BCMA, and b) an intracellular signaling domain (ISD). A transmembrane domain (e.g., a transmembrane domain derived from CD8α) may be present between the extracellular ligand binding domain and the ISD. The first sdAb moiety and the second sdAb moiety may bind to the same or different epitopes of BCMA. The two sdAb moieties may be arranged in tandem, optionally linked by a linker sequence, such as a linker comprising the amino acid sequence of GGGGS (SEQ ID NO: 29).

Between the extracellular ligand binding domain and the transmembrane domain of the ITAM-modified BCMA CAR, or between the ISD and the transmembrane domain of the ITAM-modified BCMA CAR, there may be a spacer domain. The spacer domain can be any oligo- or polypeptide that functions to link the transmembrane domain to the extracellular ligand binding domain or the ISD in the polypeptide chain. A spacer domain may comprise up to about 300 amino acids, including for example about 10 to about 100, about 5 to about 30 amino acids, or about 25 to about 50 amino acids.

The transmembrane domain may be the same transmembrane domain described herein for CMSD-containing functional exogenous receptors and may be derived from any membrane-bound or transmembrane protein. Exemplary transmembrane domains may be derived from (i.e. comprise at least the transmembrane region(s) of) the α, β, δ, or γ chain of the T-cell receptor, CD28, CD3ε, CD3ζ, CD45, CD4, CD5, CD8, CD9, CD16, CD22, CD33, CD37, CD64, CD80, CD86, CD134, CD137, or CD154. In some embodiments, the transmembrane domain is derived from CD8α, such as comprising the amino acid sequence of SEQ ID NO: 126. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the ISD of the ITAM-modified BCMA CAR. In some embodiments, the linker is a glycine-serine doublet.

In some embodiments, the transmembrane domain that naturally is associated with one of the sequences in the ISD of the ITAM-modified BCMA CAR is used (e.g., if an ITAM-modified BCMA CAR ISD comprises a 4-1BB co-stimulatory sequence, the transmembrane domain of the ITAM-modified BCMA CAR is derived from the 4-1BB transmembrane domain).

The intracellular signaling domain of the ITAM-modified BCMA CAR is responsible for activation of at least one of the normal effector functions of the immune cell in which the ITAM-modified BCMA CAR has been placed in. Effector function of a T cell, for example, may be cytolytic activity or helper activity including the secretion of cytokines. Thus the term “intracellular signaling domain” or “ISD” refers to the portion of a protein which transduces the effector function signal and directs the cell to perform a specialized function. While usually the entire ISD can be employed, in many cases it is not necessary to use the entire chain. To the extent that a truncated portion of the ISD is used, such truncated portion may be used in place of the intact chain as long as it transduces the effector function signal. The term “intracellular signaling sequence” is thus meant to include any truncated portion of the ISD sufficient to transduce the effector function signal.

T cell activation can be mediated by two distinct classes of intracellular signaling sequence: those that initiate antigen-dependent primary activation through the TCR (primary signaling sequences) and those that act in an antigen-independent manner to provide a secondary or co-stimulatory signal (co-stimulatory signaling sequences). The ITAM-modified BCMA CARs described herein can comprise one or both of the signaling sequences. In some embodiments, the primary signaling sequence comprises any of the CMSD described herein, such as a CMSD comprising the amino acid sequence of any of SEQ ID NOs: 41-74.

The co-stimulatory signaling sequence (also referred to as co-stimulatory signaling domain) described herein can be a portion of the intracellular signaling domain of a co-stimulatory molecule including, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like. The co-stimulatory signaling domain of the ITAM-modified BCMA CAR described herein may be any of the co-stimulatory signaling domain described herein for CMSD-containing functional exogenous receptors. In some embodiments, the co-stimulatory domain is N-terminal to the CMSD. In some embodiments, the co-stimulatory domain is C-terminal to the CMSD. In some embodiments, the co-stimulatory signaling domain is derived from CD137 (4-1BB), such as comprising the amino acid sequence of SEQ ID NO: 124.

In some embodiments, the intracellular signaling domain of the ITAM-modified BCMA CAR comprises the CMSD and the intracellular signaling sequence of 4-1BB. In some embodiments, the transmembrane domain of the ITAM-modified BCMA CAR is derived from CD8α. In some embodiments the ITAM-modified BCMA CAR further comprises a hinge sequence (e.g., a hinge sequence derived from CD8α) between the extracellular ligand binding domain and the transmembrane domain (e.g., the transmembrane domain derived from CD8α). In some embodiments, the hinge domain comprises the amino acid sequence of SEQ ID NO: 125.

In some embodiments, there is provided an ITAM-modified BCMA CAR comprising: a) an extracellular ligand binding domain comprising one or more single domain antibody (sdAb) moieties that specifically bind to BCMA (also referred to as “anti-BCMA sdAb,” such as “anti-BCMA V_{H}H”), b) an optional hinge domain (e.g., CD8α hinge); c) a transmembrane domain (e.g., CD8α TM domain); and d) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers.

In some embodiments, the extracellular ligand binding domain comprising from N′ to C′: a first anti-BCMA sdAb moiety (e.g., V_{H}H), an optional linker, and a second anti-BCMA sdAb moiety (e.g., V_{H}H).

In some embodiments, the first (and/or second) anti-BCMA sdAb moiety (e.g., V_{H}H) comprises a CDR1 comprising the amino acid sequence of SEQ ID NO: 130, a CDR2 comprising the amino acid sequence of SEQ ID NO: 131, and a CDR3 comprising the amino acid sequence of SEQ ID NO: 132. In some embodiments, the first (and/or second) sdAb moiety comprises CDR1, CDR2, and CDR3 of an anti-BCMA sdAb comprising the amino acid sequence of SEQ ID NO: 128. In some embodiments, the first (and/or second) sdAb moiety binds to the same BCMA epitope as an sdAb moiety (e.g., V_{H}H) comprising a CDR1 comprising the amino acid sequence of SEQ ID NO: 130, a CDR2 comprising the amino acid sequence of SEQ ID NO: 131, and a CDR3 comprising the amino acid sequence of SEQ ID NO: 132.

In some embodiments, the second (and/or first) anti-BCMA sdAb moiety (e.g., V_{H}H) comprises a CDR1 comprising the amino acid sequence of SEQ ID NO: 133, a CDR2 comprising the amino acid sequence of SEQ ID NO: 134, and a CDR3 comprising the amino acid sequence of SEQ ID NO: 135. In some embodiments, the second (and/or first) sdAb moiety comprises CDR1, CDR2, and CDR3 of an anti-BCMA sdAb comprising the amino acid sequence of SEQ ID NO: 129. In some embodiments, the second (and/or first) sdAb moiety binds to the same BCMA epitope as an sdAb moiety (e.g., V_{H}H) comprising a CDR1 comprising the amino acid sequence of SEQ ID NO: 133, a CDR2 comprising the amino acid sequence of SEQ ID NO: 134, and a CDR3 comprising the amino acid sequence of SEQ ID NO: 135.

In some embodiments, there is provided a ITAM-modified BCMA CAR comprising from N′ to C′: a) an extracellular ligand binding domain comprising a first anti-BCMA sdAb moiety (e.g., V_{H}H), an optional linker, and a second anti-BCMA sdAb moiety (e.g., V_{H}H); b) an optional hinge domain (e.g., CD8α hinge); c) a transmembrane domain (e.g., CD8α TM domain); and d) an ISD comprising a CMSD, wherein the CMSD comprises the amino acid sequence of any of SEQ ID NOs: 41-74; wherein the first anti-BCMA sdAb moiety comprises a CDR1 comprising the amino acid sequence of SEQ ID NO: 130, a CDR2 comprising the amino acid sequence of SEQ ID NO: 131, and a CDR3 comprising the amino acid sequence of SEQ ID NO: 132; and wherein the second anti-BCMA sdAb moiety comprises a CDR1 comprising the amino acid sequence of SEQ ID NO: 133, a CDR2 comprising the amino acid sequence of SEQ ID NO: 134, and a CDR3 comprising the amino acid sequence of SEQ ID NO: 135. In some embodiments, there is provided a BCMA CAR comprising from N′ to C′: a) an extracellular ligand binding domain comprising a first anti-BCMA sdAb moiety (e.g., V_{H}H), an optional linker, and a second anti-BCMA sdAb moiety (e.g., V_{H}H); b) an optional hinge domain (e.g., CD8α hinge); c) a transmembrane domain (e.g., CD8α TM domain); and d) an ISD comprising a CMSD, wherein the CMSD comprises the amino acid sequence of any of SEQ ID NOs: 41-74; wherein the first anti-BCMA sdAb moiety comprises the amino acid sequence of SEQ ID NO: 128, and wherein the second anti-BCMA sdAb moiety comprises the amino acid sequence of SEQ ID NO: 129. In some embodiments, the ISD further comprises a co-stimulatory signaling domain, such as a co-stimulatory signaling domain derived from CD137 (4-1BB) or CD28. In some embodiments, the co-stimulatory signaling domain comprises the amino acid sequence of SEQ ID NO: 124. In some embodiments, the optional linker comprises the amino acid sequence of SEQ ID NO: 29. In some embodiments, the hinge domain comprises the amino acid sequence of SEQ ID NO: 125. In some embodiments, the transmembrane domain comprises the amino acid sequence of SEQ ID NO: 126. In some embodiments, the ITAM-modified BCMA CAR further comprises a signal peptide at the N-terminus, comprising the amino acid sequence of SEQ ID NO: 127. Any of the hinge domains, transmembrane domains, receptor domain linkers, signal peptides, and CMSDs as described above can be used in the ITAM-modified BCMA CARs described herein. In some embodiments, there is provided an ITAM-modified anti-BCMA CAR comprising the amino acid sequence of any of SEQ ID NOs: 106-112.

In some embodiments, there is provided an ITAM-modified anti-BCMA CAR comprising the amino acid sequence of SEQ ID NO: 113.

### Co-Stimulatory Signaling Domain

Many immune effector cells (e.g., T cells) require co-stimulation, in addition to stimulation of an antigen-specific signal, to promote cell proliferation, differentiation and survival, as well as to activate effector functions of the cell. In some embodiments, the ITAM-modified CAR comprises at least one co-stimulatory signaling domain. The term “co-stimulatory molecule” or “co-stimulatory protein” refers to a cognate binding partner on an immune cell (e.g., T cell) that specifically binds with a co-stimulatory ligand, thereby mediating a co-stimulatory response by the immune cell, such as, but not limited to, proliferation and survival. The term “co-stimulatory signaling domain,” as used herein, refers to at least a portion of a co-stimulatory molecule that mediates signal transduction within a cell to induce an immune response such as an effector function. The co-stimulatory signaling domain of the ITAM-modified CAR described herein can be a cytoplasmic signaling domain from a co-stimulatory protein, which transduces a signal and modulates responses mediated by immune cells, such as T cells, NK cells, macrophages, neutrophils, or eosinophils.

In some embodiments, the ISD of the ITAM-modified CAR does not comprise a co-stimulatory signaling domain. In some embodiments, the ISD of the ITAM-modified CAR comprises a single co-stimulatory signaling domain. In some embodiments, the ISD of the ITAM-modified CAR comprises two or more (such as about any of 2, 3, 4, or more) co-stimulatory signaling domains. In some embodiments, the ISD of the ITAM-modified CAR comprises two or more of the same co-stimulatory signaling domains, for example, two copies of the co-stimulatory signaling domain of CD28 or CD137 (4-1BB). In some embodiments, the ISD of the ITAM-modified CAR comprises two or more co-stimulatory signaling domains from different co-stimulatory proteins. In some embodiments, the ISD of the ITAM-modified CAR comprises a CMSD described herein, and one or more co-stimulatory signaling domains (e.g., derived from 4-1BB). In some embodiments, the one or more co-stimulatory signaling domains and the CMSD are fused to each other via optional peptide linkers. The CMSD, and the one or more co-stimulatory signaling domains may be arranged in any suitable order. In some embodiments, the one or more co-stimulatory signaling domains are located between the transmembrane domain and the CMSD. In some embodiments, the one or more co-stimulatory signaling domains are located at the C-terminus of the CMSD. In some embodiments, the CMSD is between two or more co-stimulatory signaling domains. Multiple co-stimulatory signaling domains may provide additive or synergistic stimulatory effects. In some embodiments, the transmembrane domain, the one or more co-stimulatory signaling domains, and/or the CMSD are connected via optional peptide linkers, such as any of the peptide linkers as described in “CMSD linker” and “receptor domain linkers” subsections. In some embodiments, the peptide linker comprises the amino acid sequence of any of SEQ ID NOs: 17-39 and 116-120, such as any of SEQ ID NOs: 17-31.

Activation of a co-stimulatory signaling domain in a host cell (e.g., an immune cell such as T cell) may induce the cell to increase or decrease the production and secretion of cytokines, phagocytic properties, proliferation, differentiation, survival, and/or cytotoxicity. The type(s) of co-stimulatory signaling domain is selected for use in the ITAM-modified CARs described herein based on factors such as the type of the immune effector cells in which the ITAM-modified CAR would be expressed (e.g., T cells, NK cells, macrophages, neutrophils, or eosinophils) and the desired immune effector function (e.g., ADCC effect). Examples of co-stimulatory signaling domains for use in the ITAM-modified CARs can be cytoplasmic signaling domain of any co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, GI24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLPR, lymphocyte function associated antigen-1 (LFA-1), and NKG2C. In some embodiments, the one or more co-stimulatory signaling domains is derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, BLAME (SLAMF8), LTBR, LAT, GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof. In some embodiments, the one or more co-stimulatory signaling domains is derived from 4-1BB or CD28. In some embodiments, the co-stimulatory signaling domain comprises the amino acid sequence of SEQ ID NO: 124.

In some embodiments, the ISD of the ITAM-modified CAR comprises (e.g., consists essentially of, or consists of) a co-stimulatory signaling domain derived from 4-1BB, and a CMSD described herein. In some embodiments, the ISD of the ITAM-modified CAR comprises (e.g., consists essentially of, or consists of) a co-stimulatory signaling domain derived from CD28, and a CMSD described herein. In some embodiments, the ISD of the ITAM-modified CAR comprises (e.g., consists essentially of, or consists of) a co-stimulatory signaling domain derived from 4-1BB, a co-stimulatory signaling domain derived from CD28, and a CMSD described herein. In some embodiments, the ISD of the ITAM-modified CAR comprises (e.g., consists essentially of, or consists of) from N′ to C′: a co-stimulatory signaling domain derived from 4-1BB, and a CMSD. In some embodiments, the ISD of the ITAM-modified CAR comprises (e.g., consists essentially of, or consists of) from N′ to C′: a CMSD, and a co-stimulatory signaling domain derived from 4-1BB.

Also within the scope of the present disclosure are variants of any of the co-stimulatory signaling domains described herein, such that the co-stimulatory signaling domain is capable of modulating the immune response of the immune cell (e.g., T cell). In some embodiments, the co-stimulatory signaling domain comprises up to about 10 amino acid residue variations (e.g., about any of 1, 2, 3, 4, 5, 6, 7, 8, 9, or 10) as compared to a wildtype counterpart co-stimulatory signaling domain. Such co-stimulatory signaling domains comprising one or more amino acid variations may be referred to as co-stimulatory signaling domain variants. In some embodiments, mutation of amino acid residues of the co-stimulatory signaling domain may result in an increase in signaling transduction and enhanced stimulation of immune responses relative to co-stimulatory signaling domains that do not comprise the mutation. In some embodiments, mutation of amino acid residues of the co-stimulatory signaling domain may result in a decrease in signaling transduction and reduced stimulation of immune responses relative to co-stimulatory signaling domains that do not comprise the mutation.

### ITAM-Modified T Cell Antigen Coupler (TAC)-Like Chimeric Receptors

In some embodiments, the functional exogenous receptor comprising a CMSD described herein is an ITAM-modified TAC-like chimeric receptor. In some embodiments, the ITAM-modified TAC-like chimeric receptor comprises an ISD comprising any of the CMSDs described herein, such as a CMSD comprising the amino acid sequence of any of SEQ ID NOs: 41-74. In some embodiments, there is provided an ITAM-modified TAC-like chimeric receptor comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional first receptor domain linker, (c) an extracellular TCR binding domain that specifically recognizes the extracellular domain of a first TCR subunit (e.g., CD3ε), (d) an optional second receptor domain linker, (e) an optional extracellular domain of a second TCR subunit (e.g., CD3ε) or a portion thereof, (f) a transmembrane domain comprising a transmembrane domain of a third TCR subunit (e.g., CD3ε), and (g) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the first, second, and third TCR subunits are independently selected from the group consisting of TCRα, TCRβ, TCRγ, TCRδ, CD3ε, CD3γ, and CD3δ. In some embodiments, the ITAM-modified TAC-like chimeric receptor fusion polypeptide can be incorporated into a functional TCR complex along with other endogenous TCR subunits, e.g., by specifically recognizing the extracellular domain of a TCR subunit (e.g., CD3ε, TCRα), and confer antigen specificity to the TCR complex. In some embodiments, the second and third TCR subunits are the same, e.g., both are CD3ε. In some embodiments, the second and third TCR subunits are different. In some embodiments, the first, second, and third TCR subunits are the same, e.g., all are CD3ε. In some embodiments, the first TCR subunit and the second and third TCR subunits are different, e.g., the first TCR subunit is TCRα and the second and third TCR subunits are both CD3ε. In some embodiments, the first, second, and third TCR subunits are all different. In some embodiments, the first TCR subunit is CD3ε, and/or the second TCR subunit is CD3ε, and/or the third TCR subunit is CD3ε. In some embodiments, the first TCR subunit is CD3γ, and/or the second TCR subunit is CD3γ, and/or the third TCR subunit is CD3γ. In some embodiments, the first TCR subunit is CD3δ, and/or the second TCR subunit is CD3δ, and/or the third TCR subunit is CD3δ. In some embodiments, the first TCR subunit is TCRα, and/or the second TCR subunit is TCRα, and/or the third TCR subunit is TCRα. In some embodiments, the first TCR subunit is TCRβ, and/or the second TCR subunit is TCRβ, and/or the third TCR subunit is TCRβ. In some embodiments, the first TCR subunit is TCRγ, and/or the second TCR subunit is TCRγ, and/or the third TCR subunit is TCRγ. In some embodiments, the first TCR subunit is TCRδ, and/or the second TCR subunit is TCRδ, and/or the third TCR subunit is TCRδ. In some embodiments, the first TCR subunit and the third TCR subunit are the same. In some embodiments, the first TCR subunit and the third TCR subunit are different. In some embodiments, the first TCR subunit and the second TCR subunit are the same. In some embodiments, the first TCR subunit and the second TCR subunit are different. In some embodiments, the ITAM-modified TAC-like chimeric receptor does not comprise an extracellular domain of a second TCR subunit or a portion thereof. In some embodiments, the ITAM-modified TAC-like chimeric receptor does not comprise an extracellular domain of any TCR subunit. In some embodiments, the extracellular ligand binding domain is N-terminal to the extracellular TCR binding domain. In some embodiments, the extracellular ligand binding domain is C-terminal to the extracellular TCR binding domain. In some embodiments, the ITAM-modified TAC-like chimeric receptor further comprises a hinge domain located between the C-terminus of the extracellular TCR binding domain and the N-terminus of the transmembrane domain (e.g., when there is no extracellular domain of a TCR subunit or a portion thereof, and the extracellular TCR binding domain is at C-terminus of the extracellular ligand binding domain). In some embodiments, the ITAM-modified TAC-like chimeric receptor further comprises a hinge domain located between the C-terminus of the extracellular ligand binding domain and the N-terminus of the transmembrane domain (e.g., when there is no extracellular domain of a TCR subunit or a portion thereof, and the extracellular TCR binding domain is at N-terminus of the extracellular ligand binding domain). Any of the hinge domains and linkers described in the above “hinge,” “CMSD linker,” and “receptor domain linkers” subsections can be used in the ITAM-modified TAC-like chimeric receptor described herein. In some embodiments, the first and/or second receptor domain linkers are selected from the group consisting of SEQ ID NOs: 17-39 and 116-120. In some embodiments, the hinge domain is derived from CD8α. In some embodiments, the hinge domain comprises the sequence of SEQ ID NO: 125. In some embodiments, the extracellular ligand binding domain is monovalent and monospecific, e.g., comprising a single antigen-binding fragment (e.g., scFv, sdAb) that specifically recognizes an epitope of a target antigen (e.g., tumor antigen such as BCMA, CD19, CD20). In some embodiments, the extracellular ligand binding domain is multivalent and monospecific, e.g., comprising two or more antigen-binding fragments (e.g., scFv, sdAb) that specifically recognize the same epitope of a target antigen (e.g., tumor antigen such as BCMA, CD19, CD20). In some embodiments, the extracellular ligand binding domain is multivalent and multispecific, e.g., comprising two or more antigen-binding fragments (e.g., scFv, sdAb) that specifically recognize two or more epitopes of the same target (e.g., tumor) antigen or different target antigens (e.g., tumor antigen such as BCMA, CD19, CD20). In some embodiments, the ITAM-modified TAC-like chimeric receptor further comprises a second extracellular TCR binding domain (e.g., scFv, sdAb) that specifically recognizes a different extracellular domain of a TCR subunit (e.g., TCRα) that is recognized by the extracellular TCR binding domain (e.g., CD3ε), wherein the second extracellular TCR binding domain is situated between the extracellular TCR binding domain and the extracellular ligand binding domain. In some embodiments, the extracellular ligand binding domain comprises one or more sdAbs that specifically bind BCMA (i.e., anti-BCMA sdAb), such as any of the anti-BCMA sdAbs described herein, or any of the anti-BCMA sdAbs disclosed in PCT/CN2016/094408 and PCT/CN2017/096938, the content of which are incorporated herein by reference in their entirety. In some embodiments, the extracellular ligand binding domain comprises one or more anti-BCMA scFvs. In some embodiments, the ITAM-modified TAC-like chimeric receptor further comprises a signal peptide located at the N-terminus of the ITAM-modified TAC-like chimeric receptor, e.g., the signal peptide is at the N-terminus of the extracellular ligand binding domain if the extracellular ligand binding domain is N-terminal to the extracellular TCR binding domain, or the signal peptide is at the N-terminus of the extracellular TCR binding domain if the extracellular ligand binding domain is C-terminal to the extracellular TCR binding domain. In some embodiments, the signal peptide is derived from CD8α. In some embodiments, the signal peptide comprises the sequence of SEQ ID NO: 127. In some embodiments, the signal peptide is removed after the exportation to the cell surface of the ITAM-modified TAC-like chimeric receptor. In some embodiments, the plurality (e.g., 2, 3, 4, or more) of CMSD ITAMs are directly linked to each other. In some embodiments, the CMSD comprises two or more (e.g., 2, 3, 4, or more) CMSD ITAMs connected by one or more linkers not derived from an ITAM-containing parent molecule (e.g., G/S linker). In some embodiments, the CMSD comprises one or more CMSD linkers derived from an ITAM-containing parent molecule that is different from the ITAM-containing parent molecule from which one or more of the CMSD ITAMs are derived from. In some embodiments, the CMSD comprises two or more (e.g., 2, 3, 4, or more) identical CMSD ITAMs. In some embodiments, at least one of the CMSD ITAMs is not derived from CD3ζ. In some embodiments, at least one of the CMSD ITAMs is not ITAM1 or ITAM2 of CD3ζ. In some embodiments, the plurality of CMSD ITAMs are each derived from a different ITAM-containing parent molecule. In some embodiments, at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, at least one of the plurality of CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin. In some embodiments, the plurality of CMSD ITAMs are derived from one or more of CD3ε, CD3δ, CD3γ, CD3ζ, DAP12, Igα (CD79a), Igβ (CD79b), and FcεRIγ. In some embodiments, the CMSD does not comprise CD3ζ ITAM1 and/or CD3ζ ITAM2. In some embodiments, the CMSD comprises CD3ζ ITAM3. In some embodiments, the CMSD does not comprise any CD3ζ ITAMs. In some embodiments, the ITAM-modified TAC-like chimeric receptor is not down-modulated (e.g., down-regulated for cell surface expression and/or effector function such as signal transduction related to cytolytic activity) by a Nef protein (e.g., wildtype Nef such as wildtype SIV Nef, or mutant Nef such as mutant SIV Nef). In some embodiments, the ITAM-modified TAC-like chimeric receptor is at most about 80% (such as at most about any of 70%, 60%, 50%, 40%, 30%, 20%, 10%, or 5%) down-modulated (e.g., down-regulated for cell surface expression and/or effector function) by a Nef protein (e.g., wildtype Nef such as wildtype SIV Nef, or mutant Nef such as mutant SIV Nef) compared to when the Nef is absent. In some embodiments, the ITAM-modified TAC-like chimeric receptor is at least about 3% less (e.g., at least about any of 4%, 5%, 6%, 7%, 8%, 9%, 10%, 15%, 20%, 30%, 40%, 50%, 60%, 70%, 80%, 90%, or 95% less) down-modulated (e.g., down-regulated for cell surface expression and/or effector function such as signal transduction involved in cytolytic activity) by a Nef protein (e.g., wildtype Nef such as wildtype SIV Nef, or mutant Nef such as mutant SIV Nef) than a TAC-like chimeric receptor comprising an ISD of CD3ε, CD3δ, or CD3γ. In some embodiments, the CMSD ITAMs are derived from CD3ζ. In some embodiments, the second and third TCR subunits are both CD3ε. In some embodiments, the CMSD ITAMs are derived from one or more of CD3ε, CD3δ, and CD3γ. In some embodiments, the linkers within the CMSD are derived from CD3ε, CD3δ, or CD3γ (e.g., non-ITAM sequence of the ISD of CD3ε, CD3δ, or CD3δ, or selected from the group consisting of SEQ ID NOs: 17-39 and 116-120. In some embodiments, the CMSD consists essentially of (e.g., consists of) one CD3ε ITAM. In some embodiments, the CMSD comprises at least two CD3ε ITAMs. In some embodiments, the CMSD comprises the amino acid sequence of any of SEQ ID NO: 46, 56, 67, or 71.

In some embodiments, the CMSD ITAMs are derived from CD3ζ. Thus in some embodiments, there is provided an ITAM-modified TAC-like chimeric receptor comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional first receptor domain linker, (c) an extracellular TCR binding domain that specifically recognizes the extracellular domain of a first TCR subunit (e.g., CD3ε), (d) an optional second receptor domain linker, (e) an optional extracellular domain of a second TCR subunit (e.g., CD3ε) or a portion thereof, (f) a transmembrane domain comprising a transmembrane domain of a third TCR subunit (e.g., CD3ε), and (g) an ISD comprising a CMSD, wherein the CMSD comprises one or a plurality of CMSD ITAMs derived from CD3ε, wherein the plurality of CMSD ITAMs are optionally connected by one or more CMSD linkers, and wherein the first, second, and third TCR subunits are all independently selected from the group consisting of TCRα, TCRβ, TCRγ, TCRδ, CD3ε, CD3γ, and CD3δ. In some embodiments, the CMSD comprises a sequence selected from the group consisting of SEQ ID NOs: 41-44, 54, and 55.

In some embodiments, there is provided an ITAM-modified TAC-like chimeric receptor comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional first receptor domain linker, (c) an extracellular TCR binding domain that specifically recognizes the extracellular domain of a first TCR subunit (e.g., CD3ε), (d) an optional second receptor domain linker, (e) an optional extracellular domain of a second TCR subunit (e.g., CD3ε) or a portion thereof, (f) a transmembrane domain comprising a transmembrane domain of a third TCR subunit (e.g., CD3ε), and (g) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, wherein the ITAMs are derived from one or more of CD3ε, CD3δ, and CD3γ, and wherein the first, second, and third TCR subunits are independently selected from the group consisting of TCRα, TCRβ, TCRγ, TCRδ, CD3ε, CD3γ, and CD3δ. In some embodiments, the CMSD comprises (e.g., consists essentially of or consists of) one or a plurality of (e.g., 2, 3, or more) CD3ε ITAMs, and the second TCR subunit is CD3ε and/or the third TCR subunit is CD3ε. In some embodiments, the CMSD comprises (e.g., consists essentially of or consists of) one or a plurality of (e.g., 2, 3, or more) CD36 ITAMs, and the second TCR subunit is CD36 and/or the third TCR subunit is CD3δ. In some embodiments, the CMSD comprises (e.g., consists essentially of or consists of) one or a plurality of (e.g., 2, 3, or more) CD3γ ITAMs, and the second TCR subunit is CD3γ and/or the third TCR subunit is CD3γ. In some embodiments, the first TCR subunit is the same as the second TCR subunit and/or the third TCR subunit. In some embodiments, the second TCR subunit and the third TCR subunit are the same, but different from the first TCR subunit.

Thus in some embodiments, there is provided an ITAM-modified TAC-like chimeric receptor comprising: (a) an extracellular ligand binding domain comprising an antigen-binding fragment (e.g., scFv, sdAb) that specifically recognizes one or more epitopes of one or more target antigens (e.g., tumor antigen such as BCMA, CD20, CD19), (b) an optional first receptor domain linker, (c) an extracellular TCR binding domain that specifically recognizes the extracellular domain of a TCR subunit (e.g., TCRα), (d) an optional second receptor domain linker, (e) an optional extracellular domain of CD3ε or a portion thereof, (f) a transmembrane domain comprising a transmembrane domain of CD3ε, and (g) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the TCR subunit is selected from the group consisting of TCRα, TCRβ, TCRγ, TCRδ, CD3ε, CD3γ, and CD3δ. In some embodiments, there is provided an ITAM-modified TAC-like chimeric receptor comprising: (a) an extracellular ligand binding domain comprising an antigen-binding fragment (e.g., scFv, sdAb) that specifically recognizes one or more epitopes of one or more target antigens (e.g., tumor antigen such as BCMA, CD20, CD19), (b) an optional first receptor domain linker, (c) an extracellular TCR binding domain that specifically recognizes the extracellular domain of a TCR subunit (e.g., TCRα), (d) an optional second receptor domain linker, (e) an optional extracellular domain of CD3ε or a portion thereof, (f) a transmembrane domain comprising a transmembrane domain of CD3ε, and (g) an ISD comprising a CMSD, wherein the CMSD comprises one or a plurality of CD3ε ITAMs, wherein the plurality of CD3ε ITAMs are optionally connected by one or more CMSD linkers, and wherein the TCR subunit is selected from the group consisting of TCRα, TCRβ, TCRγ, TCRδ, CD3ε, CD3γ, and CD3δ. In some embodiments, the CMSD comprises the sequence selected from the group consisting of SEQ ID NO: 46, 56, or 67.

In some embodiments, the ITAM-modified TAC-like chimeric receptor does not comprise an extracellular domain of any TCR subunit. In some embodiments, the ITAM-modified TAC-like chimeric receptor comprises a hinge domain. Thus in some embodiments, there is provided an ITAM-modified TAC-like chimeric receptor comprising: (a) an extracellular ligand binding domain comprising an antigen-binding fragment (e.g., scFv, sdAb) that specifically recognizes one or more epitopes of one or more target antigens (e.g., tumor antigen such as BCMA, CD20, CD19), (b) an optional first receptor domain linker, (c) an extracellular TCR binding domain that specifically recognizes the extracellular domain of a first TCR subunit (e.g., TCRα), (d) an optional second receptor domain linker, (e) an optional hinge domain, (f) a transmembrane domain comprising a transmembrane domain of a second TCR subunit (e.g., CD3ε), and (g) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the first and second TCR subunits are both selected from the group consisting of TCRα, TCRβ, TCRγ, TCRδ, CD3ε, CD3γ, and CD3δ.

### ITAM-Modified TCRs

In some embodiments, the functional exogenous receptor comprising a CMSD described herein is an “ITAM-modified TCR.” In some embodiments, the ITAM-modified TCR comprises an ISD comprising any of the CMSDs described herein, such as a CMSD comprising the amino acid sequence of any of SEQ ID NOs: 41-74. In some embodiments, there is provided an ITAM-modified TCR comprising: (a) an extracellular ligand binding domain comprising a Vα and a Vβ derived from a wildtype TCR together specifically recognizing one or more epitopes of one or more target antigens (e.g., tumor antigen such as BCMA, CD19, CD20) or target antigen peptide/MHC complex (e.g., BCMA/MHC complex), wherein the Vα, the Vβ, or both, comprise one or more mutations in one or more CDRs relative to the wildtype TCR, (b) a transmembrane domain comprising a transmembrane domain of TCRα and a transmembrane domain of TCRβ, and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, the mutation leads to amino acid substitutions, such as conservative amino acid substitutions. In some embodiments, the ITAM-modified TCR binds to the same cognate peptide-MHC bound by the wildtype TCR. In some embodiments, the ITAM-modified TCR binds to the same cognate peptide-MHC with higher affinity compared to that bound by the wildtype TCR. In some embodiments, the ITAM-modified TCR binds to the same cognate peptide-MHC with lower affinity compared to that bound by the wildtype TCR. In some embodiments, the ITAM-modified TCR binds to a non-cognate peptide-MHC not bound by the wildtype TCR. In some embodiments, the ITAM-modified TCR is a single chain TCR (scTCR). In some embodiments, the ITAM-modified TCR is a dimeric TCR (dTCR). In some embodiments, the wildtype TCR binds HLA-A2. In some embodiments, the plurality (e.g., 2, 3, 4, or more) of CMSD ITAMs are directly linked to each other. In some embodiments, the CMSD comprises two or more (e.g., 2, 3, 4, or more) CMSD ITAMs connected by one or more linkers not derived from an ITAM-containing parent molecule (e.g., G/S linker). In some embodiments, the CMSD comprises one or more CMSD linkers derived from an ITAM-containing parent molecule that is different from the ITAM-containing parent molecule from which one or more of the CMSD ITAMs are derived from. In some embodiments, the CMSD comprises two or more (e.g., 2, 3, 4, or more) identical CMSD ITAMs. In some embodiments, at least one of the CMSD ITAMs is not derived from CD3ζ. In some embodiments, at least one of the CMSD ITAMs is not ITAM1 or ITAM2 of CD3ζ. In some embodiments, the plurality of CMSD ITAMs are each derived from a different ITAM-containing parent molecule. In some embodiments, at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, at least one of the plurality of CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin. In some embodiments, the plurality of CMSD ITAMs are derived from one or more of CD3ε, CD3δ, CD3γ, CD3ζ, DAP12, Igα (CD79a), Igβ (CD79b), and FcεRIγ. In some embodiments, the CMSD does not comprise CD3ζ ITAM1 and/or CD3ζ ITAM2. In some embodiments, the CMSD comprises CD3ζ ITAM3. In some embodiments, the CMSD does not comprise any CD3ζ ITAMs. In some embodiments, the ITAM-modified TCR further comprises a hinge domain located between the C-terminus of the extracellular ligand binding domain and the N-terminus of the transmembrane domain. Any of the hinge domains described in the above “hinge” subsections can be used in the ITAM-modified TCR described herein. In some embodiments, the hinge domain is derived from CD8α. In some embodiments, the hinge domain comprises the sequence of SEQ ID NO: 125. In some embodiments, the ITAM-modified TCR further comprises a signal peptide located at the N-terminus of the ITAM-modified TCR (i.e., N-terminus of the extracellular ligand binding domain). In some embodiments, the signal peptide is derived from CD8α. In some embodiments, the signal peptide comprises the sequence of SEQ ID NO: 127. In some embodiments, the signal peptide is removed after the exportation to the cell surface of the ITAM-modified TCR. In some embodiments, the ITAM-modified TCR is not down-modulated (e.g., not down-regulated for cell surface expression and/or effector function such as signal transduction related to cytolytic activity) by a Nef protein (e.g., wildtype Nef such as wildtype SIV Nef, or mutant Nef such as mutant SIV Nef). In some embodiments, the ITAM-modified TCR is at most about 80% (such as at most about any of 70%, 60%, 50%, 40%, 30%, 20%, 10%, or 5%) down-modulated (e.g., down-regulated for cell surface expression and/or effector function such as signal transduction involved in cytolytic activity) by a Nef protein (e.g., wildtype Nef such as wildtype SIV Nef, or mutant Nef such as mutant SIV Nef) compared to when the Nef is absent. In some embodiments, the ITAM-modified TCR is at least about 3% less (e.g., at least about any of 4%, 5%, 6%, 7%, 8%, 9%, 10%, 15%, 20%, 30%, 40%, 50%, 60%, 70%, 80%, 90%, or 95% less) down-modulated (e.g., down-regulated for cell surface expression and/or effector function such as signal transduction involved in cytolytic activity) by a Nef protein (e.g., wildtype Nef such as wildtype SIV Nef, or mutant Nef such as mutant SIV Nef) than a same modified TCR complexed with an endogenous CD3ζ.

ITAM-Modified Chimeric TCRs (cTCRs)

In some embodiments, the functional exogenous receptor comprising a CMSD described herein is an ITAM-modified cTCR. In some embodiments, the ITAM-modified cTCR comprises an ISD comprising any of the CMSDs described herein, such as a CMSD comprising the amino acid sequence of any of SEQ ID NOs: 41-74. In some embodiments, there is provided an ITAM-modified cTCR comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional receptor domain linker, (c) an optional extracellular domain of a first TCR subunit (e.g., CD3ε) or a portion thereof, (d) a transmembrane domain comprising a transmembrane domain of a second TCR subunit (e.g., CD3ε), and (e) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the first and second TCR subunits are independently selected from the group consisting of TCRα, TCRβ, TCRγ, TCRδ, CD3ε, CD3γ, and CD3δ. In some embodiments, the ITAM-modified cTCR fusion polypeptide can be incorporated into a functional TCR complex along with other endogenous TCR subunits and confer antigen specificity to the TCR complex. In some embodiments, the first and second TCR subunits are the same, e.g., both are CD3. In some embodiments, the first and second TCR subunits are different, e.g., the first TCR subunit is TCRα and the second TCR subunit is CD3ε. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD36 and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRγ and/or the second TCR subunit is TCRγ. In some embodiments, the first TCR subunit is TCRδ and/or the second TCR subunit is TCRδ. In some embodiments, the ITAM-modified cTCR does not comprise an extracellular domain of a first TCR subunit or a portion thereof. In some embodiments, the ITAM-modified cTCR does not comprise an extracellular domain of any TCR subunit. In some embodiments, the ITAM-modified cTCR further comprises a hinge domain located between the C-terminus of the extracellular ligand binding domain and the N-terminus of the transmembrane domain (e.g., when there is no extracellular domain of a TCR subunit or a portion thereof). Any of the hinge domains and receptor domain linkers described in the above “hinge” and “receptor domain linkers” subsections can be used in the ITAM-modified cTCR described herein. In some embodiments, the receptor domain linker is selected from the group consisting of SEQ ID NOs: 17-39 and 116-120. In some embodiments, the hinge domain is derived from CD8α. In some embodiments, the hinge domain comprises the sequence of SEQ ID NO: 125. In some embodiments, the extracellular ligand binding domain is monovalent and monospecific, e.g., comprising a single antigen-binding fragment (e.g., scFv, sdAb) that specifically recognizes an epitope of a target antigen (e.g., tumor antigen such as BCMA, CD19, CD20). In some embodiments, the extracellular ligand binding domain is multivalent and monospecific, e.g., comprising two or more antigen-binding fragments (e.g., scFv, sdAb) that specifically recognize the same epitope of a target antigen (e.g., tumor antigen such as BCMA, CD19, CD20). In some embodiments, the extracellular ligand binding domain is multivalent and multispecific, e.g., comprising two or more antigen-binding fragments (e.g., scFv, sdAb) that specifically recognize two or more epitopes of the same target antigen or different target antigens (e.g., tumor antigen such as BCMA, CD19, CD20). In some embodiments, the extracellular ligand binding domain comprises one or more sdAbs that specifically bind BCMA (i.e., anti-BCMA sdAb), such as any anti-BCMA sdAbs described herein, or any of the of the anti-BCMA sdAbs disclosed in PCT/CN2016/094408 and PCT/CN2017/096938, the contents of each of which are incorporated herein by reference in their entirety. In some embodiments, the extracellular ligand binding domain comprises one or more anti-BCMA scFvs. In some embodiments, the ITAM-modified cTCR further comprises a signal peptide located at the N-terminus of the ITAM-modified cTCR, e.g., the signal peptide is at the N-terminus of the extracellular ligand binding domain. In some embodiments, the signal peptide is derived from CD8α. In some embodiments, the signal peptide comprises the sequence of SEQ ID NO: 127. In some embodiments, the signal peptide is removed after the exportation to the cell surface of the ITAM-modified cTCR. In some embodiments, the plurality (e.g., 2, 3, 4, or more) of CMSD ITAMs are directly linked to each other. In some embodiments, the CMSD comprises two or more (e.g., 2, 3, 4, or more) CMSD ITAMs connected by one or more linkers not derived from an ITAM-containing parent molecule (e.g., G/S linker). In some embodiments, the CMSD comprises one or more CMSD linkers derived from an ITAM-containing parent molecule that is different from the ITAM-containing parent molecule from which one or more of the CMSD ITAMs are derived from. In some embodiments, the CMSD comprises two or more (e.g., 2, 3, 4, or more) identical CMSD ITAMs. In some embodiments, at least one of the CMSD ITAMs is not derived from CD3ζ. In some embodiments, at least one of the CMSD ITAMs is not ITAM1 or ITAM2 of CD3ζ. In some embodiments, the plurality of CMSD ITAMs are each derived from a different ITAM-containing parent molecule. In some embodiments, at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin. In some embodiments, at least one of the plurality of CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin. In some embodiments, the plurality of CMSD ITAMs are derived from one or more of CD3ε, CD3δ, CD3γ, CD3ζ, DAP12, Igα (CD79a), Igβ (CD79b), and FcεRIγ. In some embodiments, the CMSD does not comprise CD3ζ ITAM1 and/or CD3ζ ITAM2. In some embodiments, the CMSD comprises CD3ζ ITAM3. In some embodiments, the CMSD does not comprise any CD3ζ ITAMs. In some embodiments, the ITAM-modified cTCR is not down-modulated (e.g., not down-regulated for cell surface expression and/or effector function such as signal transduction related to cytolytic activity) by a Nef protein (e.g., wildtype Nef such as wildtype SIV Nef, or mutant Nef such as mutant SIV Nef). In some embodiments, the ITAM-modified cTCR is at most about 80% (such as at most about any of 70%, 60%, 50%, 40%, 30%, 20%, 10%, or 5%) down-modulated (e.g., down-regulated for cell surface expression and/or effector function such as signal transduction related to cytolytic activity) by a Nef protein (e.g., wildtype Nef such as wildtype SIV Nef, or mutant Nef such as mutant SIV Nef) compared to when the Nef is absent. In some embodiments, the ITAM-modified cTCR is at least about 3% less (e.g., at least about any of 4%, 5%, 6%, 7%, 8%, 9%, 10%, 15%, 20%, 30%, 40%, 50%, 60%, 70%, 80%, 90%, or 95% less) down-modulated (e.g., down-regulated for cell surface expression and/or effector function) by a Nef protein (e.g., wildtype Nef such as wildtype SIV Nef, or mutant Nef such as mutant SIV Nef) than a same cTCR comprising an ISD of CD3ε, CD3δ, or CD3γ. In some embodiments, the CMSD ITAMs are derived from CD3ζ. In some embodiments, the first and second TCR subunits are both CD3. In some embodiments, the CMSD ITAMs are derived from one or more of CD3ε, CD3δ, and CD3γ. In some embodiments, the linkers within the CMSD are derived from CD3ε, CD3δ, or CD3γ (e.g., non-ITAM sequence of the ISD of CD3ε, CD3δ, or CD3δ, or selected from the group consisting of SEQ ID NOs: 17-39 and 116-120. In some embodiments, the CMSD consists essentially of (e.g., consists of) one CD3ε ITAM. In some embodiments, the CMSD comprises at least two CD3ε ITAMs. In some embodiments, the CMSD comprises the sequence of any of SEQ ID NOs: 46, 56, 67, or 71.

In some embodiments, the ITAMs are derived from CD3ζ. Thus in some embodiments, there is provided an ITAM-modified cTCR comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional receptor domain linker, (c) an optional extracellular domain of a first TCR subunit (e.g., CD3ε) or a portion thereof, (d) a transmembrane domain comprising a transmembrane domain of a second TCR subunit (e.g., CD3ε), and (e) an ISD comprising a CMSD (e.g., CMSD comprising a sequence selected from the group consisting of SEQ ID NOs: 41-74), wherein the CMSD comprises a plurality of CMSD ITAMs derived from CD3ζ optionally connected by one or more CMSD linkers, and wherein the first and second TCR subunits are independently selected from the group consisting of TCRα, TCRβ, TCRγ, TCRδ, CD3ε, CD3γ, and CD3δ. In some embodiments, the CMSD comprises a sequence selected from the group consisting of SEQ ID NOs: 41-44, 54, and 55.

In some embodiments, there is provided an ITAM-modified cTCR comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) an optional receptor domain linker, (c) an optional extracellular domain of a first TCR subunit (e.g., CD3ε) or a portion thereof, (d) a transmembrane domain comprising a transmembrane domain of a second TCR subunit (e.g., CD3ε), and (e) an ISD comprising a CMSD, wherein the CMSD comprises one or a plurality of CMSD ITAMs, wherein the plurality of CMSD ITAMs are optionally connected by one or more CMSD linkers, wherein the CMSD ITAMs are derived from one or more of CD3ε, CD3δ, and CD3γ, and wherein the first and second TCR subunits are independently selected from the group consisting of TCRα, TCRβ, TCRγ, TCRδ, CD3ε, CD3γ, and CD3δ. In some embodiments, the CMSD comprises (e.g., consists essentially of or consists of) one or a plurality of (e.g., 2, 3, or more) CD3ε ITAMs, and the first TCR subunit is CD3ε and/or the second TCR subunit is CD3. In some embodiments, the CMSD comprises (e.g., consists essentially of or consists of) one or a plurality of (e.g., 2, 3, or more) CD36 ITAMs, and the first TCR subunit is CD36 and/or the second TCR subunit is CD3δ. In some embodiments, the CMSD comprises (e.g., consists essentially of or consists of) one or a plurality of (e.g., 2, 3, or more) CD3γ ITAMs, and the first TCR subunit is CD3γ and/or the second TCR subunit is CD3γ. In some embodiments, the first TCR subunit is the same as the second TCR subunit. In some embodiments, the first TCR subunit is different from the second TCR subunit.

Thus in some embodiments, there is provided an ITAM-modified cTCR comprising: (a) an extracellular ligand binding domain comprising an antigen-binding fragment (e.g., scFv, sdAb) that specifically recognizes one or more epitopes of one or more target antigens (e.g., tumor antigen such as BCMA, CD20, CD19), (b) an optional first receptor domain linker, (c) an optional extracellular domain of CD3ε or a portion thereof, (d) a transmembrane domain comprising a transmembrane domain of CD3ε, and (e) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, there is provided an ITAM-modified cTCR comprising: (a) an extracellular ligand binding domain comprising an antigen-binding fragment (e.g., scFv, sdAb) that specifically recognizes one or more epitopes of one or more target antigens (e.g., tumor antigen such as BCMA, CD20, CD19), (b) an optional first receptor domain linker, (c) an optional extracellular domain of CD3ε or a portion thereof, (d) a transmembrane domain comprising a transmembrane domain of CD3ε, and (e) an ISD comprising a CMSD, wherein the CMSD comprises one or a plurality of CD3ε ITAMs, wherein the plurality of CD3ε ITAMs are optionally connected by one or more CMSD linkers. In some embodiments, the CMSD comprises a sequence of SEQ ID NO: 46, 56, or 67.

In some embodiments, the ITAM-modified cTCR does not comprise an extracellular domain of any TCR subunit. In some embodiments, the ITAM-modified cTCR comprises a hinge domain. Thus in some embodiments, there is provided an ITAM-modified cTCR comprising: (a) an extracellular ligand binding domain comprising an antigen-binding fragment (e.g., scFv, sdAb) that specifically recognizes one or more epitopes of one or more target antigens (e.g., tumor antigen such as BCMA, CD20, CD19), (b) an optional receptor domain linker, (c) an optional hinge domain (e.g., derived from CD8α), (d) a transmembrane domain comprising a transmembrane domain of a TCR subunit (e.g., CD3ε), and (e) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers, and wherein the TCR subunit is selected from the group consisting of TCRα, TCRβ, TCRγ, TCRδ, CD3ε, CD3γ, and CD3δ.

### IV. Nef (Negative Regulatory Factor) Proteins

The Nef protein described herein can be wildtype Nef such as wildtype SIV Nef, or mutant Nef such as mutant SIV Nef Any of the Nef proteins (e.g., wildtype Nef, Nef subtype, mutant Nef such as non-naturally occurring mutant Nef), nucleic acids encoding thereof, vectors (e.g., viral vector) comprising the nucleic acids thereof, modified T cells (e.g., allogeneic T cell) expressing an exogenous Nef protein or comprising a nucleic acid (or vector) encoding thereof as described in PCT/CN2019/097969 and PCT/CN2018/097235 (the contents of each of which are incorporated herein by reference in their entirety), can all be employed in the present invention. In some embodiments, the modified T cell comprising a CMSD-containing functional exogenous receptor described herein can further express an exogenous Nef protein (also referred to as “Nef-containing ITAM-modified T cells” or “GvHD-minimized ITAM-modified T cells”).

Wildtype Nef is a small 27-35 kDa myristoylated protein encoded by primate lentiviruses, including Human Immunodeficiency Viruses (HIV-1 and HIV-2) and Simian Immunodeficiency Virus (SIV). Nef localizes primarily to the cytoplasm but is also partially recruited to the Plasma Membrane. It functions as a virulence factor, which can manipulate the host's cellular machinery and thus allow infection, survival or replication of the pathogen.

Nef is highly conserved in all primate lentiviruses. The HIV-2 and SIV Nef proteins are 10-60 amino acids longer than HIV-1 Nef. From N-terminus to C-terminus, a Nef protein comprises the following domains: myristoylation site (involved in CD4 down-regulation, MHC I down-regulation, and association with signaling molecules, required for inner plasma membrane targeting of Nef and virion incorporation, and thereby for infectivity), N-terminal α-helix (involved in MHC I down-regulation and protein kinase recruitment), tyrosine-based AP recruitment (HIV-2/SIV Nef), CD4 binding site (WL residue, involved in CD4 down-regulation, characterized for HIV-1 Nef), acidic cluster (involved in MHC I down-regulation, interaction with host PACS1 and PACS2), proline-based repeat (involved in MHC I down-regulation and SH3 binding), PAK (p21 activated kinase) binding domain (involved in association with signaling molecules and CD4 down-regulation), COP I recruitment domain (involved in CD4 down-regulation), di-leucine based AP recruitment domain (involved in CD4 down-regulation, HIV-1 Nef), and V-ATPase and Raf-1 binding domain (involved in CD4 down-regulation and association with signaling molecules).

CD4 is a 55 kDa type I integral cell surface glycoprotein. It is a component of the TCR on MHC class II-restricted cells such as helper/inducer T-lymphocytes and cells of the macrophage/monocyte lineage. It serves as the primary cellular receptor for HIV and SIV. CD4 is a co-receptor of TCR and assists TCR in communicating with antigen-presenting cells (APCs), and triggers TCR intracellular signaling.

CD28 expresses on T cells and provides co-stimulatory signals required for T cell activation and survival. T cell stimulation through TCR and CD28 can trigger cytokine production, such as IL-6. CD28 is the receptor for CD80 (B7.1) and CD86 (B7.2) proteins, which are expressed on APCs.

Major histocompatibility complex (MHC) class I are expressed in all cells but red blood cells. It presents epitopes to killer T cells or cytotoxic T lymphocytes (CTLs). If a CTL's TCR recognizes the epitope presented by the MHC class I molecule, which is docked through CTL's CD8 receptor, the CTL will trigger the cell to undergo programmed cell death by apoptosis. It is thus preferable to down-modulate (e.g., down-regulate expression and/or function) MHC class I molecules expressed on modified T cells described herein, to reduce/avoid GvHD response in a histoincompatible individual.

In some embodiments, the Nef protein is selected from the group consisting of SIV Nef, HIV1 Nef, HIV2 Nef, and Nef subtypes. In some embodiments, the Nef protein is a wildtype Nef. In some embodiments, the Nef subtype is HIV F2-Nef, HIV C2-Nef, or HIV HV2NZ-Nef. In some embodiments, the Nef subtype is a SIV Nef subtype.

In some embodiments, the Nef protein is obtained or derived from primary HIV-1 subtype C Indian isolates. In some embodiments, the Nef protein is expressed from F2 allele of the Indian isolate encoding the full-length protein (HIV F2-Nef). In some embodiments, the Nef protein is expressed from C2 allele the Indian isolate with in-frame deletions of CD4 binding site, acidic cluster, proline-based repeat, and PAK binding domain (HIV C2-Nef). In some embodiments, the Nef protein is expressed from D2 allele the Indian isolate with in-frame deletions of CD4 binding site (HIV D2-Nef).

In some embodiments, the Nef protein is a mutant Nef, such as a Nef protein comprising one or more of insertion, deletion, point mutation(s), and/or rearrangement. In some embodiments, the mutant Nef described herein is a non-naturally occurring mutant Nef, such as a non-naturally occurring mutant Nef that does not down-modulate (e.g., down-regulate cell surface expression and/or effector function) the functional exogenous receptor comprising a CMSD described herein (e.g., an ITAM-modified TCR, an ITAM-modified CAR, an ITAM-modified cTCR, or an ITAM-modified TAC-like chimeric receptor) when expressed in a T cell. In some embodiments, the mutant Nef (e.g., non-naturally occurring mutant Nef) results in no or less down-regulation of a functional exogenous receptor comprising a CMSD described herein compared to a wildtype Nef when expressed in a T cell. Mutant Nef may comprise one or more mutations (e.g., non-naturally occurring mutation) in one or more domains or motifs selected from the group consisting of myristoylation site, N-terminal α-helix, tyrosine-based AP recruitment, CD4 binding site, acidic cluster, proline-based repeat, PAK binding domain, COP I recruitment domain, di-leucine based AP recruitment domain, V-ATPase and Raf-1 binding domain, or any combinations thereof. In some embodiments, the mutant Nef is a mutant SIV Nef, such as a mutant SIV Nef comprising the sequence of SEQ ID NO: 121 or 122.

### V. Vectors

The present application provides vectors for cloning and expressing any of the functional exogenous receptor comprising a CMSD described herein (e.g., an ITAM-modified TCR, an ITAM-modified CAR, an ITAM-modified cTCR, or an ITAM-modified TAC-like chimeric receptor). In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors encoding functional exogenous receptor comprising a CMSD described herein (e.g., an ITAM-modified TCR, an ITAM-modified CAR, an ITAM-modified cTCR, or an ITAM-modified TAC-like chimeric receptor) can be packaged into lentiviruses with protocols known in the art. The resulting lentiviruses can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the functional exogenous receptor comprising a CMSD (e.g., an ITAM-modified TCR, an ITAM-modified CAR, an ITAM-modified cTCR, or an ITAM-modified TAC-like chimeric receptor)-encoding nucleic acid to an immune effector cell (e.g., T cell such as modified T cell, allogeneic T cell, or CTL). See, for example, Yin H. et al. Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids encoding the functional exogenous receptor comprising a CMSD described herein (e.g., an ITAM-modified TCR, an ITAM-modified CAR, an ITAM-modified cTCR, or an ITAM-modified TAC-like chimeric receptor) is introduced into an immune effector cell (e.g., T cell such as modified T cell, allogeneic T cell, or CTL) by a physical method, including, but not limited to electroporation, sonoporation, photoporation, magnetofection, hydroporation.

In some embodiments, there is provided a vector (e.g., viral vector such as lentiviral vector) comprising any one of the nucleic acids encoding the functional exogenous receptor comprising a CMSD described herein (e.g., an ITAM-modified TCR, an ITAM-modified CAR, an ITAM-modified cTCR, or an ITAM-modified TAC-like chimeric receptor). In some embodiments, there is provided a vector (e.g., viral vector such as lentiviral vector) comprising a nucleic acid encoding a functional exogenous receptor (e.g., an ITAM-modified TCR, an ITAM-modified CAR, an ITAM-modified cTCR, or an ITAM-modified TAC-like chimeric receptor), wherein the functional exogenous receptor comprises: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. The nucleic acid can be cloned into the vector using any known molecular cloning methods in the art, including, for example, using restriction endonuclease sites and one or more selectable markers. In some embodiments, the nucleic acid is operably linked to a promoter (e.g., hEF1α promoter). Varieties of promoters have been explored for gene expression in mammalian cells, and any of the promoters known in the art may be used in the present invention. Promoters may be roughly categorized as constitutive promoters or regulated promoters, such as inducible promoters.

In some embodiments, the vector (e.g., viral vector) described herein further comprises a second nucleic acid encoding an exogenous Nef protein described herein (e.g., wt, subtype, or mutant Nef), or a second nucleic acid for knocking down (e.g., via siRNA, ZFN, TALEN, or CRISPR/Cas system) endogenous locus (e.g., TCR or B2M) expression. In some embodiments, the second nucleic acid and the nucleic acid encoding the CMSD-containing functional exogenous receptor are each operably linked to a promoter (e.g., hEF1α or PGK promoter). In some embodiments, the second nucleic acid and the nucleic acid encoding the CMSD-containing functional exogenous receptor are operably linked to one promoter (e.g., hEF1α promoter). In some embodiments, the nucleic acid encoding the CMSD-containing functional exogenous receptor and the second nucleic acid are connected by one or more linking sequences, such as a nucleic acid linking sequence encoding any of P2A, T2A, E2A, F2A, BmCPV 2A, BmIFV 2A, (GS)ₙ, (GGGS)ₙ, and (GGGGS)ₙ; or a nucleic acid linking sequence of any of IRES, SV40, CMV, UBC, EF1α, PGK, and CAGG; or any combinations thereof, wherein n is an integer of at least one. In some embodiments, the linking sequence is IRES (e.g., comprising the nucleic acid sequence of SEQ ID NO: 123.

### Promoters

In some embodiments, the promoter is selected from the group consisting of a phosphoglycerate kinase (PGK) promoter (e.g., PGK-1 promoter), a Rous Sarcoma Virus (RSV) promoter, an Simian Virus 40 (SV40) promoter, a cytomegalovirus (CMV) immediate early (IE) gene promoter, an elongation factor 1 alpha (EF1-α) promoter, a ubiquitin-C (UBQ-C) promoter, a cytomegalovirus CMV) enhancer/chicken beta-actin (CAG) promoter, polyoma enhancer/herpes simplex thymidine kinase (MC1) promoter, a beta actin (β-ACT) promoter, a myeloproliferative sarcoma virus enhancer, negative control region deleted, d1587rev primer-binding site substituted (MND) promoter, an NFAT promoter, a TETON® promoter, and an NFκB promoter.

In some embodiments, the nucleic acid encoding the functional exogenous receptor comprising a CMSD (e.g., an ITAM-modified TCR, an ITAM-modified CAR, an ITAM-modified cTCR, or an ITAM-modified TAC-like chimeric receptor), and/or the exogenous Nef protein described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV IE), human elongation factors-lalpha (hEF1a), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. For example, Michael C. Milone et al. compared the efficiencies of CMV, hEF1α, UbiC and PGK to drive CAR expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the functional exogenous receptor comprising a CMSD (e.g., an ITAM-modified TCR, an ITAM-modified CAR, an ITAM-modified cTCR, or an ITAM-modified TAC-like chimeric receptor), and/or the exogenous Nef protein described herein is operably linked to a hEF1α promoter or a PGK promoter.

In some embodiments, the nucleic acid encoding the functional exogenous receptor comprising a CMSD (e.g., an ITAM-modified TCR, an ITAM-modified CAR, an ITAM-modified cTCR, or an ITAM-modified TAC-like chimeric receptor), and/or the exogenous Nef protein described herein is operably linked to an inducible promoter. Inducible promoters belong to the category of regulated promoters. The inducible promoter can be induced by one or more conditions, such as a physical condition, microenvironment of the engineered immune effector cell (e.g., T cell), or the physiological state of the engineered immune effector cell, an inducer (i.e., an inducing agent), or a combination thereof. In some embodiments, the inducing condition does not induce the expression of endogenous genes in the engineered immune effector cell (e.g., T cell), and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered immune effector cell (e.g., T cell). In some embodiments, the inducible promoter can be an NFAT promoter, a TETON® promoter, or an NFκB promoter.

In some embodiments, the vector also contains a selectable marker gene or a reporter gene to select cells expressing the functional exogenous receptor comprising a CMSD (e.g., an ITAM-modified TCR, an ITAM-modified CAR, an ITAM-modified cTCR, or an ITAM-modified TAC-like chimeric receptor), and/or the exogenous Nef protein described herein from the population of host cells transfected through vectors (e.g., lentiviral vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### VI. Methods of Producing Modified T Cells

One aspect of the present invention provides methods of producing any one of the modified T cells (e.g., allogeneic or autologous T cell) described above, such as modified T cells expressing a functional exogenous receptor comprising a CMSD described herein (e.g., ITAM-modified CAR, ITAM-modified TCR, ITAM-modified cTCR, or ITAM-modified TAC-like chimeric receptor), also referred to herein as “CMSD-containing functional exogenous receptor-T cells” or “ITAM-modified functional exogenous receptor-T cells”). Such CMSD-containing functional exogenous receptor-T cells can further be modified to express an exogenous Nef protein described herein (also referred to herein as “Nef-containing CMSD-containing functional exogenous receptor-T cells” or “Nef-containing ITAM-modified functional exogenous receptor-T cells”). The nucleic acid encoding the exogenous Nef protein can be introduced into the T cell at the same time (e.g., via separate vectors, or via the same vector), before, or after introducing the nucleic acid encoding any of the CMSD-containing functional exogenous receptors described herein into the T cell. In some embodiments, the Nef-containing ITAM-modified functional exogenous receptor-T cell elicit no or reduced (such as reduced by at least about any of 30%, 40%, 50%, 60%, 70%, 80%, 90%, or 95%) GvHD response in a histoincompatible individual as compared to the GvHD response elicited by a primary T cell isolated from the donor of the precursor T cell from which the modified T cell is derived, or elicited by an ITAM-modified functional exogenous receptor-T cell from the same donor source without Nef expression. Although below description focuses on methods of producing ITAM-modified functional exogenous receptor-T cells, it is conceivable that such methods can be used and/or modified to further express an exogenous Nef protein in said modified T cells.

The method of producing a modified T cell expressing a functional exogenous receptor comprising a CMSD described herein generally involves introducing a vector (e.g., viral vector such as lentiviral vector) carrying a nucleic acid encoding a functional exogenous receptor comprising a CMSD described herein into a native or engineered T cell (referred to herein as “precursor T cell”). The method of producing a modified T cell expressing an expressing a functional exogenous receptor comprising a CMSD described herein generally involves introducing a nucleic acid encoding the functional exogenous receptor comprising a CMSD described herein into a precursor T cell. In some embodiments, when a population of precursor T cells are used for the production of modified T cells described herein, the methods also include one or more isolation and/or enrichment steps, for example, isolating and/or enriching ITAM-modified functional exogenous receptor positive T cells (e.g., ITAM-modified CAR positive, ITAM-modified TCR positive, ITAM-modified cTCR positive, or ITAM-modified TAC-like chimeric receptor positive) T cells from T cells modified to express functional exogenous receptor comprising a CMSD. Such isolation and/or enrichment steps can be performed using any known techniques in the art, such as magnetic-activated cell sorting (MACS). Briefly, transduced/transfected cell suspension was centrifuged at room temperature, the supernatant was discarded. Cells were resuspended with DPBS then supplemented with MACSelect LNGFR MicroBeads (Miltenyi Biotec, #130-091-330), and incubated on ice for 15 min for magnetic labeling. After incubation, PBE buffer (sodium phosphate/EDTA) was added to adjust the volume. The cell suspension was then subject to magnetic separation and enrichment according to the MACS kit protocols. Also see Examples. In some embodiments, if the modified T cell further expresses an exogenous Nef protein, the method can further comprise isolating and/or enriching Nef-positive, CD3ε/γ/δ-negative, TCRα/β-negative, MHC I-negative, CD4-positive, and/or CD28-positive T cells from T cells modified to express exogenous Nef protein. It is also conceivable that a T cell can be modified to express an exogenous Nef protein, isolated and/or enriched for aforementioned markers, then used for further expressing a CMSD-containing functional exogenous receptor.

In some embodiments, the precursor T cells are derived from the blood, bone marrow, lymph, or lymphoid organs. In some embodiments, the precursor T cells are cells of the immune system, such as cells of innate or adaptive immunity. In some aspects, the cells are human cells. In some embodiments, the precursor T cells are derived from cell lines, e.g., T cell lines. The cells in some embodiments are obtained from a xenogeneic source, for example, from mouse, rat, non-human primate, or pig.

In some embodiments, the precursor T cells are CD4+/CD8-, CD4-/CD8+, CD4+/CD8+, CD4−/CD8−, or combinations thereof. In some embodiments, the T cell is a natural killer T (NKT) cell. In some embodiments, the precursor T cell is a modified T cell, such as modified T cells expressing a functional exogenous receptor comprising a CMSD described herein, modified T cells expressing an exogenous Nef protein, or T cells with modified endogenous TCR or B2M locus (e.g., via CRISPR/Cas system). In some embodiments, the precursor T cell produces IL-2, TFN, and/or TNF upon expression of the functional exogenous receptor comprising a CMSD described herein and binding to the target cells (e.g., BCMA+ or CD20+ tumor cells). In some embodiments, the CD8+ T cells lyse antigen-specific target cells (e.g., BCMA+ or CD20+ tumor cells) upon expression of the functional exogenous receptor comprising a CMSD described herein and binding to the target cells.

In some embodiments, the T cells to be modified are differentiated from a stem cell, such as a hematopoietic stem cell, a pluripotent stem cell, an iPS, or an embryonic stem cell.

In some embodiments, the functional exogenous receptor comprising a CMSD described herein (e.g., an ITAM-modified TCR, an ITAM-modified CAR, an ITAM-modified cTCR, or an ITAM-modified TAC-like chimeric receptor) is introduced to the T cells by transducing/transfecting any one of the nucleic acids or any one of the vectors (e.g., non-viral vectors, or viral vectors such as lentiviral vectors) described herein. In some embodiments, the functional exogenous receptor comprising a CMSD described herein is introduced into the T cell by inserting proteins into the cell membrane while passing cells through a microfluidic system, such as CELL SQUEEZE® (see, for example, U.S. Patent Application Publication No. 20140287509).

Methods of introducing vectors (e.g., viral vectors) or isolated nucleic acids into a mammalian cell are known in the art. The vectors described herein can be transferred into a T cell by physical, chemical, or biological methods.

Physical methods for introducing a vector (e.g., viral vector) into a T cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector (e.g., viral vector) is introduced into the cell by electroporation.

Biological methods for introducing a vector into a T cell include the use of DNA and RNA vectors. Viral vectors have become the most widely used method for inserting genes into mammalian, e.g., human cells.

Chemical means for introducing a vector (e.g., viral vector) into a T cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the functional exogenous receptor comprising a CMSD described herein (e.g., ITAM-modified CAR, ITAM-modified TCR, ITAM-modified cTCR, or ITAM-modified TAC-like chimeric receptor) may be prepared by a conventional method (e.g., in vitro transcription) and then introduced into the T cell via known methods such as mRNA electroporation. See, e.g., Rabinovich et al., Human Gene Therapy 17:1027-1035.

In some embodiments, the transduced/transfected T cell is propagated ex vivo after introduction of the vector or isolated nucleic acid. In some embodiments, the transduced/transfected T cell is cultured to propagate for at least about any of 1 day, 2 days, 3 days, 4 days, 5 days, 6 days, 7 days, 10 days, 12 days, or 14 days. In some embodiments, the transduced/transfected T cell is further evaluated or screened to select desired engineered mammalian cell, e.g., modified T cells described herein.

Reporter genes may be used for identifying potentially transfected/transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA/RNA has been introduced into the recipient cells. Suitable reporter genes may include genes encoding luciferase, beta-galactosidase, chloramphenicol acetyl transferase, secreted alkaline phosphatase, or the green fluorescent protein (GFP) gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding any of the functional exogenous receptor comprising a CMSD described herein (e.g., ITAM-modified CAR, ITAM-modified TCR, ITAM-modified cTCR, or ITAM-modified TAC-like chimeric receptor) in a modified T cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots), Fluorescence-activated cell sorting (FACS), or Magnetic-activated cell sorting (MACS) (also see Example section).

Thus in some embodiments, there is provided a method of producing a modified T cell (e.g., allogeneic T cell, endogenous TCR-deficient T cell, GvHD-minimized T cell, or autologous T cell), comprising introducing into a precursor T cell a nucleic acid encoding a functional exogenous receptor (e.g., ITAM-modified CAR, ITAM-modified TCR, ITAM-modified cTCR, or ITAM-modified TAC-like chimeric receptor), wherein the functional exogenous receptor comprises: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers. In some embodiments, there is provided a method of producing a modified T cell (e.g., allogeneic or autologous T cell), comprising introducing into a precursor T cell a nucleic acid encoding any of the CMSD-containing functional exogenous receptors described herein, such as an ITAM-modified CAR comprising the amino acid sequence of any of SEQ ID NO: 76-96, 98-104, and 106-113. In some embodiments, the modified T cell further expresses an exogenous Nef protein (e.g., wt, subtype, or mutant Nef), such as an exogenous Nef protein comprising the amino acid sequence of SEQ ID NO: 121, 122, 136, or 139.

In some embodiments, the modified T cell comprises unmodified endogenous TCR and/or B2M loci. In some embodiments, the modified T cell comprises a modified endogenous TCR locus, such as modified TCRα or TCRβ locus. In some embodiments, the modified T cell comprises a modified endogenous B2M locus. In some embodiments, the endogenous TCR (or B2M) locus is modified by a gene editing system selected from CRISPR-Cas, TALEN, and ZFN.

In some embodiments, the method further comprises isolating and/or enriching T cells comprising the nucleic acid. In some embodiments, the method further comprises isolating and/or enriching ITAM-modified functional exogenous receptor-positive T cells from the modified T cells expressing the functional exogenous receptor comprising a CMSD described herein. In some embodiments, the method further comprises isolating and/or enriching CD3ε/γ/δ-negative, TCRα/0-negative, MHC I-negative, CD4-positive, and/or CD28-positive T cells from the modified T cells expressing the functional exogenous receptor comprising a CMSD described herein.

In some embodiments, the modified T cell (e.g., co-expressing an exogenous Nef and CMSD-containing functional exogenous receptor) elicits no or reduced (such as reduced by at least about any of 30%, 40%, 50%, 60%, 70%, 80%, 90%, or 95%) GvHD response in a histoincompatible individual as compared to the GvHD response elicited by a primary T cell isolated from the donor of the precursor T cell from which the modified T cell is derived. In some embodiments, the method further comprises formulating the modified T cells (expressing functional exogenous receptor and/or exogenous Nef) with at least one pharmaceutically acceptable carrier. In some embodiments, the method further comprises administering to an individual (e.g., human) an effective amount of the modified T cells expressing the functional exogenous receptor comprising a CMSD described herein (e.g., an ITAM-modified TCR, an ITAM-modified CAR, an ITAM-modified cTCR, or an ITAM-modified TAC-like chimeric receptor), and/or the exogenous Nef protein, or an effective amount of the pharmaceutical formulation thereof. In some embodiments, the method comprises administering to an individual (e.g., human) an effective amount of the modified T cells expressing functional exogenous receptor comprising a CMSD (e.g., ITAM-modified CAR, ITAM-modified TCR, ITAM-modified cTCR, or ITAM-modified TAC-like chimeric receptor) and/or the exogenous Nef protein, or an effective amount of the pharmaceutical formulation thereof. In some embodiments, the individual has cancer. In some embodiments, the individual is a human. In some embodiments, the individual is histoincompatible with the donor of the precursor T cell from which the modified T cell is derived.

### Source of T Cells, Cell Preparation and Culture

Prior to expansion and genetic modification of the T cells (e.g., precursor T cells), a source of T cells is obtained from an individual. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. In some embodiments, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, the T cell is provided from an umbilical cord blood bank, a peripheral blood bank, or derived from an induced pluripotent stem cell (iPSC), multipotent and pluripotent stem cell, or a human embryonic stem cell. In some embodiments, the T cells are derived from cell lines. The T cells in some embodiments are obtained from a xenogeneic source, for example, from mouse, rat, non-human primate, and pig. In some embodiments, the T cells are human cells. In some aspects, the T cells are primary cells, such as those isolated directly from a subject and/or isolated from a subject and frozen. In some embodiments, the cells include one or more subsets of T cells, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some cases, the T cell is allogeneic in reference to one or more intended recipients. In some cases, the T cell is suitable for transplantation, such as without inducing GvHD in the recipient.

Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present application is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a genetically engineered antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Whether prior to or after genetic modification of the T cells with exogenous nucleic acids, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD3 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15 (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresis peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some embodiments, the methods include assessing expression of one or more markers on the surface of the modified cells or cells to be engineered. In one embodiment, the methods include assessing surface expression of TCR, MHC I, CD4, CD28, and/or CD3 (e.g., CD3ε), for example, by affinity-based detection methods such as by flow cytometry. In some embodiments, cell surface markers such as T cell exhaustion markers or memory markers are assessed. In some aspects, where the method reveals surface expression of the antigen or other marker, the gene encoding the antigen or other marker is disrupted or expression otherwise repressed for example, using the methods described herein.

### Isolation and Enrichment of Modified T Cells

In some embodiments, the method described herein further comprise isolating or enriching T cells comprising the nucleic acid. Thus in some embodiments, the method described herein comprises isolating or enriching modified T cells expressing the functional exogenous receptor comprising a CMSD described herein. In some embodiments, the method described herein further comprises isolating or enriching CD3ε/γ/δ-negative T cells from the modified T cells (e.g., further expressing an exogenous Nef protein). In some embodiments, the method described herein further comprises isolating or enriching endogenous TCRα/0-negative T cells from the modified T cell. In some embodiments, the method described herein further comprises isolating or enriching CD4+ and/or CD28+ T cells from the modified T cells. In some embodiments, the method described herein further comprises isolating or enriching MHC I-negative T cells from the modified T cells. In some embodiments, the isolation or enrichment of T cells comprises any combinations of the methods described herein.

In some embodiments, the isolation methods include the separation of different cell types based on the absence or presence in the cell of one or more specific molecules, such as surface markers, e.g., surface proteins, intracellular markers, or nucleic acid. In some embodiments, the selection marker is functional exogenous receptor comprising a CMSD (e.g., ITAM-modified CAR, ITAM-modified TCR, ITAM-modified cTCR, or ITAM-modified TAC-like chimeric receptor), CD4, CD28, CD3ε, CD3γ, CD3δ, CD3ζ, CD69, TCRα, TCRβ, and/or MHC I. In some embodiments, the selection marker is a T cell exhaustion marker such as PD-1 or LAG-3. In some embodiments, the selection marker is a T cell memory marker such as TEMRA, T_{EM}, or TC_{M}. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniIACS Prodigy system in some aspects is equipped with a cell processing unity that permits automated washing and fractionation of cells by centrifugation. The CliniIACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniIACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously. Also see “Examples” section for isolation and/or enrichment methods.

### Gene-Editing of Endogenous Loci

In some embodiments, the endogenous loci of the T cell such as endogenous TCR loci (e.g., TCRα, TCRβ) or B2M (beta-2-microglobulin; can lead to deficiency in MHC Class I molecule expression and/or depletion of CD8+ T cells) locus, is modified by a gene-editing method, prior to or simultaneously with modifying the T cell to express a functional exogenous receptor comprising a CMSD described herein (e.g., ITAM-modified CAR, ITAM-modified TCR, ITAM-modified cTCR, or ITAM-modified TAC-like chimeric receptor). In some embodiments, the modification of the endogenous loci is carried out by effecting a disruption in the gene, such as a knock-out, insertion, missense or frameshift mutation, such as a biallelic frameshift mutation, deletion of all or part of the gene, e.g., one or more exon or portion thereof, and/or knock-in. In some embodiments, such locus modification is performed using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

In some embodiments, the modification of endogenous loci (e.g., TCR or B2M) is carried out using one or more DNA-binding nucleic acids, such as disruption via an RNA-guided endonuclease (RGEN), or other form of repression by another RNA-guided effector molecule. For example, in some embodiments, the repression is carried out using clustered regularly interspaced short palindromic repeats (CRISPR) and CRISPR-associated (Cas) proteins. See Sander and Joung, Nature Biotechnology, 32 (4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a proto spacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

In some embodiments, an endogenous locus of a T cell (e.g., endogenous TCR) is modified by CRISPR/Cas system prior to modifying the T cell to express a functional exogenous receptor comprising a CMSD described herein (e.g., ITAM-modified CAR, ITAM-modified TCR, ITAM-modified cTCR, or ITAM-modified TAC-like chimeric receptor). In some embodiments, an endogenous loci of a T cell (e.g., endogenous TCR) is modified by CRISPR/Cas system simultaneously with modifying the T cell to express a functional exogenous receptor comprising a CMSD described herein. In some embodiments, the nucleic acid(s) encoding the CRISPR/Cas system and the nucleic acid(s) encoding the functional exogenous receptor comprising a CMSD described herein are on the same vector, either optionally controlled by the same promoter or different promoters. In some embodiments, the nucleic acid(s) encoding the CRISPR/Cas system and the nucleic acid(s) encoding the functional exogenous receptor comprising a CMSD described herein (e.g., ITAM-modified CAR, ITAM-modified TCR, ITAM-modified cTCR, or ITAM-modified TAC-like chimeric receptor) are on different vectors.

### VII. Pharmaceutical Compositions

Further provided by the present application are pharmaceutical compositions comprising any one of the modified T cells (e.g., allogeneic T cells or autologous T cells) expressing a functional exogenous receptor comprising a CMSD described herein (e.g., ITAM-modified CAR, ITAM-modified TCR, ITAM-modified cTCR, or ITAM-modified TAC-like chimeric receptor), and optionally a pharmaceutically acceptable carrier. In some embodiments, the modified T cells further express an exogenous Nef protein. Pharmaceutical compositions can be prepared by mixing a population of modified T cells described herein with optional pharmaceutically acceptable carriers, excipients or stabilizers (Remington's Pharmaceutical Sciences 16th edition, Osol, A. Ed. (1980)), in the form of aqueous solutions. In some embodiments, the population of modified T cells are homogenous. For example, in some embodiments, at least about 70% (such as at least about any of 75%, 80%, 85%, 90%, or 95%) of the population of modified T cells transduced/transfected with a vector carrying a nucleic acid encoding a functional exogenous receptor comprising a CMSD described herein (e.g., ITAM-modified CAR, ITAM-modified TCR, ITAM-modified cTCR, or ITAM-modified TAC-like chimeric receptor) are ITAM-modified functional exogenous receptor-positive. In some embodiments, at least about 70% (such as at least about any of 75%, 80%, 85%, 90%, or 95%) of the population of modified T cells transduced/transfected with a nucleic acid encoding a functional exogenous receptor comprising a CMSD described herein are TCRα/TCRβ negative and ITAM-modified functional exogenous receptor-positive. In some embodiments, at least about 70% (such as at least about any of 75%, 80%, 85%, 90%, or 95%) of the population of modified T cells transduced/transfected with a nucleic acid encoding a functional exogenous receptor comprising a CMSD described herein are MHC I negative and ITAM-modified functional exogenous receptor-positive. In some embodiments, at least about 70% (such as at least about any of 75%, 80%, 85%, 90%, or 95%) of the population of modified T cells transduced/transfected with a nucleic acid encoding a functional exogenous receptor comprising a CMSD described herein are CD3 (e.g., CD3ε/γ/δ) negative and ITAM-modified functional exogenous receptor-positive. In some embodiments, at least about 70% (such as at least about any of 75%, 80%, 85%, 90%, or 95%) of the population of modified T cells transduced/transfected with a nucleic acid encoding a functional exogenous receptor comprising a CMSD described herein are CD4 and/or CD28-positive, and ITAM-modified functional exogenous receptor-positive.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1 to 5%, taking into account the relative amounts of the other ingredients. In some embodiments, tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/mL to about 1.0 mg/mL, preferably about 0.07 mg/mL to about 0.2 mg/mL.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, or by sustained release or extended-release means.

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions described herein may also contain more than one active compound or agent as necessary for the particular indication being treated, preferably those with complementary activities that do not adversely affect each other. Alternatively, or in addition, the composition may comprise a cytotoxic agent, chemotherapeutic agent, cytokine, immunosuppressive agent, immune checkpoint modulators, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

### VIII. Methods of Treatment

The present application further provides methods of treating a disease (such as cancer, infectious disease, GvHD, transplantation rejection, autoimmune disorders, or radiation sickness) in an individual (e.g., human) comprising administering to the individual an effective amount of modified T cells (e.g., allogeneic T cell, endogenous TCR-deficient T cell, GvHD-minimized T cell, or autologous T cell) expressing a functional exogenous receptor comprising a CMSD described herein (e.g., ITAM-modified CAR, ITAM-modified TCR, ITAM-modified cTCR, or ITAM-modified TAC-like chimeric receptor), or pharmaceutical compositions thereof. In some embodiments, the modified T cell further expresses an exogenous Nef protein (e.g., wt, subtype, or mutant Nef), such as an exogenous Nef protein comprising the amino acid sequence of SEQ ID NO: 121, 122, 136, or 139. The present application also provides methods of treating a disease (such as cancer, infectious disease, autoimmune disorders, or radiation sickness) in an individual (e.g., human) comprising administering to the individual an effective amount of modified T cells (e.g., allogeneic or autologous T cell) expressing a functional exogenous receptor comprising a CMSD described herein, or pharmaceutical compositions thereof. In some embodiments, the modified T cell expresses an ITAM-modified CAR, e.g., ITAM-modified CD20 CAR (e.g., comprising the sequence of any of SEQ ID NOs: 98-104), or ITAM-modified BCMA CAR (e.g., comprising the sequence of any of SEQ ID NOs: 76-96 and 106-113).

The methods described herein are suitable for treating various cancers, including both solid cancer and liquid cancer. The methods are applicable to cancers of all stages, including early stage, advanced stage and metastatic cancer. The methods described herein may be used as a first therapy, second therapy, third therapy, or combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In some embodiments, the methods described herein are suitable for treating a solid cancer selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the methods described herein are suitable for treating a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the cancer is multiple myeloma. In some embodiments, the cancer is stage I, stage II or stage III, and/or stage A or stage B multiple myeloma based on the Durie-Salmon staging system. In some embodiments, the cancer is stage I, stage II or stage III multiple myeloma based on the International staging system published by the International Myeloma Working Group (IMWG). In some embodiments, the cancer is monoclonal gammopathy of undetermined significance (MGUS). In some embodiments, the cancer is asymptomatic (smoldering/indolent) myeloma. In some embodiments, the cancer is symptomatic or active myeloma. In some embodiments, the cancer is refractory multiple myeloma. In some embodiments, the cancer is metastatic multiple myeloma. In some embodiments, the individual did not respond to a previous treatment for multiple myeloma. In some embodiments, the individual has progressive disease after a previous treatment of multiple myeloma. In some embodiments, the individual has previously received at least about any one of 2, 3, 4, or more treatment for multiple myeloma. In some embodiments, the cancer is relapsed multiple myeloma.

In some embodiments, the individual has active multiple myeloma. In some embodiments, the individual has clonal bone marrow plasma cells of at least 10%. In some embodiments, the individual has a biopsy-proven bony or extramedullary plasmacytoma. In some embodiments, the individual has evidence of end organ damage that can be attributed to the underlying plasma cell proliferative disorder. In some embodiments, the individual has hypercalcemia, e.g., serum calcium >0.25 mmol/L (>1 mg/dL) higher than the upper limit of normal or >2.75 mmol/L (>11 mg/dL). In some embodiments, the individual has renal insufficiency, e.g., creatinine clearance <40 mL per minute or serum creatinine >177 mol/L (>2 mg/dL). In some embodiments, the individual has anemia, e.g., hemoglobin value of >20 g/L below the lowest limit of normal, or a hemoglobin value <100 g/L. In some embodiments, the individual has one or more bone lesions, e.g., one or more osteolytic lesion on skeletal radiography, CT, or PET/CT. In some embodiments, the individual has one or more of the following biomarkers of malignancy (MDEs): (1) 60% or greater clonal plasma cells on bone marrow examination; (2) serum involved/uninvolved free light chain ratio of 100 or greater, provided the absolute level of the involved light chain is at least 100 mg/L; and (3) more than one focal lesion on MRI that is at least 5 mm or greater in size.

In some embodiments, the methods described herein are suitable for treating an autoimmune disease. Autoimmune disease, or autoimmunity, is the failure of an organism to recognize its own constituent parts (down to the sub-molecular levels) as “self,” which results in an immune response against its own cells and tissues. Any disease that results from such an aberrant immune response is termed an autoimmune disease. Prominent examples include Coeliac disease, diabetes mellitus type 1 (IDDM), systemic lupus erythematosus (SLE), Sjögren's syndrome, multiple sclerosis (MS), Hashimoto's thyroiditis, Graves' disease, idiopathic thrombocytopenic purpura, and rheumatoid arthritis (RA).

Inflammatory diseases are commonly treated with corticosteroids and cytotoxic drugs, which can be very toxic. These drugs also suppress the entire immune system, can result in serious infection, and have adverse effects on the bone marrow, liver, and kidneys. Other therapeutics that has been used to treat Class III autoimmune diseases to date have been directed against T cells and macrophages. There is a need for more effective methods of treating autoimmune diseases, particularly Class III autoimmune diseases. In some embodiments, the methods described herein are suitable for treating an inflammatory diseases, including autoimmune diseases are also a class of diseases associated with B-cell disorders. Examples of autoimmune diseases include, but are not limited to, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans. Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis, psoriasis, and fibrosing alveolitis.

Administration of the pharmaceutical compositions may be carried out in any convenient manner, including by injection, transfusion, implantation or transplantation. The compositions may be administered to a patient transarterially, subcutaneously, intradermally, intratumorally, intranodally, intramedullary, intramuscularly, intravenously, or intraperitoneally. In some embodiments, the pharmaceutical composition is administered systemically. In some embodiments, the pharmaceutical composition is administered to an individual by infusion, such as intravenous infusion. Infusion techniques for immunotherapy are known in the art (see, e.g., Rosenberg et al., New Eng. J. of Med. 319:1676 (1988)). In some embodiments, the pharmaceutical composition is administered to an individual by intradermal or subcutaneous injection. In some embodiments, the compositions are administered by intravenous injection. In some embodiments, the compositions are injected directly into a tumor, or a lymph node. In some embodiments, the pharmaceutical composition is administered locally to a site of tumor, such as directly into tumor cells, or to a tissue having tumor cells.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

In some embodiments, for a pharmaceutical composition comprising a population of modified T cells expressing a functional exogenous receptor comprising a CMSD described herein (e.g., ITAM-modified CAR, ITAM-modified TCR, ITAM-modified cTCR, or ITAM-modified TAC-like chimeric receptor), the pharmaceutical composition is administered at a dosage of at least about any of 10⁴, 10⁵, 10⁶, 10⁷, 10⁸, or 10⁹ cells/kg of body weight of the individual. In some embodiments, the pharmaceutical composition is administered at a dosage of any of about 10⁴ to about 10⁵, about 10⁵ to about 10⁶, about 10⁶ to about 10⁷, about 10⁷ to about 10⁸, about 10⁸ to about 10⁹, about 10⁴ to about 10⁹, about 10⁴ to about 10⁶, about 10⁶ to about 10⁸, or about 10⁵ to about 10⁷ cells/kg of body weight of the individual. In some embodiments, the pharmaceutical composition is administered at a dose of at least about any 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷ cells/kg or more. In some embodiments, the pharmaceutical composition is administered at a dose of about 3×10⁵ to about 7×10⁶ cells/kg, or about 3×10⁶ cells/kg.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

Moreover, dosages may be administered by one or more separate administrations, or by continuous infusion. In some embodiments, the pharmaceutical composition is administered in split doses, such as about any one of 2, 3, 4, 5, or more doses. In some embodiments, the split doses are administered over about a week. In some embodiments, the dose is equally split. In some embodiments, the split doses are about 20%, about 30%, about 40%, or about 50% of the total dose. In some embodiments, the interval between consecutive split doses is about 1 day, 2 days, 3 days or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

In some embodiments, there is provided a method of treating an individual (e.g., human) having a disease (e.g., cancer, infectious disease, GvHD, transplantation rejection, autoimmune disorders, or radiation sickness), comprising administering to the individual an effective amount of a pharmaceutical composition comprising: (1) a modified T cell (e.g., allogeneic T cell, endogenous TCR-deficient T cell, GvHD-minimized T cell) comprising a functional exogenous receptor (e.g., ITAM-modified CAR, ITAM-modified TCR, ITAM-modified cTCR, or ITAM-modified TAC-like chimeric receptor) comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers; and (2) optionally a pharmaceutically acceptable carrier. In some embodiments, the modified T cell further expresses an exogenous Nef protein (e.g., wt, subtype, or mutant Nef), such as an exogenous Nef protein comprising the amino acid sequence of SEQ ID NO: 121, 122, 136, or 139. In some embodiments, the disease is cancer. In some embodiments, the individual is histoincompatible with the donor of the precursor T cell from which the modified T cell is derived. In some embodiments, the pharmaceutical composition is administered intravenously. In some embodiments, the functional exogenous receptor is an ITAM-modified CAR, e.g., ITAM-modified BCMA CAR or ITAM-modified CD20 CAR. In some embodiments, the ITAM-modified CAR comprise the sequence of any of SEQ ID NOs: 76-96, 98-104, and 106-113.

In some embodiments, there is provided a method of treating an individual (e.g., human) having a disease (e.g., cancer, infectious disease, autoimmune disorders, or radiation sickness), comprising administering to the individual an effective amount of a pharmaceutical composition comprising: (1) a modified T cell (e.g., allogeneic or autologous T cell) expressing a functional exogenous receptor (e.g., ITAM-modified CAR, ITAM-modified TCR, ITAM-modified cTCR, or ITAM-modified TAC-like chimeric receptor) comprising: (a) an extracellular ligand binding domain (such as antigen-binding fragments (e.g., scFv, sdAb) specifically recognizing one or more epitopes of one or more target antigens (e.g., tumor antigen such as BCMA, CD19, CD20), extracellular domains (or portion thereof) of receptors (e.g., FcR), extracellular domains (or portion thereof) of ligands (e.g., APRIL, BAFF)), (b) a transmembrane domain (e.g., derived from CD8α), and (c) an ISD comprising a CMSD (e.g., CMSD comprising a sequence selected from the group consisting of SEQ ID NOs: 41-74), wherein the CMSD comprises one or a plurality of CMSD ITAMs, wherein the plurality of CMSD ITAMs are optionally connected by one or more CMSD linkers; and (2) optionally a pharmaceutically acceptable carrier. In some embodiments, the disease is cancer. In some embodiments, the individual is histoincompatible with the donor of the precursor T cell from which the modified T cell is derived. In some embodiments, the pharmaceutical composition is administered intravenously. In some embodiments, the functional exogenous receptor is an ITAM-modified CAR, such as any of the ITAM-modified CAR described herein, e.g., ITAM-modified BCMA CAR or ITAM-modified CD20 CAR. In some embodiments, the ITAM-modified CAR comprise the amino acid sequence of any of SEQ ID NOs: 76-96, 98-104, and 106-113.

In some embodiments, the disease is cancer. In some embodiments, the cancer is multiple myeloma, such as relapsed or refractory multiple myeloma. In some embodiments, the treatment effect comprises causing an objective clinical response in the individual. In some embodiments, Stringent Clinical Response (sCR) is obtained in the individual. In some embodiments, the treatment effect comprises causing disease remission (partial or complete) in the individual. In some the clinical remission is obtained after no more than about any one of 6 months, 5 months, 4 months, 3 months, 2 months, 1 months or less after the individual receives the pharmaceutical composition. In some embodiments, the treatment effect comprises preventing relapse or disease progression of the cancer in the individual. In some embodiments, the relapse or disease progression is prevented for at least about 6 months, 1 year, 2 years, 3 years, 4 years, 5 years or more. In some embodiments, the treatment effect comprises prolonging survival (such as disease free survival) in the individual. In some embodiments, the treatment effect comprises improving quality of life in an individual. In some embodiments, the treatment effect comprises inhibiting growth or reducing the size of a solid or lymphatic tumor.

In some embodiments, the size of the solid or lymphatic tumor is reduced for at least about 10% (including for example at least about any of 20%, 30%, 40%, 60%, 70%, 80%, 90%, or 100%). In some embodiments, a method of inhibiting growth or reducing the size of a solid or lymphatic tumor in an individual is provided. In some embodiments, the treatment effect comprises inhibiting tumor metastasis in the individual. In some embodiments, at least about 10% (including for example at least about any of 20%, 30%, 40%, 60%, 70%, 80%, 90%, or 100%) metastasis is inhibited. In some embodiments, a method of inhibiting metastasis to lymph node is provided. In some embodiments, a method of inhibiting metastasis to the lung is provided. In some embodiments, a method of inhibiting metastasis to the liver is provided. Metastasis can be assessed by any known methods in the art, such as by blood tests, bone scans, x-ray scans, CT scans, PET scans, and biopsy.

The invention is also directed to methods of reducing or ameliorating, or preventing or treating, diseases and disorders using the modified T cells (e.g., allogeneic or autologous T cell) expressing a functional exogenous receptor comprising a CMSD described herein (e.g., ITAM-modified CAR, ITAM-modified TCR, ITAM-modified cTCR, or ITAM-modified TAC-like chimeric receptor), isolated populations thereof, or pharmaceutical compositions comprising the same. In some embodiments, the modified T cell further expresses an exogenous Nef protein (e.g., wt, subtype, or mutant Nef). In some embodiments, the modified T cells (e.g., allogeneic or autologous T cell) expressing a functional exogenous receptor comprising a CMSD described herein, isolated populations thereof, or pharmaceutical compositions comprising the same are used to reduce or ameliorate, or prevent or treat, cancer, infection, one or more autoimmune disorders, radiation sickness, or to prevent or treat graft versus host disease (GvHD) or transplantation rejection in a subject undergoing transplant surgery.

The modified T cells (e.g., allogeneic or autologous T cell) expressing a functional exogenous receptor comprising a CMSD described herein (e.g., ITAM-modified CAR, ITAM-modified TCR, ITAM-modified cTCR, or ITAM-modified TAC-like chimeric receptor), isolated populations thereof, or pharmaceutical compositions comprising the same are useful in altering autoimmune or transplant rejection because these T cells can be grown in TGF-β during development and will differentiate to become induced T regulatory cells. In one embodiment, the functional exogenous receptor comprising a CMSD described herein is used to give these induced T regulatory cells the functional specificity that is required for them to perform their inhibitory function at the tissue site of disease. Thus, a large number of antigen-specific regulatory T cells are grown for use in patients. The expression of FoxP3, which is essential for T regulatory cell differentiation, can be analyzed by flow cytometry, and functional inhibition of T cell proliferation by these T regulatory cells can be analyzed by examining decreases in T cell proliferation after anti-CD3 stimulation upon co-culture. In some embodiments, the modified T cell further expresses an exogenous Nef protein.

Another embodiment of the invention is directed to the use of modified T cells (e.g., allogeneic or autologous T cell) expressing a functional exogenous receptor comprising a CMSD described herein (e.g., ITAM-modified CAR, ITAM-modified TCR, ITAM-modified cTCR, or ITAM-modified TAC-like chimeric receptor), isolated populations thereof, or pharmaceutical compositions comprising the same for the prevention or treatment of radiation sickness. One challenge after radiation treatment or exposure (e.g. dirty bomb exposure, radiation leak) or other condition that ablates bone marrow cells (certain drug therapies) is to reconstitute the hematopoietic system. In patients undergoing a bone marrow transplant, the absolute lymphocyte count on day 15 post-transplant is correlated with successful outcome. Those patients with a high lymphocyte count reconstitute well, so it is important to have a good lymphocyte reconstitution. The reason for this effect is unclear, but it may be due to lymphocyte protection from infection and/or production of growth factors that favors hematopoietic reconstitution. In some embodiments, the modified T cell further expresses an exogenous Nef protein.

In some embodiments, the present invention also provides a method of increasing persistence and/or engraftment of donor T cells in an individual, comprising 1) providing an allogeneic T cell; and 2) introducing into the allogeneic T cell a first nucleic acid encoding a TCR and/or MHC I downregulating molecule, such as an exogenous Nef protein (e.g., wildtype Nef such as wildtype SIV Nef, or mutant Nef such as mutant SIV Nef), wherein the TCR and/or MHC I downregulating molecule (such as exogenous Nef protein) upon expression results in down-modulation (e.g., down-regulation of cell surface expression and/or effector function such as signal transduction) of the endogenous TCR, CD3ε, and/or MHC I of the allogeneic T cell. In some embodiments, the allogeneic T cell is an allogeneic ITAM-modified CAR-T cell, ITAM-modified TCR-T cell, ITAM-modified cTCR-T cell, or ITAM-modified TAC-like-T cell. In some embodiments, the method further comprises introducing into the allogeneic T cell a second nucleic acid encoding a functional exogenous receptor comprising a CMSD described herein. In some embodiments, the second nucleic acid encodes an ITAM-modified CAR. In some embodiments, the first nucleic acid and the second nucleic acid are on separate vectors. In some embodiments, the first nucleic acid and the second nucleic acid are on the same vector, either under control of one promoter or different promoters. Thus in some embodiments, the present invention provides a method of increasing persistence and/or engraftment of donor T cells in an individual (e.g., human), comprising 1) providing an allogeneic T cell; and 2) introducing into the allogeneic T cell a vector (e.g., viral vector, lentiviral vector) comprising a first nucleic acid encoding a TCR and/or MHC I downregulating molecule (such as an exogenous Nef protein (e.g., wildtype Nef such as wildtype SIV Nef, or mutant Nef such as mutant SIV Nef)) and a second nucleic acid encoding a CMSD-containing functional exogenous receptor (e.g., ITAM-modified CAR, ITAM-modified TCR, ITAM-modified cTCR, or ITAM-modified TAC-like chimeric receptor); wherein the exogenous Nef protein upon expression results in down-modulation (e.g., down-regulation of cell surface expression and/or effector function such as signal transduction) of the endogenous TCR, CD3ε, and/or MHC I of the allogeneic T cell. In some embodiments, the exogenous Nef protein upon expression down-modulates (e.g., down-regulates cell surface expression and/or effector function) endogenous TCR (e.g., TCRα and/or TCRβ), CD3ε/δ/γ, and/or MHC I by at least about 40% (such as at least about any of 50%, 60%, 70%, 80%, 90%, or 95%). In some embodiments, the allogeneic T cell comprising an exogenous Nef protein described herein elicit no or reduced (such as reduced by at least about any of 30%, 40%, 50%, 60%, 70%, 80%, 90%, or 95%) GvHD response in a histoincompatible individual as compared to the GvHD response elicited by the same allogeneic T cell without Nef expression. In some embodiments, the exogenous Nef comprises an amino acid sequence of SEQ ID NO: 121, 122, 136, or 139.

In some embodiments, the present invention also provides a method of treating a disease (such as cancer, infectious disease, autoimmune disorders, or radiation sickness) in an individual receiving an allogeneic T cell transplant without inducing GvHD or transplantation rejection, comprising introducing into the allogeneic T cell a first nucleic acid encoding a TCR and/or MHC I downregulating molecule (such as an exogenous Nef protein (e.g., wildtype Nef such as wildtype SIV Nef, or mutant Nef such as mutant SIV Nef)), wherein the TCR and/or MHC I downreglating molecule (such as exogenous Nef protein) upon expression results in down-modulation (e.g., down-regulation of cell surface expression and/or effector function such as signal transduction) of the endogenous TCR, CD3ε, and/or MHC I of the allogeneic T cell. In some embodiments, the allogeneic T cell is an allogeneic ITAM-modified CAR-T cell, ITAM-modified TCR-T cell, ITAM-modified cTCR-T cell, or ITAM-modified TAC-like-T cell. In some embodiments, the method further comprises introducing into the allogeneic T cell a second nucleic acid encoding a functional exogenous receptor comprising a CMSD described herein. In some embodiments, the second nucleic acid encodes an ITAM-modified CAR, e.g., ITAM-modified BCMA CAR or ITAM-modified CD20 CAR. In some embodiments, the exogenous Nef protein upon expression down-modulates (e.g., down-regulates cell surface expression and/or effector function) endogenous TCR (e.g., TCRα and/or TCRβ), CD3ε/δ/γ, and/or MHC I by at least about 40% (such as at least about any of 50%, 60%, 70%, 80%, 90%, or 95%). In some embodiments, the exogenous Nef comprises an amino acid sequence of SEQ ID NO: 121, 122, 136, or 139.

In some embodiments, the present invention also provides a method of reducing GvHD or transplantation rejection of an allogeneic ITAM-modified CAR-T cell, comprising introducing into the allogeneic ITAM-modified CAR-T cell a nucleic acid encoding a TCR and/or MHC I downregulating molecule (such as an exogenous Nef protein (e.g., wildtype Nef such as wildtype SIV Nef, or mutant Nef such as mutant SIV Nef)), wherein the TCR and/or MHC I downregulating molecule (such as exogenous Nef protein) upon expression results in down-modulation (e.g., down-regulation of cell surface expression and/or effector function such as signal transduction) of the endogenous TCR, CD3ε, and/or MHC I of the allogeneic ITAM-modified CAR-T cell. In some embodiments, the TCR and/or MHC I downregulating molecule (such as exogenous Nef protein (e.g., wildtype Nef such as wildtype SIV Nef, or mutant Nef such as mutant SIV Nef)) upon expression down-modulates (e.g., down-regulates cell surface expression and/or effector function) endogenous TCR (e.g., TCRα and/or TCRβ), CD3ε, and/or MHC I by at least about 40% (such as at least about any of 50%, 60%, 70%, 80%, 90%, or 95%). In some embodiments, the TCR and/or MHC I downregulating molecule (such as exogenous Nef protein (e.g., wildtype Nef such as wildtype SIV Nef, or mutant Nef such as mutant SIV Nef)) upon expression does not down-modulate (e.g., down-regulate cell surface expression and/or effector function) the ITAM-modified CAR, or down-modulates the ITAM-modified CAR by at most about 80% (such as at most about any of 70%, 60%, 50%, 40%, 30%, 20%, 10%, or 5%). In some embodiments, the exogenous Nef comprises an amino acid sequence of SEQ ID NO: 121, 122, 136, or 139. In some embodiments, the allogeneic ITAM-modified T cell comprising an exogenous Nef protein described herein elicit no or reduced (such as reduced by at least about any of 30%, 40%, 50%, 60%, 70%, 80%, 90%, or 95%) GvHD response in a histoincompatible individual as compared to the GvHD response elicited by an allogeneic ITAM-modified T cell from the same donor source without Nef expression.

### IX. Kits and Articles of Manufacture

Further provided are kits, unit dosages, and articles of manufacture comprising any one of the modified T cells (e.g., allogeneic or autologous T cell) expressing a functional exogenous receptor comprising a CMSD described herein (e.g., ITAM-modified CAR, ITAM-modified TCR, ITAM-modified cTCR, or ITAM-modified TAC-like chimeric receptor). In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer, autoimmune disease, or infectious disease) as described herein, or reducing/preventing GvHD or transplantation rejection when treating a disease or disorder, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples and exemplary embodiments below are intended to be purely exemplary of the invention and should therefore not be considered to limit the invention in any way. The following examples and detailed description are offered by way of illustration and not by way of limitation.

### Example 1. Evaluation of CMSD ITAM Activation Activity

### 1. Construction of ISD-Modified BCMA CARs

To test activation activity of CARs containing various intracellular signaling domains (ISDs), ISD-modified CARs were constructed. “ISD-modified CAR” is used herein to describe CARs with any modifications in the ISD, which may not necessarily be an ITAM-modified CAR described herein. For example, constructs in Table 1 are all “ISD-modified CARs”, but only M663, M665, M666, M667, M678, M679, M680, M681, M682, M683, M684, M685, and M799 are “ITAM-modified CARs” described herein.

pLVX-Puro (Clontech, #632164) is an HIV-1-based lentivirus expression vector comprising a constitutively active human cytomegalovirus immediate early promoter (PCMV IE) located just upstream of the multiple cloning site (MCS). A homemade lentivirus vector was produced by replacing the original P_{CMVIE} promoter of pLVX-Puro with a human elongation factor 1a (hEF1α) promoter sequence carrying EcoRI and ClaI restriction sites at C-terminus, hereinafter referred to as “pLVX-hEF1α-Puro lentiviral vector”. Briefly, polynucleotides encoding CD8α SP-BCMA scFv-CD8α hinge-CD8α TM-ISD with various ISD structures as shown in Table 1, and polynucleotide encoding the control construct CD8α SP-BCMA scFv-CD8α hinge-CD8α TM-CD3ζ (“M660”) and CD8α SP-BCMA scFv-CD8α hinge-CD8α TM-4-1BB-Linker 2-4-1BB-Linker 2-4-1BB (“M661”) were chemically synthesized (corresponding ISD-modified CAR construct name see Table 1), and cloned into pLVX-hEF1α-Puro lentiviral vector, respectively, for the construction of ISD-modified CAR recombinant transfer plasmids. These transfer plasmids were then subject to lentivirus packaging procedure below, respectively.

The lentivirus packaging plasmid mixture containing psPAX2 (packaging; Addgene, #12260) and pMD2.G (envelope; Addgene, #12259) was pre-mixed with the above ISD-modified CAR transfer plasmids, respectively, incubated at room temperature, then transduced into HEK 293T cells, respectively. 60 hours post-transduction, supernatant containing lentiviruses was collected by centrifuging the cell transduction mixture at 4° C., 3000 rpm for 5 min. The supernatant was filtered using 0.45 μm filter, and further concentrated using 500 KD hollow fiber membrane tangential flow filtration to obtain concentrated lentiviruses. These concentrated lentiviruses were stored at −80° C., hereinafter referred to as M660 lentivirus (control), M661 lentivirus, M662 lentivirus, M663 lentivirus, M665 lentivirus, M666 lentivirus, M667 lentivirus, M678 lentivirus, M679 lentivirus, M680 lentivirus, M681 lentivirus, M682 lentivirus, M683 lentivirus, M684 lentivirus, M685 lentivirus, and M799 lentivirus; or ISD-modified CAR lentiviruses collectively.

Jurkat cells (ATCC®, #TIB152™) were cultured in 90% RPMI 1640 medium (Life Technologies, #22400-089) and 10% Fetal Bovine Serum (FBS, Life Technologies, #10099-141). ISD-modified CAR lentiviruses from above were added into the supernatant of Jurkat cell culture for transduction, respectively (hereinafter referred to as Jurkat-ISD-modified CAR). 72 hours post transduction, positive cell clones were selected using 1 μg/mL puromycin for 2 week.

TABLE 1
Intracellular signaling domain structure of ISD-modified CARs

| CAR | Intracellular signaling domain | ISD amino |
|---|---|---|
| construct | (ISD) construct struction | acid sequence |
| M660 | CD3ζ | SEQ ID NO: 7 |
| (control) | | |
| M661 | 4-1BB-Linker 2-4-1BB-Linker 2-4-1BB | SEQ ID NO: 137 |
| (control) | | |
| M662 | (CD3ζ intracellular signaling domain | SEQ ID NO: 40 |
| | without 3 ITAMs and stop codon)-Linker | |
| | 2-(CD3ζ intracellular signaling domain | |
| | without 3 ITAMs and stop codon) | |
| M663 | Linker 6-CD3ζ ITAM1-Linker 1-CD3ζ | SEQ ID NO: 41 |
| | ITAM2-Linker 7-CD3ζ ITAM3-Linker 2 | |
| M665 | Linker 6-CD3ζ ITAM1-Linker 1-CD3ζ | SEQ ID NO: 42 |
| | ITAM1-Linker 7-CD3ζ ITAM1-Linker 2 | |
| M666 | Linker 6-CD3ζ ITAM2-Linker 1-CD3ζ | SEQ ID NO: 43 |
| | ITAM2-Linker 7-CD3ζ ITAM2-Linker 2 | |
| M667 | Linker 6-CD3ζ ITAM3-Linker 1-CD3ζ | SEQ ID NO: 44 |
| | ITAM3-Linker 7-CD3ζ ITAM3-Linker 2 | |
| M678 | Linker 6-CD3δ ITAM-Linker 1-CD3δ | SEQ ID NO: 45 |
| | ITAM-Linker 7-CD3δ ITAM-Linker 2 | |
| M679 | Linker 6-CD3ε ITAM-Linker 1-CD3ε | SEQ ID NO: 46 |
| | ITAM-Linker 7-CD3ε ITAM-Linker 2 | |
| M680 | Linker 6-CD3γ ITAM-Linker 1-CD3γ | SEQ ID NO: 47 |
| | ITAM-Linker 7-CD3γ ITAM-Linker 2 | |
| M681 | Linker 6-DAP12 ITAM-Linker 1-DAP12 | SEQ ID NO: 48 |
| | ITAM-Linker 7-DAP12 ITAM-Linker 2 | |
| M682 | Linker 6-Igα ITAM-Linker 1-Igα | SEQ ID NO: 49 |
| | ITAM-Linker 7-Igα ITAM-Linker 2 | |
| M683 | Linker 6-Igβ ITAM-Linker 1-Igβ | SEQ ID NO: 50 |
| | ITAM-Linker 7-Igβ ITAM-Linker 2 | |
| M684 | Linker 6-FcεRIβ ITAM-Linker 1- | SEQ ID NO: 51 |
| | FcεRIβ ITAM-Linker 7-FcεRIβ | |
| | ITAM-Linker 2 | |
| M685 | Linker 6-FcεRIγ ITAM-Linker 1- | SEQ ID NO: 52 |
| | FcεRIγ ITAM-Linker 7-FcεRIγ | |
| | ITAM-Linker 2 | |
| M799 | Linker 6-CNAIP/NFAM1 ITAM-Linker 1- | SEQ ID NO: 53 |
| | CNAIP/NFAM1 ITAM-Linker 7-CNAIP/NFAM1 | |
| | ITAM-Linker 2 | |

### 2. Evaluation of ISD-Modified BCMA CAR Specific Activation Activity

1×10⁶ Jurkat-ISD-modified BCMA CAR cells described above were mixed with target cell lines RPNH18226 (with CFSE label) and non-target cell lines K562 (with CFSE label), respectively, at E:T ratio of 1:1. The mixed cells were added into 24-well plate, replenished with RPMI 1640 medium (contains 10% FBS) to a final volume of 1 mL/well, and incubated in a 37° C., 500 CO₂ incubator. Sample from each co-cultured assays was collected to assess CD69 expression after 2.5 hours of incubation, CD25 expression after 24 hours of incubation, and HLA-DR expression after 144 hours of incubation in CFSE negative cells, respectively. Untransduced Jurkat cells (“Jurkat”) served as control.

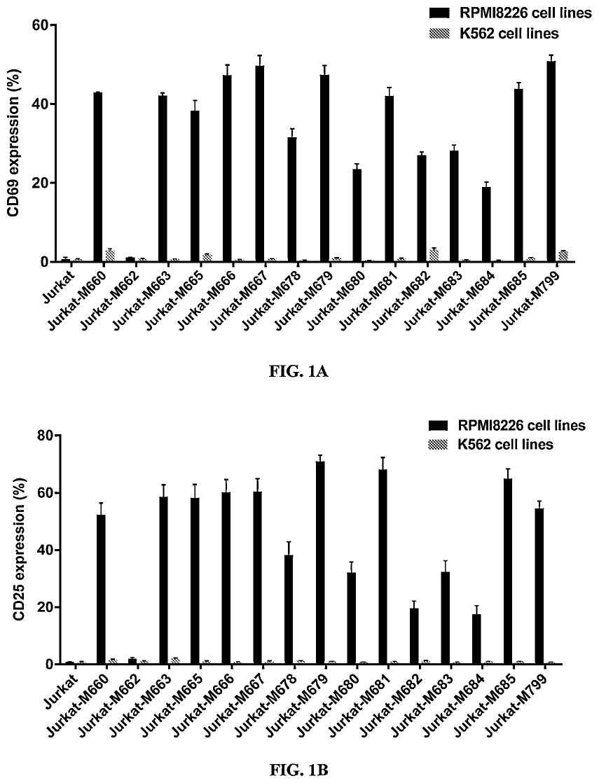

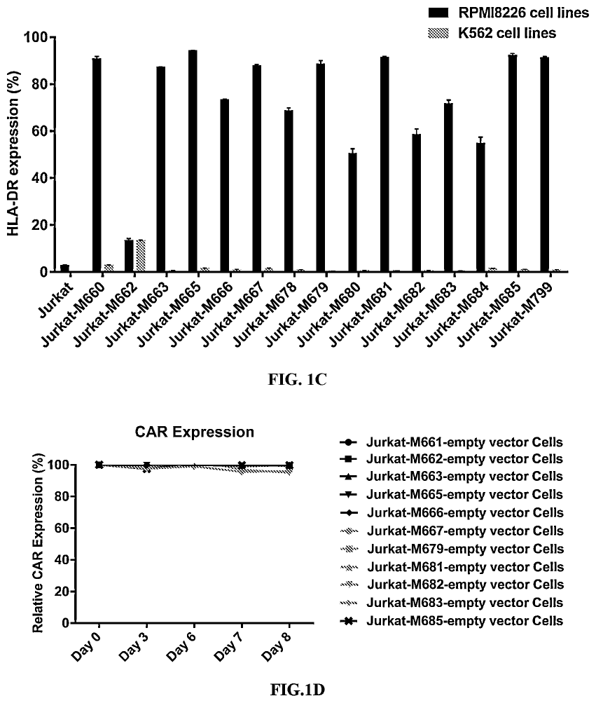

As shown in FIGS. 1A-1C, expression of activation molecular CD69, CD25, and HLA-DR in Jurkat-ITAM-modified BCMVA CAR cells significantly increased under the stimulation of target cell lines RPMI8226 (P<0.05). While, no expression of CD69, CD25, and HLA-DR was detected in Jurkat-ITAM-modified BCMVA CAR cells co-cultured with non-target cell lines K562. These data suggest that arrangement of CMSD ITAMs in CAR-T cells possesses CAR-mediated specific activation activity.

### 3. SIV Nef or SIV Nef M116 Affects CAR Expression Via CD3CTITAM1 or CD3CTITAM2

Lentiviruses carrying wildtype SIV Nef sequence, SIV Nef M116 sequence, and empty vector were added into the suspension of Jurkat-ISD-modified CAR cell cultures for transduction, respectively. 3 days, 6 days, 7 days, and 8 days post-transduction, 5×10⁵ cells were collected and centrifuged at room temperature, the supernatant was discarded. Cells were resuspended with 1 mL DPBS, 1 μL FITC-Labeled Human BCMA protein (ACROBIOSYSTEM, #BCA-HF254-200 UG) was added and the suspension was incubated for 30 min at 4° C. After incubation, the centrifugation and resuspension with DPBS step was repeated twice. Then cells were resuspended with DPBS for FACS to detect BCM1A ISD-modified CAR expression. The relative ISD-modified CAR expression rates of each Jurkat-ISD-modified CAR-SIV Nef cells and Jurkat-ISD-modified CAR-SIV Nef M116 cells are normalized with each control transduced with an empty vector at the same time point, and calculated using the formula: Relative ISD-modified CAR expression (%)=[sample (%)]/[control (%)]×100%. For instance, the relative ISD-modified CAR expression value of “Jurkat-M661-SIV Nef” on Day 3 is calculated as follows: Relative ISD-modified CAR expression (%)=[Jurkat-M661-SIV Nef (%)]/[Jurkat-M661-empty vector (%)]×100%.

As shown in FIGS. 1D-1F, ISD-modified CAR positive rates of each Jurkat-ISD-modified CAR-SIV Nef cells (FIG. 1E) and Jurkat-ISD-modified CAR-SIV Nef M116 cells (FIG. 1F) are normalized with the control of Jurkat-ISD-modified CAR-empty vector cells (FIG. 1D) at the same time points (such as day 0, day 3, day 6, day 7, and day 8 transduction of lentiviruses carrying SIV Nef sequence, SIV Nef M116 sequence, or empty vector). 3 days post-Nef/control lentivirus transduction, ISD-modified CAR positive rates of Jurkat-M663-SIV Nef cells, Jurkat-M665-SIV Nef cells, and Jurkat-M666-SIV Nef cells dropped to 46.72%, 82.31%, and 57.04%, respectively, compared to controls on day 3; ISD-modified CAR positive rates of Jurkat-M663-SIV Nef M116 cells, Jurkat-M665-SIV Nef M116 cells, and Jurkat-M666-SIV Nef M116 cells dropped to 50.92%, 70.35%, and 56.22%, respectively, compared to controls on day 3; while ISD-modified CAR positive rates of Jurkat-ISD-modified CAR-empty vector cells were all above 95%, as controls. 6 days, 7 days, and 8 days post-Nef/control lentivirus transduction, ISD-modified CAR expression became stable in each group, ISD-modified CAR positive rates of Jurkat-M663-SIV Nef cells, Jurkat-M665-SIV Nef cells, and Jurkat-M666-SIV Nef cells dropped to 41.19%-69.84%; ISD-modified CAR positive rates of Jurkat-M663-SIV Nef M116 cells, Jurkat-M665-SIV Nef M116 cells, and Jurkat-M666-SIV Nef M116 cells dropped to 44.65%-64.94%; while ISD-modified CAR positive rates of Jurkat-ISD-modified CAR-empty vector cells were still above 95%, as controls.

As shown in Table 1, ISDs of M663 (ITAM1/2/3), M665 (ITAM1/1/1), M666 (ITAM2/2/2), and M667 (ITAM3/3/3) comprise ITAMs of CD3ε, while the ISD of M662 (0 ITAM) comprise only non-ITAM sequence of CD3ζ. The down-regulation by SIV Nef or SIV Nef M116 of M663, M665, and M666, but not M662 and M667 seen above demonstrate that SIV Nef and SIV Nef M116 regulate CAR expression by interacting with CD3ζ ITAM1 and CD3ζ ITAM2, but not CD3ζ ITAM3 or non-ITAM CD3 sequence; further, SIV Nef and SIV Nef M116 seem to have stronger interaction with CD3ζ ITAM2 compared to CD3ζ ITAM1 (see CAR+ rate M663<M666<M665). Other tested ISDs do not contain any CD3 sequence, and SIV Nef and SIV Nef M116 do not seem to interact with 4-1BB co-stimulatory domain, CD3ε ITAM, DAP12 ITAM, Igα ITAM, Igβ ITAM, or FcεRIγITAM (FIGS. 1D-1F).

4. Interaction Between SIV Nef and SIV Nef M116 with CD3δ ITAM, CD3γ ITAM, FcεRβ ITAM, and CNAIP/NFAM1 ITAM, Respectively

Lentiviruses carrying wildtype SIV Nef sequence, SIV Nef M116 sequence, and empty vector were separately added into the suspension of Jurkat-ITAM-modified BCMA CAR (Jurkat-M663, Jurkat-M678, Jurkat-M680, Jurkat-M684, and Jurkat-M799) cell culture for transduction. 3 days, 6 days, 7 days, and 8 days post-transduction, 5×10⁵ cells were collected and centrifuged at room temperature, the supernatant was discarded. Cells were resuspended with 1 mL DPBS, 1 μL FITC-Labeled Human BCMA protein (Biolegend, #310906) was added and the suspension was incubated for 30 min at 4° C. After incubation, the centrifugation and resuspension with DPBS step was repeated twice. Then cells were resuspended with DPBS for FACS to detect BCMA CAR expression.

As shown in FIGS. 1G-1I, ITAM-modified BCMA CAR positive rates of each Jurkat-ITAM-modified BCMA CAR cells were above 95%; No significant down-regulation of CAR positive rates were observed in Jurkat-M678 cells, Jurkat-M680 cells, Jurkat-M684 cells, and Jurkat-M799 cells transduced with SIV Nef, SIV Nef M116, and empty vector, respectively (P>0.05). CAR positive rate of Jurkat-M663 transduced with SIV Nef and SIV Nef M116 respectively, was significantly down-regulated as the incubation time increased (P<0.05). These data suggest that SIV Nef and SIV Nef M116 do not seem to interact with M678 (CD36 ITAM), M680 (CD3γ ITAM), M684 (FcεRIP ITAM), or M799 (CNAIP/NFAM1 ITAM).

### Example 2. In Vitro Cytotoxicity Analysis of ITAM-Modified CAR-T Cells

### 1. In Vitro Cytotoxicity Assessment of ITAM-Modified BCMA CAR-T Cells

To construct ITAM-modified BCMA CARs, fusion gene sequences encoding CD8α SP-BCMA scFv-CD8α hinge-CD8α TM-4-1BB (“BCMA-BB”; only contains 4-1BB co-stimulatory signaling domain), CD8α SP-BCMA scFv-CD8α hinge-CD8α TM-4-1BB-CD3ζ (“BCMA-BBz”, SEQ ID NO: 75), CD8α SP-BCMA scFv-CD8α hinge-CD8α TM-4-1BB-ITAM007 (“BCMA-BB007”), CD8α SP-BCMA scFv-CD8α hinge-CD8α TM-4-1BB-ITAM008 (“BCMA-BB008”), CD8α SP-BCMA scFv-CD8α hinge-CD8α TM-4-1BB-ITAM009 (“BCMA-BB009”), and CD8α SP-BCMA scFv-CD8α hinge-CD8α TM-4-1BB-ITAM010 (“BCMA-BB010”) were chemically synthesized, and cloned into pLVX-hEF1α-Puro lentiviral vector (see Example 1) for the construction of recombinant transfer plasmids, respectively (see Table 2 for ITAM construct structures), hereinafter referred to as pLVX-BCMA-BB transfer plasmid (negative control), pLVX-BCMA-BBz transfer plasmid (positive control), and pLVX-BCMA-(BB007-BB010) transfer plasmids. All lentiviral transfer plasmids were purified, and packaged into lentiviruses as described in Example 1, hereinafter referred to as BCMA-BB lentivirus, BCMA-BBz lentivirus, and BCMA-(BB007-BB010) lentiviruses, respectively.

TABLE 2
ITAM construct structures of ITAM-modified BCMA CARs

| ITAM-modified | ITAM | | ITAM construct | CAR construct |
|---|---|---|---|---|
| CAR construct | construct | ITAM construct structure | amino acid sequence | amino acid sequence |
| BCMA-BB007 | ITAM007 | Linker 5-CD3ζ ITAM1-Linker 1-CD3ζ | SEQ ID NO: 54 | SEQ ID NO: 76 |
| | | ITAM2-Linker 3-CD3ζ ITAM3-Linker 4 | | |
| BCMA-BB008 | ITAM008 | Linker 1-CD3ζ ITAM1-Linker 2-CD3ζ | SEQ ID NO: 55 | SEQ ID NO: 77 |
| | | ITAM1-Linker 2-CD3ζ ITAM1-Linker 2 | | |
| BCMA-BB009 | ITAM009 | Linker 1-CD3ε ITAM-Linker 2-CD3ε | SEQ ID NO: 56 | SEQ ID NO: 78 |
| | | ITAM-Linker 2-CD3ε ITAM-Linker 2 | | |
| BCMA-BB010 | ITAM010 | Linker 1-CD3δ ITAM-Linker 2-CD3ε | SEQ ID NO: 57 | SEQ ID NO: 79 |
| | | ITAM-Linker 2-CD3γ ITAM-Linker | | |
| | | 2-DAP12 ITAM-Linker 2 | | |

50 mL peripheral blood was extracted from volunteers. Peripheral blood mononuclear cells (PBMCs) were isolated via density gradient centrifugation. Pan T Cell Isolation Kit (Miltenyi Biotec, #130-096-535) was used to magnetically label PBMCs and isolate and purify T lymphocytes. CD3/CD28 conjugated magnetic beads were used for activation and expansion of purified T lymphocytes. Activated T lymphocytes were collected and resuspended in RPMI 1640 medium (Life Technologies, #22400-089). 3 days post activation, 5×10⁶ activated T lymphocytes were transduced with lentiviruses BCMA-BB, BCMA-BBz, BCMA-BB007, BCMA-BB008, BCMA-BB009, and BCMA-BB010, respectively (hereinafter referred to as BCMA-BB T cells, BCMA-BBz T cells, and BCMA-(BB007-BB010) T cells, respectively). T cell suspension was added into 6-well plate, and incubated overnight in 37° C., 5% CO₂ incubator. 3 days post-transduction, modified T cells were mixed under 40:1 effector to target cell (E:T) ratio with multiple myeloma (MM) cell line RPMI8226.Luc (BCMA+, with luciferase (Luc) marker), respectively, incubated in Corning® 384-well solid white plate for 12 hours. ONE-Glo™ Luciferase Assay System (PROMEGA, #B6110) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells.

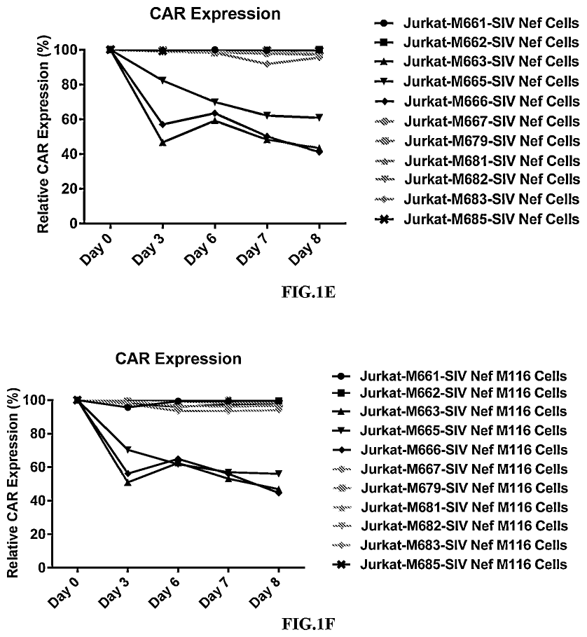

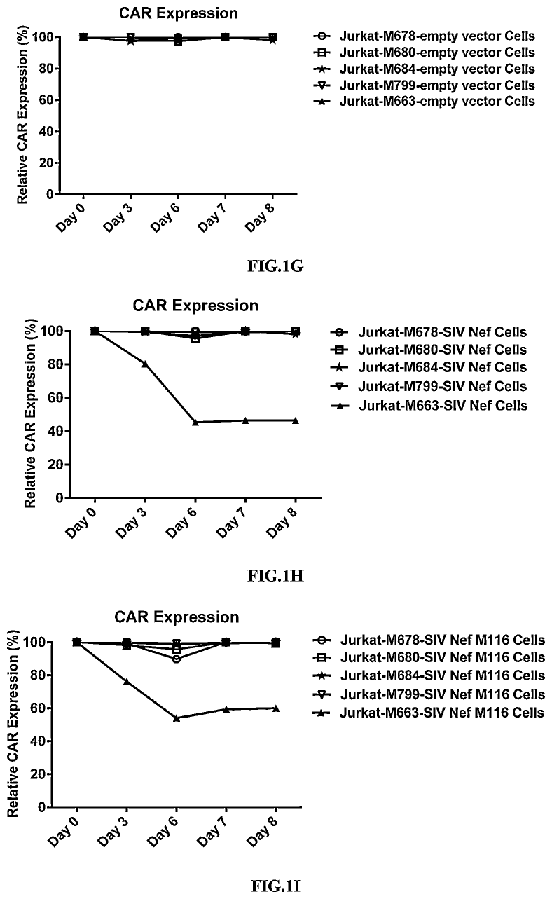

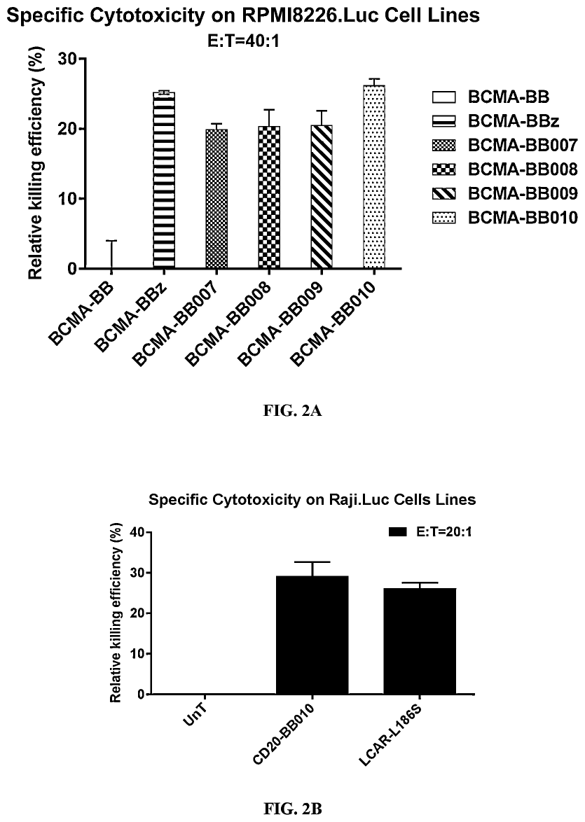

As shown in FIG. 2A, negative control BCMA-BB without primary CD3ζ intracellular signaling domain failed to mediate tumor cell killing. ITAM-modified BCMA CARs (BCMA-BB007, BCMA-BB008, BCMA-BB009, and BCMA-BB010) were all capable of mediating tumor cell killing on RPMI8226.Luc cell lines compared to UnT (P<0.05). No significant difference in cytotoxicity (P>0.05) was observed among ITAM-modified BCMA CARs (BCMA-(BB007-BB010)) and BCMA CAR with traditional CD3ζ intracellular signaling domain (BCMA-BBz). These data suggest that chimeric signaling domains described herein (e.g., ITAM007-ITAM010) may provide a promising strategy for constructing ITAM-modified CARs that retain tumor cell killing.

### 2. In Vitro Cytotoxicity Assessment of ITAM-Modified CD20 CAR-T Cells

To construct ITAM-modified CD20 CARs, fusion gene sequences encoding CD8α SP-CD20 scFv (Leu16)-CD8α hinge-CD8α TM-4-1BB-CD3ζ (“LCAR-L186S”, SEQ ID NO: 97), and CD8α SP-CD20 scFv (Leu16)-CD8α hinge-CD8α TM-4-1BB-ITAM010 (“CD20-BB010”, SEQ ID NO: 98) were chemically synthesized, and cloned into pLVX-hEF1α-Puro lentiviral vector (see Example 1) for the construction of pLVX-LCAR-L186S and pLVX-CD20-BB010 recombinant transfer plasmids, respectively. Lentiviral transfer plasmids were purified, and packaged into lentiviruses as described in Example 1, hereinafter referred to as LCAR-L186S lentivirus and CD20-BB010 lentivirus, respectively.

PBMCs and T lymphocytes were prepared according to the method described above. 3 days post activation, 5×10⁶ activated T lymphocytes were transduced with lentiviruses LCAR-L186S (referred to as LCAR-L186S T cells) and CD20-BB010 (referred to as CD20-BB010 T cells), respectively. T cell suspension was added into 6-well plate, and incubated overnight in 37° C., 5% CO₂ incubator. 3 days post-transduction, modified T cells were mixed with lymphoma Raji.Luc (CD20+, with luciferase (Luc) marker) cell lines at E:T ratio of 20:1, respectively, incubated in Corning® 384-well solid white plate for 12 hours. ONE-Glo™ Luciferase Assay System (PROMEGA, #B6110) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (UnT) served as control.

As shown in FIG. 2B, both ITAM-modified CD20 CAR (CD20-BB010) and LCAR-L186S exhibited much stronger cytotoxicity compared to UnT (P<0.05), and ITAM-modified CD20 CAR (CD20-BB010) shows similar cytotoxicity as CD20 CAR with traditional CD3ζ intracellular signaling domain (LCAR-L186S; P>0.05).

In summary, the above data suggest that chimeric signaling domains described herein (e.g., ITAM007-ITAM010) may provide a promising strategy for constructing ITAM-modified CARs that retain tumor cell killing.

### Example 3. Impact of CMSD Linker of Chimeric Signaling Domain on CAR-T Cells Activity

### 1. Construction of ITAM-Modified BCMA CARs

The CMSD linkers of ITAM010 intracellular signaling domain were deleted or replaced, to form ITAM024 construct, ITAM025 construct, ITAM026 construct, ITAM027 construct, ITAM028 construct, and ITAM029 construct (corresponding ITAM construct see Table 3). To construct ITAM-modified BCMA CARs, the CD3ζ intracellular signaling domain of BCMA-BBz (CD8α SP-BCMA scFv-CD8α hinge-CD8α TM-4-1BB-CD3ζ) was replaced with above construct for the construction of pLVX-BCMA-BB024, pLVX-BCMA-BB025, pLVX-BCMA-BB026, pLVX-BCMA-BB027, pLVX-BCMA-BB028, and pLVX-BCMA-BB029 transfer plasmid, respectively. These transfer plasmids were then purified and packaged into lentiviruses as described in Example 1, hereinafter referred to as BCMA-BB024 lentivirus, BCMA-BB025 lentivirus, BCMA-BB026 lentivirus, BCMA-BB027 lentivirus, BCMA-BB028 lentivirus, and BCMA-BB029 lentivirus, respectively.

TABLE 3
ITAM construct structures of ITAM-modified BCMA CARs

| ITAM-modified | ITAM | | ITAM construct | CAR construct |
|---|---|---|---|---|
| CAR construct | construct | ITAM construct structure | amino acid sequence | amino acid sequence |
| BCMA-BB024 | ITAM024 | CD3δ ITAM-CD3ε ITAM- | SEQ ID NO: 58 | SEQ ID NO: 80 |
| | | CD3γ ITAM-DAP12 ITAM | | |
| BCMA-BB025 | ITAM025 | Linker 14-CD3δ ITAM-Linker 13- | SEQ ID NO: 59 | SEQ ID NO: 81 |
| | | CD3ε ITAM-Linker 13-CD3γ | | |
| | | ITAM-Linker 13-DAP12 ITAM- | | |
| | | Linker 13 | | |
| BCMA-BB026 | ITAM026 | Linker 15-CD3δ ITAM-Linker 11- | SEQ ID NO: 60 | SEQ ID NO: 82 |
| | | CD3ε ITAM-Linker 11-CD3γ | | |
| | | ITAM-Linker 11-DAP12 ITAM- | | |
| | | Linker 11 | | |
| BCMA-BB027 | ITAM027 | Linker 8-CD3δ ITAM-Linker 9- | SEQ ID NO: 61 | SEQ ID NO: 83 |
| | | CD3ε ITAM-Linker 9-CD3γ | | |
| | | ITAM-Linker 9-DAP12 ITAM- | | |
| | | Linker 9 | | |
| BCMA-BB028 | ITAM028 | Linker 6-CD3δ ITAM-Linker 10- | SEQ ID NO: 62 | SEQ ID NO: 84 |
| | | CD3ε ITAM-Linker 12-CD3γ | | |
| | | ITAM-Linker 11-DAP12 ITAM- | | |
| | | Linker 9 | | |
| BCMA-BB029 | ITAM029 | Linker 8-CD3δ ITAM-Linker 9- | SEQ ID NO: 63 | SEQ ID NO: 85 |
| | | CD3ε ITAM-Linker 11-CD3γ | | |
| | | ITAM-Linker 10-DAP12 ITAM- | | |
| | | Linker 12 | | |

### 2. Cytotoxicity of ITAM-Modified BCMA CAR-T Cells In Vitro Assay

PBMCs and T lymphocytes were prepared according to the method described in Example 2. 3 days post activation, 5×10⁶ activated T lymphocytes were transduced with lentiviruses encoding ITAM-modified BCMA CARs (BCMA-BB010 lentiviruses from Example 2, BCMA-BB024 lentiviruses, BCMA-BB025 lentiviruses, BCMA-BB026 lentiviruses, BCMA-BB027 lentiviruses, BCMA-BB028 lentiviruses, and BCMA-BB029 lentiviruses), and control BCMA-BBz lentiviruses, respectively. T cell suspension was added into 6-well plate, and incubated overnight in a 37° C., 5% CO₂ incubator. 3 days post-transduction, modified T cells were mixed with multiple myeloma (MM) cell line RPMI8226.Luc at E:T ratio of 2.5:1, respectively, incubated in Corning® 384-well solid white plate for 12 hours. ONE-Glo™ Luciferase Assay System (TAKARA, #B6120) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (“UnT”) served as control.

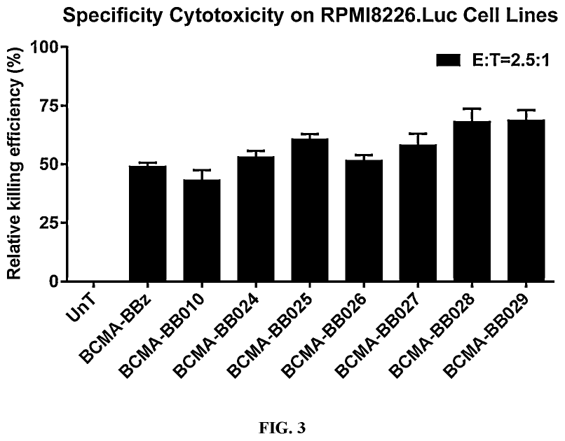

As shown in FIG. 3, BCMA-BB024, the CMSD ITAMs were directly linked to each other; BCMA-BB010, BCMA-BB025, BCMA-BB026, BCMA-BB027, BCMA-BB028, and BCMA-BB029, the CMSD ITAMs were connected by different CMSD linkers; were all capable of mediating significant specific tumor cell killing on RPMI8226.Luc cell lines compared to UnT (P<0.05). BCMA-BB025, BCMA-BB028, and BCMA-BB029 showed significantly CAR-specific cytotoxicity compared to BCMA-BBz (P<0.05). No significant difference in cytotoxicity (P>0.05) was observed among BCMA-BB010, BCMA-BB024, BCMA-BB026, BCMA-BB027, and BCMA CAR with traditional CD3ζISD (BCMA-BBz). These data suggests that CMSD linker of chimeric signaling domain dose not compromise with CAR-mediated specific cytotoxicity of CAR-T cells.

### Example 4. Impact of Order of CMSD ITAMs on CAR-T Cells Activity

### 1. Construction of ITAM-Modified BCMA CARs

To construct ITAM-modified BCMA CARs, the ITAM010 intracellular signaling domain of BCMA-BB010 (CD8α SP-BCMA scFv-CD8α hinge-CD8α TM-4-1BB-ITAM010) was replaced with ITAM construct comprising different order of ITAMs from ITAM010, such as ITAM030, ITAM031, and ITAM032 (corresponding ITAM construct see Table 4) for the construction of pLVX-BCMA-BB030, pLVX-BCMA-BB031, and pLVX-BCMA-BB032 transfer plasmid, respectively. These transfer plasmids were then purified and packaged into lentiviruses as described in Example 1, hereinafter referred to as BCMA-BB030 lentivirus, BCMA-BB031 lentivirus, and BCMA-BB032 lentivirus, respectively.

TABLE 4
ITAM construct structures of ITAM-modified BCMA CARs

| ITAM-modified | ITAM | | ITAM construct | CAR construct |
|---|---|---|---|---|
| CAR construct | construct | ITAM construct structure | amino acid sequence | amino acid sequence |
| BCMA-BB030 | ITAM030 | Linker 1-CD3ε ITAM-Linker 2- | SEQ ID NO: 64 | SEQ ID NO: 86 |
| | | CD3δ ITAM-Linker 2-DAP12 ITAM- | | |
| | | Linker 2-CD3γ ITAM-Linker 2 | | |
| BCMA-BB031 | ITAM031 | Linker 1-CD3γ ITAM-Linker 2- | SEQ ID NO: 65 | SEQ ID NO: 87 |
| | | DAP12 ITAM-Linker 2-CD3δ ITAM- | | |
| | | Linker 2-CD3ε ITAM-Linker 2 | | |
| BCMA-BB032 | ITAM032 | Linker 1-DAP12 ITAM-Linker 2- | SEQ ID NO: 66 | SEQ ID NO: 88 |
| | | CD3γ ITAM-Linker 2-CD3ε ITAM- | | |
| | | Linker 2-CD3δ ITAM-Linker 2 | | |

### 2. Cytotoxicity of ITAM-Modified BCMA CAR-T Cells In Vitro Assay

PBMCs and T lymphocytes were prepared according to the method described in Example 2. 3 days post activation, 5×10⁶ activated T lymphocytes were transduced with lentiviruses encoding ITAM-modified BCMA CARs (including BCMA-BB010 lentiviruses from Example 2, BCMA-BB030 lentiviruses, BCMA-BB031 lentiviruses, and BCMA-BB032 lentiviruses), and control BCMA-BBz lentiviruses, respectively. T cell suspension was added into 6-well plate, and incubated overnight in a 37° C., 5% CO₂ incubator. 3 days post-transduction, modified T cells were mixed with multiple myeloma (MM) cell line RPMI8226.Luc at E:T ratio of 2.5:1, respectively, incubated in Corning® 384-well solid white plate for 12 hours. ONE-Glo™ Luciferase Assay System (TAKARA, #B6120) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (“UnT”) served as control.

As shown in FIG. 4, ITAM-modified BCMA CAR-T cells (BCMA-BB030˜BCMA-BB032) were all capable of mediating significant specific tumor cell killing on RPMI8226.Luc cell lines compared to UnT (P<0.05). BCMA-BB031 and BCMA-BB032 showed significantly CAR-specific cytotoxicity compared to BCMA-BBz (P<0.05). No significant difference in cytotoxicity (P>0.05) was observed between BCMA-BB010 and BCMA-BB030 with BCMA-BBz. These results suggest that rearrangement of CMSD ITAMs dose not compromise with CAR-mediated specific cytotoxicity of CAR-T cells.

### Example 5. Impact of Quantity and Source of CMSD ITAM on CAR-T Cells Activity

### 1. Construction of ITAM-Modified BCMA CARs

ITAM-modified BCMA CARs, the intracellular signaling domain consist of 1, 2, 3, or 4 CMSD ITAMs, respectively, while different sources were tested. To construct ITAM-modified BCMA CARs, the CD3ζ intracellular signaling domain of BCMA-BBz (CD8α SP-BCMA scFv-CD8α hinge-CD8α TM-4-1BB-CD3ζ) was replaced with ITAM033 construct, ITAM034 construct, ITAM035 construct, ITAM036 construct, ITAM037 construct, or ITAM038 construct (corresponding ITAM construct see Table 5) for the construction of pLVX-BCMA-BB033, pLVX-BCMA-BB034, pLVX-BCMA-BB035, pLVX-BCMA-BB036, pLVX-BCMA-BB037, or pLVX-BCMA-BB038 transfer plasmids, respectively. These transfer plasmids were then purified and packaged into lentiviruses as described in Example 1, hereinafter referred to as BCMA-BB033 lentivirus, BCMA-BB034 lentivirus, BCMA-BB035 lentivirus, BCMA-BB036 lentivirus, BCMA-BB037 lentivirus, and BCMA-BB038 lentivirus, respectively.

TABLE 5
ITAM construct structures of ITAM-modified BCMA CARs

| ITAM-modified | ITAM | | ITAM construct | CAR construct |
|---|---|---|---|---|
| CAR construct | construct | ITAM construct structure | amino acid sequence | amino acid sequence |
| BCMA-BB033 | ITAM033 | Linker 1-CD3ε ITAM-Linker 2 | SEQ ID NO: 67 | SEQ ID NO: 89 |
| BCMA-BB034 | ITAM034 | Linker 1-CD3δ ITAM-Linker 2 | SEQ ID NO: 68 | SEQ ID NO: 90 |
| BCMA-BB035 | ITAM035 | Linker 1-CD3δ ITAM-Linker 2- | SEQ ID NO: 69 | SEQ ID NO: 91 |
| | | CD3ε ITAM-Linker 2 | | |
| BCMA-BB036 | ITAM036 | Linker 1-CD3γ ITAM-Linker 2- | SEQ ID NO: 70 | SEQ ID NO: 92 |
| | | DAP12 ITAM-Linker 2 | | |
| BCMA-BB037 | ITAM037 | Linker 1-CD3δ ITAM-Linker 2- | SEQ ID NO: 71 | SEQ ID NO: 93 |
| | | CD3ε ITAM-Linker 2-CD3ε | | |
| | | ITAM-Linker 2 | | |
| BCMA-BB038 | ITAM038 | Linker 1-CD3δ ITAM-Linker 2- | SEQ ID NO: 72 | SEQ ID NO: 94 |
| | | CD3ε ITAM-Linker 2-CD3γ | | |
| | | ITAM-Linker 2 | | |

### 2. Evaluation of Quantity and Source of CMSD ITAM Impact on BCMA CAR-T Cells Activity

PBMCs and T lymphocytes were prepared according to the method described in Example 2. 3 days post activation, 5×10⁶ activated T lymphocytes were transduced with lentiviruses encoding ITAM-modified BCMA CARs (including BCMA-BB033 lentiviruses, BCMA-BB034 lentiviruses, BCMA-BB035 lentiviruses, BCMA-BB036 lentiviruses, BCMA-BB037 lentiviruses, BCMA-BB038 lentiviruses, BCMA-BB010 lentiviruses form Example 2, BCMA-BB030 lentiviruses form Example 4, BCMA-BB031 lentiviruses form Example 4, and BCMA-BB032 lentiviruses form Example 4), and control BCMA-BBz lentiviruses from Example 2, respectively. T cell suspension was added into 6-well plate, and incubated overnight in a 37° C., 5% CO₂ incubator. 3 days post-transduction, modified T cells were mixed with multiple myeloma (MM) cell line RPMI8226.Luc at E:T ratio of 2.5:1, respectively, incubated in Corning® 384-well solid white plate for 12 hours. ONE-Glo™ Luciferase Assay System (TAKARA, #B6120) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (“UnT”) served as control.

As shown in FIG. 5, ITAM-modified BCMA CAR-T cells (BCMA-BB030˜BCMA-BB038), the intracellular signaling domain consist of 1 to 4 quantities and 1 to 4 sources of CMSD ITAMs, were all capable of mediating significant specific tumor cell killing on RPMI8226.Luc cell lines compared to UnT (P<0.05). BCMA-BB037, BCMA-BB038, BCMA-BB031, and BCMA-BB032 showed significantly CAR-specific cytotoxicity compared to BCMA-BBz (P<0.05). No significant difference in cytotoxicity (P>0.05) was observed among BCMA-BB010, BCMA-BB030, BCMA-BB035, BCMA-BB036, and BCMA CAR with traditional CD3ζISD (BCMA-BBz). These data suggest that rearrangement of 1 to 4 quantities and 1 to 4 sources CMSD ITAMs does not compromise with CAR-mediated specific cytotoxicity of CAR-T cells.

### Example 6. Evaluation of CMSD ITAM Biological Activity

### 1. In Vitro Re-Challenge Model Establishment

Fusion genes encoding CD8α SP-BCMA scFv-CD8α hinge-CD8α TM-4-1BB-ITAM045 (“BCMA-BB045”, SEQ ID NO: 95), ITAM045 construct with “Linker 6-DAP12 ITAM-Linker 1-CD3ε ITAM-Linker 7-CD36 ITAM-Linker 2” (SEQ ID NO: 73); CD8α SP-BCMA scFv-CD8α hinge-CD8α TM-4-1BB-ITAM046 (“BCMA-BB046”, SEQ ID NO: 96), ITAM046 construct with “Linker 6-DAP12 ITAM-Linker 1-CD36 ITAM-Linker 7-CD3ε ITAM-Linker 2” (SEQ ID NO: 74); were chemically synthesized, then cloned into pLVX-hEF1α-Puro lentiviral vector (see Example 1) for the construction of pLVX-BCMA-BB045 and pLVX-BCMA-BB046 ransfer plasmids, respectively. These transfer plasmids were then purified and packaged into lentiviruses as described in Example 1, hereinafter referred to as BCMA-BB045 lentivirus and BCMA-BB046 lentivirus, respectively.

PBMCs and T lymphocytes were prepared according to the method described in Example 2. 3 days post activation, 5×10⁶ activated T lymphocytes were separately transduced with lentiviruses BCMA-BB, BCMA-BBz, BCMA-BB010, BCMA-BB030, BCMA-BB032, BCMA-BB035, BCMA-BB036, BCMA-BB045 and BCMA-BB046. T cell suspension was added into 6-well plate, and incubated overnight in a 37° C., 5% CO₂ incubator. 3 days post-transduction, 5×10⁵ cells were collected and centrifuged at room temperature, the supernatant was discarded. Cells were resuspended with 1 mL DPBS, 1 μL FITC-Labeled Human BCMA protein (Biolegend, #310906) was added and the suspension was incubated for 30 min at 4° C. After incubation, the centrifugation and resuspension with DPBS step was repeated twice. Then cells were resuspended with DPBS for FACS to detect BCMA CAR expression. Untransduced T cells (UnT) served as control. Then CAR positive rate in each group was adjusted to be consistent. Modified T cells were separately mixed with multiple myeloma (MM) cell line RPMI8226 at E:T ratio of 1:1 (denoted as day 0), and incubated overnight in a 37° C., 5% CO₂ incubator. On day 3, day 5, and day 7, supplemented RPMI8226 cell lines at E:T ratio of 1:1 after cell counting.

Post target tumor cells re-challenge, ITAM-modified BCMA CAR-T cells described above (including BCMA-BBz, BCMA-BB030, BCMA-BB032, BCMA-BB035, BCMA-BB036, BMCA-BB045, and BCMA-BB046) were counted on day 0, day 3, day 7, day 9, and day 11, respectively, T cell proliferation curve was made. Untransduced T cells (UnT) served as control.

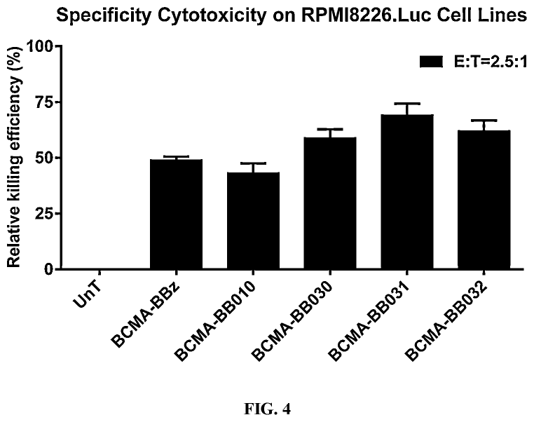

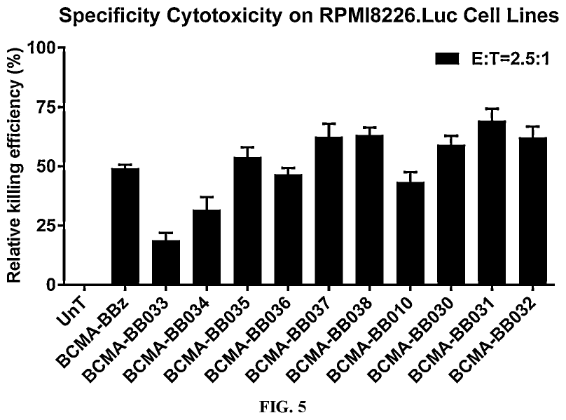

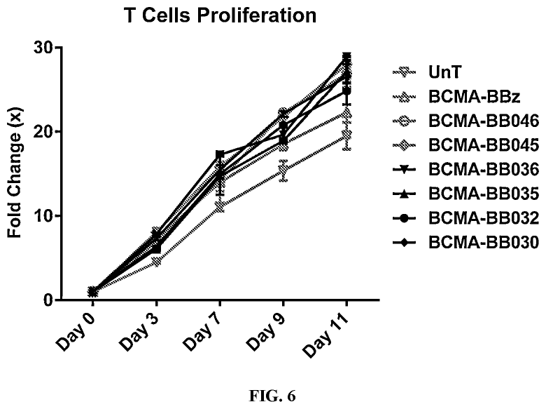

As shown in FIG. 6, ITAM-modified BCMA CAR-T cells (BCMA-BB030, BCMA-BB032, BCMA-BB035, BCMA-BB036, BCMA-BB045, and BCMA-BB046) exhibited typically CAR dependent cell proliferation post target tumor cells re-challenge. No significant difference in cell proliferation (P>0.05) was observed among ITAM-modified BCMA CARs (BCMA-BB030, BCMA-BB032, BCMA-BB035, BCMA-BB036, BCMA-BB045, and BCMA-BB046) and BCMA CAR with traditional CD3ζ intracellular signaling domain (BCMA-BBz). These data suggest that CMSD ITAMs containing modified T cells provide a higher in vitro cell proliferation activity.

Post target tumor cells re-challenge, 5×10⁵ ITAM-modified BCMA CAR-T cells described above (including BCMA-BB, BCMA-BBz, BCMA-BB035, BCMA-BB036, BCMA-BB045, BCMA-BB046, BCMA-BB010, BCMA-BB030, and BCMA-BB032) were collected and centrifuged at room temperature, the supernatant was discarded. Cells were resuspended with 1 mL DPBS, 1 μL FITC-Labeled Human BCMA protein (Biolegend, #310906), 1 μL APC anti-human CD279 (PD-1) antibody (Biolegend, #621610), and 1 μL APC anti-human D223 (LAG-3) antibody (Biolegend, #369212) were added and the suspension was incubated for 30 min at 4° C. After incubation, the centrifugation and resuspension with DPBS step was repeated twice. Then cells were resuspended with DPBS for FACS detection, in order to analyze CAR positive/PD-1 positive (CAR+/PD-1+) and CAR positive/LAG-3 positive (CAR+/LAG-3+) cell ratio, respectively.

Post target tumor cells re-challenge, 5×10⁵ ITAM-modified BCMA CAR-T cells described above (including BCMA-BB, BCMA-BBz, BCMA-BB035, BCMA-BB036, BCMA-BB045, BCMA-BB046, BCMA-BB010, BCMA-BB030, and BCMA-BB032) were collected and centrifuged at room temperature, the supernatant was discarded. Cells were resuspended with 1 mL DPBS, 1 μL FITC-Labeled Human BCMA protein (Biolegend, #310906), 1 μL PE/Cy7 anti-human CD4 antibody (Biolegend, #357409), 1 μL PerCP/Cy5.5 anti-human CD8 antibody (Biolegend, #344709), 1 μL PE anti-human CD197 (CCR7) antibody (Biolegend, #353204), and 1 μL APC anti-human CD45RA antibody (Biolegend, #304150) were added and the suspension was incubated for 30 min at 4° C. After incubation, the centrifugation and resuspension with DPBS step was repeated twice. Then cells were resuspended with DPBS for FACS detection, in order to analyze TEMRA cells (terminally differentiated effector T cells, CD45RA positive/CCR7 negative (CD45RA+/CCR7−)) ratio, TEM cells (effector memory T cells, CD45RA negative/CCR7 negative (CD45RA−/CCR7−)) ratio, TC_{M} cells (central memory T cells, CD45RA negative/CCR7 positive (CD45RA−/CCR7+)) ratio, and Naive cells (naive T cells, CD45RA positive/CCR7 positive (CD45RA+/CCR7+)) ratio in CAR positive T cells.

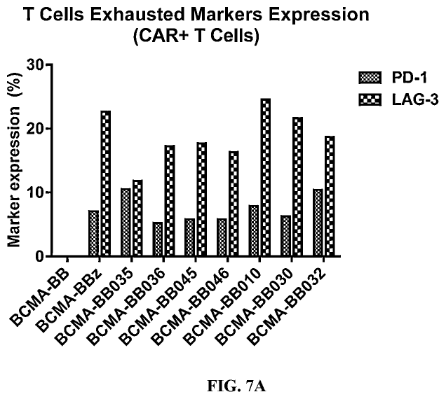

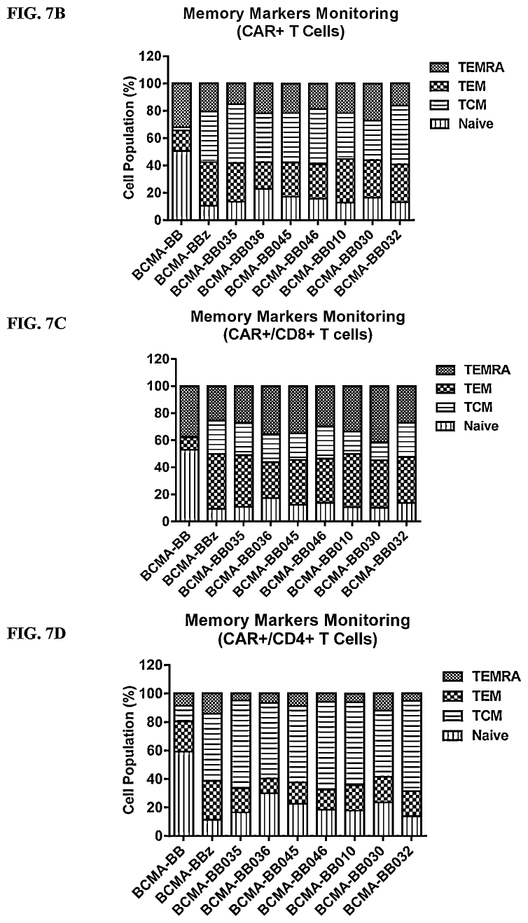

As shown in FIG. 7A, among CAR positive BCMA-BBz T cells, BCMA-BB035 T cells, BCMA-BB036 T cells, BCMA-BB045 T cells, BCMA-BB046 T cells, BCMA-BB010 T cells, BCMA-BB030 T cells, and BCMA-BB032 T cells, PD-1 expression of T cells exhausted marker was 7.07%, 10.50%, 5.24%, 5.81%, 5.80%, 7.88%, 6.26%, and 10.42%, respectively; LAG-3 expression of T cells exhausted marker was 22.64%, 11.81%, 17.20%, 17.66%, 16.29%, 24.54%, 21.61%, and 18.68%, respectively. Further study indicated (Table 6 and Table 7) difference in expression profiles of TEMRA cells, T_{EM} cells, TC_{M} cells and Naive cells was observed in each CAR+ T cells group (FIG. 7B) and CAR+/CD8+ T cells group (FIG. 7C), and significant difference (P<0.05) in CAR+/CD4+ T cells group (FIG. 7D). These results suggest CMSD ITAMs have significant influence in CAR-T cells phenotype, and provide a promising strategy for cell/gene therapy.

TABLE 6
T cells phenotype monitoring containing CMSD ITAMs

| | TEMRA cells | | | TEM cells | | |
|---|---|---|---|---|---|---|
| | (CD45RA+/CCR7−) | | | (CD45RA−/CCR7−) | | |
| | | CAR+/ | CAR+/ | | CAR+/ | CAR+/ |
| CAR construct | CAR+ | CD8+ | CD4+ | CAR+ | CD8+ | CD4+ |
| BCMA-BB | 32.02% | 37.26% | 8.50% | 15.31% | 9.17% | 21.19% |
| BCMA-BBz | 20.48% | 25.44% | 14.29% | 31.86% | 40.61% | 27.22% |
| BCMA-BB035 | 15.13% | 27.15% | 4.90% | 28.28% | 38.07% | 16.86% |
| BCMA-BB036 | 21.67% | 35.69% | 6.43% | 19.58% | 26.47% | 10.32% |
| BCMA-BB045 | 21.53% | 34.84% | 8.62% | 24.94% | 33.01% | 14.98% |
| BCMA-BB046 | 18.63% | 29.79% | 5.84% | 25.14% | 32.59% | 14.18% |
| BCMA-BB010 | 21.59% | 33.51% | 5.98% | 31.85% | 39.44% | 18.01% |
| BCMA-BB030 | 27.13% | 41.76% | 11.87% | 27.63% | 35.00% | 17.97% |
| BCMA-BB032 | 15.95% | 26.99% | 5.04% | 27.65% | 33.92% | 17.72% |

TABLE 7
T cells phenotype monitoring containing CMSD ITAMs

| | TCM cells | | | Naive cells | | |
|---|---|---|---|---|---|---|
| | (CD45RA−/CCR7+) | | | (CD45RA+/CCRR7+) | | |
| | | CAR+/ | CAR+/ | | CAR+/ | CAR+/ |
| CAR construct | CAR+ | CD8+ | CD4+ | CAR+ | CD8+ | CD4+ |
| BCMA-BB | 2.16% | 0.48% | 11.01% | 50.51% | 53.09% | 59.30% |
| BCMA-BBz | 36.97% | 24.57% | 47.20% | 10.69% | 9.37% | 11.29% |
| BCMA-BB035 | 43.04% | 23.90% | 61.65% | 13.55% | 10.89% | 16.59% |
| BCMA-BB036 | 35.85% | 20.52% | 53.33% | 22.90% | 17.33% | 29.92% |
| BCMA-BB045 | 36.40% | 19.70% | 53.73% | 17.12% | 12.45% | 22.68% |
| BCMA-BB046 | 40.52% | 23.73% | 61.62% | 15.71% | 13.89% | 18.36% |
| BCMA-BB010 | 33.57% | 16.51% | 58.29% | 12.99% | 10.54% | 17.71% |
| BCMA-BB030 | 28.82% | 12.99% | 46.50% | 16.42% | 10.25% | 23.66% |
| BCMA-BB032 | 43.30% | 25.43% | 63.61% | 13.11% | 13.65% | 13.64% |

### Example 7. In Vitro Analysis of CD20 CAR-T and ITAM-Modified CD20 CAR-T Cytotoxicity and Cytokine Release Induction

### 1. Cytotoxicity In Vitro Assay

Anti-CD20 scFv (Leu16) is a mouse antibody. Fusion gene sequences CD8α SP-CD20 scFv (Leu16)-CD8α hinge-CD8α TM-4-1BB-CD3 (hereinafter referred to as “LCAR-L186S”, SEQ ID NO: 97), and SIV Nef M116-IRES-CD8α SP-CD20 scFv (Leu16)-CD8α hinge-CD8α TM-4-1BB-ITAM010 (hereinafter referred to as “LCAR-UL186S”, SEQ ID NO: 98), were chemically synthesized, then cloned into pLVX-hEF1α-Puro lentiviral vector (see Example 1) for the construction of LCAR-L186S and LCAR-UL186S lentiviral transfer plasmids, respectively. Lentiviral transfer plasmids were purified, then mixed with lentivirus packaging plasmid mixture containing psPAX2 (packaging; Addgene, #12260) and pMD2.G (envelope; Addgene, #12259), incubated at room temperature, then transduced into HEK 293T cells, respectively. 60 hours post-transduction, supernatant containing lentiviruses was collected by centrifugating the cell transduction mixture at 4° C., 3000 rpm for 5 min. The supernatant was filtered using 0.45 m filter, and further concentrated using 500 KD hollow fiber membrane tangential flow filtration to obtain concentrated lentiviruses, which were then stored at −80° C.

PBMCs and T lymphocytes were prepared according to the method described in Example 2. 5×10⁶ activated T lymphocytes were transduced with lentiviruses encoding LCAR-L186S (referred to as “LCAR-L186S T cell”) and LCAR-UL186S (referred to as “LCAR-UL186S T cell”), respectively, and incubated overnight in a 37° C., 5% CO₂ incubator. 3 days post-transduction, 5×10⁵ cell suspension was collected and centrifuged at room temperature, supernatant was discarded. Cells were resuspended with 1 mL DPBS and 1 μL Goat F(ab′)2 anti-Mouse IgG (Fab′)2 (FITC) (Abcam, #AB98658) was added into the suspension, then incubated for at 4° C. for 30 min. After incubation, the centrifugation and resuspension with DPBS step was repeated twice. Then cells were resuspended with DPBS and supplemented with 1 μL Streptavidin (NEW ENGLAND BIOLABS, #N7021S), then incubated at 4° C. for 30 min. After incubation, the centrifugation and resuspension with DPBS step was repeated twice. Then cells were resuspended with DPBS and subject to FACS for CD20 CAR expression detection.

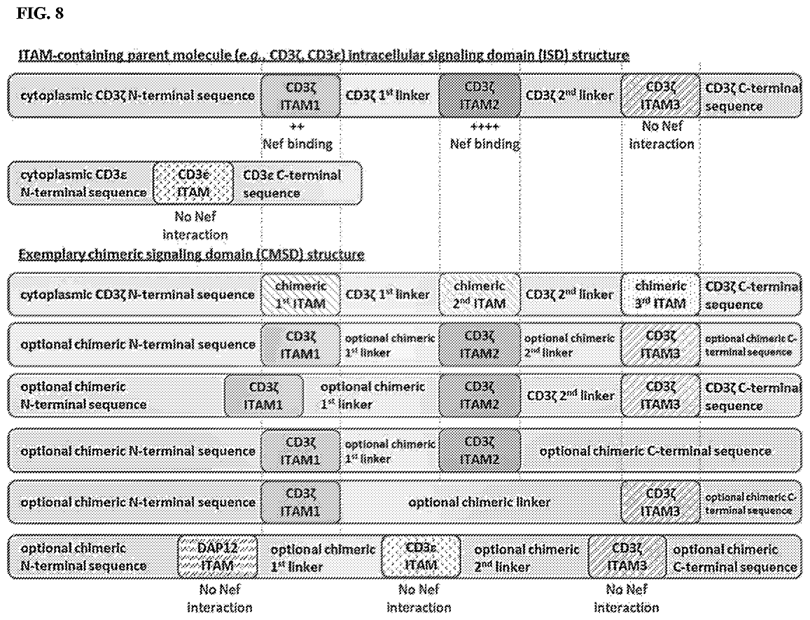

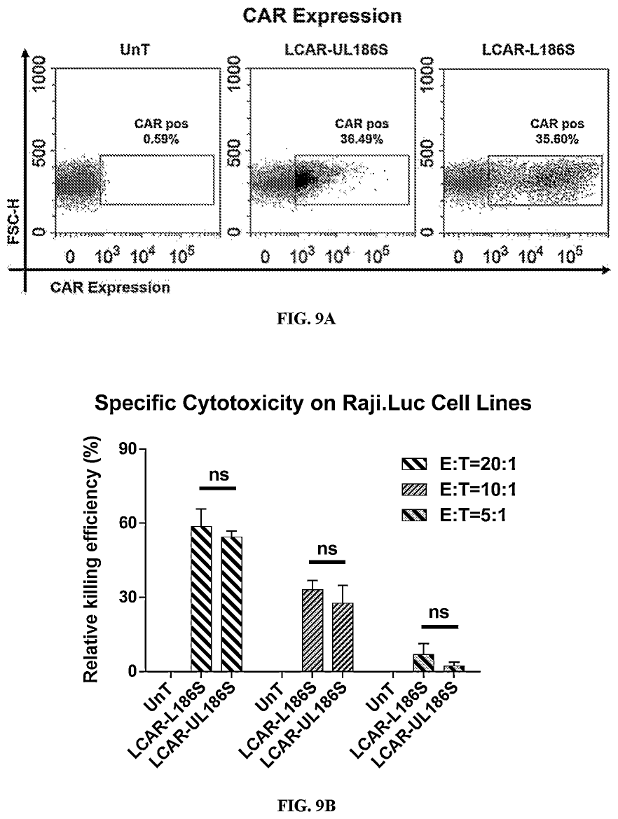

As shown in FIG. 9A, primary T lymphocytes transduced with LCAR-L186S lentiviruses and LCAR-UL186S lentiviruses show 35.60% and 36.49% CAR positive rates, respectively. Untreated T lymphocytes served as negative control (0.59% CAR pos). This result demonstrates that SIV Nef M116 co-expression does not affect the expression of ITAM-modified CD20 CAR comprising an ITAM110 chimeric signaling domain (LCAR-UL186S); the CAR expression level is similar to that of a CD20 CAR with traditional CD3ζ intracellular signaling domain (LCAR-L186S).

LCAR-L186S T cells and LCAR-UL186S T cells were mixed with lymphoma Raji.Luc cell line (CD20 positive, with luciferase marker) at different effector to target cell (E:T) ratios of 20:1, 10:1 and 5:1, respectively. Untreated T cells served as control (“UnT”). The mixed cells were incubated in 384-well plates for 12-24 hours. Cytotoxicity of different T lymphocytes on target cells were detected according to similar method described in Example 2.

As shown in FIG. 9B, primary T lymphocytes transduced with LCAR-L186S lentiviruses and LCAR-UL186S lentiviruses both exhibit strong cytotoxicity on Raji.Luc cell line, and is E:T concentration dependent. There is no significant cytotoxicity difference between LCAR-L186S T cells and LCAR-UL186S T cells at all E:T ratios, while both LCAR-L186S T cells and LCAR-UL186S T cells exhibit much stronger cytotoxicity on day 3 of the cell killing assay compared to untransduced T cells (“UnT”, P<0.05). This result demonstrates that SIV Nef M116 co-expression does not affect the cytotoxicity of ITAM-modified CD20 CAR comprising an ITAM010 chimeric signaling domain (LCAR-UL186S); and ITAM-modified CD20 CAR shows similar cytotoxicity as CD20 CAR with traditional CD3ζ intracellular signaling domain (LCAR-L186S).

### 2. Cytokine Release In Vitro Assay

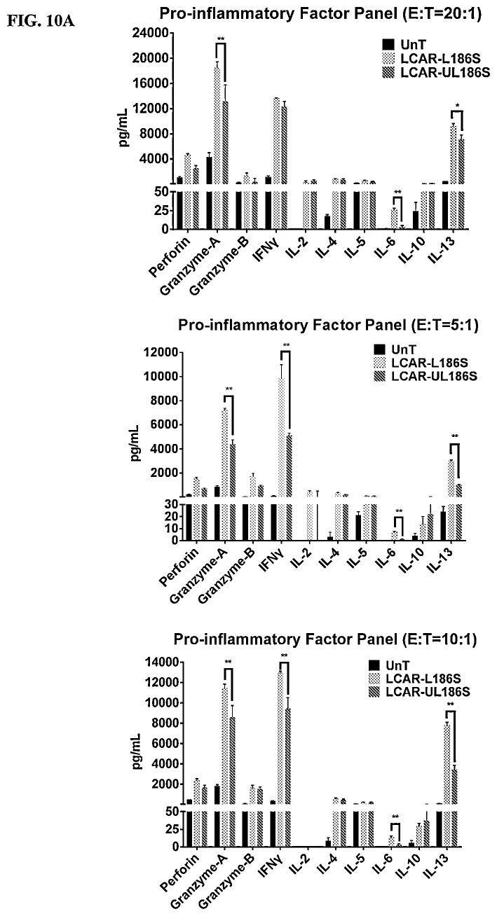

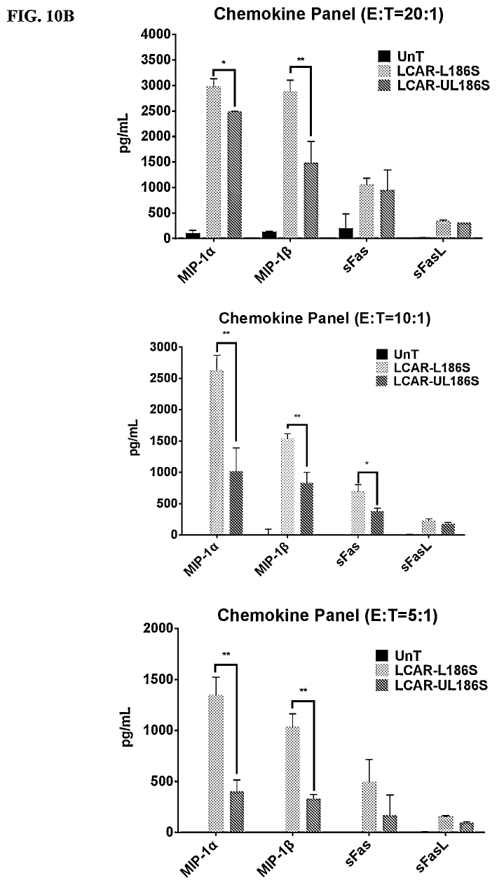

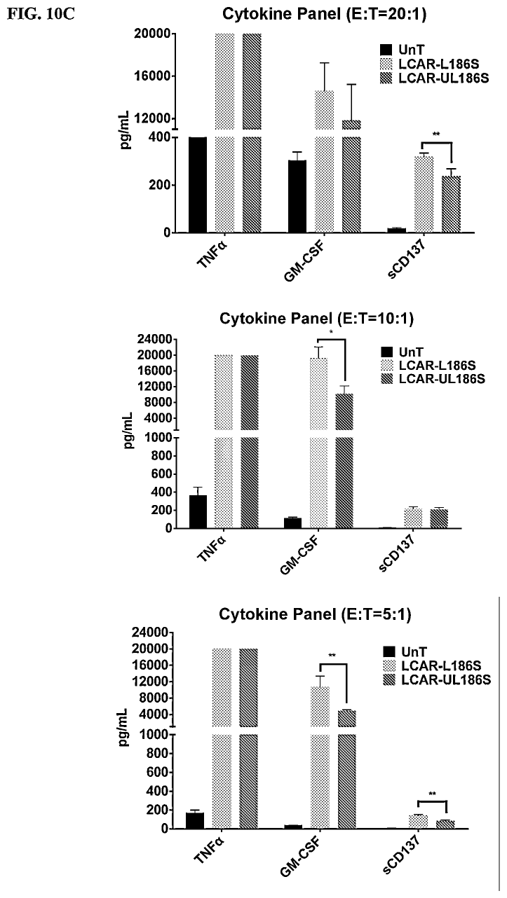

LCAR-L186S T cells and LCAR-UL186S T cells were incubated with lymphoma Raji.Luc cell line at different E:T ratios described above, respectively. Supernatants from the co-culture assays were collected to assess CAR-induced cytokine release of 17 cytokine molecules, including pro-inflammatory factors (FIG. 10A), chemokines (FIG. 10B), and cytokines (FIG. 10C). Untransduced T (“UnT”) cells served as control.

As shown in FIG. 10A, after LCAR-L186S T cells or LCAR-UL186S T cells were co-cultured with CD20 positive Raji.Luc cells at different E:T ratios for 20-24 hours, the secretion of pro-inflammatory factors (such as Perform, Granzyme A, Granzyme B, IFNγ, IL-4, IL-5, IL-6, IL-10 and IL-13) was significantly increased compared with UnT cells (P<0.05), and the levels of secretion was E:T ratio dependent, suggesting that both LCAR-L186S T cells and LCAR-UL186S T cells can initiate strong Raji-targeted cytotoxic effects. Among these pro-inflammatory factors, Granzyme A, IFNγ, IL-6, and IL-13 showed significantly higher secretion in LCAR-L186S T cells than in LCAR-UL186S T cells (P<0.05), suggesting that ITAM-modified CD20 CAR/SIV Nef M116 co-expression may induce less pro-inflammatory factor release and lower risk of cytokine release syndrome (CRS).

As shown in FIG. 10B, after LCAR-L186S T cells or LCAR-UL186S T cells were co-cultured with CD20 positive Raji.Luc cells at different E:T ratios for 20-24 hours, the secretion of chemokines (such as MIP-1α, MIP-1β, sFas and sFasL) was significantly increased compared with UnT cells (P<0.05), and the levels of secretion was E:T ratio dependent, suggesting that both LCAR-L186S T cells and LCAR-UL186S T cells can initiate strong Raji-targeted cytotoxic effects. Among these chemokines, MIP-1α and MIP-1β (and in some cases sFas as well) showed significantly higher secretion in LCAR-L186S T cells than in LCAR-UL186S T cells (P<0.05), suggesting that ITAM-modified CD20 CAR/SIV Nef M116 co-expression may induce less chemokine release and lower risk of CRS.

As shown in FIG. 10C, after LCAR-L186S T cells or LCAR-UL186S T cells were co-cultured with CD20 positive Raji.Luc cells at different E:T ratios for 20-24 hours, the secretion of cytokines (such as TNFα, GM-CSF and sCD137) was significantly increased compared with UnT cells (P<0.05), suggesting that both LCAR-L186S T cells and LCAR-UL186S T cells can initiate strong Raji-targeted cytotoxic effects. Among these cytokines, TNFα secretion reached the detection limit; GM-CSF and sCD137 secretion was significantly higher in LCAR-L186S T cells than in LCAR-UL186S T cells (P<0.05), suggesting that ITAM-modified CD20 CAR/SIV Nef M126 co-expression may induce less cytokine release and lower risk of CRS.

In summary, the above results indicate that there is no significant difference in cytotoxicity on target cells between LCAR-L186S T cells and LCAR-UL186S T cells, while pro-inflammatory factor, chemokine and cytokine release induced by LCAR-UL186S T cells are significant lower than LCAR-L186S T cells, suggesting that ITAM-modified CD20 CAR/SIV Nef M116 co-expression construct is effective and safer with lower cytokine release, demonstrating more extensive clinical application prospect.

### Example 8. In Vivo Efficacy Evaluation of LCAR-L186S T Cells and LCAR-UL186S CAR+/TCRαβ− T Cells

### 1. Lymphoma Xenograft Mouse Model Establishment and Survival Index Monitoring

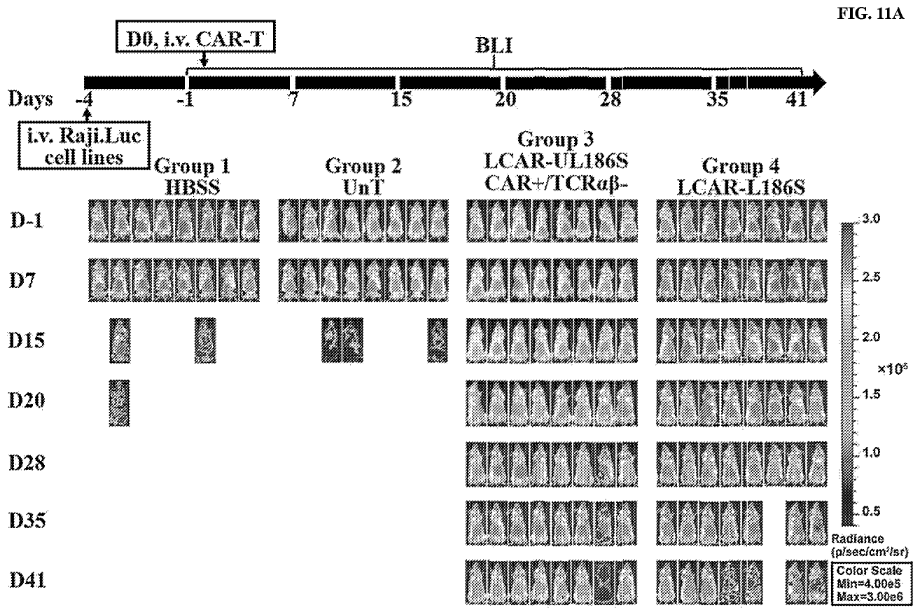

The in vivo cytotoxicity of CD20 CAR-T cells or ITAM-modified CD20 CAR-T cells on tumor cells were investigated using severe immune-deficient mouse model. LCAR-UL186S T cells from Example 3 were MACS enriched for TCRαβ− cells, resulting in TCRαβ− MACS sorted “LCAR-UL186S CAR+/TCRαβ− T cells.” LCAR-L186S T cells (not MACS enriched, from Example 3) and TCRαβ− MACS sorted LCAR-UL186S CAR+/TCRαβ− T cells were used in this Example. Immune-deficient NCG mice were engrafted with CD20+ tumor cells (3×10⁴ human Raji.Luc cells per mouse) on day −4 via tail vein, then each mouse received a single injection of 2×10⁶ LCAR-L186S T cells (Group 4 mice, 8 mice) or LCAR-UL186S CAR+/TCRαβ− T cells (Group 3 mice, 8 mice) on day 0. Group 1 mice (8 mice) received HBSS injection, Group 2 mice (8 mice) received untransduced T cells (UnT) injection, serving as negative controls. Mice were monitored every day, and assessed by bioluminescence imaging on a weekly basis to monitor tumor growth and body weight. See FIG. 11A. Mouse survival was monitored and recorded by Kaplan-Meier survival plots.

### 2. In Vivo Efficacy of LCAR-L186S T Cells and LCAR-UL186S CAR+/TCRαβ− T Cells

As shown in FIGS. 11A-11D, after Raji.Luc (CD20+) cell engrafting, vehicle (HBSS, Hank's Balanced Salt Solution; Group 1) or untransduced T cell treatment (Group 2) did not inhibit the growth of tumor cells. Mice in these 2 groups were euthanized starting from day 15 of receiving treatment, because of tumor burden, sputum, weight loss (FIG. 11C), body chills and other symptoms. Compared to these control mice, no bioluminescence was observed in mice treated with LCAR-L186S T cells or LCAR-UL186S CAR+/TCRαβ− T cells within 20 days since treatment. These results indicate that LCAR-L186S T cells and LCAR-UL186S CAR+/TCRαβ− T cells can effectively inhibit the growth of B cell lymphoma in vivo.

28 days post CAR-T cell injection, some mice in Group 3 (LCAR-UL186S CAR+/TCRαβ−) and Group 4 (LCAR-L186S) showed tumor recurrence (FIGS. 11A-111B). ⅛ mouse in Group 4 was euthanized on day 31 due to relapsed tumor (FIGS. 11A and 11D). The bioluminescence imaging on day 41 showed that ⅛ mouse in Group 3 and 4/7 mice (one euthanized on day 31) in Group 4 had tumor recurrence with high number of photons (FIGS. 11A-11B). These mice were euthanized due to paralysis and weight loss. The survival curve reflects the overall activity of CAR-T cells. As shown in FIG. 11D, both LCAR-UL186S CAR+/TCRαβ− T cells and LCAR-L186S T cells can significantly prolong the survival of tumor-grafted mice, demonstrating superior in vivo anti-tumor efficacy, with little or no effect on weight loss (FIG. 11C). Further, LCAR-UL186S CAR+/TCRαβ− T cells (ITAM-modified CAR/SIV Nef M116 co-expression) seem to exhibit better treatment efficacy and survival rate compared to LCAR-L186S T cells (CAR with traditional CD3ζ intracellular signaling domain).

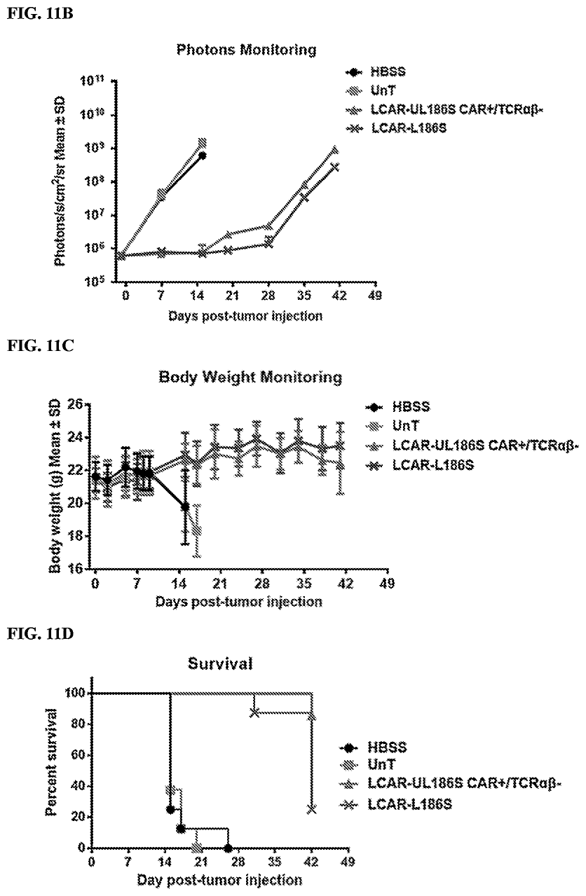

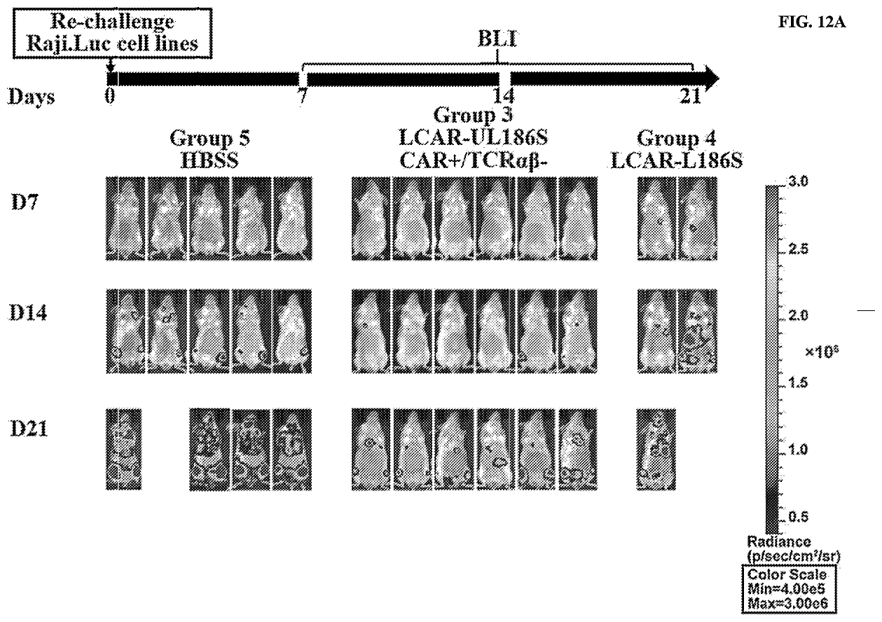

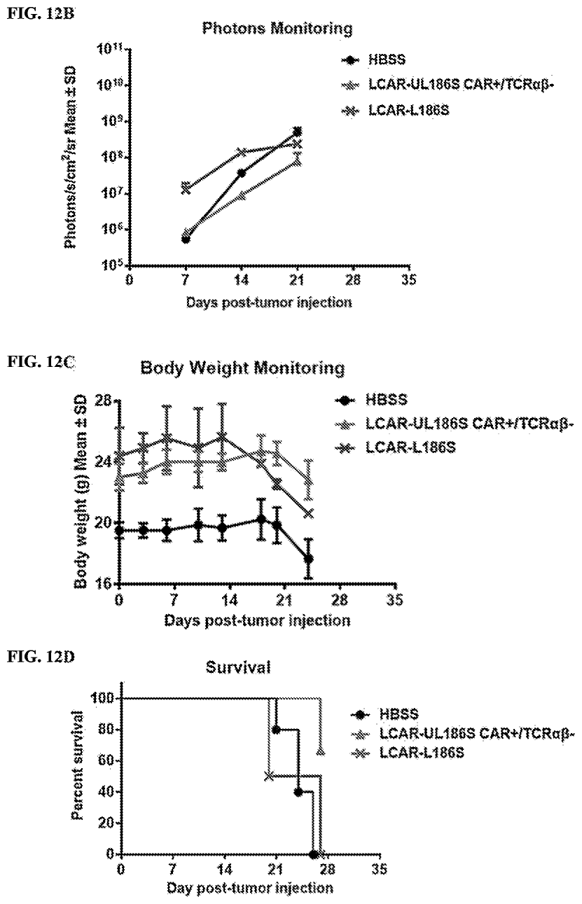

In order to further study the long-term anti-tumor activity of LCAR-L186S T cells and LCAR-UL186S CAR+/TCRαβ− T cells, mice that did not relapse after 41 days of CAR-T administration (Group 3 LCAR-UL186S-treated 6 mice, Group 4 LCAR-L186S-treated 2 mice) were subsequently re-challenged with 3×104 Raji.Luc cells (denoted as day 0; FIG. 12A). 5 healthy immune-deficient NCG mice were engrafted with 3×10⁴ Raji.Luc cells and injected with HBSS on day 0 (Group 5), as control. The condition of tumor cell transplanted mice was monitored and recorded weekly (see FIGS. 12A-12C). On day 14 after re-challenge, all Group 4 mice (2/2) that received LCAR-L186S T cell treatment had tumor recurrence (FIG. 12A) and the number of Raji.Luc photons increased (FIG. 12B). One mouse (½) in Group 4 was euthanized due to paralysis and weight loss on day 20 (FIGS. 12A, 12B, and 12D), all mice died on day 27 (FIG. 12D). On day 14 after re-challenge, only 3/6 of Group 3 mice that received LCAR-UL186S CAR+/TCRαβ− T cell treatment had increased Raji.Luc light intensity (FIG. 12B) and tumor burden expansion (FIG. 12A). Although the tumor burden in Group 3 increased on day 21 (FIGS. 12A-12B), no death occurred due to paralysis or weight loss (FIGS. 12C-12D). Even on day 27, Group 3 mice still had 67% survival rate (FIG. 12D). For the control Group 5 mice received HBSS, 14 days post tumor re-challenge, tumor burden began to increase gradually (FIGS. 12A-12B), and 5 mice were euthanized due to paralysis and weight loss on days 21-26 (FIGS. 12A-12D).

These results suggest that both LCAR-UL186S CAR+/TCRαβ− T cells and LCAR-L186S T cells can effectively inhibit the growth of B lymphoma cells in vivo. Further, LCAR-UL186S CAR+/TCRαβ− T cells can prolong mouse survival in both tumor models and tumor recurrence models, with little or no effect on weight loss (FIGS. 11C and 12C), and demonstrate stronger in vivo efficacy and persistence than LCAR-L186S T cells. These suggest that LCAR-UL186S CAR+/TCRαβ− T cells (ITAM-modified CAR/SIV Nef M116 co-expression) may provide a more promising treatment regime compared to CARs with traditional CD3ζ intracellular signaling domain.

### Example 9. Specific Cytotoxicity of LIC948A22 CAR-T Cells and LUC948A22 UCAR-T Cells on Multiple Myeloma (MM) Cell Lines

Fusion gene sequences CD8α SP-BCMA V_{H}H1-Linker-BCMA V_{H}H2-CD8α hinge-CD8α TM-4-1BB-CD3ζ (“LIC948A22 CAR”, SEQ ID NO: 10⁵ for the CAR construct), and SIV Nef M116-IRES-CD8α SP-BCMA V_{H}H1-Linker-BCMA V_{H}H2-CD8α hinge-CD8α TM-4-1BB-ITAM010 (“LUC948A22 UCAR”, SEQ ID NO: 106 for the CAR construct) were chemically synthesized, and cloned into pLVX-hEF1α-Puro lentiviral vector for the construction of recombinant transfer plasmids, respectively. All lentiviral transfer plasmids were purified, and packaged into lentiviruses.

Peripheral blood mononuclear cells (PBMCs) were purchased from TPCS®. Pan T Cell Isolation Kit (Miltenyi Biotec, #130-096-535) was used to magnetically label thawed PBMCs and isolate and purify T lymphocytes. CD3/CD28 conjugated magnetic beads were used for activation and expansion of purified T lymphocytes. Activated T lymphocytes were incubated in 37° C., 5% CO₂ incubator for 24 hours. Then transduced T lymphocytes with lentiviruses encoding LIC948A22 CAR and LUC948A22 UCAR, respectively. 12 days post transduction, cells were collected and subject to magnetic-activated cell sorting (MACS). LIC948A22 CAR-T cells were produced after BCMA+ MACS enrichment, and LUC948A22 UCAR-T cells were produced after TCRαβ− MACS enrichment. Each 5×10⁵ MACS sorted cell suspension was collected and centrifuged at room temperature, supernatant was discarded. Cells were resuspended with DPBS and 1 μL FITC-Labeled Human BCMA protein (Biolegend, #310906) and 1 μL APC anti-human TCRαβ antibody (Biolegend, #B259839) were added into the suspension, then incubated at 4° C. for 30 min. After incubation, the centrifugation and resuspension with 1 mL DPBS step was repeated twice. Then cells were resuspended with DPBS and subject to fluorescence-activated cell sorting (FACS) for positive rates detection of CAR and TCRαβ.

LIC948A22 CAR-T cells, TCRαβ MACS sorted LUC948A22 UCAR-T cells (CAR+/TCRαβ−) or untreated T cells (UnT) obtained from the above steps were mixed under 2.5:1 or 1.25:1 effector to target cell ratios (E:T) with multiple myeloma (MM) cell lines RPMI8226.Luc (with Luciferase (Luc) marker, BCMA+) respectively, and incubated in Corning® 384-well solid white plate for 18-20 hours. ONE-Glo™ Luciferase Assay System (TAKARA, #B6120) was used to measure luciferase activity. 25 μL ONE-Glo™ Reagent was added to each well of the 384-well plate. After incubation, fluorescence was measured using Spark™ 10M multimode microplate reader (TECAN), in order to calculate cytolytic effects of different T lymphocytes on target cells.

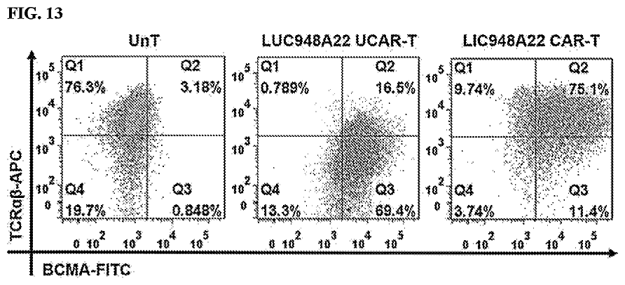

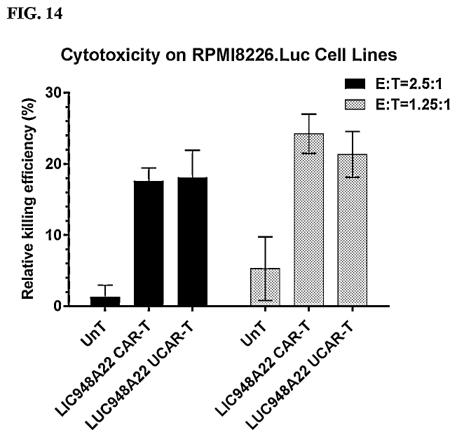

As shown in FIG. 13, BCMA CAR positive rate of LIC948A22 CAR-T cells and LUC948A22 UCAR-T cells (CAR+/TCRαβ−) were 86.5% and 85.9%, respectively. Specific killing activity of LIC948A22 CAR-T cells and LUC948A22 UCAR-T cells (CAR+/TCRαβ−) on RPMI8226.Luc cell lines were further evaluated, respectively. As shown in FIG. 14, LIC948A22 CAR-T cells and LUC948A22 UCAR-T cells (CAR+/TCRαβ−) can both effectively mediated CAR-specific tumor cell killing on RPMI8226.Luc cell lines with relative killing efficiency of above 15%, and no significant cytotoxicity difference was observed between them.

### Example 10. In Vitro Analysis of LIC948A22 CAR-T Cells and LUC948A22 UCAR-T Cells Cytokine Release

LIC948A22 CAR-T cells and LUC948A22 UCAR-T cells (CAR+/TCRαβ−) were incubated with multiple myeloma cell lines RPMI8226.Luc at different E:T ratios (2.5:1 and 1.25:1) for 18-20 hours, respectively. Supernatants from the co-culture assays were collected to assess CAR-induced cytokine release of 17 cytokine molecules using MILLIPORE MILLIPLEX® MAP Human CD8+ T-Cell Magnetic Bead Panel according to the manufacturer's instructions, including pro-inflammatory factors (FIG. 15A), chemokines (FIG. 15B), and cytokines (FIG. 15C). Untreated T cells (UnT) served as control.

As shown in FIG. 15A, after LIC948A22 CAR-T cells or LUC948A22 UCAR-T cells (CAR+/TCRαβ−) were co-cultured with RPMI8226.Luc cell lines at different E:T ratios, the secretion of pro-inflammatory factors (such as Perform, Granzyme A, Granzyme B, IFNγ, IL-2, IL-4, IL-5, IL-10, and IL-13) was significantly increased compared with UnT group (P<0.05). LUC948A22 UCAR-T cells secret higher IL-2 than LIC948A22 CAR-T cells.

As shown in FIG. 15B, after LIC948A22 CAR-T cells or LUC948A22 UCAR-T cells (CAR+/TCRαβ−) were co-cultured with RPMI8226.Luc cell lines at different E:T ratios, the secretion of chemokines (such as MIP-1α, MIP-1β, sFas and sFasL) was significantly increased compared with UnT group (P<0.05). Meanwhile, LUC948A22 UCAR-T cells secret higher sFasL than LIC948A22 CAR-T cells.

As shown in FIG. 15C, after LIC948A22 CAR-T cells and LUC948A22 UCAR-T cells (CAR+/TCRαβ−) were co-cultured with RPMI8226.Luc cell lines at different E:T ratios, the secretion of cytokines (such as TNFα, GM-CSF and sCD137) was significantly increased compared with UnT group (P<0.05). Meanwhile, LUC948A22 UCAR-T cells secret higher TNFα than LIC948A22 CAR-T cells.

In summary, the above results show that LUC948A22 UCAR-T cells (CAR+/TCRαβ−) have comparable effects with autologous LIC948A22 CAR-T cells, such as cytotoxicity and cytokine release, suggesting that LUC948A22 UCAR-T cell will be effective and safe with extensive clinical application prospect.

### Example 11. Use of SIV Nef M116 in CD20 CAR-T Cell Immunotherapy

### 1. Construction of SIV Nef M116+CAR All-In-One Vectors

Fusion gene sequences in Table 8 were chemically synthesized, then cloned into pLVX-hEF1α vector (see Example 1) for the construction of recombinant transfer plasmids pLVX-M1185, pLVX-M1218, pLVX-M1219, pLVX-M1124, pLVX-M1125, pLVX-M1126, and pLVX-M1127, respectively. These transfer plasmids were then purified and packaged into lentiviruses as described in Example 1, hereinafter referred to as M1185 lentivirus, M1218 lentivirus, M1219 lentivirus, M1124 lentivirus, M1125 lentivirus, M1126 lentivirus, and M1127 lentivirus, respectively.

TABLE 8
Exemplary SIV Nef M116 + CAR all-in-one vectors

| | Fusion | | CAR amino |
|---|---|---|---|
| Vector name | gene | Fusion gene structure | acid sequence |
| pLVX-M1185 | M1185 | SIV Nef M116-IRES-CD8α SP-CD20 scFv | SEQ ID NO: 97 |
| | | (Leu16)-CD8α hinge-CD8α TM-4-1BB- | |
| | | CD3ζ | |
| pLVX-M1218 | M1218 | SIV Nef M116-IRES-CD8α SP-CD20 scFv | SEQ ID NO: 99 |
| | | (Leu16)-CD8α hinge-CD8α TM-4-1BB- | |
| | | ITAM035 | |
| pLVX-M1219 | M1219 | SIV Nef M116-IRES-CD8α SP-CD20 scFv | SEQ ID NO: 100 |
| | | (Leu16)-CD8α hinge-CD8α TM-4-1BB- | |
| | | ITAM036 | |
| pLVX-M1124 | M1124 | SIV Nef M116-IRES-CD8α SP-CD20 scFv | SEQ ID NO: 101 |
| | | (Leu16)-CD8α hinge-CD8α TM-4-1BB- | |
| | | ITAM045 | |
| pLVX-M1125 | M1125 | SIV Nef M116-IRES-CD8α SP-CD20 scFv | SEQ ID NO: 102 |
| | | (Leu16)-CD8α hinge-CD8α TM-4-1BB- | |
| | | ITAM046 | |
| pLVX-M1126 | M1126 | SIV Nef M116-IRES-CD8α SP-CD20 scFv | SEQ ID NO: 103 |
| | | (Leu16)-CD8α hinge-CD8α TM-4-1BB- | |
| | | ITAM030 | |
| pLVX-M1127 | M1127 | SIV Nef M116-IRES-CD8α SP-CD20 scFv | SEQ ID NO: 104 |
| | | (Leu16)-CD8α hinge-CD8α TM-4-1BB- | |
| | | ITAM032 | |

### 2. Evaluation of SIV Nef M116 Regulation on TCR

Lentiviruses M1185, M1218, M1219, M1124, M1125, M1126, M1127, and LCAR-UL186S from Example 3, were added into the suspension of Jurakt cell culture for transduction, respectively. 3 days post-transduction, 5×10⁵ cell suspension was collected and centrifuged at room temperature, the supernatant was discarded. Cells were resuspended with 1 mL DPBS, 1 μL PE/Cy5 anti-human TCRα/β antibody (Biolegend, #306710) was added and the suspension was incubated for 30 min at 4° C. After incubation, the centrifugation and resuspension with DPBS step was repeated twice. Then cells were resuspended with DPBS for FACS to detect TCRαβ expression. Untransduced Jurkat cells (“Jurkat”) served as control.

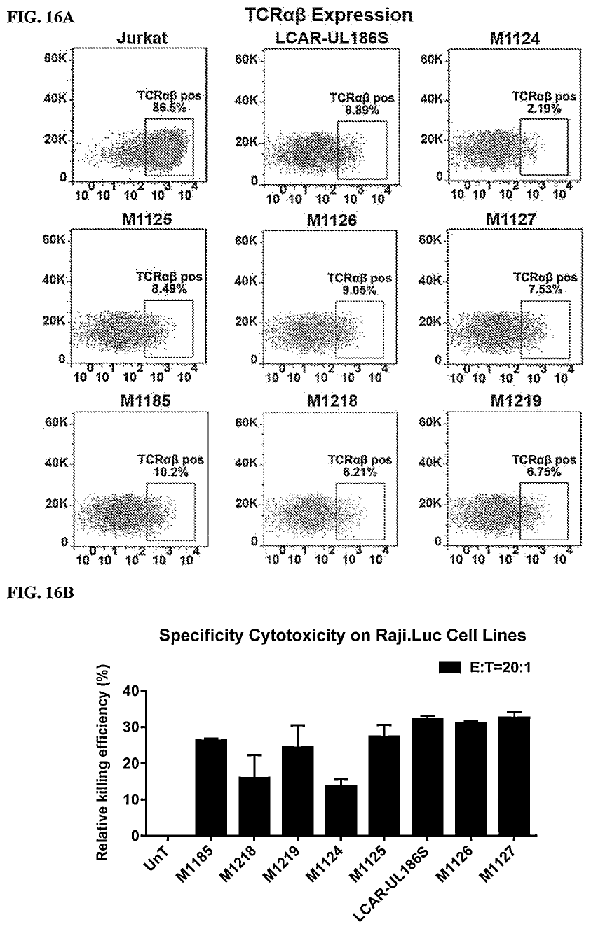

As shown in FIG. 16A, SIV Nef M116+ITAM-modified CD20 CARs all-in-one construct transduced Jurkat cells significantly down-regulated TCRα3 expression compared to untransduced Jurkat cells (P<0.05).

### 3. Cytotoxicity of CD20 CAR-T Cells In Vitro Assay

PBMCs and T lymphocytes were prepared according to the method described in Example 2. 3 days post activation, 5×10⁶ activated T lymphocytes were transduced with lentiviruses carrying all-in-one construct (including M1185, M1218, M1219, M1124, M1125, M1126, M1127, and LCAR-UL186S), respectively. T cell suspension was added into 6-well plate, and incubated overnight in a 37° C., 5% CO₂ incubator. 3 days post-transduction, modified T cells were mixed with lymphoma cell line Raji.Luc at E:T ratio of 20:1, respectively, incubated in Corning® 384-well solid white plate for 12 hours. ONE-Glo™ Luciferase Assay System (TAKARA, #B6120) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (“UnT”) served as control.

As shown in FIG. 16B, SIV Nef M116+ITAM-modified CD20 CARs all-in-one construct transduced T cells showed significant CAR-mediated specific killing activity on Raji.Luc cell lines compared to UnT (P<0.05). No significant difference in cytotoxicity (P>0.05) was observed among M1219, M1125-M1127, LCAR-UL186S, and CD20 CAR with traditional CD3ζISD (M1185).

### Example 12. Use of SIV Nef M116 in BCMA CAR-T Cell Immunotherapy

### 1. Construction of SIV Nef M116+CAR All-In-One Vectors

Fusion gene sequences in Table 9 were chemically synthesized, then cloned into pLVX-hEF1α vector (see Example 1) for the construction of recombinant transfer plasmids pLVX-M1215, pLVX-M1216, pLVX-M1217, pLVX-M985, pLVX-M986, pLVX-M989, and pLVX-M990, respectively. These transfer plasmids were then purified and packaged into lentiviruses as described in Example 1, hereinafter referred to as M1215 lentivirus, M1216 lentivirus, M1217 lentivirus, M985 lentivirus, M986 lentivirus, M989 lentivirus, and M990 lentivirus, respectively.

TABLE 9
Exemplary SIV Nef M116 + CAR all-in-one vectors

| | Fusion | | CAR amino |
|---|---|---|---|
| Vector name | gene | Fusion gene structure | acid sequence |
| pLVX-M1215 | M1215 | SIV Nef M116-IRES-CD8α SP-BCMA | SEQ ID NO: 105 |
| | | VHH1-Linker-BCMA VHH2-CD8α | |
| | | hinge-CD8α TM-4-1BB-CD3ζ | |
| pLVX-LUC948A22 | LUC94 | SIV Nef M116-IRES-CD8α SP-BCMA | SEQ ID NO: 106 |
| UCAR | 8A22 | VHH1-Linker-BCMA VHH2-CD8α | |
| | UCAR | hinge-CD8α TM-4-1BB-ITAM010 | |
| pLVX-M1216 | M1216 | SIV Nef M116-IRES-CD8α SP-BCMA | SEQ ID NO: 107 |
| | | VHH1-Linker-BCMA VHH2-CD8α | |
| | | hinge-CD8α TM-4-1BB-ITAM035 | |
| pLVX-M1217 | M1217 | SIV Nef M116-IRES-CD8α SP-BCMA | SEQ ID NO: 108 |
| | | VHH1-Linker-BCMA VHH2-CD8α | |
| | | hinge-CD8α TM-4-1BB-ITAM036 | |
| pLVX-M985 | M985 | SIV Nef M116-IRES-CD8α SP-BCMA | SEQ ID NO: 109 |
| | | VHH1-Linker-BCMA VHH2-CD8α | |
| | | hinge-CD8α TM-4-1BB-ITAM045 | |
| pLVX-M986 | M986 | SIV Nef M116-IRES-CD8α SP-BCMA | SEQ ID NO: 110 |
| | | VHH1-Linker-BCMA VHH2-CD8α | |
| | | hinge-CD8α TM-4-1BB-ITAM046 | |
| pLVX-M989 | M989 | SIV Nef M116-IRES-CD8α SP-BCMA | SEQ ID NO: 111 |
| | | VHH1-Linker-BCMA VHH2-CD8α | |
| | | hinge-CD8α TM-4-1BB-ITAM030 | |
| pLVX-M990 | M990 | SIV Nef M116-IRES-CD8α SP-BCMA | SEQ ID NO: 112 |
| | | VHH1-Linker-BCMA VHH2-CD8α | |
| | | hinge-CD8α TM-4-1BB-ITAM032 | |

### 2. Evaluation of SIV Nef M116 Regulation on TCR

Lentiviruses M1215, M1216, M1217, M985, M986, M989, M990, and LUC948A22 UCAR (see Example 7), were added into the suspension of Jurakt cell culture for transduction, respectively. 3 days post-transduction, 5×10⁵ cell suspension was collected and centrifuged at room temperature, the supernatant was discarded. Cells were resuspended with 1 mL DPBS, 1 μL PE/Cy5 anti-human TCRα/β antibody (Biolegend, #306710) was added and the suspension was incubated for 30 min at 4° C. After incubation, the centrifugation and resuspension with DPBS step was repeated twice. Then cells were resuspended with DPBS for FACS to detect TCRαβ expression. Untransduced Jurakt cells (“Jurkat”) served as control.

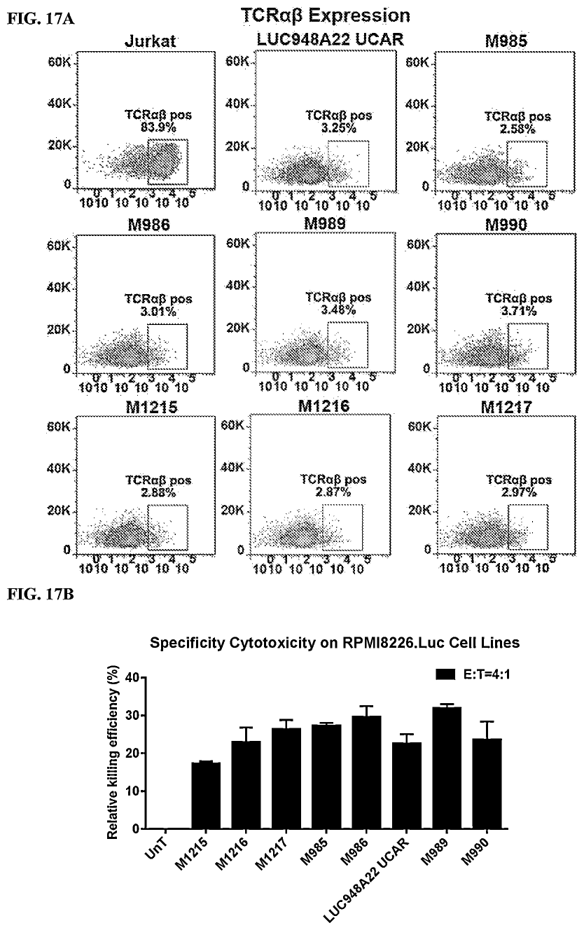

As shown in FIG. 17A, SIV Nef M116+ITAM-modified BCMA CARs all-in-one construct transduced Jurkat cells significantly down-regulated TCRα3 expression compared to untransduced Jurkat cells (P<0.05).

### 3. Cytotoxicity of BCMA CAR-T Cells In Vitro Assay

PBMCs and T lymphocytes were prepared according to the method described in Example 2. 3 days post activation, 5×10⁶ activated T lymphocytes were transduced with lentiviruses carrying all-in-one construct (including M1215, M1216, M1217, M985, M986, M989, M990, and LUC948A22 UCAR (see Example 9), respectively. T cell suspension was added into 6-well plate, and incubated overnight in a 37° C., 5% CO₂ incubator. 3 days post-transduction, modified T cells were mixed with multiple myeloma (MM) cell line RPMI8226.Luc at E:T ratio of 4:1, respectively, incubated in Corning® 384-well solid white plate for 12 hours. ONE-Glo™ Luciferase Assay System (TAKARA, #B6120) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (“UnT”) served as control.

As shown in FIG. 17B, SIV Nef M116+ITAM-modified BCMA CARs all-in-one construct transduced T cells show significant CAR-mediated specific killing activity on RPMI8226.Luc cell lines compared to UnT (P<0.05). M1217, M985, M986, and M989 showed significantly CAR-specific cytotoxicity compared to BCMA-BBz (P<0.05). No significant difference in cytotoxicity (P>0.05) was observed among M1216, LUC948A22 UCAR, M990, and BCMA CAR with traditional CD3ζISD (M1215).

### Example 13. Use of SIV Nef M708 in BCMA CAR-T Cell Immunotherapy

### 1. Construction of SIV Nef M708+ITAM-Modified CAR All-In-One Vector

Fusion gene sequences SIV Nef M708-IRES-CD8α SP-BCMA VHH1-linker-BCMA V_{H}H2-CD8α hinge-CD8α TM-4-1BB-ITAM010 (hereinafter referred to as M598, SIV Nef M708 comprises the sequence of SEQ ID NO: 122) were chemically synthesized, then cloned into pLVX-hEF1α vector (see Example 1) for the construction of recombinant transfer plasmids pLVX-M598. Then transfer plasmids were purified and packaged into lentiviruses as described in Example 1, hereinafter referred to as M598 lentivirus. The ITAM-modified BCMA CAR construct “CD8α SP-BCMA VHH1-linker-BCMA V_{H}H2-CD8α hinge-CD8α TM-4-1BB-ITAM010” is herein referred to as “M598 ITAM010-modified BCMA CAR” or “M598 BCMA CAR,” comprising the sequence of SEQ ID NO: 113. Anti-BCMA VHH1 and VHH2 of the M598 BCMA CAR, as well as CDRs contained therein, have been disclosed in PCT/CN2016/094408 and PCT/CN2017/096938, the contents of each of which are incorporated herein by reference in their entirety.

### 2. In Vitro TCRαβ Regulation and Cytotoxicity Analysis of SIV Nef M708+CAR All-In-One Vector

PBMCs and T lymphocytes were prepared according to the method described in Example 2. 3 days post activation, 5×10⁶ activated T lymphocytes were transduced with lentiviruses carrying M598. T cell suspension was added into 6-well plate, and incubated overnight in a 37° C., 5% CO₂ incubator, resulting in M598-T cells. 3 days post-transduction, TCRαβ expression and CAR expression was detected using FACS. 5 days post-transduction, the cell suspension was then subject a separation and enrichment according to the TCRα/β separation kit protocols (TCRα/0-Biotin, CliniMACS, #6190221004; Anti-Biotin Reagent, CliniMACS, #6190312010), resulting in MACS sorted TCRαβ negative M598-T cells. TCRαβ expression and CAR expression of MACS sorted TCRαβ negative M598-T cells was detected using FACS. MACS sorted TCRαβ negative M598-T cells were mixed with multiple myeloma (MM) cell line RPMI8226.Luc at different E:T ratios of 2.5:1, 1.25:1, and 1:1.25, respectively, incubated in Corning® 384-well solid white plate for 18-24 hours. ONE-Glo™ Luciferase Assay System (TAKARA, #B6120) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (“UnT”) served as control.

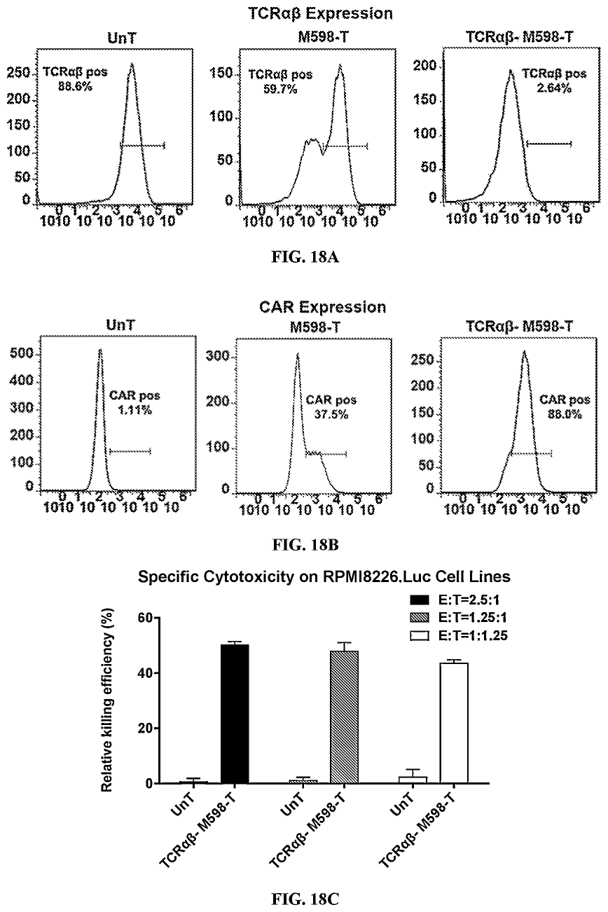

As shown in FIGS. 18A-18B, TCRαβ positive rate of M598-T cells (TCRαβ positive rate of 59.7%) was significantly lower than UnT (TCRαβ positive rate of 88.6%); CAR positive rate of M598-T cells (CAR positive rate of 37.5%) was significantly higher than UnT (CAR positive rate of 1.11%); MACS sorted TCRαβ positive M598-T cells exhibited 2.64% TCRαβ positive rate and 88.0% CAR positive rate. These results suggest M598 transduced T cells expresses CAR, meanwhile, effectively inhibits TCRαβ expression.

As shown in FIG. 18C, MACS sorted TCRαβ negative M598-T cells showed significant CAR-mediated specific killing activity on RPMI8226.Luc cell lines compared to UnT at different E:T ratios (P<0.05), with 50.32±2.56% killing efficiency.

In summary, the above results indicate that SIV Nef M708 of truncated SIV Nef combined with CAR expressing T cells, can effectively inhibit TCRαβ expression, meanwhile, does not affect CAR-mediated specific cytotoxicity activity.

### Example 14. SIV Nef Subtype with Dual Regulation on TCRαβ and MH-C Expression in CAR-T Cell Immunotherapy

### 1. Construction of SIV Nef M1275+ITAM-Modified CD20 CAR All-In-One Vector

Fusion genes SIV Nef M1275-IRES-CD8α SP-CD20 scFv (Leu16)-CD8α Hinge-CD8α TM-4-1BB-ITAM010 (hereinafter referred to as M1392, SIV Nef M1275 comprises the sequence of SEQ ID NO: 136) were then cloned into pLVX-hEF1α vector (see Example 1) for the construction of recombinant transfer plasmids pLVX-M1392. The transfer plasmids were then purified and packaged into lentiviruses as described in Example 1, hereinafter referred to as M1392 lentivirus. The encoded ITAM-modified CD20 CAR construct “CD80 SP-CD20 scFv (Leu16)-CD8u Hinge-CD8α TM-4-1BB-ITAM010” comprises the sequence of SEQ ID NO: 98, also referred to as “ITAM010-modified CD20 CAR.”

### 2. TCRαβ and MHC Class I Molecular Expression of SIV Nef M1275+ITAM-Modified CD20 CAR All-In-One Construct Transduced CAR-T Cells

PBMCs and T lymphocytes were prepared according to the method described in Example 2. 3 days post activation, 5×10⁶ activated T lymphocytes were transduced with lentiviruses M1392 (hereinafter referred to as M1392-T cells) and LCAR-UL186S (from Example 3; hereinafter referred to as LCAR-UL186S T cells), respectively. T cell suspension was added into 6-well plate, and incubated overnight in a 37° C., 5% CO₂ incubator. 3 days post-transduction, 5×10⁵ cell suspension of M1392-T and LCAR-UL186S T was separately collected and centrifuged at room temperature, the supernatant was discarded. Cells were resuspended with 1 mL DPBS and 1 μL Goat F(ab′)2 anti-Mouse IgG (Fab′)2 (FITC) (Abcam, #AB98658) was added into the suspension, then incubated at 4° C. for 30 min. After incubation, the centrifugation and resuspension with DPBS step was repeated twice. Then cells were resupended with 1 mL DPBS, then 1 μL Streptavidin (NEW ENGLAND BIOLABS, #N7021S) and 1 μL APC anti-human TCRα/β antibody (Biolegend, #306718) were added, the suspension was incubated for 30 min at 4° C. After incubation, the centrifugation and resuspension with DPBS step was repeated twice. Then cells were resuspended with DPBS for FACS to detect expression of TCRαβ and CD20 CAR. 3 days post-transduction, 5×10⁵ cell suspension of M1392-T and LCAR-UL186S T was separately collected and centrifuged at room temperature, the supernatant was discarded. Cells were resuspended with 1 mL DPBS, then 1 μL APC anti-human TCRα/β antibody (Biolegend, #306718) and 1 μL PE anti-human HLA-B7 antibody (Biolegend, #372404) were added, and the suspension was incubated for 30 min at 4° C. After incubation, the centrifugation and resuspension with DPBS step was repeated twice. Then cells were resuspended with DPBS for FACS to detect expression of TCRαβ and HLA-B7. Untransduced T cells (“UnT”) served as control.

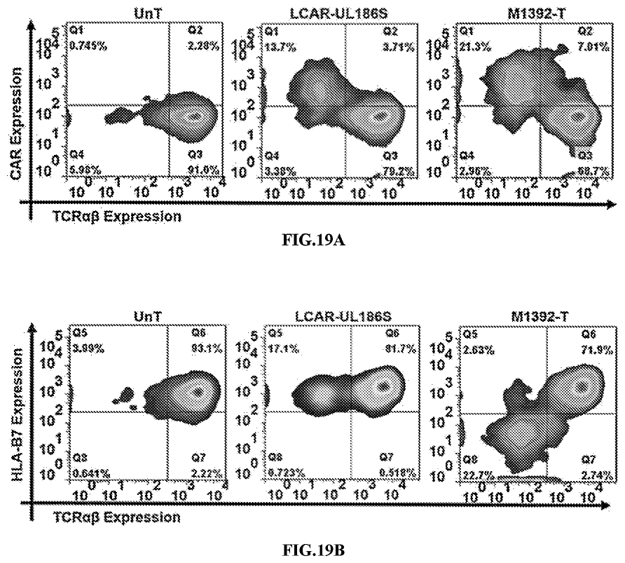

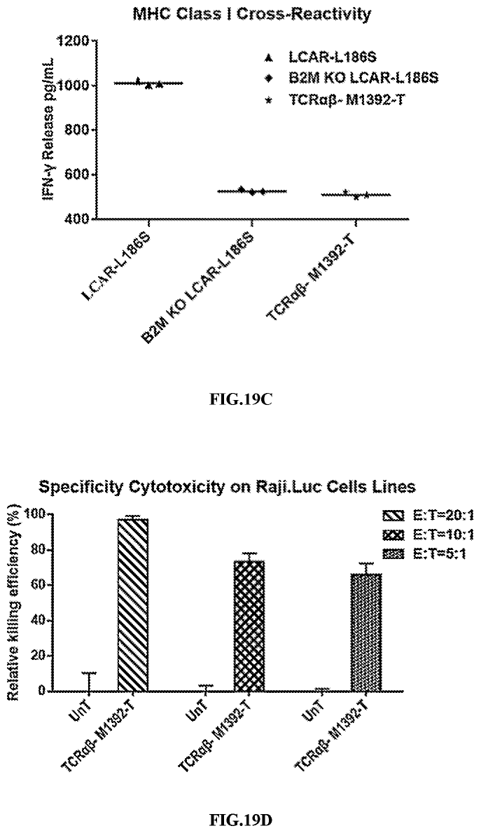

As shown in FIG. 19A, CAR positive and TCRαβ negative (CAR+/TCRαβ−) rate of UnT, LCAR-UL186S T cells, and M1392-T cells was 0.745%, 13.7%, and 21.3%, respectively. As shown in FIG. 19B, HLA-B7 negative and TCRα3 negative (HLA-B7-/TCRα3-) rate of UnT, LCAR-UL186S T cells, and M1392-T cells was 0.641%, 0.723%, and 22.7%, respectively. These results suggests that SIV Nef M1275+ITAM-modified CD20 CAR construct (M1392) transduced T cells expresses CAR, meanwhile, effectively down-regulates expression of TCRα3 and MHC class I molecule.

3. Evaluation of MHC Class I Cross-Reactivity in CAR-T Cells Transduced with SIV Nef 1275+ITAM-Modified CD20 CAR All-In-One Construct

PBMCs and T lymphocytes were prepared according to the method described in Example 2. 3 days post activation, 5×10⁶ activated T lymphocytes were transduced with lentiviruses LCAR-L186S (from Example 3 hereinafter referred to as LCAR-L186S T cells).

3 days post-transduction, 50% LCAR-L186S T cells were subject to CRISPR/Cas9 technology (SEQ ID NO: 138) and separation to construct B2M knock out (B2M KO) cells (hereinafter referred to as B2M KO LCAR-L186S T cells). The M1392-T cell suspension obtained above was then subject a separation and enrichment according to the TCRα/3 separation kit protocols (TCRα/0-Biotin, CliniMACS, #6190221004; Anti-Biotin Reagent, CliniMACS, #6190312010), resulting in MACS sorted TCRαβ negative M1392-T cells (hereinafter referred to as TCRαβ− M1392-T cells). Evaluation of MHC class I cross-reactivity of LCAR-L186S T cells, B2M KO LCAR-L186S T cells, and TCRαβ− M1392-T cells was performed with reference to Mixed Lymphocyte Reaction (MLR, see Jiangtao Ren, 2017).

As shown in FIG. 19C, 48 hours post incubation with effector cells at E:T ratio of 1:1, level of IFN-γ released by TCRαβ− M1392-T cells was significant less than LCAR-L186S (P<0.05), and similar to B2M KO LCAR-L186S T cells (P>0.05). These results suggest that M1392 (SIV Nef M1215/ITAM010-modified CD20 CAR co-expression) can significantly reduce MHC class I cross-reactivity of effector cells.

4. In Vitro Cytotoxicity Assay of CAR-T Cells Transduced with SIV Nef M1275+ITAM-Modified CD20 CAR All-In-One Construct

MACS sorted TCRαβ− M1392-T cells obtained above were mixed with lymphoma Raji.Luc cell lines at different E:T ratios of 20:1, 10:1, and 5:1, respectively, incubated in Corning® 384-well solid white plate for 12 hours. ONE-Glo™ Luciferase Assay System (TAKARA, #B6120) was used to measure luciferase activity. 25 μL ONE-Glo™ Reagent was added to each well of the 384-well plate, incubated, then placed onto Spark™ 10M multimode microplate reader (TECAN) for fluorescence measurement, in order to calculate cytotoxicity of different T lymphocytes on target cells. Untransduced T cells (“UnT”) served as control.

As shown in FIG. 19D, MACS sorted TCRαβ− M1392-T cells showed significant CAR-mediated specific killing activity on Raji.Luc cell lines compared to UnT (P<0.05).

We claim:

1. A modified T cell comprising:
a functional exogenous receptor comprising:
(a) an extracellular ligand binding domain,
(b) a transmembrane domain, and
(c) an intracellular signaling domain (“ISD”) comprising a chimeric signaling domain (“CMSD”),
wherein the CMSD comprises one or a plurality of Immune-receptor Tyrosine-based Activation Motifs (“CMSD ITAMs”), wherein the plurality of CMSD ITAMs are optionally connected by one or more linkers (“CMSD linkers”).

2. The modified T cell of claim 1, wherein:
(a) the plurality of CMSD ITAMs are directly linked to each other;
(b) the CMSD comprises two or more CMSD ITAMs connected by one or more CMSD linkers not derived from an ITAM-containing parent molecule;
(c) the CMSD comprises one or more CMSD linkers derived from an ITAM-containing parent molecule that is different from the ITAM-containing parent molecule from which one or more of the CMSD ITAMs are derived from;
(d) the CMSD comprises two or more identical CMSD ITAMs;
(e) at least one of the CMSD ITAMs is not derived from CD3ζ;
(f) at least one of the CMSD ITAMs is not ITAM1 or ITAM2 of CD3ζ;
(g) the plurality of CMSD ITAMs are each derived from a different ITAM-containing parent molecule; and/or
(h) at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin.

3. The modified T cell of claim 1, wherein at least one of the CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin.

4. The modified T cell of any one of claims 1-3, wherein the CMSD does not comprise ITAM1 and/or ITAM2 of CD3ζ.

5. The modified T cell of any one of claims 1-4, wherein the CMSD comprises ITAM3 of CD3ζ.

6. The modified T cell of any one of claims 1-5, wherein at least two of the CMSD ITAMs are derived from the same ITAM-containing parent molecule.

7. The modified T cell of claim 6, wherein the at least two of the CMSD ITAMs are identical to each other.

8. The modified T cell of any one of claims 1-6, wherein at least two of the CMSD ITAMs are different from each other.

9. The modified T cell of claim 8, wherein the two different CMSD ITAMs are each derived from a different ITAM-containing parent molecule.

10. The modified T cell of any one of claims 1-9, wherein at least one of the CMSD linkers is derived from CD3ζ.

11. The modified T cell of any one of claims 1-10, wherein at least one of the CMSD linkers is heterologous to the ITAM-containing parent molecule.

12. The modified T cell of any one of claims 1-11, wherein the CMSD further comprises a C-terminal sequence at the C-terminus of the most C-terminal CMSD ITAM (“CMSD C-terminal sequence”).

13. The modified T cell of any one of claims 1-12, wherein the CMSD further comprises an N-terminal sequence at the N-terminus of the most N-terminal CMSD ITAM (“CMSD N-terminal sequence”).

14. The modified T cell of any one of claims 1-13, wherein the one or more CMSD linkers, the CMSD C-terminal sequence, and/or the CMSD N-terminal sequence are independently selected from the group consisting of SEQ ID NOs: 17-39 and 116-120.

15. The modified T cell of any one of claims 1-14, wherein the functional exogenous receptor is an ITAM-modified T cell receptor (TCR), an ITAM-modified chimeric antigen receptor (CAR), an ITAM-modified chimeric TCR (cTCR), or an ITAM-modified T cell antigen coupler (TAC)-like chimeric receptor.

16. The modified T cell of claim 15, wherein the functional exogenous receptor is an ITAM-modified CAR.

17. The modified T cell of claim 16, wherein the transmembrane domain is derived from CD8α.

18. The modified T cell of claim 16 or 17, wherein the ISD further comprises a co-stimulatory signaling domain.

19. The modified T cell of claim 18, wherein the co-stimulatory signaling domain is derived from CD137 (4-1BB) or CD28.

20. The modified T cell of claim 18 or 19, wherein the co-stimulatory signaling domain comprises the amino acid sequence of SEQ ID NO: 124.

21. The modified T cell of any one of claims 18-20, wherein the co-stimulatory domain is N-terminal to the CMSD.

22. The modified T cell of any one of claims 18-20, wherein the co-stimulatory domain is C-terminal to the CMSD.

23. The modified T cell of claim 15, wherein the functional exogenous receptor is an ITAM-modified cTCR.

24. The modified T cell of claim 23, wherein the ITAM-modified cTCR comprises:
(a) an extracellular ligand binding domain,
(b) an optional receptor domain linker,
(c) an optional extracellular domain of a first TCR subunit or a portion thereof,
(d) a transmembrane domain comprising a transmembrane domain of a second TCR subunit, and
(e) an ISD comprising the CMSD,
wherein the first and second TCR subunits are selected from the group consisting of TCRα, TCRβ, TCRγ, TCRδ, CD3ε, CD3γ, and CD3δ.

25. The modified T cell of claim 24, wherein the first and second TCR subunits are both CD3ε.

26. The modified T cell of claim 24 or 25, wherein the one or plurality of CMSD ITAMs are derived from one or more of CD3ε, CD3δ, and CD3γ.

27. The modified T cell of claim 15, wherein the functional exogenous receptor is an ITAM-modified TAC-like chimeric receptor.

28. The modified T cell of claim 27, wherein the ITAM-modified TAC-like chimeric receptor comprises:
(a) an extracellular ligand binding domain,
(b) an optional first receptor domain linker,
(c) an extracellular TCR binding domain that specifically recognizes the extracellular domain of a first TCR subunit,
(d) an optional second receptor domain linker,
(e) an optional extracellular domain of a second TCR subunit or a portion thereof,
(f) a transmembrane domain comprising a transmembrane domain of a third TCR subunit, and
(g) an ISD comprising the CMSD,
wherein the first, second, and third TCR subunits are all selected from the group consisting of TCRα, TCRβ, TCRγ, TCRδ, CD3ε, CD3γ, and CD3δ.

29. The modified T cell of claim 28, wherein the second and third TCR subunits are both CD3ε.

30. The modified T cell of claim 28 or 29, wherein the one or plurality of CMSD ITAMs are derived from one or more of CD3ε, CD3δ, and CD3γ.

31. The modified T cell of any one of claims 1-30, wherein the extracellular ligand binding domain comprises one or more antigen-binding fragments that specifically recognizing one or more epitopes of one or more target antigens.

32. The modified T cell of claim 31, wherein the antigen-binding fragment is an sdAb or an scFv.

33. The modified T cell of claim 31 or 32, wherein the target antigen is BCMA, CD19, or CD20.

34. The modified T cell of any one of claims 1-33, further comprising a hinge domain located between the C-terminus of the extracellular ligand binding domain and the N-terminus of the transmembrane domain.

35. The modified T cell of claim 34, wherein the hinge domain is derived from CD8α.

36. The modified T cell of any one of claims 1-35, wherein the effector function of the functional exogenous receptor comprising the ISD that comprises the CMSD is at most about 80% less than a functional exogenous receptor comprising an ISD that comprises an intracellular signaling domain of CD3ζ.

37. A method of producing a modified T cell, comprising introducing into a precursor T cell a nucleic acid encoding a functional exogenous receptor,
wherein the functional exogenous receptor comprises:
(a) an extracellular ligand binding domain,
(b) a transmembrane domain, and
(c) an ISD comprising a CMSD,
wherein the CMSD comprises one or a plurality of CMSD ITAMs, wherein the plurality of CMSD ITAMs are optionally connected by one or more CMSD linkers.

38. The method of claim 37, wherein the nucleic acid is on a viral vector.

39. The method of claim 37 or 38, further comprising isolating and/or enriching functional exogenous receptor-positive T cells from the modified T cells.

40. The method of any one of claims 37-39, further comprising formulating the modified T cell with at least one pharmaceutically acceptable carrier.

41. The method of any one of claims 37-40, wherein:
(a) the plurality of CMSD ITAMs are directly linked to each other;
(b) the CMSD comprises two or more CMSD ITAMs connected by one or more CMSD linkers not derived from an ITAM-containing parent molecule;
(c) the CMSD comprises one or more CMSD linkers derived from an ITAM-containing parent molecule that is different from the ITAM-containing parent molecule from which one or more of the CMSD ITAMs are derived from;
(d) the CMSD comprises two or more identical CMSD ITAMs;
(e) at least one of the CMSD ITAMs is not derived from CD3ζ;
(f) at least one of the CMSD ITAMs is not ITAM1 or ITAM2 of CD3ζ;
(g) the plurality of CMSD ITAMs are each derived from a different ITAM-containing parent molecule; and/or
(h) at least one of the CMSD ITAMs is derived from an ITAM-containing parent molecule selected from the group consisting of CD3ε, CD3δ, CD3γ, Igα (CD79a), Igβ (CD79b), FcεRIβ, FcεRIγ, DAP12, CNAIP/NFAM1, STAM-1, STAM-2, and Moesin.

42. The method of any one of claims 37-41, wherein at least one of the CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin.

43. A modified T cell obtained by the method of any one of claims 37-42.

44. A pharmaceutical composition comprising the modified T cell of any one of claims 1-36 and 43, and a pharmaceutically acceptable carrier.

45. A method of treating a disease in an individual, comprising administering to the individual an effective amount of the modified T cell of any one of claims 1-36 and 43, or the pharmaceutical composition of claim 44.

46. The method of claim 45, wherein the disease is cancer.

47. The method of claim 45 or 46, wherein the individual is histoincompatible with the donor of the precursor T cell from which the modified T cell is derived.

48. The method of any one of claims 45-47, wherein the individual is a human.

49. An isolated nucleic acid encoding a functional exogenous receptor,
wherein the functional exogenous receptor comprises:
(a) an extracellular ligand binding domain,
(b) a transmembrane domain, and
(c) an ISD comprising a CMSD,
wherein the CMSD comprises one or a plurality of CMSD ITAMs, wherein the plurality of CMSD ITAMs are optionally connected by one or more CMSD linkers.

50. The isolated nucleic acid of claim 49, wherein at least one of the CMSD ITAMs is derived from an ITAM-containing parent molecule selected from the group consisting of CD3ε, CD3δ, CD3γ, CD3ζ, Igα (CD79a), Igβ (CD79b), FcεRIβ, FcεRIγ, DAP12, CNAIP/NFAM1, STAM-1, STAM-2, and Moesin.

51. A vector comprising the nucleic acid of claim 49 or 50.

52. The vector of claim 51, which is a viral vector.
